(19) United States
(12) Patent Application Publication
(10) Pub. No.: US 2008/0132505 A1
(43) Pub. Date: Jun. 5, 2008

(54) Combination Of Cb2 Modulators And Pde4 Inhibitors For Use In Medicine

(75) Inventors: Andrew James Brown, Essex (GB); Helen Elizabeht Connor, Hertfordshire (GB); Andrew John Eatherton, Essex (GB); Gerard Martin Paul Giblin, Essex (GB); Richard Howard Green, Hertfordshire (GB); Jennifer Margaret Doughty, Gorham, ME (US); Karamjit Singh Jandu, Essex (GB); Richard Graham Knowles, Hertfordshire (GB); William Leonard Mitchell, Essex (GB); Alan Naylor, Essex (GB); Celestine Theresa O'Shaughnessy, Essex (GB); Giovanni Palombi, Milan (IT); Derek Anthony Rawlings, Essex (GB); Brian Peter Slingsby, Essex (GB); Catherine Jane Tralau-Stewart, Hertfordshire (GB); Andrew Richard Whittington, Hertfordshire (GB); Richard Alexander Williamson, Hertfordshire (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Glaxo Group Limited

(21) Appl. No.: 10/597,527

(22) PCT Filed: Feb. 1, 2005
(86) PCT No.: PCT/GB05/00348

§ 371 Date: Nov. 2, 2006

(30) Foreign Application Priority Data
Feb. 3, 2004 (GB) 0402355.2

(51) Int. Cl. A61K 31/505; A61K 31/5377; A61K 31/4406; A61K 31/44; A61P 37/00; A61P 29/00; A61P 25/00; A61P 11/00

(52) U.S. Cl. 514/235.8; 514/275; 514/355; 514/352

(57) ABSTRACT

Combination of one or more CB2 modulators and one or more PDE4 inhibitors, and method of treating conditions which are mediated by the activity of CB2 receptors or conditions which are mediated by PDE4.

The present invention relates to combinations of cannabinoid 2 modulators with PDE4 inhibitors, pharmaceutical compositions comprising these combinations and their use in the treatment of diseases, particularly pain.

Cannabinoids are a specific class of psychoactive compounds present in Indian cannabis (Cannabis sativa), including about sixty different molecules, the most representative being cannabinol, cannabidiol and several isomers of tetrahydrocannabinol. Knowledge of the therapeutic activity of cannabis dates back to the ancient dynasties of China, where, 5,000 years ago, cannabis was used for the treatment of asthma, migraine and some gynaecological disorders. These uses later became so established that, around 1850, cannabis extracts were included in the US Pharmacopaeia and remained there until 1947.

Cannabinoids are known to cause different effects on various systems and/or organs, the most important being on the central nervous system and on the cardiovascular system. These effects include alterations in memory and cognition, euphoria, and sedation. Cannabinoids also increase heart rate and vary systemic arterial pressure. Peripheral effects related to bronchial constriction, immunomodulation, and inflammation have also been observed. The capability of cannabinoids to reduce intraocular pressure and to affect respiratory and endocrine systems is also well documented. See e.g. L. E. Hollister, Health Aspects of Cannabis, Pharmacological Reviews, Vol. 38, pp. 1-20, (1986). More recently, it was found that cannabinoids suppress the cellular and humoral immune responses and exhibit anti-inflammatory properties. Wirth et al., Anti-inflammatory Properties of Cannabichrome, Life Science, Vol. 26, pp. 1991-1995, (1980).

In spite of the foregoing benefits, the therapeutic use of cannabis is controversial, both due to its relevant psychoactive effects (causing dependence and addiction), and due to manifold side effects that have not yet been completely clarified. Although work in this field has been ongoing since the 1940's, evidence indicating that the peripheral effects of cannabinoids are directly mediated, and not secondary to a CNS effect, has been limited by the lack of receptor characterisation, the lack of information concerning an endogenous cannabinoid ligand and, until recently, the lack of receptor subtype selective compounds.

The first cannabinoid receptor was found to be mainly located in the brain, in neural cell lines, and, only to a lesser extent, at the peripheral level. In view of its location, it was called the central receptor (“CB1”). See Matsuda et al., “Structure of a Cannabinoid Receptor and Functional Expression of the Cloned cDNA,” Nature Vol. 346, pp. 561-564 (1990. The second cannabinoid receptor (“CB2”) was identified in the spleen, and was assumed to modulate the non psychoactive effects of the cannabinoids. See Munro et el., “Molecular Characterization of a Peripheral Receptor for Cannabinoids,” Nature, Vol. 365, pp. 61-65 (1993).

Recently, some compounds have been prepared which are capable of acting as agonists on both the cannabinoid receptors. For example, use of derivatives of dihydroxypyrrole-(1,2,3-d,e)-1,4-benzoxazine in the treatment of glaucoma and the use of derivatives of 1,5-diphenyl-pyrazole as immunomodulators or psychotropic agents in the treatment of various neuropathologies, migraine, epilepsy, glaucoma, etc are known. See U.S. Pat. No. 5,112,820 and EP 576357, respectively. However, because these compounds are active on both the CB1 and CB2 receptor, they can lead to serious psychoactive effects.

The foregoing indications and the preferential localisation of the CB2 receptor in the immune system confirms a specific role of CB2 in modulating the immune and anti-inflammatory response to stimuli of different sources.

The total size of the patient population suffering from pain is vast (almost 300 million), dominated by those suffering from back pain, osteo-arthritic pain and post-operative pain. Neuropathic pain (associated with neuronal lesions such as those induced by diabetes, HIV, herpes infection, or stroke) occurs with lower, but still substantial prevalence, as does cancer pain.

The pathogenic mechanisms that give rise to pain symptoms can be grouped into two main categories:
those that are components of inflammatory tissue responses (Inflammatory Pain);
those that result from a neuronal lesion of some form (Neuropathic Pain).

Chronic inflammatory pain consists predominantly of osteo-arthritis, chronic low back pain and rheumatoid arthritis. The pain results from acute and on-going injury and/or inflammation. There may be both spontaneous and provoked pain.

There is an underlying pathological hypersensitivity as a result of physiological hyperexcitability and the release of inflammatory mediators which further potentiate this hyperexcitability. CB2 receptors are expressed on inflammatory cells (T cells, B cells, macrophages, mast cells) and mediate immune suppression through inhibition of cellular interaction/inflammatory mediator release. CB2 receptors may also be expressed on sensory nerve terminals and therefore directly inhibit hyperalgesia.

The role of CB2 in immunomodulation, inflammation, osteoporosis, cardiovascular, renal and other disease conditions is now being examined. In light of the fact that cannabinoids act on receptors capable of modulating different functional effects, and in view of the low homology between CB2 and CB1, the importance of developing a class of drugs selective for the specific receptor sub-type is evident. The natural or synthetic cannabinoids currently available do not fulfil this function because they are active on both receptors.

Based on the foregoing, compounds which are capable of selectively modulating the receptor for cannabinoids offer a unique approach toward the pharmacotherapy of immune disorders, inflammation, osteoporosis, renal ischemia and other pathophysiological conditions.

Enzymes known as phosphodiesterases (PDEs) function in vivo to hydrolytically cleave the 3′-phosphodiester bond of cyclic nucleotides to thereby form the corresponding 5′-monophosphate. For instance, certain PDEs can hydroylze the 3′-phosphodiester bond of adenosine 3′,5′-cyclic monophosphate (cAMP) so as to form 5′-adenosine monophosphate (5′-AMP), and/or can hydrolyze the 3′-phosphodiester bond of guanosine 3′,5′-cyclic monophosphate (cGMP) so as to form 5′-guanosine monophosphate (5′-GMP). These cyclic nucleotides exert a significant impact on cellular processes by, for example, converting inactive protein kinase enzymes into an active form. The active form of the protein kinase catalyzes various phosphorylation processes that impact on fundamental cellular processes including transcriptional regulation, ion channel function, and signaling protein activity.

Researchers investigating PDEs generally agree that there are at least eleven distinct PDE families, differentiated on the basis of amino acid sequence, substrate specificity and sensitivity to endogenous and exogenous regulators. These families are commonly known as PDE1 through PDE11. In addition, researchers found that cyclic nucleotide concentration is a significant factor in the course of the in vivo inflammatory response. Accordingly, much research has been directed to methods for influencing the concentration of cyclic nucleotides as a means to influence the inflammatory response, and particular attention has been directed at PDE4 activity. One promising area of research is the development of small organic molecules that inhibit PDE activity. By inhibiting PDE activity, these small molecules reduce the amount of cyclic nucleotide that is converted into the (inactive) corresponding 5′-monophosphate, thereby elevating cyclic nucleotide concentration, and indirectly increasing protein kinase activity within the cell.

ROLIPRAM® (Schering AG) is an example of an early attempt to develop such a composition directed to PDE4. However, while ROLIPRAM® exhibited marked anti-inflammatory activity, it was also found to demonstrate unwanted side effects including emesis (also known as nausea and vomiting) and potentiation of gastric acid secretion. These undesired side effects caused ROLIPRAM® to be withdrawn from development as an anti-inflammatory pharmaceutical.

The present invention provides combinations of cannabioid 2 modulators and pharmaceutically acceptable derivatives thereof and PDE4 inhibitors and pharmaceutically acceptable derivatives thereof, pharmaceutical compositions containing these combinations and their use.

In one aspect the present invention provides a method of treating a human or animal subject suffering from a condition which is mediated by the activity of CB2 receptors or a condition which is mediated by PDE4 which comprises administering to said subject a therapeutically effective combination of one or more CB2 modulators or a pharmacetical acceptable derivative thereof and one or more PDE4 inhibitors or a pharmacetical acceptable derivative thereof.

In another aspect the present invention provides the use of a combination of one or more CB2 modulators or a pharmacetical acceptable derivative thereof and one or more PDE4 inhibitors or a pharmacetical acceptable derivative thereof in the treatment of a disease mediated by CB2 receptors or PDE4.

In another aspect the present invention provides the use of a combination of one or more CB2 modulators and one or more PDE4 inhibitors in the manufacture of a medicament for treating a disease mediated by CB2 receptors or PDE4.

Suitable cannbinoid 2 modulators are described in co-pending International Patent Applications WO 04/018433, WO 04/018434, WO 04/029027 and WO 04/029026. These compounds are referred to herein as compounds of formula (I), (II), (III), and (IV) respectively.

### Compounds of Formula (I)

In compounds of formula (I):

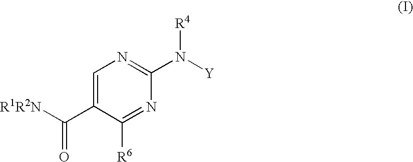

Y is phenyl, optionally substituted with one, two or three substituents;

R¹ is selected from hydrogen, C₁₋₆ alkyl, C₃₋₆ cycloalkyl and halosubstitutedC₁— alkyl;

R² is (CH₂)ₘR³ where m is 0 or 1;

or R¹ and R² together with N to which they are attached form an optionally substituted 4- to 8-membered non-aromatic heterocyclyl ring;

R³ is an optionally substituted 4- to 8-membered non-aromatic heterocyclyl group, an optionally substituted C₃₋₈ cycloalkyl group, an optionally substituted straight or branched C₁₋₁₀ alkyl, an optionally substituted C₅₋₇ cycloalkenyl or R⁵;

R⁴ is selected from hydrogen, C₁₋₆ alkyl, C₃₋₆ cycloalkyl, or halosubstitutedC₁— alkyl, COCH₃, and SO₂Me;

R⁵ is

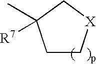

wherein p is 0, 1 or 2 and X is CH₂ or O;

R⁶ is methyl, chloro or CHxFn wherein n is 1, 2, or 3, x is 0, 1 or 2 and n and x add up to 3;

R⁷ is OH, C₁₋₆alkoxy, NR^{8a}R^{8b}, NHCOR⁹, NHSO₂R⁹, SOqR⁹;

R^{8a} is H or C₁₋₆alkyl;

R^{8b} is H or C₁₋₆alkyl;

R⁹ is C₁₋₆alkyl; and

q is 0, 1 or 2.

In one particular embodiment Y is a substituted phenyl.

In one particular embodiment Y is substituted by 1 or 2 substituents. If mono-substituted, in one particular embodiment the substituent is in the 3 position. If di-substituted, in one particular embodiment the substituents are in the 2- and 4-positions.

When Y is substituted, the substituent or substituents are preferably selected from C₁₋₆ alkyl, halosubstitutedC₁₋₆ alkyl, C₁₋₆ alkoxy, a hydroxy group, a cyano group, halo, a C₁₋₆alkylsulfonyl group, —CONH₂, —NHCOCH₃, —COOH, halosubstitutedC₁₋₆ alkoxy, SC₁₋₆alkyl or SO₂NR^{8a}R^{8b} wherein R^{8a} and R^{8b} are as defined above.

In one particular embodiment Y is substituted by chloro, fluoro, bromo, cyano, CF₃, methyl, CF₃O— or SCH₃ and methoxy; more particularly halo, cyano or methoxy.

In one particular embodiment the compound of formula (I) is a compound of formula (Ia)

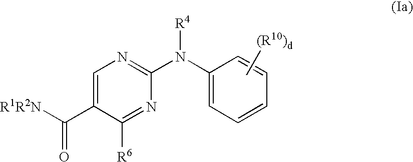

wherein;

R¹ is selected from hydrogen, C₁₋₆ alkyl, C₃₋₆ cycloalkyl and halosubstitutedC₁₋₆ alkyl;

R² is (CH₂)ₘR³ where m is 0 or 1;

or R¹ and R² together with N to which they are attached form a 4- to 8-membered non-aromatic ring selected from azetidinyl, pyrrolidinyl, morpholinyl, piperizinyl, piperidinyl, tetrahydropyridinyl, azapine, oxapine, azacyclooctanyl, azaoxacyclooctanyl and azathiacyclooctanyl any of which can be unsubstituted or substituted by one, two or three substituents selected from C₁₋₆ alkyl, C₁₋₆ alkoxy, a hydroxy group, a cyano group, halo, sulfonyl group, methylsulfonyl, NR^{8a}R^{8b}, NHCOCH₃, (═O), and —CONHCH₃;

R³ is 2- or 3-azetidinyl, oxetanyl, thioxetanyl, thioxetanyl-s-oxide, thioxetanyl-s,s-dioxide, dioxalanyl, pyrrolidinyl, tetrahydrofuranyl, tetrahydrothiophenyl, morpholinyl, piperidinyl, piperazinyl, tetrahydropyranyl, tetrahydrothiopyranyl, thiomorpholinyl, thiomorpholinyl-s,s-dioxide, tetrahydropyridinyl, azapine, oxapine, azacyclooctanyl, azaoxacyclooctanyl, azathiacyclooctanyl, oxacylcooctanyl, thiacyclooctanyl, a C₃₋₈ cycloalkyl group, a straight or branched C₁₋₁₀ alkyl, a C₅₋₇ cycloalkenyl or R⁵, any of which can be unsubstituted or substituted by one, two or three substituents selected from C₁₋₆ alkyl, C₁₋₆ alkoxy, a hydroxy group, a cyano group, halo, sulfonyl group, methylsulfonyl, NR^{8a}R^{8b}, NHCOCH₃, (═O), and —CONHCH₃;

R¹⁰ is selected from C₁₋₆ alkyl, halosubstitutedC₁₋₆ alkyl, C₁₋₆ alkoxy, a hydroxy group, a cyano group, halo, a C₁₋₆alkyl sulfonyl group, —CONH₂, —NHCOCH₃, —COOH, halosubstitutedC₁₋₆ alkoxy, SC₁₋₆alkyl and SO₂NR^{8a}R^{8b};

R⁴ is selected from hydrogen, C₁₋₆ alkyl, C₃₋₆ cycloalkyl, or halosubstitutedC₁₋₆ alkyl, COCH₃ and SO₂Me;

R⁵ is

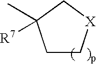

wherein p is 0, 1 or 2 and X is CH₂ or O;

R⁶ is methyl, chloro or CHxFn wherein n is 1, 2, or 3, x is 0, 1 or 2 and n and x add up to 3;

R⁷ is OH, C₁₋₆alkoxy, NR^{8a}R^{8b}, NHCOR⁹, NHSO₂R⁹, SOqR⁹;

R^{8a} is H or C₁₋₆alkyl;

R^{8b} is H or C₁₋₆alkyl;

R⁹ is C₁₋₆alkyl;

q is 0, 1 or 2; and

d is 0, 1, 2 or 3.

In one particular embodiment R¹ is hydrogen.

In one particular embodiment R⁴ is C₁₋₆alkyl or hydrogen, more preferably methyl or hydrogen even more preferably hydrogen.

Alternatively R¹ and R² together with N to which they are attached form an optionally substituted 5- or 6-membered non-aromatic heterocyclyl ring.

When R¹ and R² together with N to which they are attached form a 4- to 8-membered non-aromatic heterocyclyl ring which is substituted, or when R³ is substituted, the substituent or substituents are preferably selected from: C₁₋₆ alkyl, C₁₋₆ alkoxy, a hydroxy group, a cyano group, halo, a sulfonyl group, methylsulfonyl, NR^{8a}R^{8b}, NHCOCH₃, (═O), or —CONHCH₃.

In one particular embodiment R⁶ is CHxFn, for example CF₃, CHF₂, CH₂F, more preferably CF₃.

In one particular embodiment R⁵ is

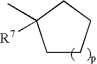

wherein p is 0, 1 or 2;

In one particular embodiment R⁷ is OH.

In one particular embodiment R³ is an optionally substituted 4- to 8-membered non-aromatic heterocyclyl group, an optionally substituted C₃₋₈ cycloalkyl group, an optionally substituted straight or branched C₁₋₁₀ alkyl or R⁵.

In one particular embodiment when R³ is an optionally substituted C₃₋₈cycloalkyl group or an optionally substituted 4- to 8-membered nonaromatic heterocyclyl, m is 1.

In one particular embodiment R³ is an optionally substituted C₃₋₆cycloalkyl group or an optionally substituted 4- or 6-membered nonaromatic heterocyclyl.

In one particular embodiment R¹ and R² together with N to which they are attached form a 4- to 8-membered non-aromatic heterocyclyl ring which is selected from pyrrolidinyl, morpholinyl, piperizinyl, piperidinyl and tetrahydropyridinyl.

In one particular embodiment when R³ is nonaromatic heterocyclyl it is selected from pyrrolidinyl, tetrahydrofuranyl, tetrahydrothiophenyl, morpholinyl, piperidinyl, piperazinyl, tetrahydropyranyl, tetrahydrothiopyranyl, thiomorpholinyl, thiomorpholinyl-s,s-dioxide, tetrahydropyridinyl.

In one particular embodiment the compound of formula (I) is a compound of formula (Ib)

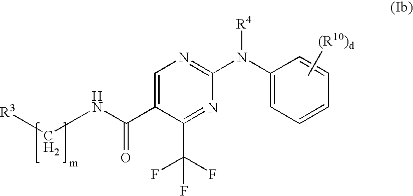

wherein;

R³ is pyrrolidinyl, tetrahydrofuranyl, tetrahydrothiophenyl, morpholinyl, piperidinyl, piperazinyl, tetrahydropyranyl, tetrahydrothiopyranyl, thiomorpholinyl, thiomorpholinyl-s,s-dioxide, tetrahydropyridinyl, a C₃₋₈ cycloalkyl group, any of which can be unsubstituted or substituted by one, two or three substituents selected from C₁₋₆ alkyl, C₁₋₆ alkoxy, a hydroxy group, a cyano group, halo, sulfonyl group, methylsulfonyl, NR^{8a}R^{8b}, NHCOCH₃, (═O), and —CONHCH₃;

R¹⁰ is selected from chloro, fluoro, bromo, cyano, CF₃, methyl, CF₃O— or SCH₃ and methoxy;

R⁴ is selected from hydrogen or methyl;

R^{8a} is H or C₁₋₆alkyl;

R^{8b} is H or C₁₋₆alkyl;

m is 0 or 1 and

d is 0, 1, 2 or 3.

In one particular embodiment m is 1.

In one particular embodiment the compound of formula (I) is a compound of formula (Ic)

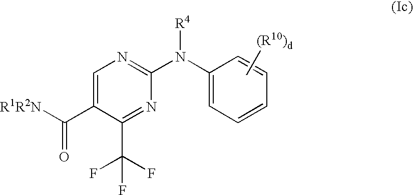

wherein;

R¹ and R² together with N to which they are attached form a 5- to 6-membered non-aromatic ring selected from pyrrolidinyl, morpholinyl, piperizinyl, piperidinyl and tetrahydropyridinyl, any of which can be unsubstituted or substituted by one, two or three substituents selected from C₁₋₆ alkyl, C₁₋₆ alkoxy, a hydroxy group, a cyano group, halo, sulfonyl group, methylsulfonyl, NR^{8a}R^{8b}, NHCOCH₃, (═O), and —CONHCH₃;

R¹⁰ is selected from chloro, fluoro, bromo, cyano, CF₃, methyl, CF₃O— or SCH₃ and methoxy;

R⁴ is hydrogen or methyl;

R^{8a} is H or C₁₋₆alkyl;

R^{8b} is H or C₁₋₆alkyl; and

d is 0, 1, 2 or 3.

Compounds of formula (II)

In compounds of formula (II):

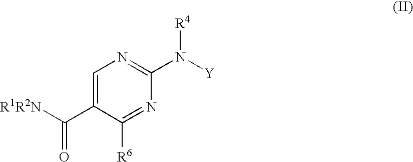

Y is phenyl, substituted with one, two or three substituents;

R¹ is selected from hydrogen, C₁₋₆ alkyl, C₃₋₈ cycloalkyl, and halosubstitutedC₁₋₆ alkyl;

R² is C(R⁷)₂R³;

R³ is an optionally substituted 5- to 6-membered aromatic heterocyclyl group, or group A:

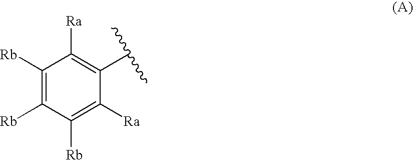

R⁴ is selected from hydrogen, C₁₋₆ alkyl, C₃₋₇ cycloalkyl, and halosubstitutedC₁₋₆ alkyl, COCH₃, or SO₂Me;

R⁶ is methyl, chloro or CHxFn wherein n is 1, 2, or 3, x is 0, 1 or 2 and n and x add up to 3;

Ra can be independently selected from hydrogen, fluoro, chloro or trifluoromethyl;

Rb can be independently be selected from hydrogen, C₁₋₆ alkyl, C₁₋₆ alkoxy, haloC₁₋₆ alkoxy, hydroxy, cyano, halo, sulfonyl, CONH₂, COOH or NHCOOC₁₋₆alkyl; and

R⁷ can be independently hydrogen or C₁₋₆ alkyl, with the proviso that the compound is not 2-(4-tert-butyl-phenylamino)-4-trifluoromethyl-pyrimidine-5-carboxylic acid benzylamide;
2-(4-tert-butyl-phenylamino)-4-trifluoromethyl-pyrimidine-5-carboxylic acid benzyl-methyl-amide;
2-(3-Chloro-phenylamino)-4-trifluoromethyl-pyrimidine-5-carboxylic acid 2-methoxy-benzylamide; or
2-(3-Chloro-phenylamino)-4-trifluoromethyl-pyrimidine-5-carboxylic acid 2-bromo-benzylamide.

In one particular embodiment Y is substituted by 1 or 2 substituents. If mono-substituted, in one particular embodiment the substituents is in the 3 position; if disubstituted, in one particular embodiment, the substituents are in the 2,4-positions.

Substituents for Y are selected from: C₁₋₆ alkyl, halosubstitutedC₁₋₆ alkyl, C₁₋₆ alkoxy, hydroxy group, cyano group, halo, C₁₋₆alkyl sulfonyl group, COOH, halosubstituted C₁₋₆ alkoxy, CONH₂, —NHCOC₁₋₆alkyl, CH₂COOH, SO₂NR^{8a}R^{8b} wherein R^{8a} and R^{8b} are independently selected from H or C₁₋₆alkyl as defined above.

In one particular embodiment Y is substituted by halo, cyano or methoxy.

In one particular embodiment R¹ is hydrogen or C₁₋₆alkyl, more preferably hydrogen.

In one particular embodiment R⁴ is C₁₋₆ alkyl or hydrogen, more preferably methyl or hydrogen, even more preferably hydrogen.

In one particular embodiment R² is CH₂R³.

In one particular embodiment R³ is group A, pyridinyl, or pyrimidinyl, any of which can be optionally substituted.

When R³ is a substituted 5- to 6-membered aromatic heterocyclyl group, the substituent or substituents is/are preferably selected from: C₁₋₆ alkyl, C₁₋₆ alkoxy, haloC₁₋₆ alkoxy, hydroxy, cyano, halo, sulfonyl, CONH₂, COOH, NCOCH₃, (═O), CONHCH₃, methylsulfonyl, NR^{8a}R^{8b} wherein R^{8a} and R^{8b} are independently selected from H or C₁₋₆alkyl,

In one embodiment the halo is fluoro.

In one particular embodiment substituents when R³ is an 5- to 6-membered aromatic heterocyclyl group are halo, methoxy, and cyano.

In one particular embodiment Rb is selected from hydrogen, halo, methoxy, and cyano.

In one particular embodiment R⁶ is CHxFn, more preferably CF₃.

We have found that at least in the CB2 assay described herein the following compounds are inactive;
2-(4-tert-butyl-phenylamino)-4-trifluoromethyl-pyrimidine-5-carboxylic acid benzylamide;
2-(4-tert-butyl-phenylamino)-4-trifluoromethyl-pyrimidine-5-carboxylic acid benzyl-methyl-amide;
2-(3-Chloro-phenylamino)-4-trifluoromethyl-pyrimidine-5-carboxylic acid 2-methoxy-benzylamide; and
2-(3-Chloro-phenylamino)-4-trifluoromethyl-pyrimidine-5-carboxylic acid 2-bromo-benzylamide.

### Compounds of Formula (III)

In compounds of formula (III):

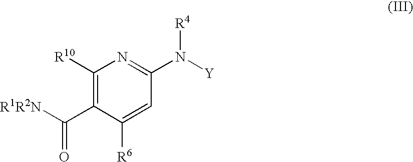

Y is phenyl, substituted with one, two or three substituents;

R¹ is selected from hydrogen, C₁₋₆ alkyl, C₃₋₇ cycloalkyl, or halosubstitutedC₁₋₆ alkyl;

R² is (CH₂)mR³;

R³ is an unsubstituted or substituted 5- to 6-membered aromatic heterocyclyl group, or group A:

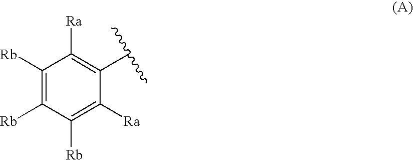

R⁴ is selected from hydrogen, C₁— alkyl, C₃₋₇ cycloalkyl, or halosubstitutedC₁₋₆ alkyl, COCH₃, and SO₂Me;

R⁶ is unsubstituted or substituted (C₁₋₆)alkyl or chloro and R¹⁰ is hydrogen or R¹⁰ is unsubstituted or substituted (C₁₋₆)alkyl or chloro and R⁶ is hydrogen;

Ra can be independently selected from hydrogen, fluoro, chloro or trifluoromethyl;

Rb can independently be selected from hydrogen, C₁₋₆ alkyl, C₁₋₆ alkoxy, halo substituted C₁₋₆ alkoxy, hydroxy, cyano, halo, sulfonyl, CONH₂, COOH, SO₂CH₃, NHCOCH₃, NHSO₂CH₃ and CONHCH₃; and

m is 1 or 2.

In one particular embodiment Y is substituted by 1 or 2 substituents. If mono-substituted, in one particular embodiment, the substituent is in the 3 position.

Substituents for Y are selected from: C₁₋₆ alkyl, halosubstitutedC₁— alkyl, C₁₋₆ alkoxy, hydroxy, cyano, halo, C₁₋₆alkylsulfonyl, COOH, halosubstitutedC₁₋₆ alkoxy, CONH₂, NHCOCH₃, C₁₋₆alkynyl, C₁₋₆alkyenyl SO₂NR^{8a}R^{8b} wherein R⁸¹ and R^{8b} are independently selected from H and C₁₋₆alkyl.

In one particular embodiment Y is substituted by halo, cyano, methoxy, methyl, trifluoromethyl or trifluoromethoxy.

In one particular embodiment R² is CH₂R³.

In one particular embodiment the compound of formula (III) is a compound of formula (IIIa):

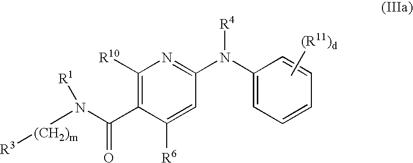

wherein:

R¹ is selected from hydrogen, C₁₋₆ alkyl, C₃₋₇ cycloalkyl, or halosubstitutedC₁₋₆ alkyl;

R³ is furanyl, dioxalanyl, pyrrolyl, oxazolyl, thiazolyl, imidazolyl, oxadiazolyl, thiadiazolyl, triazolyl, triazinyl, isothiazolyl, isoxazolyl, thienyl, pyrazolyl, tetrazolyl, pyridyl, pyrizinyl, pyrimidinyl, pyrazinyl, triazinyl, or tetrazinyl which can be unsubstituted or substituted with 1, 2 or 3 substitutents selected from C₁₋₆ alkyl, C₁₋₆ alkoxy, halosubstitutedC₁₋₆ alkoxy, halosubstitutedC₁₋₆ alkyl, hydroxy, cyano, halo, sulfonyl, CONH₂ and COOH, or R³ is group A:

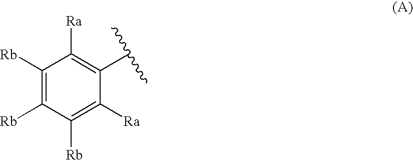

R⁴ is selected from hydrogen, C₁₋₆ alkyl, C₃₋₇ cycloalkyl, or halosubstitutedC₁₋₆ alkyl, COCH₃, and SO₂Me;

R⁶ is unsubstituted or substituted (C₁₋₆)alkyl, chloro and R¹⁰ is hydrogen or R¹⁰ is unsubstituted or substituted (C₁₋₆)alkyl or chloro and R⁶ is hydrogen;

Ra can be independently selected from hydrogen, fluoro, chloro or trifluoromethyl;

Rb can independently be selected from hydrogen, C₁₋₆ alkyl, C₁₋₆ alkoxy, halosubstitutedC₁₋₆ alkoxy, hydroxy, cyano, halo, sulfonyl, CONH₂, COOH, SO₂CH₃, NHCOCH₃, NHSO₂CH₃ and CONHCH₃;

R¹¹ is C₁₋₆ alkyl, halosubstitutedC₁₋₆ alkyl, C₁₋₆ alkoxy, hydroxy, cyano, halo, C₁₋₆alkylsulfonyl, CONH₂, NHCOCH₃, COOH, halosubstitutedC₁₋₆ alkoxy, C₁₋₆alkynyl, C₁₋₆alkynyl, SO₂NR^{8a}R^{8b};

d is 1, 2, or 3:

m is 1 or 2; and

R^{8a} and R^{8b} are independently selected from hydrogen or C₁₋₆alkyl.

In one particular embodiment R¹ is hydrogen or C₁₋₆alkyl, more particularly hydrogen.

In one particular embodiment R⁴ is hydrogen or methyl, more particularly hydrogen.

In one particular embodiment R³ is pyridinyl, pyrimidinyl, imidazoyl, oxadiazoyl, triazolyl or pyrazinyl any of which can be unsubstituted or substituted or is group A, In one particular embodiment R³ is group A, pyridinyl or pyrimidinyl. In a further particular embodiment R³ is group A or pyridinyl

When R³ is a substituted 5- to 6-membered aromatic heterocyclyl group, the substituent or substituents is/are preferably selected from: C₁₋₆ alkyl, C₁₋₆ alkoxy, halosubstituted C₁₋₆ alkoxy, hydroxy, cyano, halo, sulfonyl, CONH₂, and COOH. Preferably the halo is fluoro.

In one particular embodiment when R³ is an 5- to 6-membered aromatic heterocyclyl group the substituents are halo, methoxy, and cyano.

When R⁶ or R¹⁰ are substituted alkyl groups, they can be substituted with 1, 2 or 3 substitutents selected from hydroxy, C₁₋₆alkyloxy, cyano, halo, NR^{8a}R^{8b}, CONR^{8a}R^{8b}, SO₂NR^{8a}R^{8b}, NR^{8a}COR^{8b} or NR^{8a}SO₂R^{8b}, preferably hydroxy or fluorine.

In one particular embodiment R⁶ is a substituted or unsubstituted (C₁₋₆)alkyl, chloro or CHxFn wherein n is 1, 2, or 3, x is 0, 1 or 2 and n and x add up to 3 and R¹⁰ is hydrogen or R¹⁰ is a substituted or unsubstituted (C₁₋₆)alkyl, chloro or CHxFn wherein n is 1, 2, or 3, x is 0, 1 or 2 and n and x add up to 3 and R⁶ is hydrogen

In one particular embodiment R⁶ is t-butyl, isopropyl or CHxFn, more preferably R⁶ is isopropyl or CHxFn even more preferably isopropyl or CF₃ and R¹⁰ is hydrogen or R¹⁰ is t-butyl, isopropyl or CHxFn, more preferably R¹⁰ is isopropyl or CHxFn, more preferably isopropyl or CF₃ and R⁶ is hydrogen.

In one particular embodiment Rb is selected from halo, methoxy, and cyano.

In one particular embodiment R⁶ is (C₁₋₆)alkyl, chloro or CHxFn wherein n is 1, 2, or 3, x is 0, 1 or 2 and n and x add up to 3 and R¹⁰ is hydrogen.

Alternatively compounds of formula (III) are compounds of formula (IIIb)

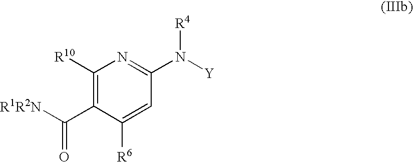

wherein:

Y is phenyl, substituted with one, two or three substituents;

R¹ is selected from hydrogen, C₁₋₆ alkyl, C₃₋₇ cycloalkyl, or halosubstitutedC₁₋₆ alkyl;

R² is CH₂R³;

R³ is an optionally substituted 5- to 6-membered aromatic heterocyclyl group, or group A:

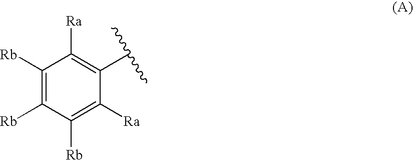

R⁴ is selected from hydrogen, C₁₋₆ alkyl, C₃₋₇ cycloalkyl, or halosubstitutedC₁₋₆ alkyl, COCH₃, or SO₂Me;

R⁶ is (C₁₋₆)alkyl, chloro or CHxFn wherein n is 1, 2, or 3, x is 0, 1 or 2 and n and x add up to 3 and R¹⁰ is hydrogen or R¹⁰ is (C₁₋₆)alkyl, chloro or CHxFn wherein n is 1, 2, or 3, x is 0, 1 or 2 and n and x add up to 3 and R⁶ is hydrogen;

Ra can be independently selected from hydrogen, fluoro, chloro or trifluoromethyl; and

Rb can independently be selected from hydrogen, C₁₋₆ alkyl, C₁₋₆ alkoxy, haloC₁₋₆ alkoxy, a hydroxy group, a cyano group, halo, a sulfonyl group, CONH₂, or COOH.

### Compounds of Formula (IV)

In compounds of formula (IV):

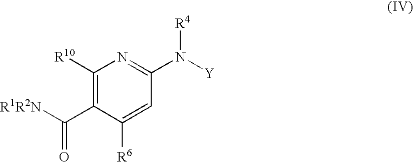

Y is phenyl, unsubstituted or substituted with one, two or three substituents;

R¹ is selected from hydrogen, C₁₋₆ alkyl, C₃₋₆ cycloalkyl, or halosubstitutedC₁₋₆ alkyl;

R² is (CH₂)ₘR³ where m is 0 or 1;

or R¹ and R² together with N to which they are attached form an optionally substituted 4- to 8-membered non-aromatic heterocyclyl ring;

R³ is a 4- to 8-membered non-aromatic heterocyclyl group, a C₃₋₈ cycloalkyl group, a straight or branched C₁₋₁₀ alkyl, a C₂₋₁₀alkenyl, a C₃₋₈cycloalkenyl, a C₂₋₁₀alkynyl, or a C₃₋₈cycloalkynyl any of which can be unsubstituted or substituted or R⁵;

R⁴ is selected from hydrogen, C₁₋₆ alkyl, C₃₋₆ cycloalkyl, or halosubstitutedC₁₋₆ alkyl, COCH₃, or SO₂Me;

R⁵ is

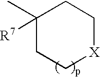

wherein p is 0, 1 or 2, and X is CH₂, O, or S;

R⁶ is a substituted or unsubstituted (C₁₋₆)alkyl or chloro and R¹⁰ is hydrogen or R¹⁰ is a substituted or unsubstituted (C₁₋₆)alkyl or chloro and R⁶ is hydrogen;

R⁷ is OH, C₁₋₆alkoxy, NR^{8a}R^{8b}, NHCOR⁹, NHSO₂R⁹ or SOqR⁹;

R^{8a} is H or C₁₋₆alkyl;

R^{8b} is H or C₁₋₆alkyl;

R⁹ is C₁₋₆alkyl; and

q is 0, 1 or 2.

In one particular embodiment Y is a substituted phenyl. In one particular embodiment Y is substituted by 1 or 2 substituents. If mono-substituted, in one particular embodiment, the substituents is in the 3 position.

When Y is substituted, the substituent or substituents are preferably selected from: C₁₋₆alkyl, halosubstitutedC₁₋₆ alkyl, C₁₋₆ alkoxy, a hydroxy group, a cyano group, halo, a C₁₋₆alkylsulfonyl group, —CONH₂, —NHCOCH₃, —COOH, C₁₋₆ alkynyl, halosubstitutedC₁₋₆ alkoxy, or SO₂NR^{8a}R^{8b} wherein R^{8a} and R^{8b} are as defined above.

In one particular embodiment Y is substituted by halo, cyano, methoxy, trifluoromethoxy or methyl.

In one particular embodiment the compound of formula (IV) is a compound of formula (IVa):

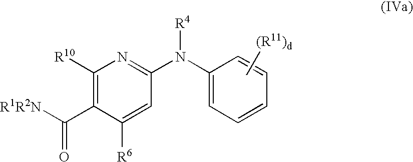

wherein:

R¹ is selected from hydrogen, C₁₋₆ alkyl, C₃₋₆ cycloalkyl, or halosubstitutedC₁₋₆ alkyl;

R² is (CH₂)ₘR³ where m is 0 or 1;

or R¹ and R² together with N to which they are attached form a non-aromatic heterocyclyl ring selected from azetidinyl, pyrrolidinyl, morpholinyl, piperazinyl, piperidinyl, tetrahydropyridinyl, azapine, oxapine, azacyclooctanyl, azaoxacyclooctanyl and azathiacyclooctanyl, any of which can be unsubstituted or substituted with 1, 2 or 3 substituents selected from; C₁₋₆ alkyl, C₁₋₆ alkoxy, hydroxy, cyano, halo, sulfonyl, methylsulfonyl, NR^{8a}R^{8b}, CH₂-phenyl, NHCOCH₃, (═O), CONHCH₃ and NHSO₂CH₃;

R³ is 2- or 3-azetidinyl, oxetanyl, thioxetanyl, thioxetanyl-s-oxide, thioxetanyl-s,s-dioxide, dioxalanyl, pyrrolidinyl, tetrahydrofuranyl, tetrahydrothiophenyl, tetrahydrothiophenyl-s,s-dioxide, morpholinyl, piperidinyl, piperazinyl, tetrahydropyranyl, tetrahydrothiopyranyl, thiomorpholinyl, thiomorpholinyl-s,s-dioxide, tetrahydropyridinyl, dioxanyl, tetrahydro-thiopyran 1,1 dioxide, azapine, oxapine, azacyclooctanyl, azaoxacyclooctanyl, azathiacyclooctanyl, oxacylcooctanyl, thiacyclooctanyl, a C₃₋₈ cycloalkyl group, a straight or branched C₁₋₁₀ alkyl, a C₂₋₁₀alkenyl, a C₃₋₈cycloalkenyl, a C₂₋₁₀alkynyl, or a C₃₋₈cycloalkynyl or R⁵; any of which can be unsubstituted or substituted with 1, 2 or 3 substituents selected from C₁₋₆ alkyl, C₁₋₆ alkoxy, hydroxy, cyano, halo, sulfonyl, methylsulfonyl, NR^{8a}R^{8b}, CH₂-phenyl, NHCOCH₃, (═O), CONHCH₃ and NHSO₂CH₃;

R⁴ is selected from hydrogen, C₁₋₆ alkyl, C₃₋₆ cycloalkyl, or halosubstitutedC₁₋₆ alkyl, COCH₃, or SO₂Me;

R⁵ is

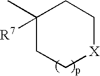

wherein p is 0, 1 or 2, and X is CH₂, O or S;

R⁶ is a substituted or unsubstituted (C₁₋₆)alkyl or chloro and R¹⁰ is hydrogen or R¹⁰ is a substituted or unsubstituted (C₁₋₆)alkyl or chloro and R⁶ is hydrogen;

R⁷ is OH, C₁₋₆alkoxy, NR^{8a}R^{8b}, NHCOR⁹, NHSO₂R⁹ or SOqR⁹;

R^{8a} is H or C₁₋₆alkyl;

R^{8b} is H or C₁₋₆alkyl;

R⁹ is C₁₋₆alkyl;

R¹¹ is C₁₋₆ alkyl, halosubstitutedC₁₋₆ alkyl, C₁₋₆ alkoxy, hydroxy, cyano, halo, C₁₋₆alkylsulfonyl group, —CONH₂, —NHCOCH₃, —COOH, halosubstituted C₁₋₆ alkoxy SO₂NR^{8a}R^{8b} or C₁₋₆ alkynyl;

q is 0, 1 or 2; and

d is 0, 1, 2, or 3.

In one particular embodiment R¹ is hydrogen.

In one particular embodiment R⁴ is C₁₋₆ alkyl or hydrogen, more preferably methyl or hydrogen, even more preferably hydrogen.

In one particular embodiment X is CH₂ or O.

When R¹ and R² together with N to which they are attached form a 4- to 8-membered non-aromatic heterocyclyl ring which is substituted, or when R³ is substituted, they may be substituted with 1, 2 or 3 substituents preferably selected from: C₁₋₆ alkyl, C₁₋₆ alkoxy, a hydroxy group, a cyano group, halo, a sulfonyl group, methylsulfonyl, NR^{8a}R^{8b}, CH₂-phenyl, NHCOCH₃, (═O), CONHCH₃ or NHSO₂CH₃ wherein R^{8a} and R^{8b} are as defined for formula (IV).

When R⁶ or R¹⁰ are substituted alkyl groups, they can be substituted with 1, 2 or 3 substitutents selected from hydroxy, C₁₋₆alkyoxy, cyano, halo, NR^{1a}R^{8b}, CONR^{8a}R^{8b}, SO₂NR^{8a}R^{8b}, NR^{8a}COR^{8b} or NR^{8a}SO₂R^{8b}, preferably hydroxy or fluorine.

In one particular embodiment R¹ and R² together with the N to which they are attached form an optionally substituted 5- or 6-membered non-aromatic heterocyclyl ring.

In one particular embodiment R⁶ is a substituted or unsubstituted (C₁₋₆)alkyl, chloro or CHxFn wherein n is 1, 2, or 3, x is 0, 1 or 2 and n and x add up to 3 and R¹⁰ is hydrogen or R¹⁰ is a substituted or unsubstituted (C₁₋₆)alkyl, chloro or CHxFn wherein n is 1, 2, or 3, x is 0, 1 or 2 and n and x add up to 3 and R⁶ is hydrogen

In one particular embodiment R⁶ is t-butyl, isopropyl or CHxFn, more preferably R⁶ is isopropyl or CHxFn even more preferably isopropyl or CF₃ and R¹⁰ is hydrogen or R¹⁰ is t-butyl, isopropyl or CHxFn, more preferably R¹⁰ is isopropyl or CHxFn, more preferably isopropyl or CF₃ and R⁶ is hydrogen.

In one particular embodiment R¹⁰ is hydrogen.

In one particular embodiment R⁷ is OH.

In one particular embodiment R⁵ is

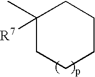

wherein p is 0, 1 or 2.

In one particular embodiment when R³ is an optionally substituted C₃₋₈cycloalkyl group or an optionally substituted 4- to 8-membered nonaromatic heterocyclyl, m is 1.

In one particular embodiment R³ is an optionally substituted C₃₋₆cycloalkyl group or an optionally substituted 4- or 6-membered nonaromatic heterocyclyl.

In one particular embodiment when R¹ and R² taken together with the N to which they are attached form an optionally substituted heterocyclyl ring, the ring may be selected from pyrrolidinyl, morpholinyl, piperazinyl, piperidinyl and tetrahydropyridinyl.

In one particular embodiment when R³ is an optionally substituted non-aromatic heterocyclyl group selected from dioxalanyl, pyrrolidinyl, tetrahydrofuranyl, tetrahydrothiophenyl, tetrahydrothiophenyl-s,s-dioxide, morpholinyl, piperidinyl, piperazinyl, tetrahydropyranyl, tetrahydrothiopyranyl, dioxanyl, thiomorpholinyl, dioxanyl, thiomorpholinyl-s,s-dioxide and tetrahydropyridinyl.

Alternatively compounds of formula (IV) are compounds of formula (IVb):

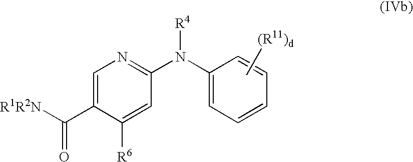

wherein:

R¹ is selected from hydrogen;

R² is (CH₂)ₘR³ where m is 0 or 1;

or R¹ and R² together with N to which they are attached form pyrrolidinyl, morpholinyl, piperazinyl, piperidinyl, tetrahydropyridinyl, any of which can be unsubstituted or substituted with 1, 2 or 3 substituents selected from C₁₋₆ alkyl, C₁₋₆ alkoxy, hydroxy, cyano, halo, sulfonyl, methylsulfonyl, NR^{8a}R^{8b}, CH₂-phenyl, NHCOCH₃, (═O), CONHCH₃ and NHSO₂CH₃;

R³ is dioxalanyl, pyrrolidinyl, tetrahydrofuranyl, tetrahydrothiophenyl, tetrahydrothiophenyl-s,s-dioxide, morpholinyl, piperidinyl, piperazinyl, tetrahydropyranyl, tetrahydrothiopyranyl, thiomorpholinyl, thiomorpholinyl-s,s-dioxide, dioxanyl, tetrahydropyridinyl, a C₃₋₈ cycloalkyl group, a straight or branched C₁₋₁₀ alkyl; any of which can be unsubstituted or substituted with 1, 2 or 3 substituents selected from C₁₋₆ alkyl, C₁₋₆ alkoxy, hydroxy, cyano, halo, sulfonyl, methylsulfonyl, NR^{8a}R^{8b}, CH₂-phenyl, NHCOCH₃, (═O), CONHCH₃ or NHSO₂CH₃; or R⁵.

R⁴ is selected from hydrogen, C₁₋₆ alkyl, C₃₋₆ cycloalkyl, or halosubstitutedC₁₋₆ alkyl, COCH₃, or SO₂Me;

R⁵ is

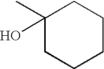

R⁶ is a substituted or unsubstituted (C₁₋₆)alkyl or chloro;

R^{8a} is H or C₁₋₆alkyl;

R^{8a} is C₁₋₆alkyl, halosubstitutedC₁₋₆ alkyl, C₁₋₆ alkoxy, hydroxy, cyano, halo, C₁₋₆alkylsulfonyl group, —CONH₂, —NHCOCH₃, —COOH, halosubstituted C₁₋₆ alkoxy, SO₂NR^{8a}R^{8b} or C₁₋₆ alkynyl; and

d is 0, 1, 2, or 3.

Alternatively compounds of formula (IV) can be selected from compounds of formula (IVc);

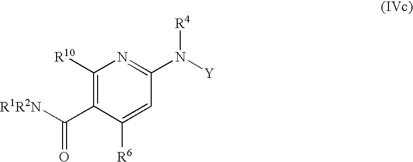

wherein:

Y is phenyl, optionally substituted with one, two or three substituents;

R¹ is selected from hydrogen, C₁₋₆ alkyl, C₃₋₆ cycloalkyl, or halosubstitutedC₁₋₆ alkyl;

R² is (CH₂)ₘR³ where m is 0 or 1;

or R¹ and R² together with N to which they are attached form an optionally substituted 5- or 6-membered non-aromatic heterocyclyl ring;

R³ is an optionally substituted 4- to 8-membered non-aromatic heterocyclyl group, an optionally substituted C₃₋₈ cycloalkyl group, an optionally substituted straight or branched C₁₋₁₀ alkyl or R⁵;

R⁴ is selected from hydrogen, C₁₋₆ alkyl, C₃₋₆ cycloalkyl, or halosubstitutedC₁₋₆ alkyl, COCH₃, or SO₂Me;

R⁵ is

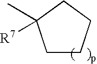

wherein p is 0, 1 or 2;

R⁶ is (C₁₋₆)alkyl, chloro or CHxFn wherein n is 1, 2, or 3, x is 0, 1 or 2 and n and x add up to 3 and R¹⁰ is hydrogen or R¹⁰ is (C₁₋₆)alkyl, chloro or CHxFn wherein n is 1, 2, or 3, x is 0, 1 or 2 and n and x add up to 3 and R⁶ is hydrogen;

R⁷ is OH, C₁₋₆alkoxy, NR^{8a}R^{8b}, NHCOR⁹, NHSO₂R⁹, SOqR⁹;

R^{8a} is H or C₁₋₆alkyl;

R^{8b} is H or C₁₋₆alkyl;

R⁹ is C₁₋₆alkyl; and

q is 0, 1 or 2.

It is to be understood that reference herein to CB2 modulators such as compounds of formula (I)-(IV) includes pharmaceutically acceptable derivatives thereof. In one particular embodiment the cannabinoid 2 modulators are selective for CB2 over CB1. Preferably the cannabinoid 2 modulators are 100 fold selective. Preferably compounds of formula (I) have an EC50 value at the cloned human cannabinoid CB2 receptor of at least 100 times the EC50 values at the cloned humna cannabinoid CB1 receptor or have less than 10% efficacy at the CB1 receptor.

The PDE4 inhibitors useful in this invention may be any compound that is known to inhibit the PDE4 enzyme or which is discovered to act in as PDE4 inhibitor, and which is only or essentially only a PDE4 inhibitor, not compounds which inhibit to a degree of exhibiting a therapeutic effect other members of the PDE family as well as PDE4. Generally it is preferred to use a PDE4 antagonists which has an IC₅₀ ratio of about 0.1 or greater as regards the IC₅₀ for the PDE4 catalytic form which binds rolipram with a high affinity divided by the IC₅₀ for the form which binds rolipram with a low affinity. Combinations of the present invention can be used in treating inflammation and as bronchodilators.

It turns out that there are at least two binding forms on human monocyte recombinant PDE 4 (hPDE 4) at which inhibitors bind. One explanation for these observations is that hPDE 4 exists in two distinct forms. One binds the likes of rolipram and denbufylline with a high affinity while the other binds these compounds with a low affinity. The preferred PDE4 inhibitors of for use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

Reference is made to U.S. Pat. No. 5,998,428, which describes these methods in more detail. It is incorporated herein in full as though set forth herein.

Most preferred are those PDE4 inhibitors which have an IC₅₀ ratio of greater than 0.5, and particularly those compounds having a ratio of greater than 1.0.

Preferred PDE4 compounds are cis[cyano-4-(3-cyclopentyloxy-4-methoxyphenyl)cyclohexan-1-carboxylate] also known as cilomilast or Ariflo®, 2-carbomethoxy-4-cyano-4-(3-cyclopropylmethoxy-4-difluoromethoxyphenyl)cyclohexan-1-one, and cis[4-cyano-4-(3-cyclopropylmethoxy-4-difluoromethoxyphenyl)cyclohexan-1-ol]. They can be made by the processes described in U.S. Pat. Nos. 5,449,686 and 5,552,438. Other PDE4 inhibitors, specific inhibitors, which can be used in this invention are AWD-12-281 from ASTA MEDICA (Hofgen, N. et al. 15th EFMC Int Symp Med Chem (September 6-10, Edinburgh) 1998, Abst P. 98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (September 19-23, Geneva) 1998] 1998, 12(Suppl. 28): Abst P2393); roflumilast (CAS reference No 162401-32-3) and a pthalazinone (WO 99/47505) from Byk-Gulden (now Altana); or a compound identified as T-440 (Tanabe Seiyaku; Fuji, K. et al. J Pharmacol Exp Ther, 1998, 284(1): 162).

Additional PDE4 inhibitors are disclosed on pages 2 to 15 of WO01/13953. Specifically selected are arofylline, atizoram, BAY-19-8004, benafentrine, BYK-33043, CC-3052, CDP-840, cipamfylline, CP-220629, CP-293121, D-22888, D-4396, denbufylline, filaminast, GW-3600, ibudilast, KF-17625, KS-506-G, laprafylline, NA-0226A, NA-23063A, ORG-20241, ORG-30029, PDB-093, pentoxifylline, piclamilast, rolipram, RPR-117658, RPR-122818, RPR-132294, RPR-132703, RS-17597, RS-25344-000, SB-207499, SB210667, SB211572, SB-211600, SB212066, SB212179, SDZ-ISQ-844, SDZ-MNS-949, SKF-107806, SQ-20006, T-2585, tibenelast, tolafentrine, UCB-29646, V-111294A, YM-58997, YM-976 and zardaverine.

Preferably the PDE4 inhibitor is selected from cilomilast, AWD-12-281, NCS-613, D-4418, CI-1018, V-11294A, roflumilast or T-440.

The invention is described using the following definitions unless otherwise indicated.

The term “pharmaceutically acceptable derivative” means any pharmaceutically acceptable salt, ester, salt of such ester or solvate of a CB2 modulator or PDE4 modulator or any other compound which upon administration to the recipient is capable of providing (directly or indirectly) a CB2 modulator or a PDE4 inhibitor as applicable or an active metabolite or residue thereof.

It will be appreciated by those skilled in the art that compounds described above may be modified to provide pharmaceutically acceptable derivatives thereof at any of the functional groups in the compounds, and that the compounds may be derivatised at more than one position.

It will be appreciated that, for pharmaceutical use, the salts referred to above will be physiologically acceptable salts, but other salts may find use, for example in the preparation of compounds of formula (I)-(IV) and the physiological acceptable salts thereof. Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropyl amine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Preferred examples of pharmaceutically acceptable salts include the ammonium, calcium, magnesium, potassium, and sodium salts, and those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The terms ‘halogen or halo’ are used to represent fluorine, chlorine, bromine or iodine.

The term ‘alkyl’ as a group or part of a group means a straight or branched chain alkyl group or combinations thereof, for example a methyl, ethyl, n-propyl, i-propyl, n-butyl, s-butyl, t-butyl, pentyl, hexyl, 1,1-dimethylethyl, or combinations thereof.

The term ‘alkoxy’ as a group or as part of a group means a straight, branched or cyclic chain alkyl group having an oxygen atom attached to the chain, for example a methoxy, ethoxy, n-propoxy, i-propoxy, n-butoxy, s-butoxy, t-butoxy group, pentoxy, hexyloxy group, cyclopentoxy or cyclohexyloxy group.

The term ‘cycloalkyl’ means a closed 3-8 membered non-aromatic ring, for example cyclobutyl, cyclopentyl, cyclohexyl or cycloheptyl, or cyclooctyl.

The term ‘alkenyl’ as a group or part of a group means a straight or branched chain carbon chain or combinations containing 1 or more double bonds for example an ethenyl, n-propenyl, i-propenyl, butenyl, pentenyl, hexenyl or combinations thereof.

The term ‘cycloalkenyl’ as a group or part of a group means a closed non-aromatic carbon ring, containing one or more double bonds for example cyclobutenyl, cyclopentenyl, cyclohexenyl or cycloheptenyl, or cyclooctenyl.

The term ‘alkynyl’ as a group or part of a group means a straight or branched chain carbon chain or combinations containing 1 or more triple carbon bonds for example a ethynyl, propynyl, butynyl, pentynyl, hexynyl or combinations thereof.

The term ‘cycloalkynyl’ means a closed non-aromatic carbon ring containing 1 or more triple bonds, for example cyclobutynyl, cyclopentynyl, cyclohexynyl or cycloheptynyl, or cyclooctynyl.

The term ‘aryl’ means a 5- or 6-membered aromatic ring, for example phenyl, or a 7- to 12-membered bicyclic ring system where at least one of the rings is aromatic, for example naphthyl.

In compounds of formulas (I) to (IV) when R¹ and R² taken together with the N to which they are attached form an optionally substituted heterocyclyl ring, the ring may optionally contain 1, 2, 3 or 4 further heteroatoms. The ring may be saturated or unsaturated. Preferably the further heteroatoms are selected from oxygen, nitrogen or sulphur. An example of a 4-membered heterocyclyl ring is azetidinyl. Examples of 5-membered heterocyclyl rings include pyrrolidinyl. Examples of 6-membered heterocyclyl rings are morpholinyl, piperizinyl or piperidinyl. An additional example is tetrahydropyridinyl. Examples of a 7-membered heterocyclyl ring are azapine or oxapine. Examples of 8-membered heterocyclyl rings are azacyclooctanyl, azaoxacyclooctanyl or azathiacyclooctanyl.

In compounds of formula (I) and (IV), when R³ is an optionally substituted non-aromatic heterocyclyl group, the ring may contain 1, 2, 3, or 4 heteroatoms. Preferably the heteroatoms are selected from oxygen, nitrogen or sulphur. Examples of 4-membered groups are 2- or 3-azetidinyl, oxetanyl, thioxetanyl, thioxetanyl-s-oxide, thioxetanyl-s,s-dioxide. Examples of 5-membered heterocyclyl groups in this instance include dioxalanyl, pyrrolidinyl, tetrahydrofuranyl or tetrahydrothiophenyl. Additionally it can be tetrahydrothiophenyl-s,s-dioxide. Examples of 6-membered heterocyclyl groups are morpholinyl, piperidinyl, piperazinyl, tetrahydropyranyl, tetrahydrothiopyranyl, thiomorpholinyl or thiomorpholinyl-s,s-dioxide. Additional examples are tetrahydropyridinyl, dioxanyl, and tetrahydrothiopyran-1,1-dioxide. Examples of a 7-membered heterocyclyl ring are azapine or oxapine. Examples of 8-membered groups are azacyclooctanyl, azaoxacyclooctanyl or azathiacyclooctanyl, oxacylcooctanyl, or thiacyclooctanyl.

In compounds of formula (II) and (III), when R³ is an (optionally substituted) aromatic heterocyclyl group, the ring may contain 1, 2, 3, or 4 hetero atoms. Preferably the hetero atoms are selected from oxygen, nitrogen or sulphur. Examples of 5-membered hleterocyclyl groups in this instance include furanyl, dioxalanyl, pyrrolyl, oxazolyl, thiazolyl, imidazolyl, oxadiazolyl, thiadiazolyl, triazolyl, triazinyl, isothiazolyl, isoxazolyl, thienyl, pyrazolyl or tetrazolyl. Examples of 6-membered heterocyclyl groups are pyridinyl, pyrizinyl, pyrimidinyl, pyrazinyl, triazinyl, or tetrazinyl.

Preferably compounds of formula (I) to (IV) can be selected from the examples hereinbelow and pharmaceutically acceptable derivatives thereof.

Compounds of formula (I) and (II) can be prepared as set forth in scheme 1.

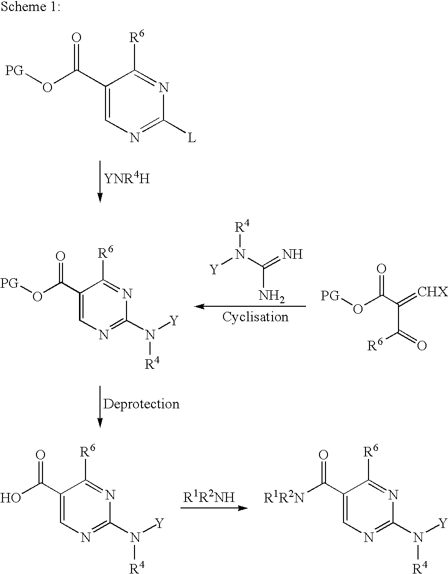

wherein L is a leaving group, for example halo, PG is a protecting group for example methyl, ethyl or benzyl, X is a leaving group for example halo, OC₁₋₆alkyl, e.g. O-methyl or O-ethyl or NR^{a}R^{b} wherein R^{a} and R^{b} are independently selected from C₁₋₆ alkyl, e.g. methyl, and R¹, R², R⁴, R⁶ and Y are as defined for compounds of formula (I) or (II).

Compounds of formula (I) can also be prepared as set forth in scheme 2.

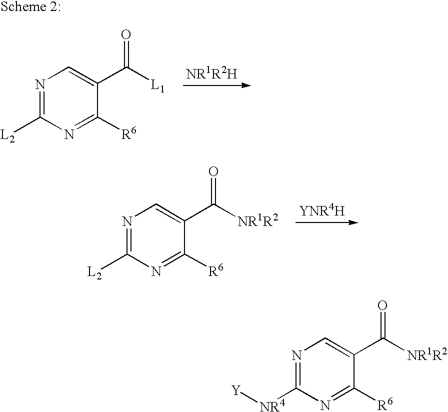

wherein L₁ and L₂ are leaving groups independently selected from halo, for example chloro, R¹, R², R⁴, R⁶ and Y are as defined for compounds of formula (I).

Compounds of formula (III) and (IV) can be prepared as set forth in scheme 3.

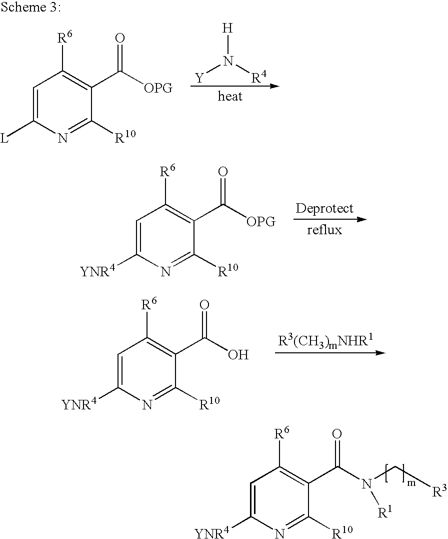

wherein R¹, R³, R⁴, R⁶, Y, m and R¹⁰ are as defined for compounds of formula (III) or (IV), wherein L is a leaving group, for example halo, PG is a protecting group for example methyl, ethyl or benzyl.

Furthermore compounds of formula (III) or (IV) when R¹⁰ is unsubstituted or substituted (C₁₋₆)alkyl or chloro and R⁶ is hydrogen can be prepared as shown in scheme 4.

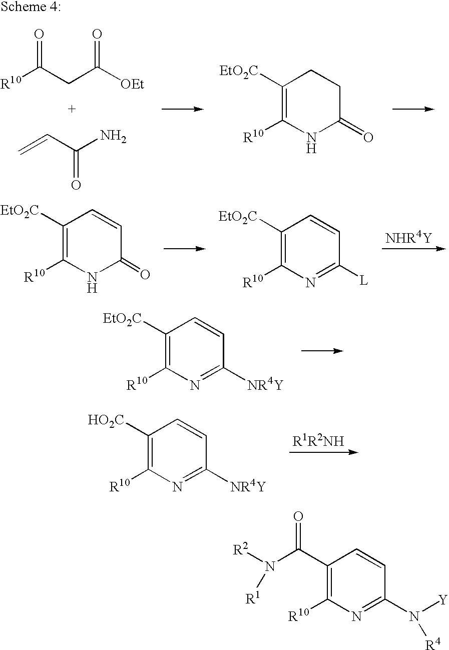

wherein L is a leaving group for example halogen, e.g. chloro, R¹, R², Y, R⁴ are as defined for compounds of formula (III) or (IV).

Furthermore compounds of formula (III) or (IV) when R¹⁰ is unsubstituted or substituted (C₁₋₆)alkyl or chloro and R⁶ is hydrogen can be prepared as shown in scheme 5.

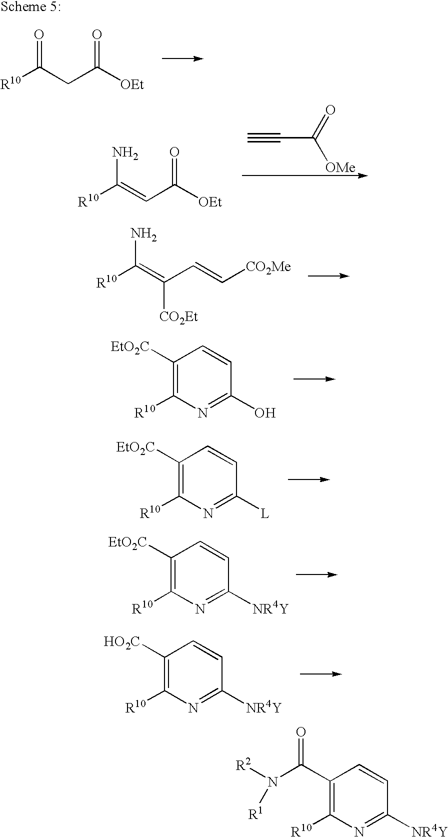

wherein L is a leaving group for example halogen, e.g. chloro, R¹, R², Y, R⁴ are as defined for compounds of formula (III) or (IV).

Furthermore compounds of formula (III) or (IV) can be prepared as shown in scheme 6.

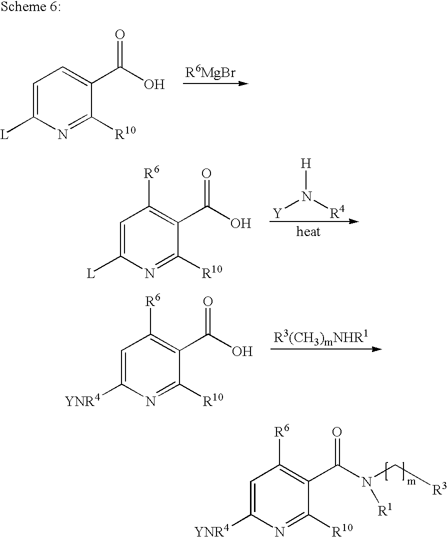

wherein L is a leaving group for example halogen, e.g. chloro, R¹, R³, R⁴, Y, R¹⁰ and m are as defined for compounds of formula (III) or (V).

Furthermore compounds of formula (III) can be prepared as shown in scheme 7.

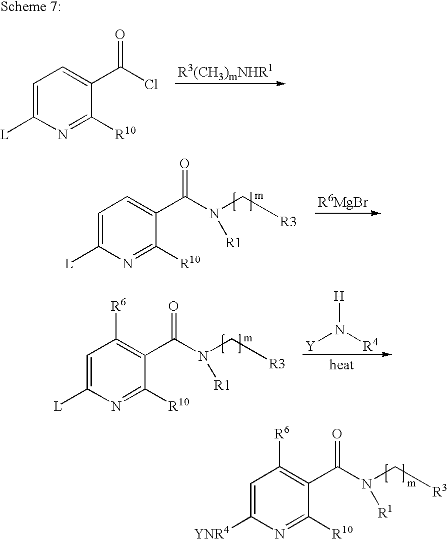

wherein L is a leaving group for example halogen, e.g. chloro, R¹, R³, R⁴, Y, R¹⁰ and m are as defined for compounds of formula (III).

It is to be understood that references herein to compounds of formula (I), (I), (III) and (IV) encompass all isomers, including all geometric, tautomeric and optical forms, and mixtures thereof (e.g. racemic mixtures). Where additional chiral centres are present in compounds of formula (I), all possible diastereoismers, including mixtures thereof are included. The different isomeric forms may be separated or resolved one from the other by conventional methods, or any given isomer may be obtained by conventional synthetic methods or by stereospecific or asymmetric syntheses.

The compounds of formula (I)-(IV) may be prepared in crystalline or non-crystalline form, and, if crystalline, may optionally be hydrated or solvated. This invention includes within its scope the use of stoichiometric hydrates or solvates as well as compounds containing variable amounts of water and/or solvent.

The combinations of the present invention may be useful in the treatment of the disorders that follow. Thus, the combinations of the invention may be useful as analgesics. For example they may be useful in the treatment of chronic inflammatory pain (e.g. pain associated with rheumatoid arthritis, osteo-arthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

The combinations of the invention may also be useful disease modification or joint structure preservation in multiple sclerosis, rheumatoid arthritis, osteo-arthritis, rheumatoid spondylitis, gouty arthritis and juvenile arthritis.

The combinations of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The combinations of the invention may also be useful in the treatment of fever.

The combinations of the invention may also be useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastro esophageal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, polymyositis, gingivitis, myocardial ischemia, pyrexia, systemic lupus erythematosus, tendinitis, bursitis, and Sjogren's syndrome.

The combinations of the invention may also be useful in the treatment of bladder hyperrelexia following bladder inflammation.

The combinations of the invention may also be useful in the treatment of immunological diseases such as autoimmune diseases, immunological deficiency diseases or organ transplantation. The combinations of the invention are also effective in increasing the latency of HIV infection.

The combinations of the invention may also be useful in the treatment of diseases of abnormal platelet function (e.g. occlusive vascular diseases).

The combinations of the invention may also be useful in the treatment of neuritis, heart burn, dysphagia, pelvic hypersensitivity, urinary incontinence, cystitis or pruritis.

The combinations of the invention may also be useful for the preparation of a drug with diuretic action.

The combinations of the invention may also be useful in the treatment of impotence or erectile dysfunction.

The combinations of the invention may also be useful for attenuating the hemodynamic side effects of non-steroidal anti-inflammatory drugs (NSAID's) and cyclooxygenase-2 (COX-2) inhibitors.

The combinations of the invention may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with ageing, particularly Age Associated Memory Impairment. The compounds may also be useful for the treatment of amyotrophic lateral sclerosis (ALS) and neuroinflamation.

The combinations of the invention may also be useful in neuroprotection and in the treatment of neurodegeneration following stroke, cardiac arrest, pulmonary bypass, traumatic brain injury, spinal cord injury or the like.

The combinations of the invention may also be useful in the treatment of tinnitus.

The combinations of the invention may also be useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), panic disorder, agoraphobia, social phobia, obsessive compulsive disorder and post-traumatic stress disorder, memory disorders, including dementia, amnesic disorders and age-associated memory impairment, disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof.

The combinations of the invention may also be useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

The combinations of the invention may also be useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

According to a further aspect of the invention we provide a method of treating a human or animal subject suffering from an immune disorder, an inflammatory disorder, pain, rheumatoid arthritis, multiple sclerosis, osteoarthritis, osteoporosis, lung disorders, for example asthma, bronchitis, emphysema, allergic rhinitis, respiratory distress syndrome, pigeon fancier's disease, farmer's lung, chronic obstructive pulmonary disease, (COPD) and cough, or a disorder which can be treated with a bronchodilator which method comprises administering to said subject an effective combination of one or more CB2 modulators or a pharmaceutically acceptable derivative thereof and one or more PDE4 inhibitors or a pharmaceutically acceptable derivate thereof.

According to another aspect of the invention is provided the use of a combination of one or more CB2 modulators a pharmaceutically acceptable derivate thereof and one or more PDE4 inhibitors a pharmaceutically acceptable derivate thereof for the manufacture of a therapeutic agent for the treatment or prevention of a condition such as an immune disorder, an inflammatory disorder, pain, rheumatoid arthritis, multiple sclerosis, osteoarthritis, osteoporosis, lung disorders, for example asthma, bronchitis, emphysema, allergic rhinitis, respiratory distress syndrome, pigeon fancier's disease, farmer's lung, chronic obstructive pulmonary disease, (COPD) and cough, or a disorder which can be treated with a bronchodilator.

Preferably the pain is selected from inflammatory pain, viseral pain, cancer pain, neuropathic pain, lower back pain, muscular sceletal, post operative pain, acute pain and migraine. More preferably the inflammatory pain is pain associated with rheumatoid arthritis or osteoarthritis.

When used herein cough can have a number of forms and includes productive, non-productive, hyper-reactive, asthma and COPD associated.

In order to use a combination of the invention for the treatment of humans and other mammals it is normally formulated in accordance with standard pharmaceutical practice as a pharmaceutical composition. Therefore in another aspect of the invention is provided a pharmaceutical composition comprising one or more CB2 modulators a pharmaceutically acceptable derivate thereof and one or more PDE4 inhibitors a pharmaceutically acceptable derivate thereof adapted for use in human or veterinary medicine.

As used herein, “modulator” means both antagonist, full or partial agonist and inverse agonist. In one embodiment of the invention modulators are agonists.

The term “treatment” or “treating” as used herein includes the treatment of established disorders and also includes the prophylaxis thereof. The term “prophylaxis” is used herein to mean preventing symptoms in an already afflicted subject or preventing recurrance of symptoms in an afflicted subject and is not limited to complete prevention of an afflication.

Combinations of the invention may be administered in a standard manner for the treatment of the indicated diseases, for example orally, parentarally, sub-lingually, dermally, intranasally, transdermally, rectally, via inhalation or via buccal administration.

Combinations of the invention which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavouring or colouring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or derivative in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a combination of the invention which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointmnent, lotion or paste or are in the form of a medicated plaster, patch or membrane.

Preferably the composition is in unit dosage form, for example a tablet, capsule or metered aerosol dose, so that the patient may administer a single dose.

When one therapeutic agent is used in combination with, a second therapeutic agent active against the same disease state the dose of each compound may differ from that when the compound is used alone. Appropriate doses will be readily appreciated by those skilled in the art. It may be advantageous to prepare the compounds used in the present invention as nanoparticles. Numerous processes for the synthesis of solid particles in nanoparticulate form are known. Typically these processes involve a milling process, preferably a wet milling process in the presence of a surface modifying agent that inhibits aggregation and/or crystal growth of the nanoparticles once created. Alternatively these processes may involve a precipitation process, preferably a process of precipitation in an aqueous medium from a solution of the drug in a non-aqueous solvent. Representative processes for the preparation of solid particles in nanoparticulate form are described in the patents and publications listed below.

U.S. Pat. No. 4,826,689 to Violanto & Fischer, U.S. Pat. No. 5,145,684 to Liversidge et al U.S. Pat. No. 5,298,262 to Na & Rajagopalan, U.S. Pat. No. 5,302,401 Liversidge et al U.S. Pat. No. 5,336,507 to Na & Rajagopalan, U.S. Pat. No. 5,340,564 to Illig & Sarpotdar U.S. Pat. No. 5,346,702 to Na Rajagopalan, U.S. Pat. No. 5,352,459 to Hollister et al U.S. Pat. No. 5,354,560 to Lovrecich, U.S. Pat. No. 5,384,124 to Courteille et al, U.S. Pat. No. 5,429,824 to June, U.S. Pat. No. 5,503,723 to Ruddy et al, U.S. Pat. No. 5,510,118 to Bosch et al, U.S. Pat. No. 5,518 to Bruno et al, U.S. Pat. No. 5,518,738 to Eickhoff et al, U.S. Pat. No. 5,534,270 to De Castro, U.S. Pat. No. 5,536,508 to Canal et al, U.S. Pat. No. 5,552,160 to Liversidge et al, U.S. Pat. No. 5,560,931 to Eickhoff et al, U.S. Pat. No. 5,560,932 to Bagchi et al, U.S. Pat. No. 5,565,188 to Wong et al, U.S. Pat. No. 5,571,536 to Eickhoff et al, U.S. Pat. No. 5,573,783 to Desieno & Stetsko, U.S. Pat. No. 5,580,579 to Ruddy et al, U.S. Pat. No. 5,585,108 to Ruddy et al, U.S. Pat. No. 5,587,143 to Wong, U.S. Pat. No. 5,591,456 to Franson et al, U.S. Pat. No. 5,622,938 to Wong, U.S. Pat. No. 5,662,883 to Bagchi et al, U.S. Pat. No. 5,665,331 to Bagchi et al, U.S. Pat. No. 5,718,919 to Ruddy et al, U.S. Pat. No. 5,747,001 to Wiedmann et al, WO93/25190, WO96/24336, WO 97/14407, WO 98/35666, WO 99/65469, WO 00/18374, WO 00/27369, WO 00/30615 and WO 01/41760, WO02/00196 (SmithKline Beecham plc).

Preferably, the pharmaceutical composition as hereinbefore defined, further comprises HPMC present in less than 15% w/w, preferably in the range 0.1 to 10% w/w.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

A further aspect of the invention is a patient pack comprising an effective combination of one or more CB2 modulators and one or more PDE4 inhibitors.

### Determination of Cannabinoid CB1 Receptor Agonist Activity

The cannabinoid CB1 receptor agonist activity of the compounds of formula (I)-(IV) was determined in accordance with the following experimental method.

### Experimental Method

Yeast (Saccharomyces cerevisiae) cells expressing the human cannabinoid CB1 receptor were generated by integration of an expression cassette into the ura3 chromosomal locus of yeast strain MMY23. This cassette consisted of DNA sequence encoding the human CB1 receptor flanked by the yeast GPD promoter to the 5′ end of CB1 and a yeast transcriptional terminator sequence to the 3′ end of CB1. MMY23 expresses a yeast/mammalian chimeric G-protein alpha subunit in which the C-terminal 5 amino acids of Gpa1 are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using dilutions of between 3- and 5-fold (BiomekFX, Beckman) into DMSO. Agonist solutions in DMSO (1% final assay volume) were transferred into black, clear bottom, microtitre plates from NUNC (96- or 384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20 μM fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluor microtitre plate reader (Tecan; excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation

Eₘₐₓ=Max_{[compound X}]−Min_{[compound X}]/Max_{[RU210}]−Min_{[HU210}]×100%

where Max_{[compound S]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC_{50 [compound X]}/EC_{50 [HU210]}

Where EC_{50 [compound X]} is the EC₅₀ of compound X and EC_{50 [HU210]} is the EC₅₀ of HU210.

Compounds of Examples 1 to 379 tested according to this method had EC₅₀ values >2000 nM and/or efficacy values of <50% at the cloned human cannabinoid CB1 receptor. Compounds of Examples 380 to 764 tested according to this method had EC₅₀ values >30,000 nM at the cloned human cannabinoid CB1 receptor.

### Determination of Cannabinoid CB2 Receptor Agonist Activity

The cannabinoid CB2 receptor agonist activity of the compounds of formula (I)-(IV) was determined in accordance with the following experimental method.

### Experimental Method

Yeast (Saccharomyces cerevisiae) cells expressing the human cannabinoid CB2 receptor were generated by integration of an expression cassette into the ura3 chromosomal locus of yeast strain MMY23. This cassette consisted of DNA sequence encoding the human CB2 receptor flanked by the yeast GPD promoter to the 5′ end of CB2 and a yeast transcriptional terminator sequence to the 3′ end of CB2. MMY23 expresses a yeast/mammalian chimeric G-protein alpha subunit in which the C-terminal 5 amino acids of Gpa1 are replaced with the C-terminal 5 amino acids of human Gαi3 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM stocks in DMSO. EC₅₀ values (the concentration required to produce 50% maximal response) were estimated using dilutions of between 3- and 5-fold (BiobmekFX, Beckman) into DMSO. Agonist solutions in DMSO (1% final assay volume) were transferred into black, clear bottom, microtitre plates from NUNC (96- or 384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 20M fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluor microtitre plate reader (Tecan; excitation wavelength: 485 nm; emission wavelength: 535 nm). Fluorescence was plotted against compound concentration and iteratively curve fitted using a four parameter fit to generate a concentration effect value. Efficacy (Eₘₐₓ) was calculated from the equation

Eₘₐₓ=Max_{[compound X]}−Min_{[compound X]}/Max_{[HU210}]−Min_{[HU210]}×100%

where Max_{[compound X]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC_{50 [compound X]}/EC_{50 [HU210]}

Where EC_{50 [compound X]} is the EC₅₀ of compound X and EC_{50 [HU210]} is the EC₅₀ of HU210.

Compounds of Examples 1 to 23, 31 to 56, 68, 163-256 tested according to this method had EC₅₀ values 20 to 300 nM and efficacy values of >50% at the cloned human cannabinoid CB2 receptor.

Compounds of Examples 24 to 30 and 73-113, and 257-259 tested according to this method had EC₅₀ values 300 to 1000 nM and efficacy values of >50% at the cloned human cannabinoid CB2 receptor.

Compounds of Examples 57-67, 69-72, 114-162, and 260-265 tested according to this method had EC₅₀ values >1000 nM and/or efficacy values of >50% at the cloned human cannabinoid CB2 receptor.

Compounds of Examples 266 to 294 and 344 to 369 tested according to this method had EC₅₀ values 20 to 300 nM and efficacy values of >50% at the cloned human cannabinoid CB2 receptor.

Compounds of Examples 295 to 307 tested according to this method had EC₅₀ values >300 nM but <1000 nM and efficacy values of >50% at the cloned human cannabinoid CB2 receptor.

Compounds of Examples 308 to 343 and 370 to 379 tested according to this method had EC₅₀ values >1000 nM and/or efficacy values of <50% at the cloned human cannabinoid CB2 receptor.

Compounds of Examples 380 to 384, 394, 396 to 403, 423 to 437, 449 to 452 and 457 tested according to this method had EC₅₀ values of less than 300 nM and efficacy values of >50% at the cloned human cannabinoid CB2 receptor.

Compounds of Examples 385 to 388, 393, 395, and 404 to 411, 453 to 455 had EC₅₀ >300 nM but <1000 nM and efficacy >50% at the cloned human cannabinoid CB2 receptor.

Compounds of Examples 389 to 392, 412 to 422 and 438 to 448, 456 and 458 had EC₅₀>1000 nM and/or efficacy <50% at the cloned human cannabinoid CB2 receptor.

Compounds of Examples 459 to 496, 508 to 513, 527 to 551, 562 to 630, 662, 666 to 678, 681, 682, 692 to 737, 751, 753 to 755 tested according to this method had an EC₅₀ values of 40<300 nM and efficacy value of >50% at the cloned human cannabinoid CB2 receptor.

Compounds of Examples 497 to 503, 514 to 520, 552 to 560, 631 to 635, 738 to 750, 752 and 756 to 762 tested according to this method had an EC₅₀ values of >300 nM but <1000 nM and efficacy value of >50% at the cloned human cannabinoid CB2 receptor.

The compounds of Examples 504 to 507, 521 to 526, 561, 636 to 661, 663 to 665, 680, 683 to 691 and 763 tested according to this method had an EC₅₀ values of >1000 nM and/or efficacy value of <50% at the cloned human cannabinoid CB2 receptor.

The compound of Example 679 tested according to this method had an EC₅₀ value of between 300 and 1000 nM and an efficacy value of <30% at the cloned human cannabinoid CB2 receptor.

PDE 4 Versus Rolipram High Affinity Binding-Phosphodiesterase and Rolipram Binding Assay

Isolated human monocyte PDE 4 and hrPDE (human recombinant PDE4) was determined to exist primarily in the low affinity form. Hence, the activity of test compounds against the low affinity form of PDE 4 can be assessed using standard assays for PDE 4 catalytic activity employing 1 μM [³H]cAMP as a substrate (Torphy et al., J. of Biol. Chem., Vol. 267, No. 3 pp 1798-1804, 1992).

Rat brain high-speed supernatants were used as a source of protein. Enantionmers of [³H]-rolipram were prepared to a specific activity of 25.6 Ci/mmol. Standard assay conditions were modified from the published procedure to be identical to the PDE assay conditions, except for the last of the cAMP: 50 mM Tris HCl (pH 7.5), 5 mM MgCl₂, and 1 nanoM of [³H]-rolipram (Torphy et al., J. of Biol. Chem., Vol. 267, No. 3 pp 1798-1804, 1992). The assay was run for 1 hour at 30° C. The reaction was terminated and bound ligand was separated from free ligand using a Brandel cell harvester. Competition for the high affinity binding site was assessed under conditions that were identical to those used for measuring low affinity PDE activity, expect that [³H]-cAMP and [³H]5′-AMP were not present.

Measurement of Phosphodiesterase Activity

PDE activity was assayed using a [³H]cAMP scintillation proximity assay (SPA) or [³H]cGMP SPA enzyme assay as described by the supplier (Amersham Life Sciences). The reactions were conducted in 96-well plates at room temperature, in 0.1 ml of reaction buffer containing (final concentrations): 50 mM Tris-HCl, pH 7.5, 8.3 mM MgCl₂, 1.7 mM EGTA, [³H]cAMP or [31] cGMP (approximately 2000 dpm/μmol), enzyme and various concentrations of the inhibitors. The assay was allowed to proceed for 1 hr and was terminated by adding 50 μl of SPA yttrium silicate beads in the presence of zinc sulfate. The plates were shaken and allowed to stand at room temperature for 20 min. Radiolabeled product formation was assessed by scintillation spectrometry. Activities of PDE3 and PDE7 were assessed using 0.05 μM [³H]cAMP, whereas PDE4 was assessed using 1 μM [³H]cAMP as a substrate. Activity of PDE1B, PDE1C, PDE2 and PDE5 activities were assessed using 1 μM [³H]cGMP as a substrate.

[³H]R-Rolipram Binding Assay

The [³]R-rolipram binding assay was performed by modification of the method of Schneider and co-workers, see Nicholson, et al., Trends Pharmacol. Sci., Vol. 12, pp. 19-27 (1991) and McHale et al., Mol. Pharinacol., Vol. 39, 109-113 (1991). R-rolipram binds to the catalytic site of PDE4 see Torphy et al., Mol. Pharmacol., Vol. 39, pp. 376-384 (1991). Consequently, competition for [³H]R-rolipram binding provides an independent confnmation of the PDE4 inhibitor potencies of unlabeled competitors. The assay was performed at 30° C. for 1 hr in 0.5 μl buffer containing (final concentrations): 50 nM Tris-HCl, pH 7.5, 5 mM MgCl₂, 0.05% bovine serum albumin, 2 nM [³H]R-rolipram (5.7×104 dpm/pmol) and various concentrations of non-radiolabeled inhibitors. The reaction was stopped by the addition of 2.5 ml of ice-cold reaction buffer (without [³H]-R-rolipram) and rapid vacuum filtration (Brandel Cell Harvester) through Whatman GF/1 filters that had been soaked in 0.3% polyethylenimine. The filters were washed with an additional 7.5-ml of cold buffer, dried, and counted via liquid scintillation spectrometry.

The following examples are illustrative, but not limiting of the embodiments of the present invention.

The following abbreviations are used herein
MDAP represents mass-directed auto-purification;
THF represents tetrahydrofuran;
DCM represents dichloromethane;
DMSO represents dimethyl sulfoxide;
TFA represents trifluoroacetic acid.
DDQ is 2,3,-dichloro-5,6-dicyano-1,4-benzoquinone;
PTFE is polytetrafluoroethylene;
HPLC is high performance liquid chromatography;

### DMF is N,N-dimethylforamide

EtOH is ethanol

### Conditions Hardware, and Software Used for Mass-Directed Autopurification Hardware

Waters 600 gradient pump, Waters 2700 Sample Manager, Waters Reagent Manager, Micromass ZMD mass spectrometer, Gilson 202—fraction collector, Gilson Aspec—waste collector.

### Software

Micromass Masslynx version 3.5

### Column

The column used is typically a Supelco ABZ+column whose dimensions are 10 mm internal diameter by 100 mm in length. The stationary phase particle size is 5 μm.

### Solvents

A. Aqueous solvent=Water+0.1% Formic Acid
B. Organic solvent=MeCN:Water 95:5+0.05% Formic Acid
Make up solvent=MeOH:Water 80:20+50 mMol Ammonium Acetate
Needle rinse solvent=MeOH: Water:DMSO 80:10:10

### Methods

Five methods are used depending on the analytical retention time of the compound of interest. They all have a flow rate of 20 ml/min and a 15-minute runtime, which comprises of a 10-minute gradient followed by a 5-minute column flush and re-equilibration step.

### Method 1 MDP 1.5-2.2=0-30% B

### Method 2 MDP 2.0-2.8=5-30% B

### Method 3 MDP 2.5-3.0=15-55% B

### Method 4 MDP 2.8-4.0=30-80% B

### Method 5 MDP 3.8-5.5=50-90% B

### Method Used for Purification Using the Biotage Horizon System.

### Column: Biotage C18HS 25+S

Fraction volume: 9 ml; UV Threshold: 0.03AU

### Solvent A=Water, B=Acetonitrile, Gradient:

| Volume (ml) | A | B |
|---|---|---|
| 0 | 70% | 30% |
| 240 | 0% | 100% |

Conditions used for Analytical LCMS Systems

### Hardware

Agilent 1100 gradient pump

### Agilent 1100 Autosampler

### Agilent 1100 PDA Dectector

### Agilent 1100 Degasser

Micromass ZQ mass spectrometer

### PL-ELS1000

### Software

Micromass Masslynx versions 3.5/4.0

### Column

The column used is a Supelcosil ABZ+PLUS, the dimensions of which are 4.6 mm×33 mm. The stationary phase particle size is 3 m.

### Solvents

A: Aqueous solvent=10 mMol Ammonium Acetate+0.1% Formic Acid B: Organic solvent=95% Acetonitrile+0.05% Formic Acid

### Method

The generic method used has 5.5 minute runtime, which comprises of a 4.7-minute gradient (0-100% B) followed by a 0.6 minute column flush and 0.2 minute re-equilibration step.

### Flow Rate

The above method has a flow rate of 3 ml/mins
Conditions used for NMR

### Hardware

### Bruker 400 MHz Ultrashield

### Bruker B-ACS60 Autosampler

### Bruker Advance 400 Console

### Software

User interface—NMR Kiosk
Controlling software—XWin NMR version 3.0

### REFERENCE EXAMPLE 1

### 2-(3-Chlorophenylamino)-4-trifluoromethylpyrimidine-5-carboxylic acid benzylamide

(a). To a solution of benzyl 2-chloro-4-trifluoromethylpyrimidine-5-carboxylate (0.50 g, ex Maybridge) in 1,4-dioxan (5 ml) was added 3-chloroaniline (0.85 ml) and the solution stirred at room temperature for 15 h. 1,4-Dioxan was removed under reduced pressure and ethyl acetate (15 ml) added. The solution was washed sequentially with 2N hydrochloric acid (10 ml) and water (3×10 ml), dried (MgSO₄), evaporated and triturated with hexane to afford benzyl 2-(3-chlorophenylamino)-4-trifluoromethylpyrimidine-5-carboxylate (524 mg). NMR (DMSO-d6) δ 5.35 (2H, s), 7.14 (1H, d), 7.35-7.45 (6H, m), 7.68 (1H, m), 7.98 (1H, s), 9.13 (1H, s), 10.95 (1H, s). LC/MS, t=3.70 min, [MH⁺] 408 and 410.

(b). To a solution of benzyl 2-(3-chlorophenylamino)-4-trifluoromethyl-pyrimidine-5-carboxylate (0.50 g) in ethanol (15 ml) was added a solution of potassium hydroxide (205 mg) in ethanol (10 ml) and the solution stirred at reflux for 15 h. Ethanol was removed under reduced pressure and water (15 ml) added. The solution was washed with ether and concentrated hydrochloric acid added to adjust the acidity to pH 1. The precipitated solid was filtered, washed with water and dried in vacuo at 50° C. to afford 2-(3-chlorophenylamino)-4-trifluoromethylpyrimidine-5-carboxylic acid (366 mg).

NMR (DMSO-d6) δ 7.49 (1H, d), 7.71 (1H, t), 7.98 (1H, d), 8.33 (1H, s), 9.42 (1H, s), 11.15 (1H, s), 14.0 (1H, br s). LC/MS, t=3.44 min, [MH⁺] 318 and 320.

(c). To a solution of 2-(3-chlorophenylamino)-4-trifluoromethylpyrimidine-5-carboxylic acid (35 mg) in dimethylformamide (2 ml) was added successively N-ethylmorpholine (42 μl), benzylamine (15 μl), 1-hydroxybenzotriazole hydrate (23 mg) and 1-(3-dimethylamino-propyl)-3-ethylcarbodiimide hydrochloride (25 mg). The solution was stirred for 3 h and allowed to stand overnight. Dimethylformamide was removed under reduced pressure and ethyl acetate (5 ml) added. The solution was washed sequentially with 5% sodium bicarbonate solution (2.5 ml), water (2.5 ml), 5% citric acid solution (2.5 ml) and brine (2×2.5 ml), dried (MgSO₄) and evaporated to afford the title compound (45 mg).

NMR (DMSO-d6) δ 4.47 (2H, d), 7.10 (1H, d), 7.25 (1H, m), 7.36 (5H, m), 7.69 (1H, d), 7.98 (1H, s), 8.89 (1H, s), 9.12 (1H, t), 10.65 (1H, s). LC/MS, t=3.23 min, [MH⁺] 407 and 409.

### Description 1: Methyl 6-(3-chlorophenylamino)-4-(trifluoromethyl)-nicotinate

A mixture of methyl 6-chloro-4-(trifluoromethyl)-nicotinate (0.7 g, ex Fluorochem) and 3-chloroaniline (0.62 mL) was heated at 120° C. for 6 h. The reaction mixture solidified and the crude crystals were used for the next step without further purification. LC-MS (ESI+): t=10.20 min, (MH+) 331 and 333.

### Description 2: 6-(3-Chlorophenylamino)-4-(trifluoromethyl)-nicotinic acid hydrochloride

To a suspension of methyl 6-(3-chlorophenylamino)-4-(trifluoromethyl)-nicotinate (Description 1) (1.0 g) in ethanol (5 mL) was added a solution of potassium hydroxide (510 mg) in water (5 mL) and the solution was stirred at reflux for 30 min. After removal of the ethanol under reduced pressure, the mixture was diluted with water (10 mL) and washed twice with dichloromethane. Concentrated hydrochloric acid was added to adjust pH to 1 and the precipitated solid was filtered and dried in vacuo at 60° C. to afford 6-(3-chlorophenylamino)-4-(trifluoromethyl)-nicotinic acid as its hydrochloride salt (0.62 g).

LC-MS (ESI+): t=8.51 min, (MH+) 317 and 319.

### Description 3: 6-Chloro-4-isopropyl-nicotinic acid

2M isopropylmagnesium bromide in tetrahydrofuran (48 ml) was added dropwise over 1 hour to a solution of 6-chloronicotinic acid (Aldrich) (6.0 g) in dry tetrahydrofuran (100 ml) at 0° under nitrogen and the solution stirred at 0° for 3 hours then at room temperature for 15 hours. It was cooled to −60° and acetic acid (48 ml), tetrahydrofuran (40 ml) and manganese (III) acetate dihydrate (20.4 g) added successively. The mixture was stirred at −70° for 30 minutes, then at room temperature for 1 hour. The suspension was filtered through Celite and the filtrate evaporated under reduced pressure. The residue was partitioned between dichloromethane (150 ml) and water (120 ml) and the aqueous layer separated and washed with dichloromethane (2×50 ml). The combined organic layers were dried (MgSO₄) and evaporated under reduced pressure to afford, after silica gel chromatography using 3:1 isohexane:ethyl acetate, 6-chloro-4-isopropyl-nicotinic acid (2.31 g,).

NMR (DMSO-d⁶) δ 1.21 (6H, d), 3.76 (1H, m), 7.60 (1H, s), 8.67 (1H, s), 13.55 (1H, br s).

LC/MS t=2.6 min, [MH⁺] 200 consistent with molecular formula C₉H₁₀³⁵ClNO₂

### Description 4: 6-(3-Chlorophenylamino)-4-isopropyl-nicotinic acid

A mixture of 6-chloro-4-isopropyl-nicotinic acid (Description 3) (0.50 g) and 3-chloroaniline (265 mg) was stirred at 120° for 1.5 hours. Isopropanol was added and the mixture chilled. Insoluble solid was filtered off, washed successively with isopropanol and ether and dried in vacuo at 50° to afford 6-(3-chlorophenylamino)-4-isopropyl-nicotinic acid (0.51 g).

NMR (DMSO-d⁶) δ 1.19 (6H, d), 3.93 (1H, m), 6.85 (1H, s), 6.99 (1H, d), 7.31 (1H, t), 7.53 (1H, d), 8.00 (1H, s), 8.64 (1H, s), 9.73 (1H, s), 12.6 (1H, br s).

LC/MS t=3.63 min, [MH⁺] 291, consistent with molecular formula C₁₅H₁₅³⁵ClNO₂

### Description 5: 6-Chloro-N-(4-fluoro-benzyl)-nicotinamide

A solution of 4-fluorobenzylamine (4.6 g) and triethylamine (5.57 g) in dichloromethane (60 ml) was added over 1 hour to a stirred solution of 6-chloronicotinoyl chloride (Lancaster Synthesis) (6.46 g) in dichloromethane (60 ml) at 0° under nitrogen. Stirring was continued for 1 hour, and the reaction allowed to warm to ambient temperature. The solution was diluted with dichloromethane, washed with aqueous 1M hydrochloric acid, aqueous saturated sodium bicarbonate, and water. The dried (Na₂SO₄) organic layer was evaporated to dryness, and triturated with dichloromethane to give 6-chloro-N-(4-fluoro-benzyl)-nicotinamide (6.83 g).

NMR (d⁶-DMSO) δ 4.47 (2H, d), 7.18 (2H, t), 7.37 (2H, m), 7.66 (1H, d), 8.28 (1H, d), 8.85 (1H, s), 9.31 (1H, t). LC/MS t=2.50 min, [MH⁺] 265

### Description 6: 6-Chloro-N-(4-fluoro-benzyl)-4-isopropyl-nicotinamide

Isopropylmagnesium chloride (2M in tetrahydrofuran, 38 ml) was added dropwise over 30 min to a stirred solution of 6-chloro-N-(4-fluoro-benzyl)-nicotinamide (Description 5) (6.83 g) in THF (35 ml) at 0° under nitrogen. After stirring at ambient temperature for 16 h, the solution was cooled to 0°, and treated with dry methanol (6 ml) over 3 min. After 15 min, DDQ (6.45 g) was added and stirring continued for 30 min. The mixture was concentrated under reduced pressure to 6 to 7 ml. The oil was warmed to 50°, treated with t-butyl methyl ether (120 ml), and stirred at 55° for 1 h. The mixture was filtered, and the solid washed with t-butyl methyl ether. The combined filtrates were evaporated, and the residue purified by Biotage chromatography over silica gel (40 g), eluting with isohexane/ethyl acetate (7:3) to give 6-chloro-N-(4-fluoro-benzyl)-4-isopropyl-nicotinamide (4.45 g).

NMR (d⁶-DMSO) δ 1.20 (6H, d), 3.22 (1H, multiplet), 4.46 (2H, d), 7.18 (2H, t), 7.39 (92H, m), 7.55 (1H, s), 8.37 (1H, s), 9.15 (1H, t). LC/MS t=3.0 min, [MH⁺] 307

### Description 7: 6-(3-Chloro-phenylamino)-4-trifluoromethyl-nicotinic acid

A solution of KOH (1.68 g, 31 mmol) in 30 mL of EtOH/H₂O (1:1) was added to the crude mixture from Description 1 and the resulting mixture was stirred under reflux for 3 h. The solution was concentrated in vacuo, diluted with water and washed three times (3×15 mL) with diethyl ether. Upon acidification of the aqueous layer to pH1 with 37% HCl the title compound precipitated out as the hydrochloride salt, which was filtered and dried under vacuum. The solid (2.05 g, 5.82 mmol) was then suspended in dichloromethane (25 mL), in the presence of PS-diisopropylethylamine (1.5 g, 5.8 mmol, loading 3.88 mmol/g, ex Argonaut Technologies) and stirred at room temperature for 30 min. After filtration of the resin and evaporation in vacuo of the solvent, the title compound was isolated as a white solid (1.5 g).

¹H NMR (300 MHz, DMSO-d₆) δ: 13.16 (s br, 1H); 10.28 (s, 1H); 8.80 (s, 1H); 8.01 (dd, 1H); 7.58 (ddd, 1H); 7.35 (dd, 1H); 7.28, (s, 1H); 7.06 (ddd, 1H). MS m/z (ESI+): 317 (MH⁺).

### Description 8: C-(2-Fluoro-pyridin-4-yl)-methylamine dihydrochloride

### (a). 4-Bromomethyl-2-fluoro-pyridine

To a solution of 2-fluoro-4-methylpyridine (1.0 g, ex Lancaster) in carbon tetrachloride (10 ml) was added N-bromosuccinimide (1.6 g, ex Lancaster) and 1,1′-azobis (cyclohexanecarbonitrile) (100 mg, ex Aldrich). The mixture was then refluxed for 24 h. Carbon tetrachloride was removed under reduced pressure and the crude oily solid was used in the next stage without purification.

LC/MS, t=2.38 min, [MH⁺] 190 and 192.

### (b). (2-Fluoro-pyridin-4-ylmethyl)-carbamic acid tert-butyl ester

To crude 4-bromomethyl-2-fluoro-pyridine in an ice bath was added 25% ammonia solution (10 ml, ex BDH) and the mixture stirred at 0° for 5 h. Ammonia solution was removed under reduced pressure and the yellow oily solid residue dissolved in dichloromethane (10 ml) and dimethylformamide (1 ml). The solution was cooled in an ice bath and triethylamine (1.5 ml, ex BDH) was added followed by di-tert-butyl dicarbonate (1.0 g, ex Avocado). The solution was stirred at 0° for 1 h and then the dichloromethane removed under reduced pressure. The residue was dissolved in ethyl acetate and washed twice with water, dried (MgSO₄) and evaporated to give a yellow oil. This was purified by Biotage chromatography (100 g, silica column) eluting with 30% ethyl acetate in hexane to afford the title compound as a white solid (358 mg).

NMR (DMSO-d6) δ 1.40 (9H, s), 4.20 (2H, d), 6.97 (1H, s), 7.20 (1H, d), 7.60 (1H, t), 8.17 (1H, d) ¹H NMR (300 MHz, DMSO-d₆) δ: 13.16 (s br, 1H); 9.49 (s, 1H); 8.67 (s, 1H); 7.94 (d, 1H); 7.67 (d, 1H); 7.43 (dd, 1H); 7.40 (s, 1H). MS m/z (ESI+): 351 (MH+).

### Description 13: (6-Methyl-pyridin-3-yl)-methylamine-dihydrochloride

A mixture of 5-cyano-2-methylpyridine (ex Lancaster) (0.5 g), Raney nickel (0.5 g) and acetic acid (15 ml) was hydrogenated at 50 psi for 24 hours. The catalyst was filtered off and the filtrate evaporated under reduced pressure. Water (20 ml) was added and the solution basified to pH 9 with sodium carbonate. The mixture was extracted with dichloromethane (25 ml then 2×10 ml) and the combined extracts washed with water, dried (MgSO₄), and evaporated under reduced pressure. The residue was dissolved in ether and the solution treated with 4N hydrogen chloride in dioxan (1.5 ml). The solvent was removed under reduced pressure to give, after trituration with hot isopropanol, (6-methyl-pyridin-3-yl)-methylamine dihydrochloride (35 mg).

NMR (DMSO-d6) δ 2.71 (3H, s), 4.19 (2H, d), 7.84 (1H, d), 8.43 (1H, d), 8.66 (3H, br s), 8.86 (1H, s).

### Description 14: 6-Hydroxy-2-trifluoromethyl-4,5-dihydro-pyridine-3-carboxylic acid ethyl ester

A mixture of ethyl 4,4,4-trifluoroacetoacetate (14.7 mL, 0.1 mol, 1.6 eq), acrylamide (4.5 g, 0.063 mol, 1.0 eq) and p-toluenesulphonic acid (0.156 g, 0.82 mmol, 0.013 eq) in toluene (60 mL) was refluxed for 38 h with azeotropic removal of water (Dean-Stark conditions). The reaction mixture was then concentrated to a small volume, by slow distillation of toluene at atmospheric pressure. Toluene (60 mL) was added and again the reaction mixture was concentrated, through slow distillation of toluene. After repeating this operation three times, the reaction mixture was concentrated in vacuo and the solid residue was purified by flash chromatography (silica gel, eluent gradient: from hexane/ethyl acetate 9:1 to hexane/ethyl acetate 8:2). The title compound was obtained as a brownish solid (3.8 g, yield=25%).

LC-MS (ESI+), MH+: 238, 210, 190.

### Description 15. 6-Hydroxy-2-trifluoromethyl-nicotinic acid ethyl ester

A solution of 6-hydroxy-2-trifluoromethyl-4,5-dihydro-pyridine-3-carboxylic acid ethyl ester (Description 14) (4.7 g, 19.8 mmol, 1 eq) and N-bromo succinimide (3.51 g, 19.8 mmol, 1 eq) in 15 mL of carbon tetrachloride was heated under reflux for 20 h. The resulting precipitate was filtered off and the filtrate was concentrated under reduced pressure to afford a brownish solid that was purified by flash chromatography (silica gel, eluent gradient: from hexane/ethyl acetate 9:1 to hexane/ethyl acetate 8:2). The title compound was obtained as a white solid (4.3 g, yield=92%).

LC-MS (ESI+), MH+: 236.

### Description 16. 6-Chloro-2-trifluoromethyl-nicotinic acid ethyl ester

A mixture of 6-hydroxy-2-trifluoromethyl-nicotinic acid ethyl ester (Description 15) (2.6 g, 11.0 mmol, 1.0 eq) and phenyl dichlorophosphate (2.47 mL, 16.5 mmol, 1.5 eq) was heated under microwaves irradiation for 30 min (170° C., power=70 W). The reaction mixture was poured into ice, stirred for 20 min and diluted with ethyl acetate (50 mL). The pH was adjusted to 10, by addition of a saturated aqueous solution of sodium bicarbonate (50 mL) and then the organic layer was separated, washed with water, dried over Na₂SO₄ and concentrated in vacuo. The resulting solid residue was purified by flash chromatography (silica gel, eluent gradient: from hexane to hexane/ethyl acetate 98:2) to give 1.7 g of the title compound (yield=61%).

LC-MS (ESI+), MH+: 254 and 256.

### Description 17. 6-(3-Chloro-phenylamino)-2-trifluoromethyl-nicotinic acid ethyl ester

A mixture of 6-chloro-2-trifluoromethyl-nicotinic acid ethyl ester (Description 16) (1.4 g, 5.53 mmol, 1.0 eq) and 3-chloroaniline (2.91 mL, 27.6 mmol, 5.0 eq) was heated at 160° C. for 52 h to afford a black solid which was used for the next step without further purification.

LC-MS (ESI+), MH+: 345 and 347.

### Description 18. 6-(3-Chloro-phenylamino)-2-trifluoromethyl-nicotinic acid hydrochloride

A solution of KOH (1.18 g) in water (25 mL) was added to a mixture of crude 6-(3-chloro-phenylamino)-2-trifluoromethyl-nicotinic acid ethyl ester from Description 17 in ethanol (25 mL) and refluxed for 8 h. After evaporation of ethanol under reduced pressure, the reaction mixture was diluted with water (35 mL) and repeatedly washed with diethyl ether (200 mL×5 times). The aqueous layer was treated with conc. HCl to adjust the pH to 3 and the title compound precipitated out as its hydrochloride salt, was filtered and dried at 40° C. in oven (1.71 g).

LC-MS (ESI+), MH+: 317 and 319.

### Description 19: 6-Chloro-N-cyclohexylmethyl-nicotinamide

To a solution of 6-chloronicotinoyl chloride (1.5 g, ex Lancaster) in dry dichloromethane (15 ml) was added dropwise at 0° under nitrogen a solution of cyclohexanemethanamine (1.11 ml, ex Lancaster) and triethylamine (1.5 ml) in dry dichloromethane (15 ml) over 1 hour. The solution was stirred at 0° for 1 hour. Dichloromethane was removed under reduced pressure and ethyl acetate (30 ml) added. The solution was washed with water (3×20 ml), dried (MgSO₄) and evaporated to afford 6-chloro-IV-cyclohexylmethyl-nicotinamide (1.96 g).

NMR (DMSO-d6) δ 0.85-1.0 (2H, m), 1.1-1.25 (3H, m), 1.54 (1H, m), 1.55-1.75 (5H, m), 3.11 (2H, t), 7.64 (1H, d), 8.23 (1H, d of d), 8.69 (1H, t), 8.82 (1H, s).

LC/MS t=2.9 min, Molecular ion observed [MH⁺] 253 consistent with molecular formula C₁₃H₁₇³⁵ClN₂O

### Description 20: 6-Chloro-N-cyclohexylmethyl-4-isopropyl-nicotinamide

To a solution of 6-chloro-N-cyclohexylmethyl-nicotinamide (Description 19) (0.89 g) in dry tetrahydrofuran (5 ml) was added dropwise at 0° under nitrogen a 2.0M solution of isopropylmagnesium chloride (5.3 ml, ex Aldrich) and the solution stirred at room temperature for 15 hours. It was cooled to 0° and dry methanol (0.86 ml) added dropwise and the solution stirred for 15 minutes. 2,3-Dichloro-5,6-dicyano-1,4-benzoquinone (0.88 g) was added and the mixture stirred at room temperature for 30 minutes then evaporated under reduced pressure to ca. 6 ml. The residual liquid was warmed to 50° and t-butyl methyl ether (20 ml) added. The mixture was stirred under reflux for 1 hour then at room temperature for 1 hour and filtered. The filtrate was evaporated and the residue purified using Biotage chromatography (Merck 9385 silica gel) with 1:4 ethyl acetate:isohexane to afford 6-chloro-N-cyclohexylmethyl-4-isopropyl-nicotinamide (886 mg).

NMR (DMSO-d6) δ 0.85-1.0 (2H, m), 1.1-1.25 (3H, m), 1.19 (6H, d), 1.50 (1H, m), 1.55-1.75 (5H, m), 3.08 (2H, t), 3.22 (1H, m), 7.53 (1H, s), 8.24 (1H, s), 8.57 (1H, t).

LC/MS, t=3.2 min, Molecular ion observed [MH⁺]=295 consistent with the molecular formula C₁₆H₂₃³⁵ClN₂O.

### Description 21: 6-Chloro-N-cyclobutylmethyl-nicotinamide

Prepared in a manner similar to Description 19 from 6-chloronicotinoyl chloride (1.9 g, ex-Lancaster), C-cyclobutyl-methylamine hydrochloride (1.52 g), and triethylamine (3.4 ml), to give the title compound (2.02 g).

NMR (DMSO-d6) δ 1.71 (2H, m), 1.82 (2H, m), 1.99 (2H, m), 2.52 (1H, m excess), 3.31 (2H, t), 7.64 (1H, d), 8.22 (1H, d of d), 8.71 (1H, t), 8.81 (1H, d).

LC/MS t=2.51 min, Molecular ion observed [MH⁺+]=225 consistent with the molecular formula C₁₁H₁₃³⁵ClN₂O

### Description 22: 6-Chloro-N-cyclobutylmethyl-4-isopropyl-nicotinamide

Prepared in a manner similar to Description 20 from 6-chloro-N-cyclobutylmethyl-nicotinamide (Description 19) (2.00 g), and 2.0M isopropylmagnesium chloride in THF (13.5 ml), to give the title compound (1.31 g).

NMR (DMSO-d6) δ 1.19 (6H, d), 1.72 (2H, m), 1.82 (2H, m), 1.98 (2H, m), 2.50 (1H, m excess), 3.20 (1H, m), 3.27 (2H, t), 7.53 (1H, s), 8.23 (1H, s), 8.58 (1H, t).

LC/MS t=3.07 min, [MH+]=267 consistent with the molecular formula C₁₄H₁₉³⁵ClN₂O

Description 23: 6-Chloro-N-(tetrahydro-pyran-4-ylmethyl)-nicotinamide In a manner similar to Description 21, 6-chloronicotinoyl chloride (1.90 g) and C-(tetrahydro-pyran-4-yl)-methylamine (1.65 g) afforded the title compound (1.46 g).

NMR (DMSO-d6) δ 1.1-1.25 (2H, m), 1.60 (2H, d), 1.79 (1H, m), 3.17 (2H, t), 3.26 (2H, t), 3.83 (2H, d of d), 7.64 (1H, d), 8.23 (1H, d of d), 8.75 (1H, t), 8.82 (1H, s).

LC/MS t=2.1 min, [MH⁺] 255 consistent with the molecular formula C₁₂H₁₅³⁵ClN₂O₂

### Description 24: 6-Chloro-4-isopropyl-N-(tetrahydro-pyran-4-ylmethyl)-nicotinamide

In a manner similar to Description 20, 6-chloro-N-(tetrahydro-pyran-4-ylmethyl)-nicotinamide (Description 23) (1.46 g) and 2.0M isopropylmagnesium chloride in tetrahydrofuran (8.5 ml) afforded the title compound (624 mg).

NMR (DMSO-d6) δ 1.1-1.25 (2H, m), 1.19 (6H, d), 1.60 (2H, d), 1.75 (1H, m), 3.14 (2H, t), 3.21 (1H, m), 3.27 (2H, t), 3.85 (2H, d of d), 7.54 (1H, d), 8.26 (1H, s), 8.63 (1H, t).

LC/MS t=2.4 min, [MH⁺] 297 consistent with the molecular formula C₁₂H₁₅³⁵ClN₂O₂

### Description 25: 6-Chloro-N-cyclopentylmethyl-nicotinamide

In a manner similar to Description 19, 6-chloronicotinoyl chloride (0.50 g) and cyclopentanemethylamine hydrochloride (385 mg) afforded the title compound (534 mg).

NMR (DMSO-d6) δ 1.2-1.3 (2H, m), 1.45-1.65 (4H, m), 1.65-1.75 (2H, m), 2.13 (1H, m), 3.20 (2H, t), 7.64 (1H, d), 8.23 (1H, d of d), 8.74 (1H, t), 8.82 (1H, s).

LC/MS t=2.7 min, [MH⁺] 239, consistent with the molecular formula C₁₂H₁₅³⁵ClN₂O

### Description 26: 6-Chloro-N-cyclopentylmethyl-4-isopropyl-nicotinamide

In a manner similar to Description 20, 6-chloro-N-cyclopentyl-nicotinamide (Description 25) (532 mg) and 2.0M isopropylmagnesium chloride in tetrahydrofuran (3.4 ml) afforded the title compound (166 mg).

NMR (DMSO-d6) δ 1.19 (6H, d), 1.2-1.3 (2H, m), 1.45-1.65 (4H, m), 1.65-1.75 (2H, m), 2.10 (1H, m), 3.17 (2H, t), 3.21 (1H, m), 7.53 (1H, s), 8.23 (1H, s), 8.61 (1H, t).

LC/MS t=3.1 min, [NH⁺] 281, consistent with the molecular formula C₁₅H₂₁³⁵ClN₂O.

### Description 27: 1-(6-Chloro-4-isopropyl-pyridin-3-yl)-1-morpholin-4-yl-methanone

In a manner similar to Description 20, 1-(6-chloro-pyridin-3-yl)-1-morpholin-4-yl-methanone (534 mg, Ref: US Patent Application 2002183309 (2002), and 2.0M isopropyl-magnesium chloride in tetrahydrofuran (3.6 ml) afforded the title compound (169 mg).

NMR (DMSO-d6) δ 1.19 (6H, t), 2.89 (1H, m), 3.1-3.25 (2H, m), 3.45 (1H, m), 3.55-3.75 (5H, m), 7.60 (1H, s), 8.26 (1H, s).

LC/MS t=2.3 min, [MH⁺] 269, consistent with the molecular formula C₁₃H₁₇³⁵ClN₂O₂

### Description 28: 4-tert-Butyl-6-chloro-N-cyclohexylmethyl-nicotinamide

1.6 M n-Butyllithium in hexane (2.7 ml) was added dropwise to a stirred solution of 6-chloro-N-cyclohexylmethyl-4-isopropyl-nicotinamide (Description 20) (0.50 g) in dry tetrahydrofuran (3 ml) at −70° under nitrogen. The solution was stirred for 15 minutes then warmed to 0° and a solution of methyl iodide (0.11 ml) in dry tetrahydrofuran (2 ml) added, followed by stirring for a further 30 minutes. Solvent was removed under reduced pressure and ethyl acetate (10 ml) added. The solution was washed with water (10 ml), dried (MgSO₄) and evaporated under reduced pressure. The residue was purified using silica gel chromatography with 17:3 isohexane:ethyl acetate and further purified by MDAP to afford the title compound (83 mg).

NMR (CDCl₃) δ 0.95-1.05 (2H, m), 1.15-1.3 (4H, m), 1.42 (9H, s), 1.65-1.8 (5H, m), 3.28 (2H, t), 5.81 (1H, br s), 7.36 (1H, s), 8.21 (1H, s).

LC/MS t=3.6 min, [MH⁺] 309, consistent with C₁₇H₂₅³⁵ClN₂O

Description 29: 4-tert-Butyl-6-chloro-N-(tetrahydro-pyran-4-ylmethyl)-nicotinamide In a manner similar to Description 28, 6-chloro-4-isopropyl-N-(tetrahydro-pyran-4-ylmethyl)-nicotinamide (Description 24) (1.0 g), 1.6 M n-butyllithium in hexane (2.7 ml) and methyl iodide (0.22 ml) afforded, after silica gel chromatography, eluting with 1:1 isohexane:ethyl acetate and MDAP purification, the title compound (116 mg).

NMR (CDCl₃) δ 1.3-1.45 (2H, m), 1.42 (9H, s), 1.68 (2H, d), 1.91 (1H, m), 3.34 (2H, t), 3.40 (2H, t), 4.00 (2H, d of d), 6.04 (1H, br s), 7.36 (1H, s), 8.18 (1H, s).

LC/MS t=2.4 min, [MH⁺] 311 consistent with molecular formula C₁₆H₂₃³⁵ClN₂O₂

### Description 30: 4-Aminomethyltetrahydropyran-4-ol hydrochloride

To a solution of 11.0M lithium aluminium hydride in tetrahydrofuran (20 ml) was added under a nitrogen atmosphere a solution of 4-hydroxytetra-hydropyran-4-carbonitrile (0.50 g, prepared as described in Eiden et al., Arch. Pharm., 320, 348, (1987)) in tetrahydrofuran (2 ml) and the solution stirred at reflux for 6 hours. Water (1 ml) and 2N sodium hydroxide solution (1 ml) were added cautiously and the resultant solid filtered and washed with ether. The filtrate was dried (MgSO₄), evaporated and the residue dissolved in ethanol (3 ml) and concentrated hydrochloric acid (0.5 ml) added. Solvent was removed under reduced pressure and the resultant solid washed with ether and dried in vacuo at 40° C. to afford the title compound (234 mg).

NMR (DMSO-d6) 1.45-1.6 (4H, m), 2.78 (2H, q), 3.61 (4H, m). 5.07 (1H, br s), 7.89 (3H, br s).

### Description 31: 6-(3-Chloro-phenylamino)-4-trifluoromethyl-nicotinic acid methyl ester

A mixture of methyl-6-chloro-4-(trifluoromethyl)-nicotinate (2.5 g, 10.5 mmol) and 3-chloroaniline (2.2 mL, 20.1 mmol) was heated at 120° C. for 18 h, to afford the title compound.

MS m/z (ESI+): 331 (MH+).

### Description 32: 4-Aminomethyl-pyrrolidin-2-one

Sodium (0.1 g, 4.34 mmol) was added portionwise to a solution of 4-aminomethyl-1-benzyl-pyrrolidin-2-one (0.3 g, 1.47 mmol, CAS Registry N.: 97205-34-0) in 10 mL of liquid ammonia, at −50° C. and the mixture was stirred at −50° C. for 1 h. EtOH (10 mL) was slowly added and the reaction mixture was allowed to reach room temperature and stirred for 1 h at RT. Evaporation of the solvent in vacuo afforded the title compound (0.21 g), which was used for coupling with the acids above mentioned, without further purification.

¹H NMR (300 MHz, DMSO-d₆) δ: 3.28 (dd, 1H); 2.89 (dd, 1H); 2.45 (m, 2H); 2.18-1.93 (m, 2H); 1.68 (m, 1H).

### Description 33: 6-Hydroxy-2-trifluoromethyl-4,5-dihydro-pyridine-3-carboxylic acid ethyl ester

A mixture of ethyl 4,4,4-trifluoroacetoacetate (14.7 mL, 0.1 mol, 1.6 eq), acrylamide (4.5 g, 0.063 mol, 1.0 eq) and p-toluenesulphonic acid (0.156 g, 0.82 mmol, 0.013 eq) in toluene (60 mL) was refluxed for 38 h with azeotropic removal of water (Dean-Stark conditions). The reaction mixture was then concentrated to a small volume, by slow distillation of toluene at atmospheric pressure. Toluene (60 mL) was added and again the reaction mixture was concentrated, through slow distillation of toluene. After repeating this operation three times, the reaction mixture was concentrated in vacuo and the solid residue was purified by flash chromatography (silica gel, eluent gradient: from hexane/ethyl acetate 9:1 to hexane/ethyl acetate 8:2). The title compound was obtained as a brownish solid (3.8 g, yield=25%).

LC-MS (ESI+), MH+: 238, 210, 190.

### Description 34: 3-Amino-4-methyl-pent-2-enoic acid ethyl ester

Ammonium acetate (2.44 g, 31.6 mol, 5 eq) was added to a solution of 4-methyl-3-oxo-pentanoic acid ethyl ester (1.0 g, 6.32 mol, 1 eq) in methanol (10 mL) and the mixture was stirred at room temperature for 3 days. Solvent was evaporated in vacuo and the solid residue was triturated with dichloromethane (20 mL) and filtered off. The filtrate was then washed with water and brine, dried over Na₂SO₄ and concentrated in vacuo to afford the title compound as a yellow oil (0.85 g, yield=85%).

### Description 35: 4-(1-Amino-2-methyl-propylidene)-pent-2-enedioic acid 5-ethyl ester 1-methyl ester

A solution of 3-amino-4-methyl-pent-2-enoic acid ethyl ester (Description 34) (5.0 g, 31.84 mmol, 1 eq) and methyl propiolate (3.08 mL, 36.8 mmol, 1.15 eq) in dry DMSO (20 mL) was heated under microwave irradiation at 170° C. (1^{st} cycle: 20 min, 2^{nd} cycle: 10 min). The reaction mixture was diluted with water (140 mL) and extracted twice with ethyl acetate (80 mL). The organic phase was washed with a saturated aqueous solution of NaHCO₃ and with brine, dried over sodium sulphate and concentrated in vacuo to afford 9.5 g of yellow solid, used for the next step without further purification.

LC-MS (ESI+), MH+: 242, 196.

### Description 36: 6-Hydroxy-2-isopropyl-nicotinic acid ethyl ester

A catalytic amount of sodium tert-butoxide (100 mg) was added to a suspension of crude 4-(1-amino-2-methyl-propylidene)-pent-2-enedioic acid 5-ethyl ester 1-methyl ester (Description 35) (9.5 g) in anhydrous ethanol (100 mL) and the resulting mixture was refluxed for 28 h. Solvent was removed in vacuo, the residue was taken up with ethyl acetate and then washed subsequently with NaHCO₃ (aq) and with brine. The organic layer was dried over Na₂SO₄ and concentrated in vacuo to afford a reddish resin. Trituration of the resin with hexane/diethyl ether 1:1 yielded the title compound as a solid that was filtered off and dried in oven (1.97 g). The mother liquor was concentrated and purified by flash chromatography (silica gel, eluent gradient: from hexane/ethyl acetate 9:1 to hexane/ethyl acetate 7:3) to yield a second crop of pure title compound (1.6 g, total yield of Descriptions 35 and 36=54%).

LC-MS (ESI+), MH+: 210.

### Description 37: 6-Chloro-2-isopropyl-nicotinic acid ethyl ester

A mixture of 6-hydroxy-2-isopropyl-nicotinic acid ethyl ester (Description 36) 1.0 g, 4.78 mmol, 1.0 eq) and phenyl dichlorophosphate (1.13 mL, 7.56 mmol, 1.5 eq) was heated under microwaves irradiation at 170° C. for 1 min. The reaction mixture was poured into ice-water (25 mL), stirred for 20 min and diluted with ethyl acetate (40 mL). The pH was adjusted to 10, by addition of a saturated aqueous solution of sodium bicarbonate (50 mL) and then the organic layer was separated, washed with water, dried over Na₂SO₄ and concentrated in vacuo to give 1.11 g of the crude title compound as a black resin (yield=99%).

LC-MS (ESI+), MH+: 228 and 230.

### Description 38: 6-(3-Chloro-phenylamino)-2-isopropyl-nicotinic acid ethyl ester

A mixture of 6-chloro-2-isopropyl-nicotinic acid ethyl ester (Description 37) (1.1 g, 4.84 mmol, 1.0 eq) and 3-chloro aniline (1.54 mL, 14.5 mmol, 3.0 eq) was heated at 120° C. for 4 h to afford a solid residue which was used for the next step without further purification.

LC-MS (ESI+), MH+: 319 and 321.

### Description 39: 6-(3-Chloro-phenylamino)-2-isopropyl-nicotinic acid hydrochloride

A solution of KOH (1.08 g) in water (10 mL) was added to a mixture of crude 6-(3-chloro-phenylamino)-2-isopropyl-nicotinic acid ethyl ester (Description 38) in ethanol (10 mL) and refluxed for 4 h. After evaporation of ethanol under reduced pressure, the reaction mixture was diluted with water (15 mL) and repeatedly washed with diethyl ether (40 mL×4 times). The aqueous layer was treated with conc. HCl to adjust the pH to 1 and the title compound precipitated out as its hydrochloride salt, was filtered and dried at 40° C. in oven (0.68 g). The aqueous mother liquor was treated with NaCl (s) and repeatedly extracted with ethyl acetate (30 mL×3 times), the organic layer was dried over sodium sulphate and evaporated in vacuo. The residue was treated with conc. HCl and the title compound that precipitated out was filtered and dried in oven (0.681 g, total yield of Description 38 and 39=85%).

LC-MS (ESI+), MH+: 291 and 293.

### Description 40: 6-Chloro-N-(1,1-dioxo-tetrahydro-1⁶-thiophen-3-ylmethyl)-4-isopropyl-nicotinamide

To a solution of 6-chloro-4-isopropyl-nicotinic acid (Description 3) (100 mg) in dimethylformamide (7 ml) was added successively N-ethylmorpholine (0.22 ml), C-(1,1-dioxo-tetrahydro-1⁶-thiophen-3-ylmethyl)-methylamine hydrochloride (111 mg, Ref.: Argyle et al., J. Chem. Soc., (C), 2156, (1967)), 1-hydroxybenzotriazole hydrate (120 mg) and 1-(3-dimethylamino-propyl)-3-ethylcarbodiimide hydrochloride (120 mg). The solution was stirred for 5 h and allowed to stand overnight. Dimethylformamide was removed under reduced pressure and ethyl acetate (20 ml) added. The solution was washed sequentially with 5% sodium bicarbonate solution (12 ml), water (12 ml) and brine (2×12 ml), dried (MgSO₄) and evaporated to afford the title compound (150 mg).

LC/MS t=2.1 min, [MH⁺] 331 consistent with the molecular formula C₁₄H₁₉³⁵ClN₂O₃S.

The amines which are coupled with acids to make the following Examples are all commercially available, except, C-(2-fluoro-pyridin-4-yl)-methylamine dihydrochloride, (Description 8), and C-(1H-imidazol-2-yl)-methylamine which has the CAS-Registry number 53332-80-2, and for which a synthetic procedure is disclosed in the literature, 4-aminomethyl-benzamide (Example 387)—UpJohn Patent application WO97/45403 (1997), N-(4-aminomethyl)-phenyl)-methansulfonamide (Example 391) Schering patent application WO90/00548, 4-aminomethyl-N-methyl-benzamide (Example 392) where the freebase is prepared as in WO94/17035 which can be converted to the hydrochloride salt by known means, and the following amines known in the literature

| Structure | CAS Registry Number |
|---|---|
| 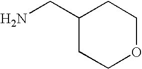 | 130290-79-8 |
| 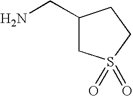 | 45697-13-0 |
| 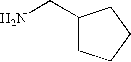 | 6053-81-2 |
| 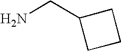 | 4415-83-2 |
| 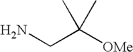 | 89282-70-2 |
| 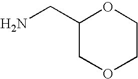 | 88277-83-2 |
| 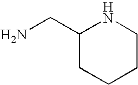 | 22990-77-8 |
| 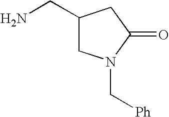 | 97205-34-0 |
| 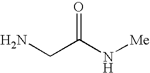 | 22356-89-4 |
| 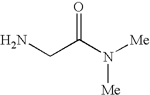 | 1857-19-8 |

### EXAMPLE 1

### 1-[2-(3-Chlorophenylamino)-4-trifluoromethylpyrimidin-5-yl]-1-piperidin-1-ylmethanone

In a manner similar to Reference Example 1(c) 2-(3-chlorophenylamino)-4-trifluoromethylpyrimidine-5-carboxylic acid (35 mg) and piperidine (13 μl) afforded the title compound (38 mg).

NMR (DMSO-d6) δ 1.3-1.65 (6H, m), 3.28 (2H, s), 3.6 (2H, br s), 7.10 (1H, d), 7.37 (1H, t), 7.68 (1H, d), 7.96 (1H, s), 8.78 (1H, s), 10.55 (1H, s) LC/MS, t=3.63 min, [MH⁺] 385 and 387.

### EXAMPLE 2

### 2-(3-Chlorophenylamino)-4-trifluoromethylpyrimidine-5-carboxylic acid cyclopentylmethyl-amide

In a manner similar to Reference Example 1(c) 2-(3-chlorophenylamino)-4-trifluoromethyl-pyrimidine-5-carboxylic acid (100 mg) and cyclopentylmethylamine hydrochloride (63 mg, prepared as described in Kelley et al., J. Med. Chem., 40 3207, (1997)) afforded the title compound (80 mg).

NMR (DMSO-d6) δ 1.20-1.26 (2H, m), 1.48-1.67 (4H, m), 1.67-1.73 (2H, m), 2.06-2.10 (1H, quintuplet), 3.15-3.18 (2H, t), 7.09 (1H, dt), 7.37 (1H, q), 7.67 (1H, d), 7.96 (1H, d), 8.60-8.63 (1H, t), 8.79 (1H, s), 10.60 (1H, s). LC/MS, t=3.73 min [MH⁺] 399.

### EXAMPLE 3

### 1-[2-(3-Chlorophenylamino)-4-trifluoromethylpyrimidin-5-yl]-1-morpholin-4-yl-methanone

In a manner similar to Reference Example 1(c) 2-(3-chlorophenylamino)-4-trifluoromethylpyrimidine-5-carboxylic acid (35 mg) and morpholine (11.5 μl) afforded the title compound (43 mg).

NMR (DMSO-d6) δ 3.4-3.75 (8H, m), 7.10 (1H, d), 7.38 (1H, t), 7.68 (1H, d), 7-98 (1H, s), 8.80 (1H, s), 10.60 (1H, s). LC/MS, t=3.29 min [MH⁺] 387 and 389.

### EXAMPLE 4

### 2-(3-Chlorophenylamino)-4-trifluoromethylpyrimidine-5-carboxylic acid cyclohexylmethylamide

In a manner similar to Reference Example 1(c) 2-(3-chlorophenylamino)-4-trifluoromethylpyrimidine-5-carboxylic acid (35 mg) and cyclohekanemethylamine (15 mg) afforded the title compound (27 mg).

NMR (DMSO-d6) δ 0.85-1.0 (2H, m), 1.1-1.3 (3H, m), 1.5 (1H, m), 1.55-1.8 (5H, m), 3.06 (2H, t), 7.09 (1H, d), 7.37 (1H, t), 7.68 (1H, d), 7.97 (1H, s), 8.58 (1H, t), 8.79 (1H, s), 10.6 (1H, s).

LC/MS, t=3.87 min, [MH⁺] 413 and 415.

### EXAMPLE 5

### 2-Phenylamino-4-trifluoromethylpyrimidine-5-carboxylic acid cyclohexyl-methyl-amide

In a manner similar to Reference Example 1(c) 2-phenylamino-4-trifluoromethylpyrimidine-5-carboxylic acid (32 mg) and cyclohexanemethylamine (15 mg) afforded the title compound (33 mg).

NMR (DMSO-d6) δ 0.85-1.0 (2H, m), 1.05-1.3 (3H, m), 1.5 (1H, m), 1.55-1.8 (5H, m), 3.08 (2H, t), 7.06 (1H, d), 7.35 (2H, t), 7.76 (2H, d), 8.56 (1H, t), 8.74 (1H, s), 10.4 (1H, s).

LC/MS, t=3.66 min, [MH⁺] 379.

### EXAMPLE 6

### 1-[2-(2,3-Dichlorophenylamino)-4-trifluoromethylpyrimidin-5-yl]-1-morpholin-4-yl-methanone

In a manner similar to Reference Example 1(c) 2-(2,3-dichlorophenylamino)-4-trifluoromethyl-pyrimidine-5-carboxylic acid (24 mg) and morpholine (10 μl) afforded the title compound (17 mg). NMR (DMSO-d6) δ 3.4-3.8 (8H, m), 7.40 (1H, t), 7.54 (1H, d), 7.60 (1H, d), 8.78 (1H, s), 10.15 (1H, s). LC/MS, t=3.32 min, [MH⁺] 421 and 423.

### EXAMPLE 7

### 1-[2-(2,4-Dichlorophenylamino)-4-trifluoromethylpyrimidin-5-yl]-1-morpholin-4-yl-methanone

In a manner similar to Reference Example 1(c) 2-(2,4-dichlorophenylamino)-4-trifluoromethyl-pyrimidine-5-carboxylic acid (30 mg) and morpholine (10 μl) afforded the title compound (31 mg). NMR (DMSO-d6) δ 3.3-3.8 (8H, m), 7.52 (1H, d of d), 7.68 (1H, d), 7.76 (1H, d), 9.73 (1H, s), 10.05 (1H, s). LC/MS, t=3.37 min, [Mt] 421 and 423.

### EXAMPLE 8

### 1-[2-(3,4-Dichlorophenylamino)-4-trifluoromethylpyrimidin-5-yl]-1-morpholin-4-yl-methanone

In a manner similar to Reference Example 1(c) 2-(3,4-dichlorophenylamino)-4-trifluoromethyl-pyrimidine-5-carboxylic acid (30 mg) and morpholine (10 μl) afforded the title compound (36 mg). NMR (DMSO-d6) δ 3.35-3.8 (8H, m), 7.67 (1H, d), 7.76 (1H, d of d), 8.22 (1H, s), 8.90 (1H, s), 10.80 (1H, s). LC/MS, t=3.45 min [MH⁺] 421 and 423.

### EXAMPLE 9

### 1-[2-(2,5-Dichlorophenylamino)-4-trifluoromethylpyrimidin-5-yl]-1-morpholin-4-yl-methanone

In a manner similar to Reference Example 1(c) 2-(2,5-dichlorophenylamino)-4-trifluoromethyl-pyrimidine-5-carboxylic acid (35 mg) and morpholine (14.5 μl) afforded the title compound (27 mg).

NMR (DMSO-d6) δ 3.4-3.75 (8H, m), 7.32 (1H, d of d), 7.66 (1H, d), 7.78 (1H, d), 8.71 (1H, s), 10.05 (1H, s). LC/MS, t=3.31 min, [MH⁺] 421 and 423.

### EXAMPLE 10

### 1-[2-(3-Fluorophenylamino)-4-trifluoromethylpyrimidin-5-yl]-1-morpholin-4-yl-methanone

In a manner similar to Reference Example 1(c) 2-(3-fluorophenylamino)-4-trifluoromethylpyrimidine-5-carboxylic acid (35 mg) and morpholine (12 μl) afforded the title compound (31 mg).

NMR (DMSO-d6) δ 3.4-3.8 (8H, m), 6.85 (1H, t of d), 7.37 (1H, q), 7.52 (1H, d), 7.77 (1H, d of t), 8.80 (1H, s), 10.65 (1H, s). LC/MS, t=3.06 min, [MH⁺] 371.

### EXAMPLE 11

### 1-[2-(3-Bromophenylamino)-4-trifluoromethylpyrimidin-5-yl]-1-morpholin-4-yl-methanone

In a manner similar to Reference Example 1(c) 2-(3-bromophenylamino)-4-trifluoromethylpyrimidine-5-carboxylic acid (35 mg) and morpholine (10 μl) afforded the title compound (31 mg).

NMR (DMSO-d6) δ 3.4-3.8 (8H, m), 7.22 (1H, d), 7.30 (1H, t), 7.71 (1H, d), 8.11 (1H, s), 8.81 (1H, s), 10.60 (1H, s). LC/MS, t=3.25 min, [MN⁺] 431 and 433.

### EXAMPLE 12

### 1-[2-(3-Bromophenylamino)-4-trifluoromethylpyrimidin-5-yl]-1-piperidin-4-ylmethanone

In a manner similar to Reference Example 1(c) 2-(3-bromophenylamino)-4-trifluoromethylpyrimidine-5-carboxylic acid (35 mg) and piperidine (12 μl) afforded the title compound (31 mg).

NMR (DMSO-d6) δ 1.3-1.7 (6H, m), 3.26 (2H, s), 3.60 (2H, br s), 7.21 (1H, d), 7.30 (1H, t), 7.70 (1H, d), 8.11 (1H, s), 8.78 (1H, s), 10.55 (1H, s). LC/MS, t=3.57 min, [MH⁺] 429 and 431.

### EXAMPLE 13

### 1-[2-(3,5-Dichlorophenylamino)-4-trifluoromethylpyrimidin-5-yl]-1-morpholin-4-yl-methanone

In a manner similar to Reference Example 1(c) 2-(3,5-dichlorophenylamino)-4-trifluoromethyl-pyrimidine-5-carboxylic acid (35 mg) and morpholine (14.5 μl) afforded the title compound (42 mg).

NMR (DMSO-d6) δ 3.4-3.75 (8H, m), 7.35 (1H, s), 7.89 (2H, s), 8.87 (1H, s), 10.80 (1H, s).

LC/MS, t=3.52 min, [MH⁺] 421 and 423.

### EXAMPLE 14

### 2-(3-Chlorophenylamino)-4-trifluoromethylpyrimidine-5-carboxylic acid cyclopentylamide

In a manner similar to Reference Example 1(c) 2-(3-chlorophenylamino)-4-trifluoromethylpyrimidine-5-carboxylic acid (35 mg) and cyclopentylamine (13 gl) afforded the title compound (34 mg).

NMR (DMSO-d6) δ 1.5 (4H, m), 1.65 (2H, m), 1.85 (2H, m), 4.15 (1H, m), 7.09 (1H, d), 7.36 (1H, t), 7.67 (1H, d), 7.97 (1H, s), 8.55 (1H, d), 8.79 (1H, s), 10.60 (1H, s). LC/MS, t=3.55 min, [MH⁺] 385 and 387.

### EXAMPLE 15

### 2-(2,3-Dichlorophenylamino)-4-trifluoromethylpyrimidine-5-carboxylic acid cyclohexylmethyl-amide

In a manner similar to Reference Example 1(c) 2-(2,3-dichlorophenylamino)-4-trifluoromethyl-pyrimidine-5-carboxylic acid (35 mg) and cyclohexanemethylamine (16 μl) afforded the title compound (30 mg).

NMR (DMSO-d6) δ 0.85-1.0 (2H, m), 1.1-1.3 (3H, m), 1.45 (1H, m), 1.55-1.8 (5H, m), 3.05 (2H, t), 7.40 (1H, t), 7.55 (2H, d), 8.53 (1H, t), 8.65 (1H, s), 10.15 (1H, s). LC/MS, t=3.84 min, [MR⁺] 447 and 449.

### EXAMPLE 16

### 2-(2,4-Dichlorophenylamino)-4-trifluoromethylpyrimidine-5-carboxylic acid cyclohexylmethyl-amide

In a manner similar to Reference Example 1(c) 2-(2,4-dichlorophenylamino)-4-trifluoromethyl-pyrimidine-5-carboxylic acid (35 mg) and cyclohexanemethylamine (16 μl) afforded the title compound (14 mg).

NMR (DMSO-d6) δ 0.85-1.0 (2H, m), 1.1-1.3 (3H, m), 1.45 (1H, m), 1.55-1.75 (5H, m), 3.05 (2H, t), 7.46 (1H, d), 7.57 (1H, d), 7.72 (1H, s), 8.53 (1H, t), 8.64 (1H, s), 10.00 (1H, s).

LC/MS, t=3.90 min, [MH⁺] 447 and 449.

### EXAMPLE 17

### 2-(3,4-Dichlorophenylamino)-4-trifluoromethylpyrimidine-5-carboxylic acid cyclohexylmethyl-amide

In a manner similar to Reference Example 1(c) 2-(3,4-dichlorophenylamino)-4-trifluoromethyl-pyrimidine-5-carboxylic acid (35 mg) and cyclohexanemethylamine (16 μl) afforded the title compound (31 mg).

NMR (DMSO-d6) δ 0.8-1.0 (2H, m), 1.1-1.3 (3H, m), 1.5 (1H, m), 1.55-1.8 (5H, m), 3.06 (2H, t), 7.62 (1H, d), 7.69 (1H, d), 8.18 (1H, s), 8.59 (1H, t), 8.82 (1H, s), 10.70 (1H, s).

LC/MS, t=4.01 min, [MH⁺] 447 and 449.

### EXAMPLE 18

### 2-(3,5-Dichlorophenylamino)-4-trifluoromethylprimidine-5-carboxylic acid cyclohexylmethyl-amide

In a manner similar to Reference Example 1(c) 2-(3,5-dichlorophenylamino)-4-trifluoromethyl-pyrimidine-5-carboxylic acid (35 mg) and cyclohexanemethylamine (16 μl) afforded the title compound (30 mg).

NMR (DMSO-d6) δ 0.85-1,0-(2H, m), 1.1-1.3 (3H, m), 1.5 (1H, m), 1.55-1.8 (5H, m), 3.07 (2H, t), 7.26 (1H, s), 7.89 (2H, s), 8.58 (1H, t), 8.86 (1H, s), 10.80 (1H, s). LC/MS, t=4.08 min, [MH⁺] 447 and 449.

### EXAMPLE 19

### 2-(3-Fluorophenylamino)-4-trifluoromethylpyrimidine-5-carboxylic acid cyclohexylmethyl-amide

In a manner similar to Reference Example 1(c) 2-(3-fluorophenylamino)-4-trifluoromethyl-pyrimidine-5-carboxylic acid (35 mg) and cyclohexanemethylamine (18 μl) afforded the title compound (38 mg).

NMR (DMSO-d6) δ 0.85-1.0 (2H, m), 1.1-1.3 (3H, m), 1.5 (1H, m), 1.55-1.8 (5H, m), 3.09 (2H, t), 6.87 (1H, t of d), 7.39 (1H, q), 7.53 (1H, d), 7.78 (1H, d of t), 8.59 (1H, t), 8.80 (1H, s), 10.60 (1H, s). LC/MS, t=3.68 min, [MH⁺] 397.

### EXAMPLE 20

### 2-(3-Bromophenylamino)-4-trifluoromethylpyrimidine-5-carboxylic acid cyclohexylmethyl-amide

In a manner similar to Reference Example 1(c) 2-(3-bromophenylamino)-4-trifluoromethyl-pyrimidine-5-carboxylic acid (35 mg) and cyclohexanemethylamine (15 μl) afforded the title compound (36 mg).

NMR (DMSO-d6) δ 0.85-1.0 (2H, m), 1.1-1.3 (3H, m), 1.5 (1H, m), 1.55-1.8 (5H, m), 3.08 (2H, t), 7.23 (1H, d), 7.31 (1H, t), 7.71 (1H, d), 8.10 (1H, s), 8.57 (1H, t), 8.80 (1H, s), 10.60 (1H, s).

LC/MS, t=3.85 min, [MR⁺] 457 and 459.

### EXAMPLE 21

### 2-(2,6-Dichlorophenylamino)-4-trifluoromethylpyrimidine-5-carboxylic acid cyclohexylmethyl-amide

In a manner similar to Reference Example 1(c) 2-(2,6-dichlorophenylamino)-4-trifluoromethyl-pyrimidine-5-carboxylic acid (33 mg) and cyclohexanemethylamine (15 μl) afforded the title compound (9 mg).

NMR (DMSO-d6) δ 0.85-1.0 (2H, m), 1.05-1.25 (3H, m), 1.46 (1H, m), 1.55-1.8 (5H, m), 3.04 (2H, t), 7.39 (1H, t), 7.59 (2H, d), 8.56 (2H, m), 10.10 (1H, s). LC/MS, t=3.84 min, [MH⁺] 447 and 449.

### EXAMPLE 22

### 2-(3-Chlorophenylamino)-4-trifluoromethylpyrimidine-5-carboxylic acid (tetrahydro-pyran-4-ylmethyl)-amide

In a manner similar to Reference Example 1(c) 2-(3-chlorophenylamino)-4-trifluoromethyl-pyrimidine-5-carboxylic acid (30 mg) and 4-aminomethyltetrahydropyran (13 mg) afforded the title compound (25 mg).

NMR (DMSO-d6) δ 1.15-1.3 (2H, m), 1.62 (2H, d), 1.74 (1H, m), 3.13 (2H, t), 3.27 (2H, t), 3.86 (2H, d), 7.10 (1H, d), 7.37 (1H, t), 7.66 (1H, d), 7.97 (1H, s), 8.63 (1H, t), 8.82 (1H, s), 10.60 (1H, s). LC/MS, t=3.22 min, [MH⁺] 415 and 417.

### EXAMPLE 23

### 2-(3-Chlorophenylamino)-4-trifluoromethylpyrimidine-5-carboxylic acid cyclobutyl-amide

In a manner similar to Reference Example 1(c) 2-(3-chlorophenylamino)-4-trifluoromethyl-pyrimidine-5-carboxylic acid (35 mg) and cyclobutylamine (10 μl) afforded the title compound (28 mg).

NMR (DMSO-d6) δ 1.6-1.75 (2H, m), 1.9-2.05 (2H, m), 2.2-2.3 (2H, m), 4.32 (1H, m), 7.10 (1H, d), 7.37 (1H, t), 7.67 (1H, d), 7.96 (1H, s), 8.82 (2H, s), 10.60 (1H, s).

LC/MS, t=3.45 min, [MH⁺] 371 and 373.

### EXAMPLES 24 TO 30

Examples 24 to 30, were prepared in a manner similar to that in Reference Example 1.

TABLE 1

| | | LC/MS |
|---|---|---|
| | | 1 Retention |
| | | time (min) |
| Ex. | | 2 MH⁺ |
| No. | Compound name | 3 Formula |
| 24 | 1-(2-Phenylamino-4-trifluoromethyl- | 3.38 |
| | pyrimidin-5-yl)-1-piperidin-1-yl-methanone | 351 |
| | | C₁₇H₁₇F₃N₄O |
| 25 | 1-Morpholin-4-yl-1-(2-phenylamino-4- | 3.04 |
| | trifluoromethyl-pyrimidin-5-yl)-methanone | 353 |
| | | C₁₆H₁₅F₃N₄O₂ |
| 26 | 2-(3-Chloro-phenylamino)-4- | 3.27 |
| | trifluoromethyl-pyrimidine-5-carboxylic acid | 356 |
| | cyanomethyl-amide | C₁₄H₉³⁵ClF₃N₅O |
| 27 | 2-(3-Chloro-phenylamino)-4- | 3.80 |
| | trifluoromethyl-pyrimidine-5-carboxylic acid | 401 |
| | (3,3-dimethyl-butyl)-amide | C₁₈H₂₀³⁵ClF₃N₄O |
| 28 | 2-(3-Chloro-phenylamino)-4- | 3.69 |
| | trifluoromethyl-pyrimidine-5-carboxylic acid | 387 |
| | (2,2-dimethyl-propyl)-amide | C₁₇H₁₈³⁵ClF₃N₄O |
| 29 | 2-(3-Fluoro-phenylamino)-4-trifluoromethyl- | 3.29 |
| | pyrimidine-5-carboxylic acid | 355 |
| | cyclobutylamide | C₁₆H₁₄F₄N₄O |
| 30 | 2-(3,4-Dichloro-phenylamino)-4- | 3.66 |
| | trifluoromethyl-pyrimidine-5-carboxylic acid | 405 |
| | cyclobutylamide | C₁₆H₁₃³⁵Cl₂F₃N₄O |

### EXAMPLE 31

### 2-(3-Fluorophenylamino)-4-trifluoromethylpyrimidine-5-carboxylic acid (tetrahydropyran-4-ylmethyl)amide

In a manner similar to Reference Example 1(c) 2-(3-fluorophenylamino)-4-trifluoromethyl-pyrimidine-5-carboxylic acid (35 mg) and 4-aminomethyltetrahydropyran (16 mg) afforded the title compound (38 mg).

NMR (DMSO-d6) δ 1.15-1.3 (2H, m), 1.63 (2H, d), 1.75 (1H, m), 3.15 (2H, t), 3.29 (2H, t), 3.86 (2H, d), 6.88 (1H, td), 7.38 (1H, q), 7.51 (1H, d), 7.76 (1H, dt), 8.64 (1H, t), 8.82 (1H, s), 10.60 (1H, s). LC/MS, t=3.08 min, [MH⁺] 399.

### EXAMPLE 32

### 2-(3-Bromophenylamino)-4-trifluoromethylpyrimidine-5-carboxylic acid (tetrahydropyran-4-ylmethyl)amide

In a manner similar to Reference Example 1(c) 2-(3-bromophenylamino)-4-trifluoromethyl-pyrimidine-5-carboxylic acid (3 mg) and 4-aminomethyltetrahydropyran (13 5 mg) afforded the title compound (36 mg).

NMR (DMSO-d6) δ 1.15-1.3 (2H, m), 1.62 (2H, d), 1.74 (1H, m), 3.13 (2H, t), 3.27 (2H, t), 3.86 (2H, d), 7.23 (1H, d), 7.31 (1H, t), 7.71 (1H, d), 8.11 (1H, s), 8.63 (1H, t), 8.82 (1H, s), 10.60 (1H, s). LC/MS, t=3.26 min, [MH⁺] 459 and 461.

### EXAMPLE 33

### 2-(2,3-Dichlorophenylamino)-4-trifluoromethylpyrimidine-5-carboxylic acid (tetrahydropyran-4-ylmethyl)amide

In a manner similar to Reference Example 1(c) 2-(2,3-dichlorophenylamino)-4-trifluoromethyl-pyrimidine-5-carboxylic acid (30 mg) and 4-aminomethyltetrahydropyran (12 mg) afforded the title compound (25 mg).

NMR (DMSO-d6) δ 1.1-1.25 (2H, m), 1.60 (2H, d), 1.72 (1H, m), 3.11 (2H, t), 3.26 (2H, t), 3.85 (2H, d), 7.40 (1H, t), 7.55 (2H, d), 8.60 (1H, t), 8.66 (1H, s), 10.10 (1H, s). LC/MS, t=3.29 min, [MH⁺] 449 and 451.

### EXAMPLE 34

### 2-(2,4-Dichlorophenylamino)-4-trifluoromethylpyrimidine-5-carboxylic acid (tetrahydropyran-4-ylmethyl)-amide

In a manner similar to Reference Example 1(c) 2-(2,4-dichlorophenylamino)-4-trifluoromethyl-pyrimidine-5-carboxylic acid (30 mg) and 4-aminomethyltetrahydropyran (12 mg) afforded the title compound (34 mg).

NMR (DMSO-d6) δ 1.1-1.25 (2H, m), 1.59 (2H, d), 1.72 (1H, m), 3.11 (2H, t), 3.26 (2H, t), 3.85 (2H, d), 7.47 (1H, dd), 7.57 (1H, d), 7.72 (1H, s), 8.60 (1H, t), 8.65 (1H, s), 10.05 (1H, s).

LC/MS, t=3.33 min, [MH⁺] 449 and 451.

### Additional synthesis of Example 34: 2-(2,4-Dichlorophenylamino)-4-trifluoromethyl-pyrimidine-5-carboxylic acid (tetrahydro-pyran-4-ylmethyl)-amide

(a). To a solution of methyl 2-chloro-4-trifluoromethyl-pyrimidine-5-carboxylate (0.50 g, ex Maybridge) in 1,4-dioxan (5 ml) was added 2,4-dichloroaniline (1.7 g) and the solution stirred under reflux for 7 h. 1,4-Dioxan was removed under reduced pressure and ethyl acetate (15 ml) added. The solution was washed sequentially with 2N hydrochloric acid (10 ml) and water (3×10 ml), dried (MgSO₄), evaporated and triturated with hexane to afford methyl 2-(2,4-dichlorophenylamino)-4-trifluoromethylpyrimidine-5-carboxylate (358 mg).

NMR (CDCl₃) δ 3.95 (3H, s), 7.30 (1H, dd), 7.45 (1H, d), 8.00 (1H, s), 8.5 (1H, d), 9.05 (1H, s). LC/MS, t=2.60 min, [M-Me₂C═CH2+H⁺] 171

c) (2-Fluoro-pyridin-4-ylmethyl)-carbamic acid tert-butyl ester (350 mg) was treated at room temperature with 4N hydrochloric acid in 1,4-dioxan (5 ml) and stirred for 2 h. The white precipitate was filtered, washed with fresh ether and dried to afford the title compound (200 mg).

NMR (400 MHz, DMSO-d6) δ 4.14 (2H, d), 7.38 (1H, s), 7.51 (1H, d), 8.28 (1H, d), 8.82 (3H, s).

### Description 9: 6-(2,3-Dichloro-phenylamino)-4-trifluoromethyl-nicotinic acid methyl ester

A mixture of methyl-6-chloro-4-(trifluoromethyl)-nicotinate (2.0 g, 8.37 mmol, ex Fluorochem) and 2,3-dichloroaniline (4.06 g, 25 mmol) was heated at 13° C. for 18 h, to afford the title compound that was used for the next step without further purification.

MS m/z (ESI+): 365 (MH+).

### Description 10: 6-(2,3-Dichloro-phenylamino)-4-trifluoromethyl-nicotinic acid

A solution of KOH (1.4 g, 25 mmol) in 20 mL of EtOH/H₂O (1:1) was added to the crude mixture from Description 9 and the resulting mixture was stirred under reflux for 3 h. The solution was concentrated in vacuo, diluted with water and washed three times (3×15 mL) with diethyl ether. Upon acidification of the aqueous layer to pH1 with 37% HCl, the title compound precipitated out as hydrochloride salt which was filtered and dried under vacuum. The solid (2.7 g, 7 mmol) was then suspended in dichloromethane (20 mL), in the presence of PS-diisopropylethylamine (1.80 g, 7 mmol, loading 3.88 mmol/g, ex Argonaut Technologies) and stirred at room temperature for 30 min. After filtration of the resin and evaporation in vacuo of the solvent, the title compound was isolated as a white solid (2.45 g).

¹H NMR (300 MHz, DMSO-d₆) δ: 13.17 (s br, 1H); 9.61 (s, 1H); 8.68 (s, 1H); 7.88 (dd, 1H); 7.44 (dd, 1H); 7.42 (s, 1H); 7.37 (dd, 1H). MS m/z (ESI+): 351 (MH+).

### Description 11: 6-(2,4-Dichloro-phenylamino)-4-trifluoromethyl-nicotinic acid methyl ester

A mixture of methyl-6-chloro-4-(trifluoromethyl)-nicotinate (2.0 g, 8.37 mmol ex Fluorochem) and 2,4-dichloroaniline (4.05 g, 25 mmol) was heated at 130° C. for 15 h, to afford the title compound that was used for the next step without further purification.

MS m/z (ESI+): 365 (MH+).

### Description 12: 6-(2,4-Dichloro-phenylamino)-4-trifluoromethyl-nicotinic acid

A solution of KOH (1.4 g, 25 mmol) in 20 mL of EtOH/H₂O (1:1) was added to the crude mixture from Description 11 and the resulting mixture was stirred under reflux for 3 h. The solution was concentrated in vacuo, diluted with water and washed three times (3×15 mL) with diethyl ether. Upon acidification of the aqueous layer to pH1 with 37% HCl, the title compound precipitated out as hydrochloride salt that was filtered and dried under vacuum. The solid (2.89 g, 7.5 mmol) was then suspended in dichloromethane (20 mL), in the presence of PS-diisopropylethylamine (1.93 g, 7.5 mmol, loading 3.88 mmol/g, ex Argonaut Technologies) and stirred at room temperature for 30 min. After filtration of the resin and evaporation in vacuo of the solvent, the title compound was isolated as a white solid (2.62 g).

LC/MS, t=3.74 min, [MH⁺] 366.

(b). To a solution of methyl 2-(2,4-dichlorophenylamino)-4-trifluoromethyl-pyrimidine-5-carboxylate (0.358 g) in ethanol (8 ml) was added a solution of potassium hydroxide (190 mg) in ethanol (8 ml) and the solution stirred at reflux for 24 h. Ethanol was removed under reduced pressure and water (15 ml) added. The solution was washed with ether and concentrated hydrochloric acid was added to adjust the acidity to pH 1. The precipitated solid was filtered, washed with water and dried in vacuo at 50° C. to afford 2-(2,4-dichlorophenylamino)-4-trifluoromethyl-pyrimidine-5-carboxylic acid (262 mg).

NMR (DMSO-d6) δ 7.48 (1H, dd), 7.60 (1H, d), 7.73 (1H, d), 8.95 (1H, s), 10.3 (1H, s), 13.6 (1H, s).

(c). To a solution of 2-(2,4-dichlorophenylamino)-4-trifluoromethyl-pyrimidine-5-carboxylic acid (30 mg) in dimethylformamide (2 ml) was added successively N-ethylmorpholine (33 μl), 4-aminomethyltetrahydropyran (12 mg), 1-hydroxybenzotriazole hydrate (18 mg) and 1-(3-dimethylamino-propyl)-3-ethylcarbodiimide hydrochloride (20 mg). The solution was stirred for 3 h and allowed to stand overnight. Dimethylformamide was removed under reduced pressure and ethyl acetate (5 ml) added. The solution was washed sequentially with 5% sodium bicarbonate solution (2.5 ml), water (2.5 ml), 5% citric acid solution (2.5 ml) and brine (2×2.5 ml), dried (MgSO₄) and evaporated to afford the title compound (34 mg) NMR (DMSO-d6) δ 1.20 (2H, m), 1.58 (2H, d), 1.70 (1H, m), 3.10 (2H, t), 3.23 (2H, t), 3.84 (2H, dd), 7.46 (1H, dd), 7.57 (1H, d), 7.71 (1H, d), 8.59 (1H, t), 8.63 (1H, s), 10.00 (1H, s). LC/MS, t=3.33 min, [MH⁺] 449.

### Additional synthesis of Example 34: 2-(2,4-Dichlorophenylamino)-4-trifluoromethyl-pyrimidine-5-carboxylic acid (tetrahydro-pyran-4-ylmethyl)-amide

(a). To a solution of methyl 2-chloro-4-trifluoromethylpyrimidine-5-carboxylate (70 g, ex Maybridge 22 g, ex Fluorochem 48 g) in 1,4-dioxan (100 ml) was added 2,4-dichloroaniline (142 g) and the solution stirred under reflux for 10.5 h. 1,4-Dioxan was partially removed (approx 50 ml) under reduced pressure and 2N HCl (800 ml) added. The mixture was stirred with overhead stirring for 3 h and the resulting solid filtered onto a sinter. The solid was washed with 2N HCl (2×300 ml) and water (4×400 ml) then dried over sodium hydroxide in vacuo at 50° C. to afford methyl 2-(2,4-dichlorophenylamino)-4-trifluoromethyl-pyrimidine-5-carboxylate. The solid contained approximately 5% 2,4-dichloroaniline.

NMR (DMSO-d6) δ 3.84 (3H, s), 7.47 (1H, dd), 7.49 (1H, d), 7.74 (1H, d), 8.96 (1H, s), 10.45 (1H, s). LC/MS, t=3.66 min, [MH⁺] 366.

(b). To a solution of methyl 2-(2,4-dichlorophenylamino)-4-trifluoromethyl-pyrimidine-5-carboxylate (107 g) in methanol (700 ml) was added a solution of potassium hydroxide (50 g) in methanol (700 ml) and the solution stirred at reflux for 24 h. Methanol was removed under reduced pressure and water (800 ml) added. The solution was washed with ether (3×400 ml, which removed the remaining 2,4-dichloroaniline) and concentrated hydrochloric acid added to adjust the acidity to pH 1. The precipitated solid was filtered, washed with 2N HCl and water until the pH of the filtrate was neutral. The solid was dried in vacuo at 50° C. to afford 2-(2,4-dichlorophenylamino)-4-trifluoromethyl-pyrimidine-5-carboxylic acid (86.9 g)

NMR (DMSO-d6) δ 7.48 (1H, dd), 7.60 (1H, d), 7.73 (1H, d), 8.95 (1H, s), 10.3 (1H, s), 13.6 (1H, s). LC/MS, t=4.35 min, [MH⁺] 352

(c). To a solution of 2-(2,4-dichlorophenylamino)-4-trifluoromethyl-pyrimidine-5-carboxylic acid (86 g) in dimethylformamide (800 ml) was added successively N-ethylmorpholine (93 ml), 4-aminomethyltetrahydropyran (29.5 g), 1-hydroxybenzotriazole hydrate (51.5 g) and 1-(3-dimethylamino-propyl)-3-ethylcarbodiimide hydrochloride (56.2 g). The solution was stirred for 24 h. Dimethylformamide was partially removed (approx 650 ml) under reduced pressure and 5% sodium bicarbonate solution added (3×500 ml, added portionwise to control the release of carbon dioxide). The mixture was stirred with overhead stirring for 3 h and the resulting solid filtered onto a sinter. The solid was washed with 5% sodium bicarbonate (4×400 ml) and water (3×400 ml) then dried over sodium hydroxide in vacuo at 50° C. to afford the title compound (109.1 g)

NMR (DMSO-d6) δ 1.20 (2H, m), 1.58 (2H, d), 1.70 (1H, m), 3.10 (2H, t), 3.23 (2H, t), 3.84 (2H, dd), 7.46 (1H, dd), 7.57 (1H, d), 7.71 (1H, d), 8.59 (1H, t), 8.63 (1H, s), 10.00 (1H, s).

LC/MS, t=3.41 min, [MH⁺] 449.

### EXAMPLE 35

### 2-(2,5-Dichlorophenylamino)-4-trifluoromethylpyrimidine-5-carboxylic acid (tetrahydropyran-4-ylmethyl)-amide

In a manner similar to Reference Example 1(c) 2-(2,5-dichlorophenylamino)-4-trifluoromethyl-pyrimidine-5-carboxylic acid (50 mg) and 4-aminomethyltetrahydropyran (25 mg) afforded the title compound (63 mg).

NMR (DMSO-d6) δ 1.15-1.3 (2H, m), 1.60 (2H, d), 1.72 (1H, m), 3.12 (2H, t), 3.27 (2H, t), 3.85 (2H, d), 7.35 (1H, dd), 7.59 (1H, d), 7.73 (1H, s), 8.62 (1H, t), 8.70 (1H, s), 10.05 (1H, s).

LC/MS, t=3.30 min, [MH⁺] 449 and 451.

### EXAMPLE 36

### 2-(3,5-Dichlorophenylamino)-4-trifluoromethylpyrimidine-5-carboxylic acid (tetrahydropyran-4-ylmethyl)-amide

In a manner similar to Reference Example 1(c) 2-(3,5-dichlorophenylamino)-4-trifluoromethyl-pyrimidine-5-carboxylic acid (50 mg) and 4-aminomethyltetrahydropyran (25 mg) afforded the title compound (68 mg).

NMR (DMSO-d6) δ 1.15-1.35 (2H, m), 1.62 (2H, d), 1.72 (1H, m), 3.14 (2H, t), 3.28 (2H, t), 3.86 (2H, d), 7.25 (1H, s), 7.88 (2H, s), 8.66 (1H, t), 8.88 (1H, s), 10.75 (1H, s).

### EXAMPLE 37

### 2-(3-Methoxyphenylamino)-4-trifluoromethylpyrimidine-5-carboxylic acid (tetrahydropyran-4-ylmethyl)-amide

In a manner similar to Reference Example 1(c) 2-(3-methoxyphenylamino)-4-trifluoromethyl-pyrimidine-5-carboxylic acid (32 mg) and 4-aminomethyltetrahydropyran (14.5 mg) afforded the title compound (29 mg).

NMR (DMSO-d6) δ 1.1-1.25 (2H, m), 1.61 (2H, d), 1.74 (1H, m), 3.13 (2H, t), 3.27 (2H, t), 3.74 (3H, s), 3.86 (2H, d), 6.63 (1H, d), 7.25 (2H, m), 7.53 (1H, s), 8.62 (1H, t), 8.76 (1H, s), 10.35 (1H, s).

LC/MS, t=2.97 min, [MH⁺] 411.

### EXAMPLE 38

### 2-(3-Fluorophenylamino)-4-trifluoromethylpyrimidine-5-carboxylic acid cyclopentylmethyl-amide

In a manner similar to Reference Example 1(c) 2-(3-fluorophenylamino)-4-trifluoromethyl-pyrimidine-5-carboxylic acid (30 mg) and cyclopentylmethylamine hydrochloride (17 mg, prepared as described in Kelley et al., J. Med. Chem., 40, 3207, (1997)) afforded the title compound (17 mg).

NMR (DMSO-d6) δ 1.20-1.30 (2H, m), 1.45-1.68 (4H, m), 1.68-1.77 (2H, m), 2.1 (1H, quintuplet), 3.19 (2H, t), 6.89 (1H, dt), 7.40 (1H, q), 7.54 (1H, d), 7.78 (1H, d), 8.64 (1H, t), 8.80 (1H, s), 10.70 (1H, s). LC/MS, t=3.53 min, [MH⁺] 383.

### EXAMPLE 39

### 2-(3-Bromophenylamino)-4-trifluoromethylpyrimidine-5-carboxylic acid cyclopentylmethyl-amide

In a manner similar to Reference Example 1(c) 2-(3-bromophenylamino)-4-trifluoromethyl-pyrimidine-5-carboxylic acid (36.5 mg) and cyclopentylmethylamine hydrochloride (17 mg) afforded the title compound (28 mg).

NMR (DMSO-d6) δ 1.39-1.52 (2H, m), 1.69-1.90 (4H, m), 1.90-2.02 (2H, m), 2.34 (1H, quintuplet), 3.4 (2H, t), 7.48 (1H, d), 7.57 (1H, t), 7.95 (1H, d), 8.37 (1H, s). 8.86 (1H, t), 9.02 (1H, s), 10.80 (1H, s). LC/MS, t=3.33 min, [MH⁺] 443 and 445.

### EXAMPLE 40

### 2-(2,3-Dichlorophenylamino)-4-trifluoromethylpyrimidine-5-carboxylic acid cyclopentylmethyl-amide

In a manner similar to Reference Example 1(c) 2-(2,3-dichlorophenylamino)-4-trifluoromethyl-pyrimidine-5-carboxylic acid (32 mg) and cyclopentylmethylamine hydrochloride (15 mg) afforded the title compound (30 mg).

NMR (DMSO-d6) δ 1.15-1.30 (2H, m), 1.44-1.78 (6H, m), 2.10 (1H, quintuplet), 3.16 (2H, t), 7.41 (2H, t), 7.54 (1H, m), 8.58 (1H, br t), 8.78 (1H, s), 10:10 (1H, s). LC/MS, t=3.71 min, [MH⁺] 433 and 435.

### EXAMPLE 41

### 2-(2,4-Dichlorophenylamino)-4-trifluoromethylpyrimidine-5-carboxylic acid cyclopentylmethyl-amide

In a manner similar to Reference Example 1(c) 2-(2,4-dichlorophenylamino)-4-trifluoromethyl-pyrimidine-5-carboxylic acid (32 mg) and cyclopentylmethylamine hydrochloride (15 mg) afforded the title compound (27 mg).

NMR (DMSO-d6) δ 1.2-1.3 (2H, m), 1.4-1.79 (6H, m), 2.10 (1H, quintuplet), 3.17 (2H, t), 7.50 (1H, d), 7.60 (1H, d), 7.75 (1H, d), 8.68 (1H, t), 8.78 (1H, s), 10.10 (1H, s). LC/MS, t=3.76 ml, [MH⁺] 433 and 435.

### EXAMPLE 42

### 2-(2,5-Dichlorophenylamino)-4-trifluoromethylpyrimidine-5-carboxylic acid cyclopentylmethyl-amide

In a manner similar to Reference Example 1(c) 2-(2,5-dichlorophenylamino)-4-trifluoromethyl-pyrimidine-5-carboxylic acid (32 mg) and cyclopentylmethylamine hydrochloride (15 mg) afforded the title compound (23 mg).

NMR (DMSO-d6) δ 1.15-1.30 (2H, nm), 1.45-1.79 (6H, m), 2.08 (1H, quintuplet), 3.18 (2H, t), 7.38 (1H, d), 7.62 (1H, d), 7.75 (1H, s), 8.61 (1H, br t), 8.71 (1H, s), 10.05 (1H, s).

LC/MS, t=3.76 min, [MH⁺] 433 and 435.

### EXAMPLE 43

### 2-(2,6-Dichlorophenylamino)-4-trifluoromethylpyrimidine-5-carboxylic acid cyclopentylmethyl-amide

In a manner similar to Reference Example 1(c) 2-(2,6-dichlorophenylamino)-4-trifluoromethyl-pyrimidine-5-carboxylic acid (32 mg) and cyclopentylmethylamine hydrochloride (15 mg) afforded the title compound (25 mg).

NMR (DMSO-d6) δ 1.15-1.30 (2H, m), 1.45-1.78 (6H, m), 2.08 (1H, quintuplet), 3.15 (2H, t), 7.4 (1H, t), 7.6-7.68 (2H, m), 8.5-8.7 (2H, m), 10.20 (1H, s). LC/MS, t=3.49 min, [MH⁺] 433 and 435.

### EXAMPLE 44

### 2-(3,4-Dichlorophenylamino)-4-trifluoromethylpyrimidine-5-carboxylic acid cyclopentylmethyl-amide

In a manner similar to Reference Example 1(c) 2-(3,4-dichlorophenylamino)-4-trifluoromethyl-pyrimidine-5-carboxylic acid (32 mg) and cyclopentylmethylamine hydrochloride (15 mg) afforded the title compound (29 mg).

NMR (DMSO-d6) δ 1.12-1.3 (2H, m), 1.44-1.8 (6H, m), 2.1 (1H, quintuplet). 3.17 (2H, t), 7.62 (1H, br d), 7.72 (1H, d), 8.18 (1H, d), 8.60-8.69 (1H, br t), 8.83 (1H, s), 10.80 (1H, s).

LC/MS, t=3.87 min, [MH⁺] 433 and 435.

### EXAMPLE 45

### 2-(3,5-Dichlorophenylamino)-4-trifluoromethylpyrimidine-5-carboxylic acid cyclopentylmethyl-amide

In a manner similar to Reference Example 1(c) 2-(3,5-dichlorophenylamino)-4-trifluoromethyl-pyrimidine-5-carboxylic acid (32 mg) and cyclopentylmethylarmine hydrochloride (15 mg) afforded the title compound (27 mg).

NMR (DMSO-d6) 1.14-1.34 (2H, m), 1.45-1.8 (6H, m), 2.10 (1H, quintuplet), 3.20 (2H, t), 7.28 (1H, s), 7.91 (2H, s), 8.6-8.7 (1H, br t), 8.9 (1H, s), 10.75 (1H, s).

LC/MS, t=3.94 min, [MH⁺] 433 and 435.

### EXAMPLE 46

### 2-(3-Methoxyphenylamino)-4-trifluoromethylpyrimidine-5-carboxylic acid cyclopentylmethyl-amide

In a manner similar to Reference Example 1(c) 2-(3-methoxyphenylamino)-4-trifluoromethyl-pyrimidine-5-carboxylic acid (32 mg) and cyclopentylmethylamine hydrochloride (17 mg) afforded the title compound (21 mg).

NMR (DMSO-d6) 1.25-1.38 (2H, m), 1.50-1.85 (6H, m), 2.15 (1H, quintuplet), 3.25 (2H, t), 3.85 (3H, s), 6.70 (1H, br d), 7.26-7.37 (2H, m), 7.60 (1H, m), 8.68 (1H, t), 8.80 (1H, s), 10.50 (1H, s).

LC/MS, t=3.46 min, [MH⁺] 395.

### EXAMPLE 47

### 2-(3-Bromophenylamino)-4-trifluoromethylpyrimidine-5-carboxylic acid cyclobutylamide

In a manner similar to Reference Example 1(c) 2-(3-bromophenylamino)-4-trifluoromethyl-pyrimidine-5-carboxylic acid (35 mg) and cyclobutylamine (10 μl) afforded the title compound (30 mg).

NMR (DMSO-d6) δ 1.70 (2H, m), 1.97 (2H, m), 2.22 (2H, m), 4.32 (1H, m), 7.22-7.33 (2H, m), 7.70 (1H, d), 8.10 (1H, s), 8.81-8.83 (2H, m), 10.60 (1H, s). LC/MS, t=3.47 min, [MH⁺] 415 and 417.

### EXAMPLE 48

### 2-(2,3-Dichlorophenylamino)-4-trifluoromethylpyrimidine-5-carboxylic acid cyclobutylamide

In a manner similar to Reference Example 1(c) 2-(2,3-dichlorophenylamino)-4-trifluoromethyl-pyrimidine-5-carboxylic acid (25 mg) and cyclobutylamine (10 μl) afforded the title compound (20 mg).

NMR (DMSO-d6) δ 1.70 (2H, m), 1.97 (2H, m), 2.22 (2H, m), 4.32 (1H, m), 7.38-7.56 (3H, m), 8.65 (1H, s), 8.80 (1H, d), 10.10 (1H, s). LC/MS, t=3.48 min, [MH⁺] 405 and 407.

### EXAMPLE 49

### 2-(2,4-Dichlorophenylamino)-4-trifluoromethylpyrimidine-5-carboxylic acid cyclobutylamide

In a manner similar to Reference Example 1(c) 2-(2,4-dichlorophenylamino)-4-trifluoromethyl-pyrimidine-5-carboxylic acid (30 mg) and cyclobutylamine (10 μl) afforded the title compound (26 mg).

NMR (DMSO-d6) δ 1.70 (2H, m), 1.97 (2H, m), 2.22 (2H, m), 4.32 (1H, m), 7.46-7.72 (3H, m), 8.64 (1H, s), 8.80 (1H, d), 10.00 (1H, s). LC/MS, t=3.54 min, [MH⁺] 405 and 407.

### EXAMPLE 50

### 2-(2,5-Dichlorophenylamino)-4-trifluoromethylpyrimidine-5-carboxylic acid cyclobutylamide

In a manner similar to Reference Example 1(c) 2-(2,5-dichlorophenylamino)-4-trifluoromethyl-pyrimidine-5-carboxylic acid (50 mg) and cyclobutylamine (19 μl) afforded the title compound (56 mg).

NMR (DMSO-d6) δ 1.70 (2H, m), 1.97 (2H, m), 2.22 (2H, m), 4.30 (1H, m), 7.33-7.73 (3H, m), 8.70 (1H, s), 8.80 (1H, d), 10.00 (1H, s). LC/MS, t=3.52 min, [MH⁺] 405 and 407.

### EXAMPLE 51

### 2-(2,6-Dichlorophenylamino)-4-trifluoromethylpyrimidine-5-carboxylic acid N-cyclobutylamide

In a manner similar to Reference Example 1(c) 2-(2,6-dichlorophenylamino)-4-trifluoromethyl-pyrimidine-5-carboxylic acid (30 mg) and cyclobutylamine (10 μl) afforded the title compound (34 mg).

NMR (DMSO-d6) δ 1.70 (2H, m), 1.97 (2H, m), 2.22 (2H, m), 4.30 (1H, m), 7.36-7.60 (3H, m), 8.59 (1H, s), 8.80 (1H, d), 10.15 (1H, s). LC/MS, t=3.24 min, [W] 405 and 407.

### EXAMPLE 52

### 2-(3,5-Dichlorophenylamino)-4-trifluoromethylpyrimidine-5-carboxylic acid cyclobutylamide

In a manner similar to Reference Example 1(c) 2-(3,5-dichlorophenylamino)-4-trifluoromethyl-pyrimidine-5-carboxylic acid (50 mg) and cyclobutylamine (19 μl) afforded the title compound (56 mg).

NMR (DMSO-d6) δ 1.70 (2H, m), 1.97 (2H, m), 2.22 (2H, m), 4.32 (1H, m), 7.25-7.87 (3H, m), 8.85 (1H, d), 8.88 (1H, s), 10.80 (1H, s). LC/MS, t=3.73 min, [MH⁺] 405 and 407.

### EXAMPLE 53

### 2-(3-Methoxyphenylamino)-4-trifluoromethylpyrimidine-5-carboxylic acid cyclobutylamide

In a manner similar to Reference Example 1(c) 2-(3-methoxyphenylamino)-4-trifluoromethyl-pyrimidine-5-carboxylic acid (32 mg) and cyclobutylamine (10.5 μl) afforded the title compound (27 mg).

NMR (DMSO-d6) δ 1.70 (2H, m), 1.97 (2H, m), 2.22 (2H, m), 3.75 (3H, s), 4.32 (1H, m), 7.53-7.87 (4H, m), 8.76 (1H, s), 8.81 (1H, d), 10.40 (1H, s). LC/MS, t=3.20 min, [MH⁺] 367.

### EXAMPLE 54

### 2-(3-Chlorophenylamino)-4-trifluoromethylpyrimidine-5-carboxylic acid cyclobutylmethyl-amide

(a) A solution of borane-tetrahydrofuran complex (1M in tetrahydrofuran, 120 ml) was added over 10 min to a solution of cyclobutane carbonitrile (8.1 g) [Lancaster] in dry tetrahydrofuran (20 ml) under nitrogen at room temperature. The solution was refluxed overnight then cooled to 20°. Methanol (150 ml) was added dropwise over 15 mins keeping the temperature below 25°, then the mixture was cooled to 0° and dry hydrogen chloride was bubbled through for 30 min. The resulting mixture was refluxed for 90 min, evaporated and the residue re-evaporated twice from methanol. Ether (150 ml) was added and the resulting solid was filtered off. It was taken up in hot isopropanol (50 ml), filtered, and hot acetonitrile (30 ml) added. The mixture was cooled and the solid filtered off to give the C-cyclobutylmethylamine hydrochloride (5.7 g)

NMR (400 MHz, DMSO-d6) F63821.8 (4H, m), 2.0 (2H, m), 2.54 (1H, m), 2.80 (2H, d), 8.0 (3H, br s).

(b) In a manner similar to Reference Example 1(c) 2-(3-chlorophenylamino)-4-trifluoromethyl-pyrimidine-5-carboxylic acid (32 mg) and C-cyclobutylmethylamine hydrochloride (13 mg) afforded the title compound (28 mg).

NMR (DMSO-d6) δ 1.70 (2H, m), 1.82 (2H, m), 2.00 (2H, m), 2.50 (1H, m), 3.26 (2H, m), 7.08-7.95 (4H, m), 8.55 (1H, t), 8.77 (1H, s), 10.60 (1H, s).

LC/MS, t=3.56 min, [MH⁺] 385.

### EXAMPLE 55

### 2-(2,6-Dichlorophenylamino)-4-trifluoromethyl-pyrimidine-5-carboxylic acid (tetrahydro-pyran-4-ylmethyl)-amide

In a manner similar to Reference Example 1(c) 2-(2,6-dichlorophenylamino)-4-trifluoromethyl-pyrimidine-5-carboxylic acid (30 mg) and 4-aminomethyltetrahydropyran (20 mg, ex CombiBlocks) afforded the title compound (32 mg).

NMR (DMSO-d6) δ 1.16-1.22 (2H, m), 1.58 (2H, d), 1.70 (1H, m), 3.09 (2H, t), 3.23 (2H, m), 3.84 (2H, d), 7.38 (1H, t), 7.59 (2H, d), 8.61 (2H, m), 10.10 (1H, s)

LC/MS, t=3.02 min, Molecular ion observed (MH⁺)=449 consistent with the molecular formula C₁₈H₁₇³⁵Cl₂F₃N₄O₂

### EXAMPLE 56

### 2-(3,4-Dichlorophenylamino)-4-trifluoromethylpyrimidine-5-carboxylic acid (tetrahydro-pyran-4-ylmethyl)-amide

In a manner similar to Reference Example 1(c) 2-(3,4-dichlorophenylamino)-4-trifluoromethyl-pyrimidine-5-carboxylic acid (30 mg) and 4-aminomethyltetrahydropyran (20 mg, ex CombiBlocks) afforded the title compound (38 mg).

NMR (DMSO-d6) δ 1.18-1.25 (2H, m), 1.62 (2H, d), 1.74 (1H, m), 3.1 (2H, t), 3.25 (2H, m), 3.85 (2H, d), 7.60 (1H, t), 7.69 (1H, m), 8.16 (1H, dd), 8.64 (1H, t), 8.84 (1H, s), 10.70 (1H, s)

LC/MS, t=3.45 min, Molecular ion observed (MH⁺)=449 consistent with the molecular formula C₁₈H₁₇N₄O₂³⁵Cl₂F₃

### EXAMPLE 68

### 1-[2-(3,4-Dichlorophenylamino)-4-trifluoromethylpyrimidin-5-yl]-1-(morpholin-4-yl)-methanone

In a manner similar to Reference Example 1(c) 2-(3,4-dichlorophenylamino)-4-trifluoromethyl-pyrimidine-5-carboxylic acid (30 mg) and morpholine (15 mg, ex Aldrich) afforded the title compound (36 mg).

NMR (DMSO-d6) 6) 3.7 (8H, s), 7.65 (1H, d), 7.75 (1H, dd), 8.2 (1H, d), 8.9 (1H, s), 10.80 (1H, s)

LC/MS, t=3.45 min, Molecular ion observed (MH⁺)=421 consistent with the molecular formula C₁₆H₁₃N₄O₂³⁵Cl₂F₃

TABLE 2
Example 57-67 and 69-73 were prepared in a corresponding fashion to the above
compounds.

| | | Mass spec details |
|---|---|---|
| | | 1 Retention Time |
| | | 2 MH⁺ |
| Ex. | | 3 Formula consistent |
| No. | Compound Name | with MH⁺ |
| 57 | 2-(3-Chloro-phenylamino)-4-trifluoromethyl-pyrimidine- | 3.78 |
| | 5-carboxylic acid cycloheptylamide | 413 |
| | | C₁₉H₂₀³⁵ClF₃N₄O |
| 58 | 2-(3-Chloro-phenylamino)-4-trifluoromethyl-pyrimidine- | 3.25 |
| | 5-carboxylic acid [(S)-1-(tetrahydro-furan-2-y-l)methyl]- | 401 |
| | amide | C₁₇H₁₆³⁵ClF₃N₄O₂ |
| 59 | 2-(3-Fluoro-phenylamino)-4-trifluoromethyl-pyrimidine- | 3.10 |
| | 5-carboxylic acid [(S)-1-(tetrahydro-furan-2-y-l)methyl]- | 385 |
| | amide | C₁₇H₁₆F₄N₄O₂ |
| 60 | 2-(3-Bromo-phenylamino)-4-trifluoromethyl-pyrimidine- | 3.29 |
| | 5-carboxylic acid [(S)-1-(tetrahydro-furan-2-yl)methyl]- | 447 |
| | amide | C₁₇H₁⁸¹BrF₃N₄O₂ |
| 61 | 2-(3-Chloro-phenylamino)-4-trifluoromethyl-pyrimidine- | 3.22 |
| | 5-carboxylic acid (1-methanesulfonyl-piperidin-4- | 492 |
| | ylmethyl)-amide | C₁₉H₂₁³⁵ClF₃N₅O₃S |
| 62 | 2-(2,5-Dichloro-phenylamino)-4-trifluoromethyl- | 3.90 |
| | pyrimidine-5-carboxylic acid cyclohexylmethyl-amide | 447 |
| | | C₁₉H₁₉³⁵Cl₂F₃N₄O |
| 63 | 2-(3-Chloro-phenylamino)-4-trifluoromethyl-pyrimidine- | 3.60 |
| | 5-carboxylic acid (1-ethyl-propyl)-amide | 387 |
| | | C₁₇H₁₈³⁵ClF₃N₄O |
| 64 | 2-(3-Chloro-phenylamino)-4-trifluoromethyl-pyrimidine- | 3.55 |
| | 5-carboxylic acid (tert-butyl)-amide | 373 |
| | | C₁₆H₁₆³⁵ClF₃N₄O |
| 65 | 2-(3-Chloro-phenylamino)-4-trifluoromethyl-pyrimidine- | 3.18 |
| | 5-carboxylic acid (tetrahydro-pyran-4-yl)-amide | 401 |
| | | C₁₇H₁₆³⁵ClF₃N₄O₂ |
| 66 | 2-(3-Chloro-phenylamino)-4-trifluoromethyl-pyrimidine- | 3.67 |
| | 5-carboxylic acid cyclohexyl-amide | 399 |
| | | C₁₈H₁₈³⁵ClF₃N₄O |
| 67 | 1-[2-(3,5-Dichloro-phenylamino)-4-trifluoromethyl- | 3.84 |
| | pyrimidin-5-yl]-1-(piperidin-1-yl)-methanone | 419 |
| | | C₁₇H₁₅³⁵Cl₂F₃N₄O |
| 69 | 2-Phenylamino-4-trifluoromethyl-pyrimidine-5- | 3.48 |
| | carboxylic acid (2,2-dimethyl-propyl)-amide | 353 |
| | | C₁₇H₁₉F₃N₄O |
| 70 | 2-Phenylamino-4-trifluoromethyl-pyrimidine-5- | 3.60 |
| | carboxylic acid (3,3-dimethyl-butyl)-amide | 367 |
| | | C₁₈H₂₁F₃N₄O |
| 71 | 2-(3-Chloro-phenylamino)-4-trifluoromethyl-pyrimidine- | 2.46 |
| | 5-carboxylic acid (piperidin-4-ylmethyl)-amide | 414 |
| | trifluoroacetate | C₁₈H₁₉³⁵ClF₃N₅O |
| 72 | 1-[2-(3-Chloro-phenylamino)-4-trifluoromethyl- | 2.42 |
| | pyrimidin-5-yl]-1-(piperazin-1-yl)-methanone | 386 |
| | | C₁₆H₁₅³⁵ClF₃N₅O |
| 73 | 2-(3-Fluoro-phenylamino)-4-trifluoromethyl-pyrimidine- | 3.10 |
| | 5-carboxylic acid [(R)-1-(tetrahydro-furan-2-yl)methyl]- | 385 |
| | amide | C₁₇H₁₆F₄N₄O₂ |

Compounds 74 to 87 were prepared according to the conditions described for table 1, and purified by the method given in column P as follows:

Method A: refers to the procedure in part (b) of Example 166.
Method B: Mass-directed autopurification using the procedures detailed at the beginning of the experimental
Method C: Purification using Biotage Chromatography over Merck 9385 Silica Gel (25 g) eluting with 1-2% methanol in dichloromethane.

### Intermediate A: 4-Aminomethyltetrahydropyran-4-ol hydrochloride

To a solution of 1.0M lithium aluminium hydride in tetrahydrofuran (20 ml) was added under a nitrogen atmosphere a solution of 4-hydroxytetra-hydropyran-4-carbonitrile (0.50 g, prepared as described in Eiden et al., Arch. Pharm., 320, 348, (1987)) in tetrahydrofuran (2 ml) and the solution stirred at reflux for 6 hours. Water (1 ml) and 2N sodium hydroxide solution (1 ml) were added cautiously and the resultant solid filtered and washed with ether. The filtrate was dried (MgSO₄), evaporated and the residue dissolved in ethanol (3 ml) and concentrated hydrochloric acid (0.5 ml) added. Solvent was removed under reduced pressure and the resultant solid washed with ether and dried in vacuo at 40° C. to afford the title compound (234 mg).

NMR (DMSO-d6) 1.45-1.6 (4H, m), 2.78 (2H, q), 3.61 (4H, m). 5.07 (1H, br s), 7.89 (3H, br s).

TABLE 3

| | | | LC/MS 1 Retention time (min) |
|---|---|---|---|
| | | | 2 MH⁺ |
| Ex. No. | Compound name | P | 3 Formula |
| 74 | 3,4-Dichlorophenylamino-4- | A | 3.53 |
| | trifluoromethyl-pyrimidine-5-carboxylic | | 419 |
| | acid cyclopentylamide | | C₁₇H₁₅³⁵Cl₂F₃N₄O |
| 75 | 3,5-Dichlorophenylamino-4- | A | 3.60 |
| | trifluoromethyl-pyrimidine-5-carboxylic | | 419 |
| | acid cyclopentylamide | | C₁₇H₁₅³⁵Cl₂F₃N₄O |
| 76 | 3-Methoxyphenylamino-4-trifluoromethyl- | A | 3.08 |
| | pyrimidine-5-carboxylic acid | | 381 |
| | cyclopentylamide | | C₁₈H₁₉F₃N₄O₂ |
| 77 | 2,3-Dichlorophenylamino-4- | A | 3.60 |
| | trifluoromethyl-pyrimidine-5-carboxylic | | 419 |
| | acid cyclopentylamide | | C₁₇H₁₅³⁵Cl₂F₃N₄O |
| 78 | 1-[2-(3-fluorophenylamino)-4- | A | 3.39 |
| | trifluoromethyl-pyrimidin-5-yl]-1-piperidin- | | 369 |
| | 1-yl-methanone | | C₁₇H₁₆N₄F₄O |
| 79 | 1-[2-(3-chlorophenylamino)-4- | A | 3.21 |
| | trifluoromethyl-pyrimidin-5-yl]-1-(4- | | 464 |
| | methanesulfonyl-piperazin-1-yl)-methanone | | C₁₇H₁₇Cl³⁵F₃N₅O₃S |
| 80 | 2-(3-bromophenylamino)-4-trifluoromethyl- | A | 3.29 |
| | pyrimidine-5-carboxylic acid [R-1- | | 447 |
| | (tetrahydrofuran-2-yl)methyl]-amide | | C₁₇H₁₆⁸¹BrF₃N₄O₂ |
| 81 | 2-(2,3-Diclorophenylamino)-4- | C | 3.74 |
| | trifluoromethyl-pyrimidine-5-carboxylic | | 451 |
| | acid (tetrahydro-thiopyran-4-yl)-amide | | C₁₇H₁₅³⁵Cl₂F₃N₄OS |
| 82 | 2-(2,3-Dichlorophenylamino)-4- | C | 3.29 |
| | trifluoromethyl-pyrimidine-5-carboxylic | | 483 |
| | acid (1,1-dioxo-tetrahydro-2H-thiopyran-4- | | C₁₇H₁₅³⁵Cl₂F₃N₄O₃S |
| | yl)-amide | | |
| 83 | 2-(3-Fluoro-phenylamino)-4- | A | 3.32 |
| | trifluoromethyl-pyrimidine-5-carboxylic | | 355 |
| | acid cyclopropylmethyl-amide | | C₁₆H₁₄F₄N₄O |
| 84 | 2-(3-Chloro-phenylamino)-4- | B | 3.46 |
| | trifluoromethyl-pyrimidine-5-carboxylic | | 371 |
| | acid cyclopropylmethyl-amide | | C₁₆H₁₄³⁵ClF₃N₄O |
| 85 | 1-[2-(2,5-Dichloro-phenylamino)-4-tri- | A | 3.64 |
| | fluoromethyl-pyrimidin-5-yl]-1-piperidin-1- | | 419 |
| | yl-methanone | | C₁₇H₁₅³⁵Cl₂F₃N₄O |
| 86 | 2-(3-Fluorophenyl-amino)-4-trifluoro- | A | 3.25 |
| | methyl-pyrimidine-5-carboxylic acid (1- | | 411 |
| | hydroxy-cyclohexyl-methyl)-amide | | C₁₉H₂₀F₄N₄O₂ |
| 87 | 2-(3-Bromophenyl-amino)-4- | A | 3.05 |
| | trifluoromethyl-pyrimidine-5-carboxylic | | 473 |
| | acid (4-hydroxytetrahydropyran-4- | | C₁₈H₁₈⁷⁹BrF₃N₄O₃ |
| | ylmethyl)amide | | |

Examples 88 to 113 and 257 to 259 were prepared from the appropriate reactants in a manner similar to example 166.

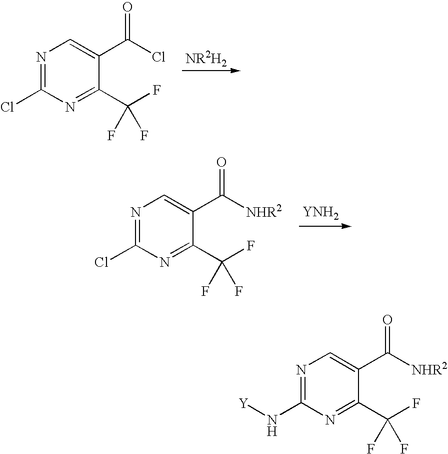

Preparation Method A: refers to the procedure give in part (b) of Example 166.

Preparation Method B: This is exemplified by the by Example 109, 2-chloro-4-trifluoromethyl-pyrimidin-5-carboxylic acid (tetrahydropyran-4-ylmethyl)amide (50 mg) and 2-chloro-2-cyanoaniline (118 mg) were irradiated in a microwave apparatus (the model used was the ‘Creator’, supplied by ‘Personal Chemistry’, operating at 300 Watts), at 190° C. for 30 min. For examples using this method, the equivalents of substituted aniline YNH₂ used, and duration of irradiation follow in brackets after the method B.

Preparation Method C—As for method B, but the solvent used was 1,4-dioxan not MeCN

The column entitled “Prep” refers to the preparation method used.

The product was then purified according to one of the following methods described below. The column entitled “Pure” refers to the purification method used

Purification method A: refers to the procedure give in part (b) of Example 166

Purification method B: mass directed autopurification using the procedures detailed at the beginning of the experimental.

Purification method C: The reaction was worked up as for part (b) of Example 166, and the crude product further purified by Biotage chromatography over Merck 9385 silica gel, eluting with isohexane/ethyl acetate.

TABLE 4

| | | | | LCMS |
|---|---|---|---|---|
| | | | | 1 Retention time |
| | | | | 2 MH⁺ |
| Ex. | | | | 3 Formula consistent |
| No. | Compound name | Prep | Pure | with MH⁺ |
| 88 | 2-(3,5-Bis-trifluoromethyl-phenylamino)-4- | A | B | 4.09 |
| | trifluoromethyl-pyrimidine-5-carboxylic acid | | | 515 |
| | cyclohexylmethyl-amide | | | C₂₁H₁₉F₉N₄O |
| 89 | 2-(3,5-Dicyano-phenylamino)-4- | A | B | 3.59 |
| | trifluoromethyl-pyrimidine-5-carboxylic acid | | | 429 |
| | cyclohexylmethyl-amide | | | C₂₁H₁₉F₃N₆O |
| 90 | 2-(3-Fluoro-5-trifluoromethyl-phenylamino)- | A | B | 3.96 |
| | 4-trifluoromethyl-pyrimidine-5-carboxylic | | | 465 |
| | acid cyclohexylmethyl-amide | | | C₂₀H₁₉F₇N₄O |
| 91 | 2-(3-Bromo-5-trifluoromethyl- | A | B | 4.13 |
| | phenylamino)-4-trifluoromethyl-pyrimidine- | | | 524 |
| | 5-carboxylic acid cyclohexylmethyl-amide | | | C₂₀H₁₉⁷⁹BrF₆N₄O |
| 92 | 2-(2-Chloro-3-methyl-phenylamino)-4- | A | B | 3.82 |
| | trifluoromethyl-pyrimidine-5-carboxylic acid | | | 427 |
| | cyclohexylmethyl-amide | | | C₂₀H₂₂³⁵ClF₃N₄O |
| 93 | 2-(3-Chloro-2-methyl-phenylamino)-4- | A | B | 3.76 |
| | trifluoromethyl-pyrimidine-5-carboxylic acid | | | 427 |
| | cyclohexylmethyl-amide | | | C₂₀H₂₂³⁵ClF₃N₄O |
| 94 | 2-(4-Chloro-2-methyl-phenylamino)-4- | A | B | 3.77 |
| | trifluoromethyl-pyrimidine-5-carboxylic acid | | | 427 |
| | cyclohexylmethyl-amide | | | C₂₀H₂₂³⁵ClF₃N₄O |
| 95 | 2-(4-Chloro-2,6-dimethyl-phenylamino)-4- | A | B | 3.79 |
| | trifluoromethyl-pyrimidine-5-carboxylic acid | | | 441 |
| | cyclohexylmethyl-amide | | | C₂₁H₂₄³⁵ClF₃N₄O |
| 96 | 2-(3,5-Difluoro-phenylamino)-4- | A | C | 3.70 |
| | trifluoromethyl-pyrimidine-5-carboxylic acid | | | 401 |
| | cyclopentylmethyl-amide | | | C₁₈H₁₇F₅N₄O |
| 97 | 2-(4-Trifluoromethyl-3-fluorophenylamino)-4- | A | B | 3.86 |
| | trifluoromethyl-pyrimidine-5-carboxylic acid | | | 451 |
| | cyclopentylmethyl-amide | | | C₁₉H₁₇F₇N₄O |
| 98 | 2-(2,4-Difluorophenylamino)-4- | A | A | 3.03 |
| | trifluoromethyl-pyrimidine-5-carboxylic acid | | | 417 |
| | (tetrahydro-pyran-4-ylmethyl)-amide | | | C₁₈H₁₇F₅N₄O₂ |
| 99 | 2-(2-Fluoro-4-chlorophenylamino)-4- | A | A | 3.23 |
| | trifluoromethyl-pyrimidine-5-carboxylic acid | | | 433 |
| | (tetrahydro-pyran-4-ylmethyl)-amide | | | C₁₈H₁₇³⁵ClF₄N₄O₂ |
| 100 | 2-(2-Trifluoromethyl-4-fluorophenylamino)-4- | A | B | 3.69 |
| | trifluoromethyl-pyrimidine-5-carboxylic acid | | | 465 |
| | cyclohexylmethyl-amide | | | C₂₀H₁₉F₇N₄O |
| 101 | 2-(2-Trifluoromethyl-4-fluorophenylamino)-4- | A | B | 3.49 |
| | trifluoromethyl-pyrimidine-5-carboxylic acid | | | 437 |
| | cyclobutylmethyl-amide | | | C₁₈H₁₅F₇N₄O |
| 102 | 2-(2-Chloro-4-trifluoromethylphenylamino)-4- | A | B | 3.79 |
| | trifluoromethyl-pyrimidine-5-carboxylic acid | | | 453 |
| | cyclobutylmethyl-amide | | | C₁₈H₁₅³⁵ClF₆N₄O |
| 103 | 2-(2-Chloro-4-cyanophenylamino)-4- | B | C. | 3.47 |
| | trifluoromethyl-pyrimidine-5-carboxylic acid | | | 410 |
| | cyclobutylmethyl-amide | | | C₁₈H₁₅³⁵ClF₃N₅O |
| 104 | 2-(2-Trifluoromethyl-4-chlorophenylamino)-4- | A | A | 3.34 |
| | trifluoromethyl-pyrimidine-5-carboxylic acid | | | 483 |
| | (tetrahydro-pyran-4-ylmethyl)-amide | | | C₁₉H₁₇³⁵ClF₆N₄O₂ |
| 105 | 2-(2-Trifluoromethyl-4-bromophenylamino)-4- | B (5 equiv, | B | 3.71 |
| | trifluoromethyl-pyrimidine-5-carboxylic acid | 45 min) | | 499 |
| | cyclobutylmethyl-amide | | | C₁₈H₁₅⁸¹BrF₆N₄O |
| 106 | 2-(2-Trifluoromethyl-4-bromophenylamino)-4- | B | B | 3.89 |
| | trifluoromethyl-pyrimidine-5-carboxylic acid | (2.5 equiv, | | 527 |
| | cyclohexylmethyl-amide | 45 min) | | C₂₀H₁₉⁸¹BrF₆N₄O |
| 107 | 2-(2,3-Difluoro-phenylamino)-4-tri- | A | B | 3.64 |
| | fluoromethyl-pyrimidine-5-carboxylic acid | | | 417 |
| | (tetrahydropyran-4-ylmethyl)-amide | | | C₁₈H₁₇F₅N₄O₂ |
| 108 | 2-(5-Chloro-2-methyl-phenylamino)-4-tri- | A | B | 3.64 |
| | fluoromethyl-pyrimidine-5-carboxylic acid | | | 429 |
| | (tetrahydropyran-4-ylmethyl)-amide | | | C₁₉H₂₀³⁵ClF₃N₄O₂ |
| 109 | 2-(3-Chloro-2-cyano-phenylamino)-4-tri- | B | B | 3.64 |
| | fluoromethyl-pyrimidine-5-carboxylic acid | (5 equiv, | | 440 |
| | (tetrahydropyran-4-ylmethyl)-amide | 30 min) | | C₁₉H₁₇³⁵ClF₃N₅O₂ |
| 110 | 2-(2-Chloro-4-methyl-phenylamino)-4-tri- | A | B | 3.27 |
| | fluoromethyl-pyrimidine-5-carboxylic acid | | | 429 |
| | (tetrahydropyran-4-ylmethyl)-amide | | | C₁₉H₂₀³⁵ClF₃N₄O₂ |
| 111 | 2-(4-Chloro-3-cyano-phenylamino)-4-tri- | A | B | 3.22 |
| | fluoromethyl-pyrimidine-5-carboxylic acid | | | 440 |
| | (tetrahydropyran-4-ylmethyl)-amide | | | C₁₉H₁₇³⁵ClF₃N₅O₂ |
| 112 | 2-(4-Chloro-2-methyl-phenylamino)-4-tri- | A | B | 3.23 |
| | fluoromethyl-pyrimidine-5-carboxylic acid | | | 429 |
| | (tetrahydropyran-4-ylmethyl)-amide | | | C₁₉H₂₀³⁵ClF₃N₄O₂ |
| 113 | 2-(2-Chloro-5-methyl-phenylamino)-4-tri- | A | B | 3.28 |
| | fluoromethyl-pyrimidine-5-carboxylic acid | | | 429 |
| | (tetrahydropyran-4-ylmethyl)-amide | | | C₁₉H₂₀³⁵ClF₃N₄O₂ |
| 257 | 2-(2-Chlorophenylamino)-4-trifluoromethyl- | C (5 | B | 3.52 |
| | pyrimidine-5-carboxylic acid | equivalents, | | 385 |
| | cyclobutylmethyl-amide | 2 × 30 min) | | C₁₇H₁₆³⁵ClF₃N₄O |
| 258 | 2-(3-Fluoro-5-trifluoromethylphenylamino)- | C (5 | B | 3.80 |
| | 4-trifluoromethyl-pyrimidine-5-carboxylic | equivalents, | | 437 |
| | acid cyclobutylmethyl-amide | 2 × 30 min) | | C₁₈H₁₅F₇N₄O |
| 259 | 2-(5-Chloro-2-methylphenylamino)-4- | C (5 | B | 3.61 |
| | trifluoromethyl-pyrimidine-5-carboxylic acid | equivalents, | | 399 |
| | cyclobutylmethyl-amide | 2 × 30 min) | | C₁₈H₁₈³⁵ClF₃N₄O |

**In Example 103 - Preparation Method B (5 equiv, 15 min) N.B. Reaction mixture also contained 0.5 ml MeCN and purification method C The product was purified by trituration with isohexane after this.

Compounds of Examples 114 to 145 and 260 were prepared as set out for table 5 and purified as follows:

Purification Method A: as for reference example 1c,

Purification Method C: The reaction was worked up as in example 1c, and the product purified by Biotage chromatography using the following solvent systems:

Sol 1 ethyl acetate
Sol 2 1% methanol in dichloromethane
Sol 3 2% methanol in dichloromethane

TABLE 5

| | | | Mass spec details |
|---|---|---|---|
| | | | 1 Retention time |
| | | | 2 MH⁺ |
| Ex. | | Purification | 3 Formula consistent |
| No. | Compound Name | method | with MH⁺ |
| 114 | 3-Fluorophenylamino-4-trifluoromethyl- | A | 3.23 |
| | pyrimidine-5-carboxylic acid cyclopentyl-amide | | 369 |
| | | | C₁₇H₁₆F₄N₄O |
| 115 | 2,6-Dichlorophenylamino-4-trifluoromethyl- | A | 3.17 |
| | pyrimidine-5-carboxylic acid cyclopentylamide | | 419 |
| | | | C₁₇H₁₅³⁵Cl₂F₃N₄O |
| 116 | 3-Chlorophenylamino-4- | A | 3.79 |
| | trifluoromethylpyrimidine-5-carboxylic acid (2- | | 401 |
| | ethylbutyl)-amide | | C₁₈H₂₀³⁵ClF₃N₄O |
| 117 | 2-Phenylamino-4-trifluoromethyl-pyrimidin-5- | A | 2.98 |
| | carboxylic acid (2-methoxy-ethyl)-amide | | 339 |
| | | | C₁₅H₁₅F₃N₄O₂ |
| 118 | 2-Phenylamino-4-trifluoromethyl-pyrimidin-5- | A | 2.32 |
| | carboxylic acid [2-(dimethyl-amino)ethyl]-amide | | 354 |
| | | | C₁₆H₁₈F₃N₅O |
| 119 | 1-[2-(3-Chlorophenyl-amino)-4-trifluoro- | A | 3.33 |
| | methylpyrimidin-5-yl]-1-(4-methoxypiperin-1-yl)- | | 415 |
| | methanone | | C₁₈H₁₈³⁵ClF₃N₄O₂ |
| 120 | 1-[2-(3-Chlorophenyl-amino)-4-trifluoro- | A | 3.16 |
| | methylpyrimidin-5-yl]-1-(1,1-dioxothiomorpholin- | | 435 |
| | 4yl)-methanone | | C₁₆H₁₄³⁵ClF₃N₄O₃S |
| 121 | N-((R)-1-{1-[2-(3-Chlorophenylamino)-4- | A | 2.91 |
| | trifluoromethyl-pyrimidin-5-yl]-methanoyl}- | | 428 |
| | pyrrolidin-3-yl)-acetamide | | C₁₈H₁₇³⁵ClF₃N₅O₂ |
| 122 | N-((S)-1-{1-[2-(3-Chlorophenylamino)-4- | A | 2.91 |
| | trifluoromethyl-pyrimidin-5-yl]-methanoyl}- | | 428 |
| | pyrrolidin-3-yl)-acetamide | | C₁₈H₁₇³⁵ClF₃N₅O₂ |
| 123 | 1-{1-[2-(3-Chloro-phenylamino)-4-tri- | C | 2.98 |
| | fluoromethyl-pyrimidin-5-yl]-methanoyl}- | Sol 1 | 442 |
| | piperidine-4-carboxylic acid methylamide | | C₁₉H₁₉³⁵ClF₃N₅O₂ |
| 124 | 2-(3-Chlorophenyl-amino)-4- | A | 3.00 |
| | trifluoromethylpyrimidine-5-carboxylic acid (4- | | 429 |
| | hydroxytetrahydropyran-4-ylmethyl)-amide | | C₁₈H₁₈³⁵ClF₃N₄O₃ |
| 125 | 2-(3-Fluorophenyl-amino)-4- | A | 2.86 |
| | trifluoromethylpyrimidine-5-Carboxylic acid (1- | | 413 |
| | hydroxytetrahydropyran-4-ylmethyl)-amide | | C₁₈H₁₈F₄N₄O₃ |
| 126 | 1-[2-(3-Chlorophenylamino)-4- | A | 2.53 |
| | trifluoromethylpyrimidin-5-yl]-1-(4- | | 400 |
| | methylpiperazin-1-yl)-methanone | | C₁₇H₁₇³⁵ClF₃N₅O |
| 127 | 2-(3-Chlorophenylamino)-4- | A | 3.23 |
| | trifluoromethylpyrimidine-5-carboxylic acid (2- | | 375 |
| | methoxy-ethyl)-amide | | C₁₅H₁₄³⁵ClF₃N₄O₂ |
| 128 | 2-(3-Chlorophenylamino)-4- | A | 2.51 |
| | trifluoromethylpyrimidine-5-carboxylic acid (2- | | 388 |
| | dimethylamino-ethyl)-amide | | C₁₆H₁₇³⁵ClF₃N₅O |
| 129 | 2-(3-chlorophenylamino)-4- | A | 3.25 |
| | trifluoromethylpyrimidine-5-carboxylic acid [R-1- | | 401 |
| | (tetrahydrofuran-2-yl)methyl]-amide | | C₁₇H₁₆³⁵ClF₃N₄O₂ |
| 130 | 2-(Phenylamino)-4-trifluoromethylpyrimidine-5- | A | 3.01 |
| | carboxylic acid (tetrahydro-pyran-4-ylmethyl)- | | 381 |
| | amide | | C₁₈H₁₉F₃N₄O₂ |
| 131 | 2-(2,3-Dichlorophenylamino)-4- | C | 3.74 |
| | trifluoromethylpyrimidine-5-carboxylic acid | Sol 2 | 433 |
| | cyclohexylamide | | C₁₈H₁₇³⁵Cl₂F₃N₄O |
| 132 | 2-(3-Fluorophenylamino)-4- | A | 3.56 |
| | trifluoromethylpyrimidine-5-carboxylic acid | | 383 |
| | cyclohexylamide | | C₁₈H₁₈F₄N₄O |
| 133 | 2-(3-Bromophenylamino)-4- | A | 3.74 |
| | trifluoromethylpyrimidine-5-carboxylic acid | | 445 |
| | cyclohexylamide | | C₁₈H₁₈⁸¹BrF₃N₄O |
| 134 | 2-(3-Fluorophenylamino)-4- | A | 3.06 |
| | trifluoromethylpyrimidine-5-carboxylic acid | | 385 |
| | (tetrahydropyran-4-yl)-amide | | C₁₇H₁₆F₄N₄O₂ |
| 135 | 2-(3-Bromophenylamino)-4- | A | 3.26 |
| | trifluoromethylpyrimidine-5-carboxylic acid | | 447 |
| | (tetrahydropyran-4-yl)-amide | | C₁₇H₁₆⁸¹BrF₃N₄O₂ |
| 136 | 2-(2,4-Dichlorophenylamino)-4- | A | 3.33 |
| | trifluoromethylpyrimidine-5-carboxylic acid | | 435 |
| | (tetrahydropyran-4-yl)-amide | | C₁₇H₁₅³⁵Cl₂F₃N₄O₂ |
| 137 | 2-(2,4-Dichlorophenylamino)-4- | A | 3.79 |
| | trifluoromethylpyrimidine-5-carboxylic acid | | 433 |
| | cyclohexylamide | | C₁₈H₁₇³⁵Cl₂F₃N₄O |
| 138 | 2-(3,4-Dichlorophenylamino)-4- | A | 3.90 |
| | trifluoromethylpyrimidine-5-carboxylic acid | | 433 |
| | cyclohexylamide | | C₁₈H₁₇³⁵Cl₂F₃N₄O |
| 139 | 2-(2,3-Dichlorophenylamino)-4- | A | 3.26 |
| | trifluoromethylpyrimidine-5-carboxylic acid | | 435 |
| | (tetrahydropyran-4-yl)-amide | | C₁₇H₁₅³⁵Cl₂F₃N₄O₂ |
| 140 | 2-(3-Fluorophenylamino)-4- | C | 3.37 |
| | trifluoromethylpyrimidine-5-carboxylic acid | Sol 2 | 401 |
| | (tetrahydro-thiopyran-4-yl)-amide | | C₁₇H₁₆F₄N₄OS |
| 141 | 2-(3-Chlorophenylamino)-4- | C | 3.51 |
| | trifluoromethylpyrimidine-5-carboxylic acid | Sol 2 | 417 |
| | (tetrahydro-thiopyran-4-yl)-amide | | C₁₇H₁₆³⁵ClF₃N₄OS |
| 142 | 2-(3-Bromophenylamino)-4- | C | 3.55 |
| | trifluoromethylpyrimidine-5-carboxylic acid | Sol 2 | 463 |
| | (tetrahydro-thiopyran-4-yl)-amide | | C₁₇H₁₆⁸¹BrF₃N₄OS |
| 143 | 2-(2,4-Dichlorophenylamino)-4- | C | 3.61 |
| | trifluoromethylpyrimidine-5-carboxylic acid | Sol 2 | 451 |
| | (tetrahydro-thiopyran-4-yl)-amide | | C₁₇H₁₅³⁵Cl₂F₃N₄OS |
| 144 | 2-(3,4-Dichlorophenylamino)-4- | C | 3.72 |
| | trifluoromethylpyrimidine-5-carboxylic acid | Sol 2 | 451 |
| | (tetrahydro-thiopyran-4-yl)-amide | | C₁₇H₁₅³⁵Cl₂F₃N₄OS |
| 145 | 2-(3,4-Dichlorophenylamino)-4- | C | 3.32 |
| | trifluoromethylpyrimidine-5-carboxylic acid (1,1- | Sol 3 | 483 |
| | dioxo-hexahydro-1l⁶-thiopyran-4-yl)-amide | | C₁₇H₁₅³⁵Cl₂F₃N₄O₃S |
| 260 | 3-Chlorophenylamino-4-trifluoromethyl- | A | 3.09 |
| | pyrimidine-5-carboxylic acid 2- | | 375 |
| | (hydroxypropyl)amide | | C₁₅H₁₄³⁵ClF₃N₄O₂ |

Compounds of Examples 146 to 162, 261 and 262 were prepared as set out in table 6.

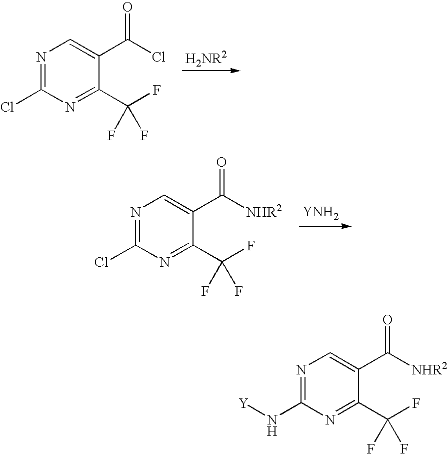

Preparation Method A: refers to the procedure give in part (b) of Example 166.

Preparation Method B: Exemplified by Example 154: A mixture of 2-chloro-4-trifluoromethyl-pyrimidin-5-carboxylic acid (tetrahydropyran-4-ylmethyl)-amide (50 mg), 3,5-dicyanoaniline (69 mg), and acetonitrile (0.5 ml) was irradiated in a microwave apparatus (the model used was the ‘Creator’, supplied by ‘Personal Chemistry’, operating at 300 Watts), at 180° C. for 60 min. The temperature, duration of irradiation, and number of equivalents of the substituted-aniline used are given after the method in the table.

Preparation Method C: exemplified by Example 162: A mixture of 2-chloro-4-trifluoromethyl-pyrimidin-5-carboxylic acid (tetrahydropyran-4-ylmethyl)-amide (80 mg) and 4-fluoro-2-(trifluoromethyl)aniline (111 mg) was irradiated in microwave apparatus (the model used was the ‘Creator’, supplied by ‘Personal Chemistry’, operating at 300 Watts), at 190° C. for 45 min.

Purification was carried out as detailed in the table to give the product.

Purification Method A: refers to the procedure give in part (b) of Example 166.

Purification Method B: mass directed autopurification usirig the procedures detailed at the beginning of the experimental.

Purification Method C: The reaction was worked up as for part (b) of Example 166, and the crude product further purified by Biotage chromatography over Merck 9385 silica gel, eluting with isohexane/ethyl acetate (7:3).

TABLE 6

| | | | | LCMS |
|---|---|---|---|---|
| | | | | 1 Retention time (min) |
| Ex. | | Prep | Purific | 2 MH⁺ |
| No. | Compound Name | Method | method | 3 Consistent Formula |
| 146 | 2-(3-Methoxy-5-trifluoromethyl- | A | B | 3.91 |
| | phenylamino)-4-trifluoromethyl-pyrimidine- | | | 477 |
| | 5-carboxylic acid cyclohexylmethyl-amide | | | C₂₁H₂₂F₆N₄O₂ |
| 147 | 2-(4-Chloro-3-methyl-phenylamino)-4- | A | B | 3.96 |
| | trifluoromethyl-pyrimidine-5-carboxylic acid | | | 427 |
| | cyclohexylmethyl-amide | | | C₂₀H₂₂³⁵ClF₃N₄O |
| 148 | 2-(3-Chloro-4-methyl-phenylamino)-4- | A | B | 3.92 |
| | trifluoromethyl-pyrimidine-5-carboxylic acid | | | 427 |
| | N-cyclohexylmethyl-amide | | | C₂₀H₂₂³⁵ClF₃N₄O |
| 149 | 2-(4-Chloro-3-cyano-phenylamino)-4- | A | B | 3.76 |
| | trifluoromethyl-pyrimidine-5-carboxylic acid | | | 438 |
| | cyclohexylmethyl-amide | | | C₂₀H₁₉³⁵ClF₃N₅O |
| 150 | 2-(2-Chloro-5-methyl-phenylamino)-4- | A | B | 3.82 |
| | trifluoromethyl-pyrimidine-5-carboxylic acid | | | 427 |
| | cyclohexylmethyl-amide | | | C₂₀H₂₂³⁵ClF₃N₄O |
| 151 | 2(3-Chloro-2,6-dimethyl-phenylamino)-4- | A | B | 3.76 |
| | trifluoromethyl-pyrimidine-5-carboxylic acid | | | 441 |
| | cyclohexylmethyl-amide | | | C₂₁H₂₄³⁵ClF₃N₄O |
| 152 | 2-(3-Chloro-4-tri-fluoromethoxyphenyl- | A | A | 4.00 |
| | amino)-4-trifluoromethyl-pyrimidine-5- | | | 497 |
| | carboxylic acid cyclo-hexylmethyl-amide | | | C₂₀H₁₉³⁵ClF₆N₄O₂ |
| 153 | 2-(3-Fluoro-4-tri-fluoromethylphenyl-amino)- | A | C | 3.89 |
| | 4-trifluoromethyl-pyrimidine-5-carboxylic | | | 465 |
| | acid cyclo-hexylmethyl-amide | | | C₂₀H₁₉F₇N₄O |
| 154 | 2-(3,5-Dicyano-phenylamino)-4- | B | B | 3.01 |
| | trifluoromethyl-pyrimidine-5-carboxylic acid | (180°, | | 431 |
| | (tetrahydropyran-4-ylmethyl)-amide | 60 mins, | | C₂₀H₁₇F₃N₆O₂ |
| | | 3 equiv) | | |
| 155 | 2-(3-Chloro-2,6-di-methylphenylamino)-4- | A | B | 3.22 |
| | trifluoromethyl-pyrimidine-5-carboxylic acid | | | 443 |
| | (tetrahydro-pyran-4-ylmethyl)-amide | | | C₂₀H₂₂³⁵ClF₃N₄O₂ |
| 156 | 2-(2-Chloro-6-methyl-phenylamino)-4-tri- | B | B | 3.05 |
| | fluoromethyl-pyrimidine-5-carboxylic acid | (180°, | | 429 |
| | (tetrahydropyran-4-ylmethyl)-amide | 60 mins, | | C₁₉H₂₀³⁵ClF₃N₄O₂ |
| | | 5 equiv) | | |
| 157 | 2-(2-Chloro-3-methyl-phenylamino)-4-tri- | A | B | 3.27 |
| | fluoromethyl-pyrimidine-5-carboxylic acid | | | 429 |
| | (tetrahydropyran-4-ylmethyl)-amide | | | C₁₉H₂₀³⁵ClF₃N₄O₂ |
| 158 | 2-(4-Chloro-2,6-dimethylphenylamino)-4- | A | B | 3.26 |
| | trifluoromethyl-pyrimidine-5-carboxylic acid | | | 443 |
| | (tetrahydropyran-4-ylmethyl)-amide | | | C₂₀H₂₂³⁵ClF₃N₄O₂ |
| 159 | 2-(5-Chloro-2-sulfamoylphenyl-amino)-4- | B (180°, | B | 3.08 |
| | trifluoromethyl-pyrimidine-5-carboxylic acid | 60 mins, 5 | | 494 |
| | (tetrahydropyran-4-ylmethyl)-amide | equiv) | | C₁₈H₁₉³⁵ClF₃N₅O₄S |
| 160 | 2-(2-Fluoro-4-trifluoromethylphenylamino)-4- | A | A | 3.67 |
| | trifluoromethyl-pyrimidine-5-carboxylic acid | | | 437 |
| | cyclobutylmethyl-amide | | | C₁₈H₁₅F₇N₄O |
| 161 | 2-(2-Chloro-4-trifluoromethylphenylamino)-4- | A | B | 3.97 |
| | trifluoromethyl-pyrimidine-5-carboxylic acid | | | 481 |
| | cyclohexylmethyl-amide | | | C₂₀H₁₉³⁵ClF₆N₄O |
| 162 | 2-(2-Trifluoromethyl-4-fluorophenylamino)-4- | C | B | 3.16 |
| | trifluoromethyl-pyrimidine-5-carboxylic acid | | | 467 |
| | (tetrahydro-pyran-4-ylmethyl)amide | | | C₁₉H₁₇N₄O₂F₇ |
| 261 | 2-(2-Fluoro-4-trifluoromethylphenylamino)-4- | B | B | 3.37 |
| | trifluoromethyl-pyrimidine-5-carboxylic acid | 180°, | | 467 |
| | (tetrahydropyran-4-ylmethyl)-amide | 2 × 42 min, 5 | | C₁₉H₁₇F₇N₄O₂ |
| | | equivalents | | |
| 262 | 2-(3,5-Bistrifluoromethylphenylamino)-4- | B | B | Molecular ion observed |
| | trifluoromethyl-pyrimidine-5-carboxylic acid | 180°, | | [M − H]⁻ 485 consistent |
| | cyclobutylmethyl-amide | 30 min, 5 | | with molecular formula |
| | | equivalents | | C₁₉H₁₅F₉N₄O |

### EXAMPLE 163

### 2-(3-Methoxyphenylamino)-4-trifluoromethyl-pyrimidine-5-carboxylic acid cyclohexylmethyl-amide

In a manner similar to Reference Example 1(c) 2-(3-methoxyphenylamino)-4-trifluoro-methylpyrimidine-5-carboxylic acid (32 mg) and cyclohexanemethanamine (16 μl, ex Lancaster) afforded the title compound (28 mg).

NMR (DMSO-d6) δ 0.85-1.0 (2H, m), 1.1-1.25 (3H, m), 1.5 (1H, m), 1.55-1.75 (5H, m), 3.06 (2H, t), 3.74 (3H, s), 6.63 (1H, d), 7.2-7.3 (2H, m), 7.54 (1H, s), 8.57 (1H, t), 8.74 (1H, s), 10.35 (1H, s).

LC/MS, t=3.57 min, Molecular ion observed [MH⁺]=409 consistent with the molecular formula C₂₀H₂₃F₃N₄O₂.

### EXAMPLE 164

### 2-(3-Chlorophenylamino)-4-trifluoromethyl-pyrimidine-5-carboxylic acid (1-hydroxycyclohexylmethyl)-amide

In a manner similar to Reference Example 1(c) 2-(3-chlorophenylamino)-4-trifluoro-methylpyrimidine-5-carboxylic acid (32 mg) and 1-aminomethyl-1-cyclohexanol hydrochloride (20 mg, ex Aldrich) afforded the title compound (29 mg).

NMR (DMSO-d6) δ 1.3 (1H, m), 1.4-1.5 (7H, m), 1.6 (2H, m), 3.28 (2H, d), 4.34 (1H, s), 7.16 (1H, d), 7.43 (1H, t), 7.73 (1H, d), 8.04 (1H, t), 8.51 (1H, t), 8.91 (1H, s), 10.65 (1H, s).

LC/MS, t=3.39 min, Molecular ion observed [M-H]⁻=427 consistent with the molecular formula C₁₉H₂₀³⁵ClF₃N₄O₂.

### EXAMPLE 165

### 2-(3-Bromophenylamino)-4-trifluoromethyl-pyrimidine-5-carboxylic acid (1-hydroxycyclohexylmethyl)-amide

In a manner similar to Reference Example 1(c) 2-(3-bromophenylamino)-4-trifluoro-methylpyrimidine-5-carboxylic acid (36.5 mg) and 1-aminomethyl-1-cyclohexanol hydro-chloride (20 mg, ex Aldrich) afforded the title compound (28 mg).

NMR (DMSO-d6) δ 1.25 (1H, m), 1.35-1.45 (7H, m), 1.6 (2H, m), 3.23 (2H, d), 4.28 (1H, s), 7.23 (1H, d), 7.31 (1H, t), 7.71 (1H, d), 8.12 (1H, s), 8.45 (1H, t), 8.85 (1H, s), 10.55 (1H, s).

LC/MS, t=3.43 min, Molecular ion observed [M-H]⁻=471 consistent with the molecular formula C₁₉H₂₀⁷⁹BrF₃N₄O₂

### EXAMPLE 166

### 2-(3-Chloro-4-fluorophenylamino)-4-trifluoromethyl-pyrimidine-5-carboxylic acid cyclohexylmethyl-amide

(a). To a solution of 2-chloro-4-trifluoromethyl-pyrimidin-5-carbonyl chloride (750 mg, ex Maybridge) in dichloromethane (15 ml) at −40° was added dropwise over 30 minutes a solution of cyclohexanemethanamine (0.35 ml, ex Lancaster) and triethylamine (0.41 ml) in dichloromethane (15 ml). Dichloromethane was removed under reduced pressure and ethyl acetate (20 ml) added. The solution was washed sequentially with water, 5% sodium bicarbonate solution and water, dried (MgSO₄), evaporated and triturated with ether:hexane to afford 2-chloro-4-trifluoromethyl-pyrimidine-5-carboxylic acid cyclohexylmethyl-amide (666 mg).

NMR (DMSO-d6) δ 0.85-1.0 (2H, m), 1.1-1.25 (3H, m), 1.5 (1H, m), 1.55-1.75 (5H, m), 3.12 (2H, t), 8.75 (1H, t), 9.18 (1H, s).

LC/MS, t=3.31 min, Molecular ion observed [MH⁺]=322 consistent with the molecular formula C₁₃H₁₅³⁵ClF₃N₃O).

(b). To a solution of 2-chloro-4-trifluoromethyl-pyrimidine-5-carboxylic acid cyclohexylmethyl-amide (100 mg) in 1,4-dioxan (1 ml) was added 3-chloro-4-fluoroaniline (228 mg, ex Lancaster) and the solution stirred at reflux for 4 hours. Dioxan was removed under reduced pressure and ethyl acetate (5 ml) added. The solution was washed sequentially with 2N hydrochloric acid (2×3 ml) and water (3×3 ml), dried (MgSO₄), evaporated and triturated with isohexane to afford the title compound (107 mg).

NMR (DMSO-d6) δ 0.85-1.0 (2H, m), 1.1-1.25 (3H, m), 1.45 (1H, m), 1.6-1.75 (5H, m), 3.06 (2H, t), 7.25 (1H, t), 7.43 (1H, t), 7.56 (1H, t), 8.56 (1H, t), 8.69 (1H, s), 10.20 (1H, s).

LC/MS, t=3.81 min, Molecular ion observed [MH⁺]=431 consistent with the molecular formula C₁₉H₁₉³⁵ClF₄N₄O.

### EXAMPLE 167

### 2-(3-Chloro-2-fluorophenylamino)-4-trifluoromethyl-pyrimidine-5-carboxylic acid cyclohexylmethyl-amide

In a manner similar to Example 166(b), 2-chloro-4-trifluoromethyl-pyrimidine-5-carboxylic acid cyclohexylmethyl-amide (100 mg) and 3-chloro-2-fluoroaniline (230 mg, ex Acros) afforded the title compound (101 mg).

NMR (DMSO-d6) δ 0.85-1.0 (2H, m), 1.1-1.25 (3H, m), 1.5 (1H, m), 1.6-1.8 (5H, m), 3.08 (2H, t), 7.43 (1H, t), 7.67 (1H, m), 8.07 (1H, d), 8.58 (1H, t), 8.80 (1H, s), 10.60 (1H, s).

LC/MS, t=3.71 min, Molecular ion observed [MH⁺]=431 consistent with the molecular formula C₁₉H₁₉³⁵ClF₄N₄O.

### EXAMPLE 168

### 2-(5-Chloro-2-fluorophenylamino)-4-trifluoromethyl-pyrimidine-5-carboxylic acid cyclohexylmethyl-amide

In a manner similar to Example 166(b), 2-chloro-4-trifluoromethyl-pyrimidine-5-carboxylic acid cyclohexylmethyl-amide (100 mg) and 5-chloro-2-fluoroaniline (230 mg, ex Avocado) afforded the title compound (116 mg).

NMR (DMSO-d6) δ 0.85-1.0 (2H, m), 1.1-1.25 (3H, m), 1.5 (1H, m), 1.6-1.75 (5H, m), 3.07 (2H, t), 7.29 (1H, m), 7.36 (1H, t), 7.77 (1H, d of d), 8.57 (1H, t), 8.72 (1H, s), 10.15 (1H, s).

LC/MS, t=3.73 min, Molecular ion observed [MH⁺]=431 consistent with the molecular formula C₁₉H₁₉³⁵ClF₄N₄O.

### EXAMPLE 169

### 2-(3,5-Difluorophenylamino)-4-trifluoromethyl-pyrimidin-5-carboxylic acid cyclohexylmethyl-amide

In a manner similar to Example 166(b) 2-chloro-4-trifluoromethyl-pyrimidine-5-carboxylic acid cyclohexylmethyl-amide (100 mg) and 3,5-difluoroaniline (200 mg, ex Lancaster) afforded the title compound (110 mg).

NMR (DMSO-d6) δ 0.85-1.0 (2H, m), 1.1-1.25 (3H, m), 1.5 (1H, m), 1.6-1.75 (5H, m), 3.09 (2H, t), 6.89 (1H, t), 7.54 (2H, d), 8.60 (1H, t), 8.85 (1H, s), 10.80 (1H, s).

LC/MS, t=3.74 min, Molecular ion observed [MH⁺]=415 consistent with the molecular formula C₁₉H₁₉F₅N₄O.

### EXAMPLE 170

### 2-(4-Chloro-2-trifluoromethylphenylamino)-4-trifluoromethyl-pyrimidine-5-carboxylic acid cyclohexylmethyl-amide

In a manner similar to Example 166(b) 2-chloro-4-trifluoromethyl-pyrimidine-5-carboxylic acid cyclohexylmethyl-amide (80 mg) and 4-chloro-2-trifluoromethylaniline (107 mg, ex Lancaster) afforded, after purification by mass-directed autopreparation technique, the title compound (6 mg).

NMR (DMSO-d6) δ 0.85-1.0 (2H, m), 1.1-1.25 (3H, m), 1.5 (1H, m), 1.55-1.75 (5H, m), 3.06 (2H, t), 7.76 (1H, d), 7.88 (1H, d), 7.97 (1H, s), 8.56 (1H, t), 8.70 (1H, s), 10.15 (1H, s).

LC/MS, t=3.97 min, Molecular ion observed [MH⁺]=481 consistent with the molecular formula C₂₀H₁₉³⁵ClF₆N₄O.

### EXAMPLE 171

### 2-(3-Cyanophenylamino)-4-trifluoromethyl-pyrimidine-5-carboxylic acid cyclohexylmethyl-amide

To a solution of 2-chloro-4-trifluoromethyl-pyrimidine-5-carboxylic acid cyclohexylmethyl-amide (50 mg) in acetonitrile (0.5 ml) was added 3-aminobenzonitrile (92 mg, ex Aldrich) and the solution heated at 200° C. under microwave conditions for 45 minutes. Acetonitrile was removed under reduced pressure and ethyl acetate (5 ml) added. The solution was washed sequentially with 2N hydrochloric acid (2×3 ml) and water (3×3 ml), dried (MgSO₄), evaporated and the residue purified using silica gel chromatography with 1:1 ethyl acetate:isohexane to afford the title compound (37 mg).

NMR (DMSO-d6) δ 0.85-1.0 (2H, m), 1.1-1.25 (3H, m), 1.5 (1H, m), 1.6-1.8 (5H, m), 3.08 (2H, t), 7.50 (1H, d), 7.57 (1H, t), 8.00 (1H, d), 8.25 (1H, s), 8.59 (1H, t), 8.83 (1H, s), 10.75 (1H, s).

LC/MS, t=3.51 min, Molecular ion observed [MH⁺]=404 consistent with the molecular formula C₂₀H₂₀F₃N₅O.

### EXAMPLE 172

### 2-(3-Cyanophenylamino)-4-trifluoromethyl-pyrimidine-5-carboxylic acid (tetrahydropyran-4-ylmethyl)-amide

In a manner similar to Reference Example 1(c) 2-(3-cyanophenylamino)-4-trifluoro-methylpyrimidine-5-carboxylic acid (32 mg) and 4-aminomethyltetrahydropyran (14 mg, ex Combi Blocks) afforded the title compound (26 mg).

NMR (DMSO-d6) δ 1.15-1.25 (2H, m), 1.62 (2H, d), 1.74 (1H, m), 3.14 (2H, t), 3.27 (2H, t), 3.86 (2H, d of d), 7.50 (1H, d), 7.57 (1H, t), 8.00 (1H, d), 8.26 (1H, s), 8.65 (1H, t), 8.85 (1H, s), 10.70 (1H, s).

LC/MS, t=−2.94 min, Molecular ion observed [MH⁺]=406 consistent with the molecular formula C₁₉H₁₈F₃N₅O₂.

### EXAMPLE 173

### 2-(3-Cyanophenylamino)-4-trifluoromethyl-pyrimidine-5-carboxylic acid cyclopentylmethyl-amide

In a manner similar to Reference Example 1(c) 2-(3-cyanophenylamino)-4-trifluoro-methylpyrimidine-5-carboxylic acid (32 mg) and cyclopentanemethanamine hydrochloride (17 mg) afforded the title compound (16 mg).

NMR (DMSO-d6) δ 1.20-1.30 (2H, m), 1.45-1.6 (4H, m), 1.65-1.75 (2H, m), 2.08 (1H, quintuplet), 3.19 (2H, t), 7.50 (1H, d), 7.57 (1H, t), 8.00 (1H, d), 8.25 (1H, s), 8.63 (1H, t), 8.82 (1H, s), 10.70 (1H, s).

LC/MS, t=3.42 min, Molecular ion observed [MH⁺] 390 consistent with the molecular formula C₁₉H₁₈F₃N₅O.

### EXAMPLE 174

### 2-(4-Cyanophenylamino)-4-trifluoromethyl-pyrimidine-5-carboxylic acid cyclohexylmethyl-amide

In a manner similar to Reference Example 1(c) 2-(4-cyanophenylamino)-4-trifluoro-methylpyrimidine-5-carboxylic acid (32 mg) and cyclohexanemethanamine (16 μl, ex Lancaster) afforded the title compound (18 mg).

NMR (DMSO-d6) δ 0.85-1.0 (2H, m), 1.1-1.25 (3H, m), 1.5 (1H, m), 1.6-1.8 (5H, m), 3.08 (2H, t), 7.81 (2H, d), 7.97 (2H, d), 8.61 (1H, t), 8.85 (1H, s), 10.90 (1H, s).

LC/MS, t=3.51 min, Molecular ion observed [MH⁺]=404 consistent with the molecular formula C₂₀H₂₀F₃N₅O.

### EXAMPLE 175

### 2-(4-Cyanophenylamino)-4-trifluoromethyl-pyrimidine-5-carboxylic acid (tetrahydropyran-4-ylmethyl)-amide

In a manner similar to Reference Example 1(c) 2-(4-cyanophenylamino)-4-trifluoro-methylpyrimidine-5-carboxylic acid (32 mg) and 4-aminomethyltetrahydropyran (14 mg, ex Combi Blocks) afforded the title compound (6 mg).

NMR (DMSO-d6) δ 1.15-1.25 (2H, m), 1.60 (2H, d), 175 (1H, m), 3.14 (2H, t), 3.27 (2H, t), 3.86 (2H, d), 7.82 (2H, d), 7.97 (2H, d), 8.67 (1H, t), 8.87 (1H, s), 10.85 (1H, s).

LC/MS, t=2.92 min, Molecular ion observed [MH⁺]=406 consistent with the molecular formula C₁₉H₁₈F₃N₅O₂.

### EXAMPLE 176

### 2-(4-Cyanophenylamino)-4-trifluoromethyl-pyrimidine-5-carboxylic acid cyclopentylmethyl-amide

In a manner similar to Reference Example 1(c) 2-(4-cyanophenylamino)-4-trifluoro-methylpyrimidine-5-carboxylic acid (32 mg) and cyclopentanemethanamine hydrochloride (17 mg) afforded the title compound (22.5 mg).

NMR (DMSO-d6) δ 1.15-1.30 (2H, m), 1.45-1.65 (4H, m), 1.65-1.75 (2H, m), 2.08 (1H, quintuplet), 3.17 (2H, t), 7.82 (2H, d), 7.97 (2H, d), 8.64 (1H, t), 8.84 (1H, s), 10.90 (1H, s).

LC/MS, t=3.40 min, Molecular ion observed [MH⁺]=390 consistent with the molecular formula C₁₉H₁₈F₃N₅O.

### EXAMPLE 177

### 2-(3-Methoxy-5-(trifluoromethyl)phenylamino)-4-trifluoromethyl-pyrimidine-5-carboxylic acid (tetrahydropyran-4-ylmethyl)-amide

(a). To a solution of 2-chloro-4-trifluoromethyl-pyrimidin-5-carbonyl chloride (1.5 g) in dichloromethane (20 ml) at −2° was added a dropwise a solution of 4-aminomethyltetra-hydropyran (0.70 g, ex Combi Blocks) and triethylamine (1.05 ml) in dichloromethane (10 ml) and the solution stirred at 0° for 1 hour. Dichloromethane was removed under reduced pressure and ethyl acetate (30 ml) added. The solution was washed with 2N hydrochloric acid (3×20 ml), dried (MgSO₄), evaporated and the residue purified using silica gel chromatography with 1:1 ethyl acetate:isohexane to afford 2-chloro-4-trifluoromethyl-pyrimidin-5-carboxylic acid (tetrahydropyran-4-yl-methyl)-amide (120 g).

NMR (DMSO-d6) δ 1.15-1.3 (2H, m), 1.61 (2H, d), 1.74 (1H, m), 3.17 (2H, t), 3.25 (2H, t), 3.86 (2H, d of d), 8.81 (1H, t), 9.20 (1H, s).

LC/MS, t=2.54 min, Molecular ion observed [MH⁺] 324 consistent with the molecular formula C₁₂H₁₃³⁵ClF₃N₃O₂.

(b). In a manner similar to Example 166(b), 2-chloro-4-trifluoromethyl-pyrimidin-5-carboxylic acid (tetrahydropyran-4-ylmethyl)-amide (50 mg) and 3-methoxy-5-(trifluoromethyl)aniline (148 mg, ex Aldrich) afforded after stirring at reflux for 24 hours the title compound (51 mg).

NMR (DMSO-d6) δ 1.15-1.3 (2H, m), 1.62 (2H, d), 1.74 (1H, m), 3.13 (2H, t), 3.27 (2H, t), 3.83 (3H, s), 3.86 (2H, d), 6.92 (1H, s), 7.73 (1H, s), 7.80 (1H, s), 8.64 (1H, t), 8.85 (1H, s), 10.65 (1H, s).

LC/MS, t=3.38 min, Molecular ion observed [MH⁺]=479 consistent with the molecular formula C₂₀H₂₀F₆N₄O₃.

### EXAMPLE 178

### 2-(3,5-Bis-trifluoromethylphenylamino)-4-trifluoromethyl-pyrimidine-5-carboxylic acid (tetrahydropyran-4-ylmethyl)-amide

In a manner similar to Example 166(b) 2-chloro-4-trifluoromethyl-pyrimidin-5-carboxylic acid (tetrahydropyran-4-ylmethyl)-amide (50 mg) and 3,5-bis(trifluoromethyl)aniline (177 mg, ex Aldrich) afforded, after stirring at reflux for 80 hours and purification by mass-directed autopreparation technique, the title compound (24.5 mg).

NMR (DMSO-d6) δ 1.15-1.3 (2H, m), 1.62 (2H, d), 1.75 (1H, m), 3.14 (2H, t), 3.28 (2H, t), 3.86 (2H, d), 7.72 (1H, s), 8.49 (2H, s), 8.67 (1H, t), 8.93 (1H, s), 11.05 (1H, s).

LC/MS, t=3.62 min, Molecular ion observed [MR⁺]=517 consistent with the molecular formula C₂₀H₁₇F₉N₄O₂.

### EXAMPLE 179

### 2-(3-Bromo-5-(trifluoromethyl)phenylamino)-4-trifluoromethyl-pyrimidine-5-carboxylic acid (tetrahydropyran-4-ylmethyl)-amide

In a manner similar to Example 166(b) 2-chloro-4-trifluoromethyl-pyrimidin-5-carboxylic acid (tetrahydropyran-4-ylmethyl)-amide (50 mg) and 3-bromo-5-(trifluoro-methyl)aniline (185 mg, ex Avocado) afforded, after stirring at reflux for 80 hours and purification by mass-directed autopreparation technique, the title compound (28 mg).

NMR (DMSO-d6) δ 1.15-1.3 (2H, m), 11.62 (2H, d), 1.74 (1H, m), 3.14 (2H, t), 3.28 (2H, t), 3.86 (2H, d), 7.60 (1H, s), 8.24 (1H, s), 8.29 (1H, s), 8.66 (1H, t), 8.99 (1H, s), 10.90 (1H, s).

LC/MS, t=3.63 min, Molecular ion observed [M-H]-=527 consistent with the molecular formula C₁₉H₁₇⁷⁹BrF₆N₄O₂.

### EXAMPLE 180

### 2-(3-Fluoro-5-(trifluoromethyl)phenylamino)-4-trifluoromethyl-pyrimidine-5-carboxylic acid (tetrahydropyran-4-ylmethyl)-amide

In a manner similar to Example 166(b) 2-chloro-4-trifluoromethyl-pyrimidin-5-carboxylic acid (tetrahydropyran-4-ylmethyl)-amide (50 mg) and 3-fluoro-5-(trifluoromethyl)aniline (138 mg, ex Fluorochem) afforded after stirring at reflux for 24 hours the title compound (44 mg).

NMR (DMSO-d6) δ 1.15-1.3 (2H, m), 1.62 (2H, d), 1.75 (1H, m), 3.14 (2H, t), 3.28 (2H, t), 3.86 (2H, d), 7.32 (1H, d), 7.96 (1H, d), 8.06 (1H, s), 8.67 (1H, t), 8.90 (1H, s), 10.90 (1H, s).

LC/MS, t=3.45 min, Molecular ion observed [MH⁺]=467 consistent with the molecular formula C₁₉H₁₇F₇N₄O₂.

### EXAMPLE 181

### 2-(2-Fluoro-3-(trifluoromethyl)phenylamino)-4-trifluoromethyl-pyrimidine-5-carboxylic acid (tetrahydropyran-4-ylmethyl)-amide

In a manner similar to Example 166(b) 2-chloro-4-trifluoromethyl-pyrimidin-5-carboxylic acid (tetrahydropyran-4-ylmethyl)-amide (50 mg) and 2-fluoro-3-(trifluoromethyl)aniline (138 mg, ex Aldrich) afforded, after stirring at reflux for 80 hours and purification by mass-directed autopreparation technique, the title compound (15 mg).

NMR (DMSO-d6) δ 1.1-1.25 (2H, m), 1.60 (2H, d), 1.73 (1H, m), 3.11 (2H, t), 3.26 (2H, t), 3.85 (2H, d), 7.43 (1H, t), 7.61 (1H, t), 7.92 (1H, s), 8.63 (1H, t), 8.72 (1H, s), 10.30 (1H, s).

LC/MS, t=3.28 min, Molecular ion observed [MH⁺]=467 consistent with the molecular formula C₁₉H₁₇F₇N₄O₂.

### EXAMPLE 182

### 2-(2-Methylthio-3-(trifluoromethyl)phenylamino)-4-trifluoromethyl-pyrimidine-5-carboxylic acid (tetrahydropyran-4-ylmethyl)-amide

2-Chloro-4-trifluoromethyl-pyrimidin-5-carboxylic acid (tetrahydropyran-4-ylmethyl)-amide (50 mg), 2-methylthio-3-(trifluoromethyl)aniline (125 mg, ex Maybridge) and acetonitrile (0.5 ml) were heated at 190° under microwave irradiation for 30 minutes. The solvent was evaporated in vacuo and the residue purified by mass-directed autopreparation technique, to give the title compound (11 mg).

NMR (DMSO-d6) δ 1.1-1.25 (2H, m), 1.60 (2H, d), 1.73 (1H, m), 2.24 (3H, s), 3.12 (2H, t), 3.26 (2H, t), 3.85 (2H, d), 7.65 (2H, d), 8.11 (1H, t), 8.64 (1H, t), 8.72 (1H, s), 9.81 (1H, s).

LC/MS, t=3.53 min, Molecular ion observed [MH⁺]=495 consistent with the molecular formula C₂₀H₂₀F₆N₄O₂S.

### EXAMPLE 183

### 2-(5-Chloro-2-methylphenylamino)-4-trifluoromethyl-pyrimidine-5-carboxylic acid (cyclopentylmethyl)-amide

(a). To a solution of 2-chloro-4-trifluoromethyl-pyrimidin-5-carbonyl chloride (1.0 g, ex Maybridge) in dichloromethane (7 ml) at −2° was added a dropwise a solution of cyclo-pentanemethanamine hydrochloride (0.55 g) and triethylamine (1.4 ml) in dichloromethane (13 ml) and the solution stirred at 0° for 1 hour. Dichloromethane was removed under reduced pressure and ethyl acetate (20 ml) added. The solution was washed with 2N hydrochloric acid (3×15 ml), dried (MgSO₄), evaporated and triturated with isohexane to afford 2-chloro-4-trifluoromethyl-pyrimidin-5-carboxylic acid (cyclopentylmethyl)-amide (838 mg).

NMR (DMSO-d6) δ 1.1-1.3 (2H, m), 1.45-1.65 (4H, m), 1.65-1.8 (2H, m), 2.07 (1H, quintuplet), 3.20 (2H, t), 8.78 (1H, t), 9.17 (1H, s).

LC/MS, t=3.22 min, Molecular ion observed [M-H]⁻=306 consistent with the molecular formula C₁₂H₁₃³⁵ClF₃N₃O.

(b). In a manner similar to Example 166(b) 2-chloro-4-trifluoromethyl-pyrimidine-5-carboxylic acid cyclopentylmethylamide (47.5 mg) and 5-chloro-2-methylaniline (110 mg, ex Aldrich) afforded after stirring at reflux for 30 hours the title compound (41 mg).

NMR (DMSO-d6) δ 1.15-1.3 (2H, m), 1.4-1.6 (4H, m), 1.65-1.75 (2H, m), 2.06 (1H, quintuplet), 2.20 (3H, s), 3.14 (2H, t), 7.19 (1H, d), 7.29 (1H, d), 7.48 (1H, s), 8.55 (1H, t), 8.63 (1H, s), 9.83 (1H, s).

LC/MS, t=3.68 min, Molecular ion observed [MH⁺]=413 consistent with the molecular formula C₁₉H₂₁³⁵ClF₃N₄O.

### EXAMPLE 184

### 2-(3-Chloro-4-methylphenylamino)-4-trifluoromethyl-pyrimidine-5-carboxylic acid (tetrahydropyran-4-ylmethyl)-amide

In a manner similar to Example 166(b) 2-chloro-4-trifluoromethyl-pyrimidine-5-carboxylic acid (tetrahydropyran-4-ylmethyl)-amide (50 mg) and 3-chloro-4-methyl-aniline (109 mg) afforded, after stirring at reflux for 24 hours and purification by mass-directed autopreparation technique, the title compound (35 mg).

NMR (DMSO-d6) δ 1.15-1.3 (2H, m), 1.61 (2H, d), 1.74 (1H, m), 2.28 (3H, s), 3.13 (2H, t), 3.27 (2H, t), 3.86 (2H, d of d), 7.31 (1H, d), 7.56 (1H, d), 7.94 (1H, s), 8.61 (1H, t), 8.79 (1H, s), 10.50 (1H, s). LC/MS, Molecular ion observed [MH⁺]=429 consistent with the molecular formula C₁₉H₂₀³⁵ClF₃N₄O₂.

### EXAMPLE 185

### 2-(3-Chloro-2-methylphenylamino)-4-trifluoromethyl-pyrimidine-5-carboxylic acid (tetrahydropyran-4-ylmethyl)-amide

In a manner similar to Example 166(b) 2-chloro-4-trifluoromethyl-pyrimidine-5-carboxylic acid (tetrahydropyran-4-ylmethyl)-amide (50 mg) and 3-chloro-2-methyl-aniline (109 mg, known compound CAS No 87-60-5) afforded, after stirring at reflux for 24 hours and purification by mass-directed autopreparation technique, the title compound (30 mg).

NMR (DMSO-d6) δ 1.1-1.25 (2H, m), 1.59 (2H, d), 1.72 (1H, m), 2.21 (3H, s), 3.10 (2H, t), 3.26 (2H, t), 3.84 (2H, d of d), 7.24 (1H, t), 7.3 (2H, m), 8.56 (1H, t), 8.61 (1H, s), 9.99 (1H, s).

LC/MS, t=3.19 min, Molecular ion observed [MH⁺]=429 consistent with the molecular formula C₁₉H₂₀³⁵ClF₃N₄O₂.

### EXAMPLE 186

### 2-(4-Chloro-3-methoxyphenylamino)-4-trifluoromethyl-pyrimidine-5-carboxylic acid (tetrahydropyran-4-ylmethyl)-amide

In a manner similar to Example 166(b) 2-chloro-4-trifluoromethyl-pyrimidine-5-carboxylic acid (tetrahydropyran-4-ylmethyl)-amide (50 mg) and 4-chloro-3-methoxy-aniline (122 mg) afforded, after stirring at reflux for 24 hours and purification by mass-directed autopreparation technique, the title compound (33 mg).

NMR (DMSO-d6) δ 1.1-1.25 (2H, m), 1.61 (2H, d), 1.73 (1H, m), 3.13 (2H, t), 3.27 (2H, t), 3.83 (3H, s), 3.86 (2H, d), 7.27 (1H, d), 7.37 (1H, d), 7.81 (1H, s), 8.63 (1H, t), 8.80 (1H, s), 10.50 (1H, s). LC/MS, t=3.26 min, Molecular ion observed [M=] 445 consistent with the molecular formula C₁₉H₂₀³⁵ClF₃N₄O₃.

### EXAMPLE 187

### 2-(4-Chloro-3-methylphenylamino)-4-trifluoromethyl-pyrimidine-5-carboxylic acid (tetrahydropyran-4-ylmethyl)-amide

In a manner similar to Example 166(b) 2-chloro-4-trifluoromethyl-pyrimidine-5-carboxylic acid (tetrahydropyran-4-ylmethyl)-amide (50 mg) and 4-chloro-3-methyl-aniline (109 mg, ex Lancaster) afforded, after stirring at reflux for 24 hours and purification by mass-directed autopreparation technique, the title compound (33 mg).

NMR (DMSO-d6) δ 1.15-1.3 (2H, m), 1.61 (2H, d), 1.73 (1H, m), 2.31 (3H, s), 3.12 (2H, t), 3.27 (2H, t), 3.86 (2H, d), 7.37 (1H, d), 7.62 (1H, d), 7.72 (1H, s), 8.61 (1H, t), 8.77 (1H, s), 10.45 (1H, s). LC/MS, t=3.41 min, Molecular ion observed [MH⁺]=429 consistent with the molecular formula C₁₉H₂₀³⁵ClF₃N₄O₂.

### EXAMPLE 188

### 2-(3-Chlorophenylamino)-4-trifluoromethyl-pyrimidine-5-carboxylic acid cyclobutylmethyl-methyl-amide

### a) N-(Cyclobutylmethyl)-2,2,2-trifluoroacetamide

C-cyclobutyl-methylamine hydrochloride (1.82 g) was added to a solution of N,N-diisopropylethylamine (4.14 g) in dry tetrahydrofuran (30 ml) at 0° C. The mixture was stirred at 0° C. for 5 mins then cooled to −20° C. A solution of trifluoroacetic anhydride (3.57 g) in tetrahydrofuran (10 ml) was added dropwise over 10 mins and the mixture was then allowed to stir at room temperature for 1 hour. The solution was diluted with ether (100 ml) and water (75 ml), separated and the organic layer washed with water, dilute hydrochloric acid, water and brine, dried (MgSO₄) and evaporated to give the title compound (2.63 g)

NMR (CDCl₃) δ 1.70 (2H, m excess), 1.93 (2H, m), 2.10 (2H, m), 2.53 (1H, m), 3.39 (2H, t), 6.2 (1H, br s).

### b) N-(Cyclobutylmethyl)-N-methylamine

N-(Cyclobutylmethyl)-2,2,2-trifluoroacetamide (2.62 g) and iodomethane (3.6 ml) were dissolved in dry acetone (75 ml). Powdered potassium hydroxide (3.2 g) was added and the mixture heated at reflux for 5 mins. The excess iodomethane and acetone were removed under reduced pressure, water (75 ml) added and the solution heated at reflux for 1 hour. The mixture was cooled and ether (75 ml) added. The layers were separated and the organic layer was extracted with dilute hydrochloric acid (75 ml). The aqueous extract was washed with ether, then made strongly basic with sodium hydroxide and extracted with ether (2×75 ml). The extracts were dried (K₂CO₃) and evaporated to give the title compound (517 mg)

NMR (CDCl₃) δ 1.3 (1H, m excess), 1.65 (2H, m), 1.9 (2H, m), 2.05 (2H, m), 2.45 (4H, m), 2.55 (2H, d).

### c) 2-(3-Chlorophenylamino)-4-trifluoromethyl-pyrimidine-5-carboxylic acid cyclobutylmethyl-methyl-amide

To a solution of N-(cyclobutylmethyl)-N-methylamine (17 mg) in dimethylformamide (1.5 ml) was added successively, 2-(3-chlorophenylamino)-4-trifluoromethyl-pyrimidine-5-carboxylic acid (35 mg), N,N-diisopropylethylamine (38 ul), 1-hydroxybenzotriazole hydrate (23 mg) and 1-(3-dimethylamino-propyl)-3-ethylcarbodiimide hydrochloride (25 mg). The solution was stirred overnight. Dimethylformamide was removed under reduced pressure and ethyl acetate (10 ml) added. The solution was washed sequentially with 10 ml portions of water, saturated sodium bicarbonate solution, water, dilute hydrochloric acid, water and brine, dried (MgSO₄) and evaporated to give the title compound (31 mg).

NMR (DMSO-d6) Rotamers in 60:40 ratio δ 1.5-2.1 (6H, m), 2.50 (0.4H, m excess), 2.65 (0.6H, m), 2.84 (1.8H, s), 2.94 (1.2H, s), 3.22 (0.4H, d), 3.50 (1.6H, br s), 7.09 (1H, d), 7.36 (1H, m), 7.66 (1H, m), 7.96 (1H, s), 8.76 (1H, d), 10.5 (1H, s).

LC/MS t=3.66 min, Molecular ion observed (MH⁺)=399 consistent with the molecular formula C₁₈H₁₈³⁵ClF₃N₄O

### EXAMPLE 189

### 2-(3-Chlorophenylamino)-4-trifluoromethyl-pyrimidine-5-carboxylic acid cyclohexylmethyl-methyl-amide

### a) N-(Cyclohexylmethyl)-2,2,2-trifluoroacetamide

In a manner similar to Example 188a) cyclohexanemethanamine (2.83 g) (Lancaster) gave the title compound (5.09 g).

NMR (CDCl₃) δ 0.95 (2H, m), 1.22 (3H, m), 1.54 (1H, m excess), 1.70 (5H, m), 3.21 (2H, t), 6.3 (1H, br s).

### b) N-(Cyclohexylmethyl)-N-methylamine

In a manner similar to Example 188b) N-(cyclohexylmethyl)-2,2,2-trifluoroacetamide (2.98 g) gave the title compound (1.41 g).

NMR (CDCl₃) δ 0.9 (2H, m), 1.23 (4H, m), 1.46 (1H, m excess), 1.72 (5H, m), 2.4 (5H, m).

### c) 2-(3-Chlorophenylamino)-4-trifluoromethyl-pyrimidine-5-carboxylic acid cyclohexylmethyl-methyl-amide

In a manner similar to Example 188c) 2-(3-chlorophenylamino)-4-trifluoromethyl-pyrimidine-5-carboxylic acid (35 mg) and N-(cyclohexylmethyl)-N-methylamine (21 mg) gave the title compound.

NMR (DMSO-d6) Rotamers in 63:37 ratio δ 0.65-1.30 (5H, m), 1.5-1.8 (6H, m), 2.87 (1.9H, s), 2.97 (1H, s), 3.03 (0.7H, d), 3.30 (1.3H, d excess), 7.09 (1H, d), 7.36 (1H, m), 7.66 (1H, d), 7.96 (1H, m), 8.73 (0.37H, s), 8.78 (0.63H, s), 10.6 (1H, s).

LC/MS t=3.87 min, Molecular ion observed (MH⁺)=427 consistent with the molecular formula C₂₀H₂₂³⁵ClF₃N₄O

### EXAMPLE 190

### 2-(3-Chlorophenylamino)-4-trifluoromethyl-pyrimidine-5-carboxylic acid cyclopentylmethyl-methyl-amide

### a) N-(Cyclopentylmethyl)-2,2,2-trifluoroacetamide

In a manner similar to Example 188a) (cyclopentylmethyl)amine (1.02 g) (Example 2) gave the title compound (1.47 g).

NMR (CDCl₃) δ 1.21 (2H, m), 1.4 (4H, m), 1.78 (2H, m), 2.10 (1H, m), 3.31 (2H, t), 6.3 (1H, br s).

### b) N-(Cyclopentylmethyl)-N-methylamine hydrochloride

In a manner similar to Example 188b) N-(cyclopentylmethyl)-2,2,2-trifluoroacetamide (1.46 g) gave, after treatment with hydrogen chloride in 1,4-dioxan, the title compound (0.77 g).

NMR (D₂O) δ 1.12 (2H, m), 1.5 (4H, m), 1.75 (2H, m), 2.08 (1H, m), 2.61 (3H, s), 2.90 (2H, d).

### c) 2-(3-Chlorophenylamino)-4-trifluoromethyl-pyrimidine-5-carboxylic acid cyclopentylmethyl-methyl-amide

In a manner similar to Example 188c) 2-(3-chlorophenylamino)-4-trifluoromethyl-pyrimidine-5-carboxylic acid (35 mg) and N-(cyclopentylmethyl)-N-methylamine hydrochloride (21 mg) together with an additional equivalent of N,N-diisopropylethylamine gave the title compound (42 mg)

NMR (DMSO-d6) Rotamers in 65:35 ratio δ 1.0-1.8 (8H, m), 2.13 (0.35H, m), 2.27 (0.65H, m), 2.88 (1.95H, s), 2.99 (1.05H, s), 3.14 (0.7H, d), 3.41 (1.3H, br s), 7.09 (1H, d), 7.36 (1H, t), 7.66 (1H, d), 7.96 (1H, m), 8.77 (1H, s), 10.6 (1H, s).

LC/MS t=3.77 min, Molecular ion observed (MH⁺)=413 consistent with the molecular formula C₁₉H₂₀³⁵ClF₃N₄O

### EXAMPLE 191

### 2-(5-Chloro-2-fluorophenylamino)-4-trifluoromethyl-pyrimidine-5-carboxylic acid cyclobutylmethyl-amide

### a) 2-Chloro-4-trifluoromethyl-pyrimidine-5-carboxylic acid cyclobutylmethyl-amide

A mixture of 2-chloro-4-trifluoromethyl-pyrimidine-5-carbonyl chloride (613 mg) (Maybridge) and C-cyclobutylmethylamine hydrochloride (304 mg) in dry dichloromethane (10 ml) was cooled to −30° C. and N,N-diisopropylethylamine (958 ul) was added dropwise. The mixture was stirred at room temp for 1 hour. Water (10 ml) was added, the layers separated and the organic layer was washed sequentially with 10 ml portions of water, dilute hydrochloric acid, water, dilute sodium bicarbonate solution and water, dried (MgSO₄) and evaporated. Purification by chromatography on silica gel (dichloromethane/ether 25:1) gave the title compound (449 mg).

NMR (CDCl₃) δ 1.75 (2H, m), 1.93 (2H, m), 2.10 (2H, m), 2.57 (1H, m), 3.50 (2H, t), 5.86 (1H, br s), 8.90 (1H, s).

### b) 2-(5-Chloro-2-fluorophenylamino)-4-trifluoromethyl-pyrimidine-5-carboxylic acid cyclobutylmethyl-amide

In a manner similar to Example 166, 5-chloro-2-fluoroaniline (109 mg) (Avacado) and 2-chloro-4-trifluoromethyl-pyrimidine-5-carboxylic acid cyclobutylmethyl-amide (44 mg) gave the title compound (45 mg).

NMR (DMSO-d6) δ 1.7 (2H, m), 1.8 (2H, m), 1.99 (2H, m), 2.47 (1H, m excess), 3.25 (2H, t), 7.3 (2H, m), 7.76 (1H, m), 8.56 (1H, t), 8.70 (1H, s), 10.2 (1H, s)

LC/MS t=3.52 min, Molecular ion observed (MH⁺)=403 consistent with the molecular formula C₁₇H₁₅³⁵ClF₄N₄O

### EXAMPLE 192

### 2-(3,5-Difluorophenylamino)-4-trifluoromethyl-pyrimidine-5-carboxylic acid cyclobutylmethyl-amide

In a manner similar to Example 166, 3,5-difluoroaniline (97 mg) (Lancaster) and 2-chloro-4-trifluoromethyl-pyrimidine-5-carboxylic acid cyclobutylmethyl-amide (44 mg) gave the title compound (46 mg).

NMR (DMSO-d6) δ 1.7 (2H, m), 1.8 (2H, m), 2.0 (2H, m), 2.47 (1H, m excess), 3.27 (2H, t), 6.88 (1H, m), 7.55 (2H, m), 8.60 (1H, t), 8.83 (1H, s), 10.8 (1H, s)

LC/MS t=3.54 min, Molecular ion observed (MH⁺)=387 consistent with the molecular formula C₁₇H₁₅F₅N₄O

### EXAMPLE 193

### 2-(3-Chloro-4-trifluoromethoxyphenylamino)-4-trifluoromethyl-pyrimidine-5-carboxylic acid cyclobutylmethyl-amide

In a manner similar to Example 166, 3-chloro-4-trifluoromethoxy aniline (159 mg) (Lancaster) and 2-chloro-4-trifluoromethyl-pyrimidine-5-carboxylic acid cyclobutylmethyl-amide (44 mg) gave the title compound (59 mg).

NMR (DMSO-d6) δ 1.7 (2H, m), 1.8 (2H, m), 2.0 (2H, m), 2.47 (1H, m excess), 3.27 (2H, t), 7.56 (1H, d), 7.76 (1H, m), 8.16 (1H, d), 8.59 (1H, t), 8.81 (1H, s), 10.8 (1H, s)

LC/MS t=3.82 min, Molecular ion observed (MH⁺)=469 consistent with the molecular formula C₁₈H₁₅³⁵ClF₆N₄O₂

### EXAMPLE 194

### 2-(3-Chloro-4-fluorophenylamino)-4-trifluoromethyl-pyrimidine-5-carboxylic acid cyclobutylmethyl-amide

In a manner similar to Example 166, 3-chloro-4-fluoroaniline (109 mg) (Lancaster) and 2-chloro-4-trifluoromethyl-pyrimidine-5-carboxylic acid cyclobutylmethyl-amide (44 mg) gave the title compound (50 mg).

NMR (DMSO-d6) δ 1.7 (2H, m), 1.8 (2H, m), 2.0 (2H, m), 2.47 (1H, m excess), 3.27 (2H, t), 7.42 (1H, t), 7.67 (1H, m), 8.04 (1H, m), 8.57 (1H, t), 8.77 (1H, s), 10.6 (1H, s)

LC/MS t=3.60 min, Molecular ion observed (MH⁺)=403 consistent with the molecular formula C₁₇H₁₅³⁵ClF₄N₄O

### EXAMPLE 195

### 2-(3-Chloro-2-fluorophenylamino)-4-trifluoromethyl-pyrimidine-5-carboxylic acid cyclobutylmethyl-amide

In a manner similar to Example 166, 3-chloro-2-fluoroaniline (109 mg) (Acros) and 2-chloro-4-trifluoromethyl-pyrimidine-5-carboxylic acid cyclobutylmethyl-amide (44 mg) gave the title compound (47 mg).

NMR (DMSO-d6) δ 1.7 (2H, m), 1.8 (2H, m), 2.0 (2H, m), 2.47 (1H, m excess), 3.23 (2H, t), 7.22 (1H, t), 7.42 (1H, t), 7.54 (1H, t), 8.55 (1H, t), 8.65 (1H, s), 10.2 (1H, s)

LC/MS t=3.49 min, Molecular ion observed (MH⁺)=403 consistent with the molecular formula C₁₇H₁₅³⁵ClF₄N₄O

### EXAMPLE 196

### 2-(3-Fluoro-4-trifluoromethylphenylamino)-4-trifluoromethyl-pyrimidine-5-carboxylic acid cyclobutylmethyl-amide

In a manner similar to Example 166, 3-fluoro-4-trifluoromethylaniline (134 mg) (ABCR) and 2-chloro-4-trifluoromethyl-pyrimidine-5-carboxylic acid C-cyclobutylmethyl-amide (44 mg) gave the title compound (41 mg).

NMR (DMSO-d6) δ 1.7 (2H, m), 1.8 (2H, m), 2.0 (2H, m), 2.47 (1H, m excess), 3.26 (2H, t), 7.67 (1H, d), 7.75 (1H, t), 8.02 (1H, d), 8.62 (1H, t), 8.87 (1H, s), 11.0 (1H, s)

LC/MS t=3.71 min, Molecular ion observed (MHW)=437 consistent with the molecular formula C₁₈H₁₅F₇N₄O

### EXAMPLE 197

### 2-(3-Chloro cyanophenylamino)-4-trifluoromethyl-pyrimidine-5-carboxylic acid cyclobutylmethyl-amide

In a manner similar to Example 166, 3-chloro-4-cyanoaniline (114 mg) (Lancaster) and 2-chloro-4-trifluoromethyl-pyrimidine-5-carboxylic acid cyclobutylmethyl-amide (44 mg) gave the title compound (26 mg).

NMR (DMSO-d6) δ 1.7 (2H, m), 1.8 (2H, m), 2.0 (2H, m), 2.47 (1H, m excess), 3.27 (2H, t), 7.83 (1H, m), 7.93 (1H, d), 8.24 (1H, s), 8.62 (1H, t), 8.89 (1H, s), 11.1 (1H, s)

LC/MS t=3.50 min, Molecular ion observed (MHt)=410 consistent with the molecular formula C₁₈H₁₅³⁵ClF₃N₅O

### EXAMPLE 198

### 2-(3-Fluorophenylamino)-4-trifluoromethyl-pyrimidine-5-carboxylic acid cyclobutylmethyl-amide

In a manner similar to Example 188, 2-(3-fluorophenylamino)-4-trifluoromethyl-pyrimidine-5-carboxylic acid (30 mg) and C-cyclobutylmethylamine hydrochloride (18 mg) gave the title compound (31 mg).

NMR (DMSO-d6) δ 1.7 (2H, m), 1.8 (2H, m), 2.0 (2H, m), 2.47 (1H, m excess), 3.26 (2H, t), 6.86 (1H, m), 7.37 (1H, m), 7.50 (1H, d), 7.76 (1H, m), 8.58 (1H, t), 8.78 (1H, s), 10.6 (1H, s)

LC/MS t=3.42 min, Molecular ion observed (MH⁺)=369 consistent with the molecular formula C₁₇H₁₆F₄N₄O

### EXAMPLE 199

### 2-(3-Bromophenylamino)-4-trifluoromethyl-pyrimidine-5-carboxylic acid cyclobutylmethyl-amide

In a manner similar to Example 188, 2-(3-bromophenylamino)-4-trifluoromethyl-pyrimidine-5-carboxylic acid (36 mg) and C-cyclobutylmethylamine hydrochloride (18 mg) gave the title compound (33 mg).

NMR (DMSO-d6) δ 1.7 (2H, m), 1.8 (2H, m), 2.0 (2H, m), 2.47 (1H, m excess), 3.26 (2H, t), 7.22 (1H, d), 7.31 (1H, t), 7.70 (1H, d), 8.10 (1H, t), 8.57 (1H, t), 8.78 (1H, s), 10.6 (1H, s)

LC/MS t=3.60 min, Molecular ion observed (MH⁺)=431 consistent with the molecular formula C₁₇H₁₆⁸¹BrF₃N₄O

### EXAMPLE 200

### 2-(2,3-Dichlorophenylamino)-4-trifluoromethyl-pyrimidine-5-carboxylic acid cyclobutylmethyl-amide

In a manner similar to Example 188, 2-(2,3-dichlorophenylamino)-4-trifluoromethyl-pyrimidine-5-carboxylic acid (36 mg) and C-cyclobutylmethylamine hydrochloride (18 mg) gave the title compound (36 mg).

NMR (DMSO-d6) δ 1.7 (2H, m), 1.8 (2H, m), 2.0 (2H, m), 2.47 (1H, m excess), 3.24 (2H, t), 7.40 (1H, t), 7.54 (2H, m), 8.54 (1H, t), 8.63 (1H, s), 10.1 (1H, s)

LC/MS t=3.61 min, Molecular ion observed (MH⁺)=419 consistent with the molecular formula C₁₇H₁₅³⁵Cl₂F₃N₄O

### EXAMPLE 201

### 2-(2,4-Dichlorophenylamino)-4-trifluoromethyl-pyrimidine-5-carboxylic acid cyclobutylmethyl-amide

In a manner similar to Example 188, 2-(2,4-dichlorophenylamino)-4-trifluoromethyl-pyrimidine-5-carboxylic acid (36 mg) and C-cyclobutylmethylamine hydrochloride (18 mg) gave the title compound (37 mg).

NMR (DMSO-d6) δ 1.7 (2H, m), 1.8 (2H, m), 2.0 (2H, m), 2.47 (1H, m excess), 3.24 (2H, t), 7.47 (1H, m), 7.58 (1H, d), 7.72 (1H, d), 8.54 (1H, t), 8.65 (1H, s), 10.0 (1H, s)

LC/MS t=3.66 min, Molecular ion observed (MH⁺)=419 consistent with the molecular formula C₁₇H₁₅³⁵Cl₂F₃N₄O

### EXAMPLE 202

### 2-(2,5-Dichlorophenylamino)-4-trifluoromethyl-pyrimidine-5-carboxylic acid cyclobutylmethyl-amide

In a manner similar to Example 188, 2-(2,5-dichlorophenylamino)-4-trifluoromethyl-pyrimidine-5-carboxylic acid (36 mg) and C-cyclobutylmethylamine hydrochloride (18 mg) gave the title compound (33 mg).

NMR (DMSO-d6) δ 1.7 (2H, m), 1.8 (2H, m), 2.0 (2H, m), 2.47 (1H, m excess), 3.24 (2H, t), 7.34 (1H, m), 7.58 (1H, d), 7.72 (1H, d), 8.55 (1H, t), 8.66 (1H, s), 10.0 (1H, s)

LC/MS t=3.65 min, Molecular ion observed (MH⁺)=419 consistent with the molecular formula C₁₇H₁₅³⁵Cl₂F₃N₄O

### EXAMPLE 203

### 2-(2,6-Dichlorophenylamino)-4-trifluoromethyl-pyrimidine-5-carboxylic acid cyclobutylmethyl-amide

In a manner similar to Example 188, 2-(2,6-dichlorophenylamino)-4-trifluoromethyl-pyrimidine-5-carboxylic acid (36 mg) and C-cyclobutylmethylamine hydrochloride (18 mg) gave the title compound (35 mg).

NMR (DMSO-d6) δ 1.7 (2H, m), 1.8 (2H, m), 2.0 (2H, m), 2.47 (1H, m excess), 3.22 (2H, t), 7.39 (1H, t), 7.59 (2H, d), 8.56 (2H, m), 10.1 (1H, s).

LC/MS t=3.38 min, Molecular ion observed (MH⁺)=419 consistent with the molecular formula C₁₇H₁₅³⁵Cl₂F₃N₄O

### EXAMPLE 204

### 2-(3,4-Dichlorophenylamino)-4-trifluoromethyl-pyrimidine-5-carboxylic acid cyclobutylmethyl-amide

In a manner similar to Example 188, (3,4-dichlorophenylamino)-4-trifluoromethyl-pyrimidine-5-carboxylic acid (36 mg) and C-cyclobutylmethylamine hydrochloride (118 mg) gave the title compound (36 mg).

NMR (DMSO-d6) δ 1.7 (2H, m), 1.8 (2H, m), 2.0 (2H, m), 2.47 (1H, m excess), 3.26 (2H, t), 7.60 (1H, d), 7.69 (1H, m), 8.16 (1H, d), 8.58 (1H, t), 8.80 (1H, s), 10.7 (1H, s)

LC/MS t=3.77 min, Molecular ion observed (MH⁺)=419 consistent with the molecular formula C₁₇H₁₅³⁵Cl₂F₃N₄O

### EXAMPLE 205

### 2-(3-Methoxyphenylamino)-4-trifluoromethyl-pyrimidine-5-carboxylic acid cyclobutylmethyl-amide

In a manner similar to Example 188, 2-(3-methoxyphenylamino)-4-trifluoromethyl-pyrimidine-5-carboxylic acid (31 mg) and C-cyclobutylmethylamine hydrochloride (18 mg) gave the title compound (38 mg).

NMR (DMSO-d6) δ 1.7 (2H, m), 1.8 (2H, m), 2.0 (2H, m), 2.47 (1H, m excess), 3.26 (2H, t), 3.74 (3H, s), 6.63 (1H, d), 7.24 (2H, m), 7.52 (1H, s), 8.56 (1H, t), 8.72 (1H, s), 10.4 (1H, s)

LC/MS t=3.35 min, Molecular ion observed (MH⁺)=381 consistent with the molecular formula C₁₈H₁₉F₃N₄O₂

### EXAMPLE 206

### 2-(3,5-Dichlorophenylamino)-4-trifluoromethyl-pyrimidine-5-carboxylic acid cyclobutylmethyl-amide

In a manner similar to Example 188, 2-(3,5-dichlorophenylamino)-4-trifluoromethyl-pyrimidine-5-carboxylic acid (36 mg) and C-cyclobutylmethylamine hydrochloride (18 mg) gave the title compound (36 mg).

NMR (DMSO-d6) δ 1.7 (2H, m), 1.8 (2H, m), 2.0 (2H, m), 2.47 (1H, m excess), 3.26 (2H, t), 7.60 (1H, d), 7.69 (1H, m), 8.16 (1H, d), 8.58 (1H, t), 8.80 (1H, s), 10.7 (1H, s)

LC/MS t=3.84 min, Molecular ion observed (MH⁺)=419 consistent with the molecular formula C₁₇H₁₅³⁵Cl₂F₃N₄O

### EXAMPLE 207

### 2-(3-Bromophenylamino)-4-trifluoromethyl-pyrimidine-5-carboxylic acid cyclopentylamide

In a manner similar to Example 188, 2-(3-bromophenylamino)-4-trifluoromethyl-pyrimidine-5-carboxylic acid (36 mg) and cyclopentylamine (18 mg) gave the title compound (28 mg).

NMR (DMSO-d6) δ 1.5 (4H, m), 1.66 (2H, m), 1.86 (2H, m), 4.16 (1H, m), 7.22 (1H, d), 7.31 (1H, t), 7.70 (1H, d), 8.10 (1H, t), 8.53 (1H, d), 8.79 (1H, s), 10.6 (1H, s)

LC/MS t=3.39 min, Molecular ion observed (MW⁺)=431 consistent with the molecular formula C₁₇H₁₆⁸¹BrF₃N₄O

### EXAMPLE 208

### 2-(2,4-Dichlorophenylamino)-4-trifluoromethyl-pyrimidine-5-carboxylic acid cyclopentylamide

In a manner similar to Example 188, 2-(2,4-dichlorophenylamino)-4-trifluoromethyl-pyrimidine-5-carboxylic acid (26 mg) and cyclopentylamine (18 mg) gave the title compound (21 mg).

NMR (DMSO-d6) δ 1.5 (4H, m), 1.63 (2H, m), 1.84 (2H, m), 4.14 (1H, m), 7.47 (1H, m), 7.56 (1H, d), 7.71 (1H, d), 8.50 (1H, d), 8.62 (1H, s), 10.0 (1H, s)

LC/MS t=3.40 min, Molecular ion observed (MH⁺)=419 consistent with the molecular formula C₁₇H₁₅³⁵Cl₂F₃N₄O

### EXAMPLE 209

### 2-(3-Chlorophenylamino)-4-trifluoromethyl-pyrimidine-5-carboxylic acid cyclopropylamide

In a manner similar to Reference Example 1 (c) 2-(3-chlorophenylamino)-4-trifluoromethyl-pyrimidine-5-carboxylic acid (35 mg) and cyclopropylamine (9 mg, ex Lancaster) afforded the title compound (32 mg).

NMR (DMSO-d6) δ 0.49-0.52 (2H, m), 0.69-0.74 (2H, m), 2.78 (1H, m), 7.09 (1H, d), 7.36 (1H, t), 7.65 (1H, d), 7.95 (1H, s), 8.65 (1H, d), 8.80 (1H s), 10.60 (1H, s)

LC/MS, t=3.25 min, Molecular ion observed (MH⁺)=357 consistent with the molecular formula C₁₅H₁₂N₄OF₃³⁵Cl

### EXAMPLE 210

### 2-(3-Chlorophenylamino)-4-trifluoromethyl-pyrimidine-5-carboxylic acid (3,3-dimethylbutyl)-amide

In a manner similar to Reference Example 1 (c) 2-(3-chlorophenylamino)-4-trifluoromethyl-pyrimidine-5-carboxylic acid (50 mg) and 3,3-dimethylbutylamine (17 mg, ex Aldrich) afforded the title compound (32 mg).

NMR (DMSO-d6) δ 0.96 (6H, d), 1.85 (1H, m), 3.12 (2H, t), 7.16 (1H, d), 7.42 (1H, t), 7.71 (1H, d), 8.02 (1H, s), 8.65 (1H, t), 8.86 (1H s), 10.70 (1H, s)

LC/MS, t=3.49 min, Molecular ion observed (MH⁺)=373 consistent with the molecular formula C₁₆H₁₆N₄OF₃³⁵Cl

### EXAMPLE 211

### 2-(3-Chlorophenylamino)-4-trifluoromethyl-pyrimidine-5-carboxylic acid methyl-(tetrahydro-pyran-4-ylmethyl)-amide

(a). To a solution of 4-aminomethyltetrahydropyran (500 mg, ex Combi-Blocks, Inc.) in dichloromethane (10 ml) at 0° C. was added triethylamine (1.2 ml) followed by a solution of di-tert-butyl dicarbonate (1. 14 g) dichloromethane (4 ml). The reaction was stirred at 0° C. for 1 h. Dichloromethane was removed under reduced pressure and ethyl acetate added (10 ml). The solution was washed sequentially with 2N hydrochloric acid (10 ml), water (10 ml), 5% sodium bicarbonate solution (10 ml), and water (10 ml), dried (MgSO₄) and evaporated. The residue was purified by chromatography eluting with 2% MeOH/CH₂Cl₂, t afford N-(tetrahydro-pyran-4-ylmethyl)-carbamic acid tert-butyl ester (809 mg).

NMR (DMSO-d6) δ 1.15 (2H, m), 1.45 (9H, s), 1.80-1.95 (3H, d, m), 2.87 (2H, t), 3.30 (2H, t), 3.90 (2H, d, d), 6.95 (1H, t).

(b). To a solution of N-(tetrahydro-pyran-4-ylmethyl)-carbamic acid tert-butyl ester (800 mg) in THF (10 ml) at room temperature under nitrogen was added 60% sodium hydride (164 mg, ex Aldrich) portionwise. The reaction was stirred until effervescence had ceased and then methyl iodide (280 μl, ex Lancaster) was added. Stirring was continued at room temperature overnight. THF was removed under reduced pressure and ethyl acetate was added (10 ml). This was washed three times with water (10 ml), dri (MgSO₄) and evaporated. The residue was purified by chromatography eluting with 3% MeOH/CH₂Cl₂, afford N-methyl-N-(tetrahydro-pyran-4-ylmethyl)-carbamic acid tert-butyl ester (745 mg).

NMR (DMSO-d6) δ 1.15 (2H, m), 1.45 (9H, s), 1.50 (2H, m), 1.80 (1H, m) 2.80 (3H, d), 3.08 (2H, d), 3.2 (2H, t), 3.85 (2H, d).

(c). A solution of N-methyl-N-(tetrahydro-pyran-4-ylmethyl)-carbamic acid tert-butyl ester (740 mg) in 41 hydrochloric acid in 1,4-dioxan (10 ml, ex Aldrich) was stirred at room temperature for 1 h. The dioxan was removed under reduced pressure and the residue triturated with ether. The solid was filtered onto a sinter, washed with ether and dried, to afford N-methyl-N-(tetrahydro-pyran-4-ylmethyl)-amine hydrochloride (460 mg).

NMR (DMSO-d6) δ 1.15 (2H, m), 1.65 (2H, d), 1.95 (1H, m) 2.50 (3H, d), 2.80 (2H, d), 3.30 (2H, t), 3.8° (2H, d), 9.0 (2H, s).

(d). In a manner similar to Reference Example 1 (c) 2-(3-chlorophenylamino)-4-trifluoromethyl-pyrimidine-5-carboxylic acid (50 mg) and N-methyl-N-(tetrahydro-pyran-4-yl methyl) amine hydrochloride (39 mg) afforded, after Biotage chromatography over silica gel, eluting with 1% MeOH/CH₂Cl₂, the title compound (33 mg).

NMR (DMSO-d6) Rotamers in 65:35 ratio δ 1.05 (0.7H, m), 1.23 (1.3H, m), 1.45 (0.7H, d), 1.58 (1.3H, d), 1.85 (0.35H, m), 2.0 (0.65H, m), 2.89 (1.95H, s), 2.98 (1.05H, s), 3.10-3.40 (4H, m), 3.80 (0.7H, d), 3.88 (1.3H, d), 7.10 (1H, d), 7.36 (1H, t), 7.65 (1H, t), 7.97 (1H, s), 8.75 (0.35H, s), 8.80 (0.65H, s), 10.6 (1H, s)

LC/MS, t=3.29 min, Molecular ion observed (MH⁺)=429 consistent with the molecular formula C₁₉H₂₀N₄O₂F₃³⁵Cl

### EXAMPLE 212

### 2-(2-Fluoro-3-chloro-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (100 mg) and 2-fluoro-3-chloroaniline (225 mg, ex Acros) afforded the title compound (85 mg) after purification by trituration with isohexane.

NMR (DMSO-d6) δ 1.14-1.23 (2H, m), 1.6 (2H, d), 1.72 (1H, m), 3.1 (2H, t), 3.25 (2H, m), 3.85 (2H, d), 7.24 (1H, t), 7.42 (1H, t), 7.55 (1H, t), 8.61 (1H, t), 8.70 (1H, s), 10.20 (1H, s)

LC/MS, t=3.14 min, Molecular ion observed (MH⁺)=433 consistent with the molecular formula C₁₈H₁₇N₄O₂F₄³⁵Cl

### EXAMPLE 213

### 2-(2-Fluoro-5-chloro-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (100 mg) and 2-fluoro-5-chloroaniline (225 mg, ex Avocado) afforded the title compound (96 mg) after purification by trituration with isohexane.

NMR (DMSO-d6) δ 1.17-1.23 (2H, m), 1.6 (2H, d), 1.72 (1H, m), 3.1 (2H, t), 3.25 (2H, m), 3.85 (2H, d), 7.27-7.37 (2H, t, m), 7.76 (1H, dd), 8.62 (1H, t), 8.73 (1H, s), 10.15 (1H, s)

LC/MS, t=3.15 min, Molecular ion observed (MH⁺)=433 consistent with the molecular formula C₁₈H₁₇N₄O₂F₄³⁵Cl

### EXAMPLE 214

### 2-(3,5-Difluorophenylamino)-4-trifluoromethyl-pyrimidine-5-carboxylic acid (tetrahydro-pyran-4-ylmethyl)-amide

In a manner similar to Example 167, 2-chloro-4-trifluoromethyl-pyrimidine-5-carboxylic acid (tetrahydro-pyran-4-ylmethyl)-amide (100 mg) and 3,5-difluoroaniline (199 mg, ex Lancaster) afforded the title compound (98 mg) after purification by trituration with isohexane.

NMR (DMSO-d6) δ 1.18-1.25 (2H, m), 1.61 (2H, d), 1.74 (1H, m), 3.13 (2H, t), 3.27 (2H, m), 3.85 (2H, d), 6.88 (1H, t,), 7.52 7.55 (2H, m), 8.66 (1H, t), 8.86 (1H, s), 10.80 (1H, s)

LC/MS, t=3.18 min, Molecular ion observed (MH⁺)=417 consistent with the molecular formula C₁₈H₁₇N₄O₂F₅

### EXAMPLE 215

### 2-(4-Fluoro-3-chloro-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (100 mg) and 4-fluoro-3-chloroaniline (225 mg, ex Lancaster) afforded the title compound (134 mg) after purification by trituration with isohexane.

NMR (DMSO-d6) δ 1.18-1.23 (2H, m), 1.61 (2H, d), 1.75 (1H, m), 3.1 (2H, t), 3.25 (2H, m), 3.85 (2H, d), 7.42 (1H, t), 7.65 (1H, m), 8.05 (1H, dd), 8.63 (1H, t), 8.80 (1H, s), 10.65 (1H, s)

LC/MS, t=3.25 min, Molecular ion observed (MH⁺)=433 consistent with the molecular formula C₁₈H₁₇N₄O₂F₄³⁵Cl

### EXAMPLE 216

### 2-(4-Trifluoromethoxy-3-chloro-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (100 mg) and 4-trifluoromethoxy-3-chloroaniline (327 mg, ex Lancaster) afforded the title compound (135 mg) after purification by trituration with isohexane.

NMR (DMSO-d6) δ 1.18-1.23 (2H, m), 1.61 (2H, d), 1.74 (1H, m), 3.13 (2H, t), 3.25 (2H, m), 3.85 (2H, d), 7.57 (1H, d), 7.75 (1H, dd), 8.14 (1H, d), 8.63 (1H, t), 8.84 (1H, s), 10.74 (1H, s)

LC/MS, t=3.51 min, Molecular ion observed (MH⁺)=499 consistent with the molecular formula C₁₉H₁₇N₄O₃F₆³⁵Cl

### EXAMPLE 217

### 2-(4-Cyano-3-chloro-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (100 mg) and 4-cyano-3-chloroaniline (236 mg, ex Lancaster) afforded the title compound (8 mg). Sample purified by mass directed auto-prep.

LC/MS, t=3.51 min, Molecular ion observed (MW⁺)=440 consistent with the molecular formula C₁₉H₁₇N₅O₂F₃³⁵Cl

### EXAMPLE 218

### 2-(4-Trifluoromethyl-3-fluoro-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (100 mg) and 4-trifluoromethyl-3-fluoroaniline (277 mg, ex ABCR) afforded the title compound (125 mg) after purification by trituration with isohexane.

NMR (DMSO-d6) δ 1.16-1.25 (2H, m), 1.61 (2H, d), 1.73 (1H, m), 3.14 (2H, t), 3.25 (2H, m), 3.85 (2H, d), 7.67 (1H, d), 7.75 (1H, t), 8.02 (1H, d), 8.68 (1H, t), 8.90 (1H, s), 11.00 (1H, s)

LC/MS, t=3.38 min, Molecular ion observed (MH⁺)=467 consistent with the molecular formula C₁₉H₁₇N₄O₂F₇

### EXAMPLE 219

### 2-(4-Cyano-3-chloro-phenylamino)-4-trifluoromethyl-pyrimidine-5-carboxylic acid cyclopentylmethyl-amide

In a manner similar to Example 166, 2-chloro-4-trifluoromethyl-pyrimidine-5-carboxylic acid cyclopentylmethylamide (70 mg) and 4-cyano-3-chloroaniline (173 mg, ex Lancaster) afforded the title compound (125 mg). Purified by chromatography eluting with 1:1 ethyl acetate:hexane.

NMR (DMSO-d6) δ 1.20-1.25 (2H, m), 1.48-1.73 (6H, m), 2.08 (1H, m), 3.18 (2H, t), 7.83 (1H, dd), 7.84 (1H, d), 8.24 (1H, d), 8.66 (1H, t), 8.90 (1H s), 11.10 (1H, s)

LC/MS, t=3.68 min, Molecular ion observed (MH⁺)=424 consistent with the molecular formula C₁₉H₁₇N₅OF₃³⁵Cl

### EXAMPLE 220

### 2-(2,4-Dichloro-phenylamino)-4-trifluoromethyl-pyrimidine-5-carboxylic acid (1,1-dioxo-hexahydro-1l⁶-thiopyran-4-yl)-amide

In a manner similar to Reference Example 1 (c) 2-(2,4-Dichlorophenylamino)-4-trifluoromethyl-pyrimidine-5-carboxylic acid (50 mg) and (1,1-dioxo-tetrahydro-2H-thiopyran-4-yl)amine hydrochloride (40 mg) (Ref. WO 02/18380) afforded the title compound (64 mg). Purified by chromatography eluting with 2% MeOH/CH₂Cl₂.

NMR (DMSO-d6) δ 1.97 (2H, m), 2.13 (2H, m), 3.13 (2H, m), 3.27 (2H, m), 4.10 (1H, m), 7.47 (1H, dd), 7.56 (1H, d), 7.72 (1H, d), 8.67 (1H t), 8.7 (1H, s), 10.05 (1H, s)

LC/MS, t=3.22 min, Molecular ion observed (MH⁺)=483 consistent with the molecular formula C₁₇H₁₅N₄O₃F₃³⁵Cl₂S

### EXAMPLE 221

### 2-(2,4-Difluoro-phenylamino)-4-trifluoromethyl-pyrimidine-5-carboxylic acid cyclohexylmethyl-amide

In a manner similar to Example 166, 2-chloro-4-trifluoromethyl-pyrimidine-5-carboxylic acid cyclohexylmethyl-amide (80 mg) and 2,4-difluoroaniline (160 mg, ex Lancaster) afforded the title compound (77 mg) after purification by trituration with isohexane/diethylether.

NMR (DMSO-d6) δ 0.89-0.95 (2H, m), 1.15-1.20 (3H, m), 1.46-1.47 (1H, m), 1.60-1.72 (5H, m), 3.05 (2H, t), 7.10 (1H, t), 7.35 (1H, m), 7.52 (1H, m), 8.53 (1H t), 8.62 (1H, s), 10.00 (1H, s)

LC/MS, t=3.63 min, Molecular ion observed (MH⁺)=433 consistent with the molecular formula C₁₉H₁₉N₄OF₅

### EXAMPLE 222

### 2-(2-Chloro-4-fluoro-phenylamino)-4-trifluoromethyl-pyrimidine-5-carboxylic acid cyclohexylmethyl-amide

In a manner similar to Example 166, 2-chloro-4-trifluoromethyl-pyrimidine-5-carboxylic acid cyclohexylmethyl-amide (80 mg) and 2-chloro-4-fluoroaniline (181 mg, ex Lancaster) afforded the title compound (91 mg).

NMR (DMSO-d6) δ 0.89-0.95 (2H, m), 1.15-1.20 (3H, m), 1.44-1.46 (1H, m), 1.62-1.72 (5H, m), 3.05 (2H, t), 7.27 (1H, m), 7.55 (2H, m), 8.52 (1H, t), 8.60 (1H, s), 10.00 (1H, s)

LC/MS, t=3.73 min, Molecular ion observed (MH⁺)=431 consistent with the molecular formula C₁₉H₁₉N₄OF₄³⁵Cl

### EXAMPLE 223

### 2-(2,4-Difluoro-phenylamino)-4-trifluoromethyl-pyrimidine-5-carboxylic acid cyclobutylmethyl-amide

In a manner similar to Example 166, 2-chloro-4-trifluoromethyl-pyrimidine-5-carboxylic acid cyclobutylmethyl-amide (80 mg) and 2,4-difluoroaniline (198 mg, ex Lancaster) afforded the title compound (82 mg) after purification by trituration with isohexane/diethylether.

NMR (DMSO-d6) δ 1.67-2.01 (6H, m), 2.47 (1H, m), 3.23 (2H, t), 7.10 (1H, t), 7.35 (1H, m), 7.52 (1H, m), 8.53 (1H, t), 8.62 (1H, s), 10.00 (1H, s)

LC/MS, t=3.40 min, Molecular ion observed (MH⁺)=386 consistent with the molecular formula C₁₇H₁₅N₄OF₅

### EXAMPLE 224

### 2-(2-Chloro-4-fluoro-phenylamino)-4-trifluoromethyl-pyrimidine-5-carboxylic acid cyclobutylmethyl-amide

In a manner similar to Example 166, 2-chloro-4-trifluoromethyl-pyrimidine-5-carboxylic acid cyclobutylmethyl-amide (80 mg) and 2-chloro-4-fluoroaniline (198 mg, ex Lancaster) afforded the title compound (80 mg) after purification by trituration with 2N hydrochloric acid.

NMR (DMSO-d6) δ 1.67-2.00 (6H, m), 2.46 (1H, m), 3.23 (2H, t), 7.27 (1H, m), 7.55 (2H, m), 8.52 (1H t), 8.58 (1H, s), 9.90 (1H, s)

LC/MS, t=3.51 min, Molecular ion observed (MH⁺)=403 consistent with the molecular formula C₁₇H₁₅N₄OF₄³⁵Cl

### EXAMPLE 225

### 2-(2-Chloro-4-bromo-phenylamino)-4-trifluoromethyl-pyrimidine-5-carboxylic acid cyclohexylmethyl-amide

In a manner similar to Example 166, 2-chloro-4-trifluoromethyl-pyrimidine-5-carboxylic acid cyclohexylmethyl-amide (80 mg) and 2-chloro-4-bromoaniline (257 mg, ex Lancaster) afforded the title compound (96 mg) after purification by trituration with 2N hydrochloric acid.

NMR (DMSO-d6) δ 0.89-0.95 (2H, m), 1.15-1.20 (3H, m), 1.44-1.46 (1H, m), 1.62-1.72 (5H, m), 3.05 (2H, t), 7.52 (1H, d), 7.58 (1H, dd), 7.82 (1H, d), 8.55 (1H t), 8.63 (1H, s), 10.00 (1H, s)

LC/MS, t=3.97 min, Molecular ion observed (MH⁺)=493 consistent with the molecular formula C₁₉H₁₉N₄OF₃³⁵Cl⁸¹Br

### EXAMPLE 226

### 2-(2-Fluoro-4-chloro-phenylamino)-4-trifluoromethyl-pyrimidine-5-carboxylic acid cyclohexylmethyl-amide

In a manner similar to Example 166, 2-chloro-4-trifluoromethyl-pyrimidine-5-carboxylic acid cyclohexylmethyl-amide (80 mg) and 2-fluoro-4-chloroaniline (180 mg, ex Lancaster) afforded the title compound (73 mg) after purification by trituration with 2N hydrochloric acid.

NMR (DMSO-d6) δ 0.95-0.98 (2H, m), 1.15-1.20 (3H, m), 1.44-1.46 (1H, m), 1.66-1.72 (5H, m), 3.05 (2H, t), 7.31 (1H, d), 7.53 (1H, dd), 7.60 (1H, t), 8.55 (1H t), 8.66 (1H, s), 10.00 (1H, s)

LC/MS, t=3.79 min, Molecular ion observed (MH⁺)=431 consistent with the molecular formula C₁₉H₁₉N₄OF₄³⁵Cl

### EXAMPLE 227

### 2-(2-Chloro-4-bromo-phenylamino)-4-trifluoromethyl-pyrimidine-5-carboxylic acid cyclobutylmethyl-amide

In a manner similar to Example 166, 2-chloro-4-trifluoromethyl-pyrimidine-5-carboxylic acid cyclobutylmethyl-amide (80 mg) and 2-chloro-4-bromoaniline (281 mg, ex Lancaster) afforded the title compound (103 mg) after purification by trituration with 2N hydrochloric acid.

NMR (DMSO-d6) δ 1.67-2.00 (6H, m), 2.45 (1H, m), 3.23 (2H, t), 7.50 (1H, d), 7.58 (1H, dd), 7.82 (1H, d), 8.53 (1H t), 8.61 (1H, s), 10.00 (1H, s)

LC/MS, t=3.77 min, Molecular ion observed (MH⁺)=465 consistent with the molecular formula C₁₇H₁₁N₄OF₃³⁵Cl⁸¹Br

### EXAMPLE 228

### 2-(2-Fluoro-4-chloro-phenylamino)-4-trifluoromethyl-pyrimidine-5-carboxylic acid cyclobutylmethyl-amide

In a manner similar to Example 166, 2-chloro-4-trifluoromethyl-pyrimidine-5-carboxylic acid cyclobutylmethyl-amide (80 mg) and 2-fluoro-4-chloroaniline (198 mg, ex Lancaster) afforded the title compound (94 mg) after purification by trituration with 2N hydrochloric acid.

NMR (DMSO-d6) δ 1.67-2.08 (6H, m), 2.45 (1H, m), 3.23 (2H, t), 7.31 (1H, d), 7.53 (1H, dd), 7.60 (1H, t), 8.53 (1H t), 8.64 (1H, s), 10.00 (1H, s)

LC/MS, t=3.59 min, Molecular ion observed (MH⁺)=403 consistent with the molecular formula C₁₇H₁N₄OF₄³⁵Cl

### EXAMPLE 229

### 2-(2-Fluoro-4-bromo-phenylamino)-4-trifluoromethyl-pyrimidine-5-carboxylic acid cyclobutylmethyl-amide

In a manner similar to Example 166, 2-chloro-4-trifluoromethyl-pyrimidine-5-carboxylic acid cyclobutylmethyl-amide (80 mg) and 2-fluoro-4-bromoaniline (259 mg, ex Lancaster) afforded the title compound (95 mg) after purification by trituration with 2N hydrochloric acid.

NMR (DMSO-d6) δ 1.67-2.00 (6H, m), 2.45 (1H, m), 3.23 (2H, t), 7.43 (1H, d), 7.54 (1H, t), 7.63 (1H, dd), 8.53 (1H t), 8.64 (1H, s), 10.00 (1H, s)

LC/MS, t=3.63 min, Molecular ion observed (MH⁺)=449 consistent with the molecular formula C₁₇H₁₅N₄OF₄⁸¹Br

### EXAMPLE 230

### 2-(2-Bromo-4-chloro-phenylamino)-4-trifluoromethyl-pyrimidine-5-carboxylic acid cyclobutylmethyl-amide

In a manner similar to Example 166, 2-chloro-4-trifluoromethyl-pyrimidine-5-carboxylic acid cyclobutylmethyl-amide (80 mg) and 2-bromo-4-chloroaniline (281 mg, ex Lancaster) afforded the title compound (105 mg) after purification by trituration with 2N hydrochloric acid.

NMR (DMSO-d6) δ 1.67-2.08 (6H, m), 2.45 (1H, m), 3.23 (2H, t), 7.52 (2H, m), 7.85 (1H, s), 8.53 (1H t), 8.60 (1H, s), 10.00 (1H, s)

LC/MS, t=3.75 min, Molecular ion observed (MH⁺)=465 consistent with the molecular formula C₁₇H₁₅N₄OF₃³⁵Cl⁸¹Br

### EXAMPLE 231

### 2-(2-Fluoro-4-bromo-phenylamino)-4-trifluoromethyl-pyrimidine-5-carboxylic acid cyclohexylmethyl-amide

In a manner similar to Example 166, 2-chloro-4-trifluoromethyl-pyrimidine-5-carboxylic acid cyclohexylmethyl-amide (80 mg) and 2-fluoro-4-bromoaniline (236 mg, ex Lancaster) afforded the title compound (96 mg) after purification by trituration with 2N hydrochloric acid.

NMR (DMSO-d6) δ 0.90-0.92 (2H, m), 1.15-1.20 (3H, m), 1.44-1.46 (1H, m), 1.63-1.72 (5H, m), 3.05 (2H, t), 7.44 (1H, d), 7.55 (1H, t), 7.64 (1H, dd), 8.55 (1H t), 8.66 (1H, s), 10.00 (1H, s)

LC/MS, t=3.83 min, Molecular ion observed (MH⁺)=477 consistent with the molecular formula C₁₉H₁₉N₄OF₄⁸¹Br

### EXAMPLE 232

### 2-(2-Fluoro-4-bromo-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (80 mg) and 2-fluoro-4-bromoaniline (235 mg, ex Lancaster) afforded the title compound (100 mg) after purification by trituration with 2N hydrochloric acid.

NMR (DMSO-d6) δ 1.16-1.23 (2H, m), 1.60 (2H, m), 1.71 (1H, m), 3.1 (2H, t), 3.25 (2H, m), 3.85 (2H, d), 7.43 (1H, d), 7.55 (1H, t), 7.64 (1H, dd), 8.60 (1H, t), 8.65 (1H, s), 10.10 (1H, s)

LC/MS, t=3.28 min, Molecular ion observed (MH⁺)=479 consistent with the molecular formula C₁₈H₁₇N₄O₂F₄⁸¹Br

### EXAMPLE 233

### 2-(2-Chloro-4-fluoro-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (80 mg) and 2-chloro-4-fluoroaniline (180 mg, ex Lancaster) afforded the title compound (95 mg) after purification by trituration with 2N hydrochloric acid.

NMR (DMSO-d6) δ 1.14-1.23 (2H, m), 1.59 (2H, d), 1.71 (1H, m), 3.1 (2H, t), 3.25 (2H, m), 3.85 (2H, d), 7.27 (1H, m), 7.55 (2H, m), 8.58 (1H, t), 8.61 (1H, s), 10.00 (1H, s)

LC/MS, t=3.14 min, Molecular-ion observed (MH⁺)=433 consistent with the molecular formula C₁₈H₁₇N₄O₂F₄³⁵Cl

### EXAMPLE 234

### 2-(2-Chloro-4-bromo-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (80 mg) and 2-chloro-4-bromoaniline (255 mg, ex Lancaster) afforded the title compound (102 mg) after purification by trituration with 2N hydrochloric acid.

NMR (DMSO-d6) δ 1.14-1.23 (2H, m), 1.58 (2H, d), 1.72 (1H, m), 3.1 (2H, t), 3.28 (2H, m), 3.84 (2H, d), 7.51 (1H, d), 7.59 (1H, dd), 7.82 (1H, d), 8.58 (1H, t), 8.63 (1H, s), 10.00 (1H, s)

LC/MS, t=3.42 min, Molecular ion observed (MH⁺)=495 consistent with the molecular formula C₁₈H₁₇N₄O₂F₃³⁵Cl⁸Br

### EXAMPLE 235

### 2-(2-Chloro-4-cyano-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (80 mg) and 2-chloro-4-cyanoaniline (188 mg, ex Lancaster) afforded the title compound (22 mg). Sample purified by mass directed auto-prep.

NMR (DMSO-d6) δ 1.14-1.23 (2H, m), 1.59 (2H, d), 1.72 (1H, m), 3.12 (2H, t), 3.23 (2H, m), 3.85 (2H, d), 7.87 (1H, d), 7.92 (1H, d), 8.14 (1H, s), 8.65 (1H, t), 8.75 (1H, s), 10.20 (1H, s)

LC/MS, t=3.11 min, Molecular ion observed (MH⁺)=440 consistent with the molecular formula C₁₉H₁₇N₅O₂F₃³⁵Cl

### EXAMPLE 236

### 2-(2-Chloro-4-trifluoromethyl-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (80 mg) and 2-chloro-4-trifluoromethylaniline (241 mg, ex Lancaster) afforded the title compound (48 mg). Sample purified by mass directed auto-prep.

NMR (DMSO-d6) δ 1.17-1.23 (2H, m), 1.59 (2H, d), 1.72 (1H, m), 3.12 (2H, t), 3.23 (2H, m), 3.85 (2H, d), 7.77 (1H, d), 7.88 (1H, d), 7.96 (1H, s), 8.63 (1H, t), 8.72 (1H, s), 10.15 (1H, s)

LC/MS, t=3.47 min, Molecular ion observed (MH⁺)=483 consistent with the molecular formula C₁₉H₁₇N₄O₂F₆³⁵Cl

### EXAMPLE 237

### 2-(2-Chloro-4-cyano-phenylamino)-4-trifluoromethyl-pyrimidine-5-carboxylic acid cyclohexylmethyl-amide

In a manner similar to Example 166, 2-chloro-4-trifluoromethyl-pyrimidine-5-carboxylic acid cyclohexylmethyl-amide (80 mg) and 2-chloro-4-cyanoaniline (189 mg, ex Lancaster) afforded the title compound (15 mg). Sample purified by mass directed auto-prep.

NMR (DMSO-d6) δ 0.90 (2H, m), 1.15-1.23 (3H, m), 1.44-1.46 (1H, m), 1.67-1.73 (5H, m), 3.06 (2H, t), 7.87 (1H, dd), 7.92 (1H, d), 8.14 (1H, d), 8.58 (1H t), 8.74 (1H, s), 10.10 (1H, s)

LC/MS, t=3.67 min, Molecular ion observed (MH⁺)=438 consistent with the molecular formula C₂₀H₁₉N₅OF₃³⁵Cl

### EXAMPLE 238

### 2-(2-Bromo-4-chloro-phenylamino)-4-trifluoromethyl-pyrimidine-5-carboxylic acid cyclohexylmethyl-amide

In a manner similar to Example 166, 2-chloro-4-trifluoromethyl-pyrimidine-5-carboxylic acid cyclohexylmethyl-amide (80 mg) and 2-bromo-4-chloroaniline (257 mg, ex Lancaster) afforded the title compound (23 mg). Sample purified by mass directed auto-prep.

NMR (DMSO-d6) δ 0.89-0.95 (2H, m), 1.15-1.20 (3H, m), 1.44-1.46 (1H, m), 1.62-1.72 (5H, m), 3.04 (2H, t), 7.52 (2H, m), 7.85 (1H, d), 8.53 (1H t), 8.61 (1H, s), 10.00 (1H, s)

LC/MS, t=3.94 min, Molecular ion observed (MH⁺)=493 consistent with the molecular formula C₁₉H₁₉N₄OF₃³⁵Cl⁸¹Br

### EXAMPLE 239

### 2-(2-Bromo-4-chloro-phenylamino)-4-trifluoromethyl-pyrimidine-5-carboxylic acid (tetrahydro-pyran-4-ylmethyl)-amide

In a manner similar to Example 166, 2-chloro-4-trifluoromethyl-pyrimidine-5-carboxylic acid (tetrahydro-pyran-4-ylmethyl)-amide (80 mg) and 2-bromo-4-chloroaniline (255 mg, ex Lancaster) afforded the title compound (6 mg). Sample purified by mass directed auto-prep.

NMR (DMSO-d6) δ 1.14-1.23 (2H, m), 1.58 (2H, d), 1.72 (1H, m), 3.1 (2H, t), 3.28 (2H, m), 3.84 (2H, d), 7.50 (2H, m), 7.82 (1H, d), 8.58 (1H, t), 8.63 (1H, s), 10.00 (1H, s)

LC/MS, t=3.40 min, Molecular ion observed (MH⁺)=495 consistent with the molecular formula C₁₈H₁₇N₄O₂F₃³⁵Cl⁸¹Br

### EXAMPLE 240

### 2-(3-Bromophenylamino)-4-trifluoromethyl-pyrimidine-5-carboxylic acid cyclopropylmethyl-amide

In a manner similar to Reference Example 1 (c) 2-(3-bromophenylamino)-4-trifluoromethyl-pyrimidine-5-carboxylic acid (80 mg) and cyclopropylmethylamine (19 mg, ex Lancaster) afforded the title compound (24 mg). Sample purified by mass directed auto-prep.

NMR (DMSO-d6) δ 0.22 (2H, m), 0.45 (2H, m), 1.67 (1H, m), 3.13 (2H, t), 7.23 (1H, d), 7.30 (1H, t), 7.72 (1H, d), 8.10 (1H, m), 8.68 (1H, t), 8.80 (1H s), 10.60 (1H, s)

LC/MS, t=3.49 min, Molecular ion observed (MH⁺)=417 consistent with the molecular formula C₁₆H₁₄N₄OF₃⁸¹Br

### EXAMPLE 241

### 2-(2,4-Dichlorophenylamino)-4-trifluoromethylpyrimidine-5-carboxylic acid cyclopropylmethyl-amide

In a manner similar to Reference Example 1 (c) 2-(2,4-dichlorophenylamino)-4-trifluoromethyl-pyrimidine-5-carboxylic acid (80 mg) and cyclopropylmethylamine (19 mg, ex Lancaster) afforded the title compound (58 mg).

NMR (DMSO-d6) δ 0.22 (2H, m), 0.45 (2H, m), 1.67 (1H, m), 3.13 (2H, t), 7.23 (1H, d), 7.30 (1H, t), 7.72 (1H, d), 8.10 (1H, m), 8.68 (1H, t), 8.80 (1H s), 10.60 (1H, s)

LC/MS, t=3.56 min, Molecular ion observed (MH⁺)=405 consistent with the molecular formula C₁₆H₁₃N₄OF₃³⁵Cl

### EXAMPLE 242

### 2-(2,3-Difluorophenylamino)-4-trifluoromethyl-pyrimidine-5-carboxylic acid cyclohexylmethyl-amide

To a solution of 2-chloro-4-trifluoromethyl-pyrimidine-5-carboxylic acid cyclohexylmethyl-amide (Example 166a) (50 mg) in 1,4-dioxan (1 ml) was added 2,3-difluoroaniline (Aldrich) (113 mg) and the mixture was stirred at reflux for 47 hours using a Radleys Greenhouse Parallel Synthesiser. The dioxan was removed using a nitrogen blow down unit. The residue was taken up into methanol (0.5 ml) and dimethylsulfoxide (0.5 ml) and purified using a mass directed auto-preparative system to give the title compound (16 mg)

NMR (Chloroform-d6) δ 0.94-1.08 (2H, m), 1.15-1.34 (3H, m), 1.5-1.6 (>1H, m & water) 1.65-1.73 (1H, m), 1.73-1.83 (4H, m), 3.30 (2H, t,), 5.91 (1H, bs) 6.88-6.98 (1H, m) 7.08-7.1 (1H, m), 7.66 (1H, bs), 8.16-8.25 (1H, m), 8.75 (1H, s).

LC/MS t=3.66 min, [MH⁺] 415 consistent with the molecular formula C₁₉H₁₉F₅N₄O

### EXAMPLE 243

### 2-(2-Fluoro-3-trifluoromethyl-phenylamino)-4-trifluoromethyl-pyrimidine-5-carboxylic acid cyclohexylmethyl-amide

In a manner similar to Example 242, 2-chloro-4-trifluoromethyl-pyrimidine-5-carboxylic acid cyclohexylmethyl-amide (Example 166a) (50 mg) in 1,4-dioxan (1 ml) and 2-fluoro-3-trifluoromethylphenylamine (Aldrich) (156 mg) were reacted to give the title compound (11 mg)

NMR (Chloroform-d6) δ 0.94-1.08 (2H, m), 1.15-1.34 (3H, m), 1.55-1.59 (1H, m), 1.65-1.73 (1H, m), 1.73-1.83 (4H, m), 3.30 (2H, t,), 5.91 (1H, bs), 7.28-7.37 (2H, m), 7.74 (1H, bs), 8.65-8.73 (1H, m), 8.77-8.80 (1H, m)

LC/MS t 3.66 min [MH⁺]=465 consistent with the molecular formula C₂₀H₁₉F₇N₄O

### EXAMPLE 244

### 2-(2-Chloro-4-methylphenylamino)-4-trifluoromethyl-pyrimidine-5-carboxylic acid cyclohexylmethyl-amide

To a solution of 2-chloro-4-trifluoromethyl-pyrimidine-5-carboxylic acid cyclohexylmethyl-amide (Example 166a) (50 mg) in 1,4-dioxan (1 ml) was added 2-chloro-4-methylphenylamine (Aldrich) (109 mg) the mixture was stirred at reflux for 24 hours using a Radleys Greenhouse Parallel Synthesiser. The dioxan was removed using a nitrogen blow down unit. The residue was taken up into methanol (0.5 ml) and dimethylsulfoxide (0.5 ml) and purified using mass directed auto-preparative system to give the title compound) (24 mg)

NMR (Methanol-d6) δ 1.50-1.60 (2H, m), 1.70-1.89 (3H, m), 2.06-215 (1H, m), 2.2-2.26 (1H, m), 2.27-2.38 (4H, m), 2.88 (3H, s), 3.71 (2H, d), 7.68 (1H, d), 7.85 (1H, s), 8.31 (1H, d), 9.10 (1H, s).

LC/MS t=3.81 min, [MH⁺]=427 consistent with the molecular formula C₂₀H₂₂³⁵Cl F₃N₄O

### EXAMPLE 245

### 2-(4-Chloro-3-methoxyphenylamino)-4-trifluoromethyl-pyrimidine-5-carboxylic acid cyclohexylmethyl-amide

In a manner similar to Example 243, 2-chloro-4-trifluoromethyl-pyrimidine-5-carboxylic acid cyclohexylmethyl-amide (Example 166a) (50 mg) in 1,4-dioxan (1 ml) and 4-chloro-3-methoxy-phenylamine (Wychem) (122 mg) were reacted to give the title compound (33 mg)

NMR (Methanol-d6) δ 0.95-1.06 (2H, m), 1.20-1.34 (3H, m), 1.55-1.64 (1H, m), 1.65-171 (1H, m), 1.72-1.85 (4H, m), 3.19 (2H, d), 3.90 (3H, s), 7.18 (1H, dd), 7.27 (1H, d), 7.80 (1H, bs), 8.64 (1H, s).

LC/MS t=3.79 min, [MH⁺] 443 consistent with the molecular formula C₂₀H₂₂³⁵Cl F₃N₄O₂

### EXAMPLE 246

### 2-(5-Chloro-2-methylphenylamino)-4-trifluoromethyl-pyrimidine-5-carboxylic acid cyclohexylmethyl-amide

In a manner similar to Example 243, 2-chloro-4-trifluoromethyl-pyrimidine-5-carboxylic acid cyclohexylmethyl-amide (Example 166a) (50 mg) in 1,4-dioxan (1 ml) and 5-chloro-2-methyl aniline (Aldrich) (110 mg) were reacted to give the title compound (36 mg)

NMR (Methanol-d6) δ1.47-1.59 (2H, m), 1.72-1.89 (3H, m), 2.05-2.18 (1H, m) 2.19-2.25 (1H, m), 2.31 (4H, t), 2.79 (3H, s), 3.71 (2H, d), 7.76 (1H, dd), 7.76 (1H, d), 8.17 (1H, d), 9.09 (1H, s)

LC/MS t=3.77 min [MH⁺]=427 consistent with the molecular formula C₂₀H₂₂³⁵Cl F₃N₄O

### EXAMPLE 247

### 2-(3-Chloro-4-fluoro-phenylamino)-4-trifluoromethyl-pyrimidine-5-carboxylic acid cyclopentylmethyl-amide

2-Chloro-4-trifluoromethyl-pyrimidine-5-carboxylic acid cyclopentylmethyl-amide (116 mg Example 183a), 3-chloro-4-fluoroaniline (ex-Aldrich, 275 mg), and 1,4-dioxan (1.2 ml) were stirred at 100° C. under nitrogen for 6 h. The cooled reaction mixture was evaporated in vacuo, treated with ethyl acetate (5 ml), washed with aqueous 2M hydrochloric acid (2×3 ml), followed by brine, and dried (Na₂SO₄). The solution was evaporated in vacuo to give the title compound (104 mg).

NMR δ (DMSO-d6) 1.15-1.32 (2H, m), 1.46-1.66 (4H, m) 1.66-1.78 (2H, m), 2.1 (1H, q), 3.17 (2H, t), 7.4 (1H, t), 7.63-7.7 (1H, m), 8.05 (1H, dd), 8.61 (1H, t), 8.79 (1H, s), 10.6 (1H, s).

LC/MS t=3.7 min, Molecular ion observed [MH+]=417 consistent with the molecular formula C₁₈H₁₇ClF₄N₄O.

### EXAMPLE 248

### 2-(3-Chloro-2-fluoro-phenylamino)-4-trifluoromethyl-pyrimidine-5-carboxylic acid cyclopentylmethyl-amide

In a manner similar to Example 247, 3-chloro-2-fluoroaniline (ex-Acros, 275 mg) was reacted for 18 h, worked up analogously, then stirred in isohexane (6 ml), and filtered off to give the title compound (82 mg).

NMR δ (CDCl₃) 1.2-1.34 (2H, m), 1.55-1.76 (>4H, m+H2O), 1.78-1.89 (2H, m), 2.16 (1H, q), 3.41 (1H, t), 5.83-5.95 (1H, brt), 7.1-7.18 (2H, m), 7.28 (1H, s), 7.66 (1H, brs), 8.3-8.4 (1H, m), 8.75 (1H, s).

LC/MS t=3.7 min, Molecular ion observed [MR⁺] 417 consistent with the molecular formula C₁₈H₁₇ClF₄N₄O.

### EXAMPLE 249

### 2-(2-Chloro-5-fluoro-phenylamino)-4-trifluoromethyl-pyrimidine-5-carboxylic acid cyclopentylmethyl-amide

2-Chloro-4-trifluoromethyl-pyrimidine-5-carboxylic acid cyclopentylmethyl-amide (100 mg Example 183a), 2-chloro-5-fluoroaniline (ex-Fluorochem, 237 mg), and 1,4-dioxan (1 ml) were stirred at 100° C. under nitrogen for 18 h. The cooled reaction mixture was evaporated in vacuo, treated with ethyl acetate (5 ml), washed with aqueous 2M hydrochloric acid (2×3 ml), followed by water (2×3 ml), and dried (Na₂SO₄). The solution was evaporated in vacuo and the residue purified by mass directed autopreparative purification to give the title compound (35 mg).

NMR δ (CDCl₃) 1.2-1.35 (2H, m), 1.53-1.76 (>4H, m+H2O), 1.78-1.90 (2H, m), 2.17 (1H, q), 3.41 (2H, dd), 5.9 (1H, brt), 7.0-7.11 (2H, m), 7.65-7.7 (1H, m) 8.56 (1H, dd), 8.79 (1H, s).

LC/MS t=3.67 min, Molecular ion observed [MH⁺] 417 consistent with the molecular formula C₁₈H₁₇ClF₄N₄O.

### EXAMPLE 250

### 2-(3,4-Dichlorophenylamino)-4-trifluoromethylpyrimidine-5-carboxylic acid (tetrahydro-pyran-4-ylmethyl)-amide

In a manner similar to Reference Example 1(c) 2-(3,4-dichlorophenylamino)-4-trifluoromethyl-pyrimidine-5-carboxylic acid (30 mg) and 4-aminomethyltetrahydropyran (20 mg, ex CombiBlocks) afforded the title compound (38 mg).

NMR (DMSO-d6) δ 1.18-1.25 (2H, m), 1.62 (2H, d), 1.74 (1H, m), 3.1 (2H, t), 3.25 (2H, m), 3.85 (2H, d), 7.60 (1H, t), 7.69 (1H, m), 8.16 (1H, dd), 8.64 (1H, t), 8.84 (1H, s), 10.70 (1H, s)

LC/MS, t=3.45 min, Molecular ion observed (MH⁺)=449 consistent with the molecular formula C₁₈H₁₇N₄O₂³⁵Cl₂F₃

### EXAMPLE 251

### 2-(Phenylamino)-4-trifluoromethyl-pyrimidine-5-carboxylic acid cyclopentylmethyl-amide

In a manner similar to Reference Example 1 (c), 2-(Phenylamino)-4-trifluoromethyl-pyrimidine-5-carboxylic acid (30 mg) and cyclopentylmethylamine hydrochloride (21 mg) afforded the title compound (32 mg) after purification by trituration with diethylether.

NMR (DMSO-d6) δ 1.20-1.25 (2H, m), 1.48-1.72 (6H, m), 2.07 (1H, m), 3.13 (2H, t), 7.04 (1H, t), 7.34 (2H, t), 7.74 (2H, d), 8.58 (1H, t), 8.70 (1H s), 10.35 (1H, s)

LC/MS, t=3.52 min, Molecular ion observed (MH⁺)=365 consistent with the molecular formula C₁₈H₁₉N₄OF₃

### EXAMPLE 252

### 2-(2-Fluoro-3-trifluoromethyl-phenylamino)-4-trifluoromethyl-pyrimidine-5-carboxylic acid cyclobutylmethyl-amide

2-Chloro-4-trifluoromethyl-pyrimidin-5-carboxylic acid cyclobutylmethyl-amide (200 mg) in 2-fluoro-3-(trifluoromethyl)aniline (0.5 ml) was heated at 180° C. under microwave irradiation for 30 minutes. The residue was dissolved in dichloromethane and purified over silica gel (Merck 9385) using the Biotage Horizon system eluting with 10% ethylacetate/isohexane to 100% ethyl acetate gradient to afford the title compound.

NMR (CDCl₃) δ1.70-1.81 (2H, m), 1.86-2.00 (2H, m), 2.07-2.17 (2H, m), 2.51-2.65 (1H, m), 3.48 (2H, dd), 5.78-5.86 (1H, m), 7.25-7.36 (2H, m), 7.70-7.76 (1H, bs), 8.64-8.72 (1H, m), 8.75-8.79 (1H, s)

LC/MS, t=3.64 min, Molecular ion observed (MH⁺)=437 consistent with the molecular formula C₁₈H₁₅F₇N₄O

### EXAMPLE 253

### 2-(2-Methyl-4-chloro-phenylamino)-4-trifluoromethyl-pyrimidine-5-carboxylic acid cyclobutylmethyl-amide

To a solution of 2-chloro-4-trifluoromethyl-pyrimidin-5-carboxylic acid cyclobutylmethyl-amide (50 mg) in 1,4-dioxan (11.0 ml) was added 2-methyl-4-chloroaniline (120 mg) and the solution heated at 180° C. under microwave irradiation for 30×2 minutes. The residue was dissolved in 1:1 DMSO: methanol (11.0 ml) and purified by Mass Directed Auto-Purification to afford the title compound (36 mg).

NMR (CDCl₃) δ 1.79-1.80 (2H, m), 1.85-1, 99 (2H, m), 2.05-2.16 (2H, m), 2.25-2.63 91H, m), 5.74-5.83 (1H, m), 7.15 (1H, bs), 7.2-7.78 (21 m), 7.81 (1H, d), 8.66 (1H, s)

LC/MS, t=3.6 min, Molecular ion observed (MH⁺)=398 consistent with the molecular formula C₁₈H₁₈ClF₃N₄O

### EXAMPLE 254

### 2-(2-Trifluoromethyl-4-bromo-phenylamino)-4-trifluoromethyl-pyrimidine-5-carboxylic acid (tetrahydro-pyran-4-ylmethyl)-amide

2-Chloro-4-trifluoromethyl-pyrimidin-5-carboxylic acid (tetrahydro-pyran-4-ylmethyl)-amide (80 mg) in 2-trifluoromethyl-4-bromoaniline (0.5 ml) was heated at 190° C. under microwave irradiation for 20 minutes. The sample was purified by mass directed auto-purification to afford the title compound (21 mg).

NMR (DMSO-d6) δ 1.15-1.23 (2H, m), 1.57 (2H, d), 1.60 (1H, m), 3.09 (2H, t), 3.26 (2H, t), 3.84 (2H, d), 7.51 (1H, d), 7.95 (2H, m), 8.58 (2H, s, t), 10.00 (1H, s)

LC/MS, t=3.41 min, Molecular ion observed (MH⁺)=529 consistent with the molecular formula C₁₉H₁₇N₄O₂F₆⁸¹Br

### EXAMPLE 255

### 2-(3-Chlorophenylamino)-4-trifluoromethyl-pyrimidine-5-carboxylic acid (tetrahydrothiopyran-4-ylmethyl) amide

### a) 4-(Aminomethyl)tetrahydrothiopyran

A solution of borane-tetrahydrofuran complex (1M in tetrahydrofuran, 11 ml) was added over 5 minutes to a solution of tetrahydro-2H-thiopyran-4-carbonitrile (1.27 g) [Heimgartner et al, Helv. Chim. Acta 80(5), 1528 (1997)] in dry tetrahydrofuran (5 ml) under nitrogen at room temperature. The solution was heated at reflux overnight, then cooled to 20° C. Methanol (15 ml) was added dropwise keeping the temperature below 25° C., then the mixture was cooled to 0° C. and dry hydrogen chloride was bubbled through for 15 mins. The resulting mixture was heated at reflux for 1.5 hours, evaporated and the residue re-evaporated twice from methanol. Ether (30 ml) was added giving a white oily solid. The ether was decanted and the residue was dissolved in water (30 ml) and extracted with dichloromethane (2×30 ml). The remaining aqueous was made strongly basic with sodium hydroxide and extracted with dichloromethane (2×30 ml). The combined extracts were dried over potassium carbonate and evaporated to give the title compound (390 mg)

NMR (DMSO) δ 1.2 (5H, m), 2.0 (2H, m), 2.36 (2H, m), 2.55 (4H, m).

### b) 2-(3-Chlorophenylamino)-4-trifluoromethyl-pyrimidine-5-carboxylic acid (tetrahydrothiopyran-4-ylmethyl) amide

In a manner similar to Reference Example 1b) 2-(3-chlorophenylamino)-4-trifluoromethylpyrimidine-5-carboxylic acid (95 mg) and 4-(aminomethyl)tetrahydrothiopyran (79 mg) (above) gave the title compound (92 mg).

NMR (DMSO-d6) δ 1.26 (2H, m), 1.55 (1H, m), 2.01 (2H, m), 2.60 (4H, m), 3.10 (2H, t), 7.09 (1H, m), 7.37 (1H, t), 7.65 (1H, m), 7.96 (1H, m), 8.63 (1H, t), 8.81 (1H, s), 10.6 (1H, s).

LC/MS CF111437, t=3.61 min, Molecular ion observed (MH⁺)=431 consistent with the molecular formula C₁₈H₁₈³⁵ClF₃N₄OS

### EXAMPLE 256

### 2-(2,4-Dichlorophenylamino)-4-trifluoromethyl-pyrimidin-5-carboxylic acid (tetrahydrothiopyran-4-ylmethyl) amide

In a manner similar to Reference Example 1b) 2-(2,4-dichlorophenylamino)-4-trifluoromethyl-pyrimidine-5-carboxylic acid (106 mg) and 4-(aminomethyl)tetrahydrothiopyran (79 mg) (Example 255a) gave the title compound (82 mg).

NMR (DMSO-d6) δ 1.27 (2H, m), 1.55 (1H, m), 2.00 (2H, m), 2.59 (4H, m), 3.08 (2H, t), 7.47 (1H, m), 7.57 (1H, d), 7.72 (1H, m), 8.59 (1H, t), 8.64 (1H, s), 10.0 (1H, s).

LC/MS CF111493, t=3.70 min, Molecular ion observed (MH⁺)=465 consistent with the molecular formula C₁₈H₁₇³⁵Cl₂F₃N₄OS

### EXAMPLE 263

### 2-(3-Chlorophenylamino)-4-trifluoromethyl-pyrimidine-5-carboxylic acid (2-oxo-propyl)-amide

To a stirred solution of 2-(3-chloro-phenylamino)-trifluoromethyl-pyrimidine-5-carboxylic acid (2-hydroxy-propyl)-amide (200 mg) in dimethylsulfoxide (6.0 ml) and triethylamine (324 mg) at 0° C. was added a solution of sulphur trioxide-pyridine complex (250 mg) in dimethylsulfoxide (6.0 ml). This was allowed to warm to room temperature and after 2 hours the mixture was diluted with dichloromethane and washed twice with 0.1N hydrochloric acid. The organic layer was dried (Na₂SO₄) and evaporated. The sample was purified by mass directed auto-purification to afford the title compound (91 mg).

NMR (DMSO-d6) δ 2.15 (3H, s), 4.13 (2H, d), 7.10 (1H, d), 7.36 (1H, t), 7.67 (1H, d), 7.96 (1H, s), 8.84 (1H, s), 8.94 (1H, t), 10.55 (1H, s)

LC/MS, t=3.18 min, Molecular ion observed (MH⁺)=373 consistent with the molecular formula C₁₅H₁₂N₄O₂F₃³⁵Cl

### EXAMPLE 264

### 2-(3-Chlorophenylamino)-4-trifluoromethylpyrimidine-5-carboxylic acid (dioxo-hexahydro-1l⁶-thiopyran-4-ylmethyl)-amide

2-(3-Chlorophenylamino)-4-trifluoromethylpyrimidine-5-carboxylic acid (tetrahydro-thiopyran-4-ylmethyl)-amide (Example 255) (82 mg) was dissolved in dichloromethane (15 ml) and cooled in an ice bath. A solution of 3-chloroperbenzoic acid (95 mg; Lancaster 50-56%) in dichloromethane (5 ml) was added dropwise over 5 mins. The resulting solution was stirred at room temp for 2 hrs then a saturated solution of sodium sulphite (10 ml) was added and the mixture was stirred for 15 mins. Dichloromethane (20 ml), saturated sodium bicarbonate solution (20 ml) and water (30 ml) were added, separated and the organics were washed with water (2×30 ml), dried over magnesium sulphate and evaporated to an oil. Purification by chromatography on silica gel (dichloromethane/methanol 10:1) gave the title compound (17 mg).

LC/MS t=3.09 min, Molecular ion observed (MH^{+x)=}463 consistent with the molecular formula C₁₈H₁₈³⁵ClF₃N₄O₃S

### EXAMPLE 265

### 2-(2,4-Dichlorophenylamino)-4-trifluoromethylpyrimidin-5-carboxylic acid (dioxo-hexahydro-1l⁶-thiopyran-4-ylmethyl)-amide

In a similar manner to Example 264, 2-(2,4-dichlorophenylamino)-4-trifluoromethylpyrimidine-5-carboxylic acid (tetrahydro-thiopyran-4-ylmethyl)-amide (Example 256) (72 mg) and 3-chloroperbenzoic acid (146 mg) gave the title compound (63 mg)

LC/MS t=3.21 min, Molecular ion observed (MH^{+x)=}497 consistent with the molecular formula C₁₈H₁₇³⁵Cl₂F₃N₄O₃S

### EXAMPLE 266

### 2-(3-Chlorophenylamino)-4-trifluoromethylpyrimidine-5-carboxylic acid benzyl-amide

(a). To a solution of benzyl 2-chloro-4-trifluoromethylpyrimidine-5-carboxylate (0.50 g, ex Maybridge) in 1,4-dioxan (5 ml) was added 3-chloroaniline (0.85 ml) and the solution stirred at room temperature for 15 h. 1,4-Dioxan was removed under reduced pressure and ethyl acetate (15 ml) added. The solution was washed sequentially with 2N hydrochloric acid (10 ml) and water (3×10 ml), dried (MgSO₄), evaporated and triturated with hexane to afford benzyl 2-(3-chlorophenylamino)-4-trifluoromethylpyrimidine-5-carboxylate (524 mg).

NMR (400 MHz, DMSO-d6) δ 5.35 (2H, s), 7.14 (1H, d), 7.35-7.45 (6H, m), 7.68 (1H, m), 7.98

(1H, s), 9.13 (1H, s), 10.95 (1H, s).

LC/MS, t=3.70 min, [MH⁺] 408 and 410.

(b). To a solution of benzyl 2-(3-chlorophenylamino)-4-trifluoromethyl-pyrimidine-5-carboxylate (0.50 g) in ethanol (15 ml) was added a solution of potassium hydroxide (205 mg) in ethanol (10 ml) and the solution stirred at reflux for 15 h. Ethanol was removed under reduced pressure and water (15 ml) added. The solution was washed with ether and concentrated hydrochloric acid added to adjust the acidity to pH 1. The precipitated solid was filtered, washed with water and dried in vacuo at 50° C. to afford 2-(3-chlorophenylamino)-4-trifluoromethylpyrimidine-5-carboxylic acid (366 mg,).

NMR (400 MHz, DMSO-d6) δ 7.49 (1H, d), 7.71 (1H, t), 7.98 (1H, d), 8.33 (1H, s), 9.42 (1H, s), 11.15 (1H, s), 14.0 (1H, br s).

LC/MS, t=3.44 min, [MH⁺] 318 and 320.

(c). To a solution of 2-(3-chlorophenylamino)-4-trifluoromethylpyrimidine-5-carboxylic acid (35 mg) in dimethylformamide (2 ml) was added successively N-ethylmorpholine (42 μl), benzylamine (15 μl), 1-hydroxybenzotriazole hydrate (23 mg) and 1-(3-dimethylamino-propyl)-3-ethylcarbodiimide hydrochloride (25 mg). The solution was stirred for 3 h and allowed to stand overnight. Dimethylformamide was removed under reduced pressure and ethyl acetate (5 ml) added. The solution was washed sequentially with 5% sodium bicarbonate solution (2.5 ml), water (2.5 ml), 5% citric acid solution (2.5 ml) and brine (2×2.5 ml), dried (MgSO₄) and evaporated to afford the title compound (45 mg).

NMR (400 MHz, DMSO-d6) δ 4.47 (2H, d), 7.10 (1H, d), 7.25 (1H, m), 7.36 (5H, m), 7.69 (1H, d), 7.98 (1H, s), 8.89 (1H, s), 9.12, 1H, t), 10.65 (1H, s).

LC/MS, t=3.23 min, [MH⁺] 407 and 409.

### EXAMPLE 267

### 2-(3-Chlorophenylamino)-4-trifluoromethylpyrimidine-5-carboxylic acid (pyridin-4-ylmethyl)amide

In a manner similar to Example 266(c) 2-(3-chlorophenylamino)-4-trifluoromethylpyrimidine-5-carboxylic acid (35 mg) and 4-(aminomethyl)pyridine (13.5 μl) afforded the title compound (32 mg).

NMR (400 MHz, DMSO-d6) δ 4.48 (2H, d), 7.10 (1H, d), 7.37 (3H, m), 7.69 (1H, d), 7.98 (1H, s), 8.55 (2H, d), 8.97 (1H, s), 9.26 (1H, t), 10.65 (1H, s).

LC/MS, t=2.90 min, [MH⁺] 408 and 410.

### EXAMPLE 268

### 2-(3-Chlorophenylamino)-4-trifluoromethylpyrimidine-5-carboxylic acid N-benzyl-N-methylamide

In a manner similar to Example 266(c) 2-(3-chlorophenylamino)-4-trifluoromethylpyrimidine-5-carboxylic acid (35 mg) and N-methylbenzylamine (17 μl) afforded the title compound (46 mg).

NMR (400 MHz, DMSO-d6) Rotamers in 65:35 ratio δ 2.88 (1.95H, s), 2.98 (1.05H, s), 4.58 (0.7H, br s), 4.75 (1.3H, br s), 7.17 (1H, t), 7.30 (1H, d), 7.35-7.5 (5H, m), 7.72 (1H, t), 8.00 (0.35H, t), 8.06 (0.65H, t), 8.89 (0.35H, s), 8.95 (0.65H, s), 10.65 (0.35H, s), 10.7 (0.65H, s).

LC/MS, t=3.35 min, [MH⁺] 421 and 423.

### EXAMPLE 269

### 2-(3-Chlorophenylamino)-4-trifluoromethylpyrimidine-5-carboxylic acid 4-methoxybenzyl-amide

In a manner similar to Example 266(c) 2-(3-chlorophenylamino)-4-trifluoromethylpyrimidine-5-carboxylic acid (35 mg) and 4-methoxybenzylamine (17 μl) afforded the title compound (18 mg).

NMR (400 MHz, DMSO-d6) δ 3.75 (3H, s), 4.40 (2H, d), 6.94 (2H, d), 7.10 (1H, d), 7.28 (2H, d), 7.38 (1H, t), 7.69 (1H, d), 7.98 (1H, s), 8.88 (1H, s), 9.08 (1H, t), 10.65 (1H, s).

LC/MS, t=3.57 min, [MH⁺] 437 and 439.

### EXAMPLE 270

### 2-(3-Chlorophenylamino)-4-trifluoromethylpyrimidine-5-carboxylic acid 4-fluorobenzyl-amide

In a manner similar to Example 266(c) 2-(3-chlorophenylamino)-4-trifluoromethylpyrimidine-5-carboxylic acid (35 mg) and 4-fluorobenzylamine hydrochloride (21 mg) afforded the title compound (35 mg).

NMR (400 MHz, DMSO-d6) δ 4.45 (2H, d), 7.10 (1H, d), 7.18 (2H, t), 7.35-7.45 (3H, m), 7.68 (1H, d), 7.97 (1H, s), 8.89 (1H, s), 9.14 (1H, t), 10.65 (1H, s).

LC/MS, t=3.68 min, [MH⁺] 425 and 427.

### EXAMPLE 271

### 2-(3-Chlorophenylamino)-4-trifluoromethylpyrimidine-5-carboxylic acid 4-cyanobenzyl-amide

In a manner similar to Example 266(c) 2-(3-chlorophenylamino)-4-trifluoromethylpyrimidine-5-carboxylic acid (35 mg) and 4-cyanobenzylamine (17.5 mg) afforded the title compound (13 mg).

NMR (400 MHz, DMSO-d6) δ 4.94 (2H, d), 7.49 (1H, d), 7.68 (1H, t), 7.95 (2H, d), 8.06 (1H, d), 8.23 (2H, d), 8.38 (1H, s), 9.32 (1H, s), 9.64 (1H, t), 11.05 (1H, s).

LC/MS, t=3.56 min, [MH⁺] 432 and 434.

### EXAMPLE 272

### 2-(2,3-Dichlorophenylamino)-4-trifluoromethylpyrimidine-5-carboxylic acid N-benzyl-N-methylamide

(a). To a solution of methyl 2-chloro-4-trifluoromethylpyrimidine-5-carboxylate (0.40 g, ex Maybridge) in 1,4-dioxan (5 ml) was added 2,3-dichloroaniline (1.27 g) and the solution stirred at reflux temperature for 24 h. 1,4-Dioxan was removed under reduced pressure and ethyl acetate (15 ml) added. The solution was washed sequentially with 2N hydrochloric acid (10 ml) and water (3×10 ml), dried (MgSO₄), evaporated and triturated with hexane to afford methyl 2-(2,3-dichlorophenylamino)-4-trifluoromethyl-pyrimidine-5-carboxylate (176 mg,).

NMR (400 MHz, CDCl₃) δ 3.97 (3H, s), 7.25 (2H, m), 8.15 (1H, s), 8.48 (1H, d), 9.07 (1H, s).

LC/MS, t=3.68 min, [MH⁺] 366 and 368.

(b). In a manner similar to Example 266(b) methyl 2-(2,3-dichlorophenylamino)-4-trifluoro-methylpyrimidine-5-carboxylate (0.18 g) afforded 2-(2,3-dichlorophenylamino)-4-trifluoro-methylpyrimidine-5-carboxylic acid (0.13 g).

NMR (400 MHz, DMSO-d6) δ 7.40 (1H, t), 7.56 (2H, d), 8.96 (1H, s), 10.45 (1H, s), 13.6 (1H, s).

LC/MS, t=4.06 min, [MH⁺—CO₂] 306 and 308.

(c). In a manner similar to Example 266(c) 2-(2,3-dichlorophenylamino)-4-trifluoromethyl-pyrimidine-5-carboxylic acid (39 mg) and N-methylbenzylamine (21.5 μl) afforded the title compound (50 mg).

NMR (400 MHz, CDCl₃) Rotamers in 65:35 ratio δ 2.79 (1.95H, s), 3.08 (1.05H, s), 4.42 (0.7H, br s), 4.78 (1.3H, br s), 7.14 (1H, d), 7.2-7.3 (2H, m), 7.3-7.45 (4H, m), 7.96 (0.35H, s), 8.01 (0.65H, s), 8.40 (0.35H, d), 8.45 (0.65H, d), 8.55 (0.35H, s), 8.59 (0.65H, s).

LC/MS, t=3.74 min, [MH^{+x]} 455 and 457.

### EXAMPLE 273

### 2-(2,4-Dichlorophenylamino)-4-trifluoromethylpyrimidine-5-carboxylic acid benzyl-amide

(a). In a manner similar to Example 272(a) methyl 2-chloro-4-trifluoromethylpyrimidine-5-carboxylate (0.5 g) and 2,4-dichloroaniline (1.7 g) afforded methyl 2-(2,4-dichlorophenyl-amino)-4-trifluoromethyl-pyrimidine-5-carboxylate (214 mg).

NMR (400 MHz, CDCl₃) δ 3.95 (3H, s), 7.33 (1H, d), 7.46 (1H, d), 7.99 (1H, s), 8.48 (1H, d), 9.06 (1H, s). LC/MS, t=3.74 min, [MH⁺] 366 and 368.

(b). In a manner similar to Example 266(b) methyl 2-(2,4-dichlorophenylamino)-4-trifluoro-methylpyrimidine-5-carboxylate (0.21 g) afforded 2-(2,4-dichlorophenylamino)-4-trifluoro-methylpyrimidine-5-carboxylic acid (0.18 g).

NMR (400 MHz, DMSO-d6) δ 7.47 (1H, d), 7.60 (1H, d), 7.75 (1H, s), 8.96 (1H, s), 10.3 (1H, s), 13.6 (1H, s). LC/MS, t=4.17 min, [MH⁺—CO₂] 306 and 308.

(c). In a manner similar to Example 266(c) 2-(2,4-dichlorophenylamino)-4-trifluoromethyl-pyrimidine-5-carboxylic acid (39 mg) and benzylamine (18 μl) afforded the title compound (41 mg).

NMR (400 MHz, CDCl₃) δ 4.64 (2H, d), 6.08 (1H, br s), 7.25-7.4 (5H, m), 7.44 (1H, d), 7.90 (1H, s), 8.43 (1H, d), 8.74 (1H, s). LC/MS, t=3.69 min, [MH⁺] 441 and 443.

### EXAMPLE 274

### 2-(3,4-Dichlorophenylamino)-4-trifluoromethylpyrimidine-5-carboxylic acid benzyl-amide

(a). In a manner similar to Example 272(a) methyl 2-chloro-4-trifluoromethylpyrimidine-5-carboxylate (0.5 g) and 3,4-dichloroaniline (1.7 g) afforded methyl 2-(3,4-dichlorophenyl-amino)-4-trifluoromethyl-pyrimidine-5-carboxylate (591 mg).

NMR (400 MHz, CDCl₃) δ 3.96 (3H, s), 7.45 (2H, m), 7.57 (1H, s), 7.98 (1H, s), 9.07 (1H, s).

LC/MS, t=3.87 min, [MH⁺] 366 and 368.

(b). In a manner similar to Example 266(b) methyl 2-(3,4-dichlorophenylamino)-4-trifluoro-methylpyrimidine-5-carboxylate (0.59 g) afforded 2-(3,4-dichlorophenylamino)-4-trifluoromethylpyrimidine-5-carboxylic acid (0.51 g).

NMR (400 MHz, DMSO-d6) δ 7.65 (1H, d), 7.72 (1H, d of d), 8.19 (1H, s), 9.12 (1H, s), 10.95 (1H, s), 13.7 (1H, s). LC/MS, t=4.49 min, [MH⁺—CO₂] 306 and 308.

(c). In a manner similar to Example 266(c) 2-(3,4-dichlorophenylamino)-4-trifluoromethyl-pyrimidine-5-carboxylic acid (39 mg) and benzylamine (18 μl) afforded the title compound (51 mg).

NMR (400 MHz, CDCl₃) δ 4.65 (2H, d), 6.10 (1H, br s), 7.3-7.4 (5H, m), 7.42 (1H, s), 7.45 (1H, s), 7.94 (1H, s), 8.78 (1H, s). LC/MS, t=3.80 min, [MH⁺] 441 and 443.

### EXAMPLE 275

### 2-(2,6-Dichlorophenylamino)-4-trifluoromethylpyrimidine-5-carboxylic acid N-benzyl-N-methylamide

(a). In a manner similar to Example 272(a) methyl 2-chloro-4-trifluoromethylpyrimidine-5-carboxylate (0.5 g) and 2,6-dichloroaniline (1.7 g) in 1,4-dioxan (5 ml) was stirred at reflux temperature for 7 days to afford methyl 2-(2,6-dichlorophenyl-amino)-4-trifluoromethyl-pyrimidine-5-carboxylate (136 mg).

LC/MS, t=3.43 min, [MH⁺] 366 and 368.

(b). In a manner similar to Example 266(b) methyl 2-(2,6-dichlorophenylamino)-4-trifluoro-methylpyrimidine-5-carboxylate (135 mg) afforded 2-(2,6-dichlorophenylamino)-4-trifluoromethylpyrimidine-5-carboxylic acid (114 mg).

NMR (400 MHz, DMSO-d6) δ 7.41 (1H, t), 7.60 (2H, d), 8.92 (1H, br s), 10.5 (1H, s), 13.6 (1H, br s).

(c). In a manner similar to Example 266(c) 2-(2,6-dichlorophenylamino)-4-trifluoromethyl-pyrimidine-5-carboxylic acid (40 mg) and N-methylbenzylamine (18 μl) afforded the title compound (49 mg).

NMR (400 MHz, DMSO-d6) Rotamers in 65:35 ratio δ 2.83 (1.95H, s), 2.98 (1.05H, s), 4.51 (0.7H, s), 4.74 (1.3H, br s), 7.26 (1H, d), 7.3-7.5 (5H, m), 7.65 (2H, t), 8.69 (0.35H, br s), 8.78 (0.65H, br s), 10.3 (1H, s). LC/MS, t=3.51 min, [MH⁺] 455 and 457.

### EXAMPLE 276

### 2-(3,5-Dichlorophenylamino)-4-trifluoromethylpyrimidine-5-carboxylic acid benzyl-amide

(a). In a manner similar to Example 272(a) methyl 2-chloro-4-trifluoromethylpyrimidine-5-carboxylate (0.5 g) and 3,5-dichloroaniline (1.7 g) afforded methyl 2-(3,5-dichlorophenyl-amino)-4-trifluoromethyl-pyrimidine-5-carboxylate (0.76 g).

LC/MS, t=3.96 min, [MH⁺] 366 and 368.

(b). In a manner similar to Example 266(b) methyl 2-(3,5-dichlorophenylamino)-4-trifluoro-methylpyrimidine-5-carboxylate (0.76 g) afforded 2-(3,5-dichlorophenylamino)-4-trifluoromethylpyrimidine-5-carboxylic acid (0.65 g).

NMR (400 MHz, DMSO-d6) δ 7.28 (1H, s), 7.90 (2H, s), 9.14 (1H, s), 10.95 (1H, s), 13.75 (1H, br s).

(c). In a manner similar to Example 266(c) 2-(3,5-dichlorophenylamino)-4-trifluoromethyl-pyrimidine-5-carboxylic acid (35 mg) and benzylamine (13 μl) afforded the title compound (29 mg).

NMR (400 MHz, DMSO-d6) δ 4.48 (2H, d), 7.25 (2H, m), 7.38 (4H, m), 7.89 (2H, s), 8.95 (1H, s), 9.16 (1H, t), 10.8 (1H, s). LC/MS, t=3.87 min, [MH⁺] 441 and 443.

### EXAMPLE 277

### 2-(3-Fluorophenylamino)-4-trifluoromethylpyrimidine-5-carboxylic acid benzyl-amide

(a). In a manner similar to Example 266(a) methyl 2-chloro-4-trifluoromethylpyrimidine-5-carboxylate (0.5 g) and 3-fluoroaniline (1.16 g) afforded methyl 2-(3-fluorophenylamino)-4-trifluoromethyl-pyrimidine-5-carboxylate (0.65 g).

NMR (400 MHz, DMSO-d6) δ 3.88 (3H, s), 6.95 (1H, t of d), 7.40 (1H, q), 7.54 (1H, d), 7.79 (1H, d of t), 9.12 (1H, s), 10.95 (1H, s). LC/MS, t=3.50 min, [MH⁺] 316.

(b). In a manner similar to Example 266(b) methyl 2-(3-fluorophenylamino)-4-trifluoro-methylpyrimidine-5-carboxylate (0.65 g) afforded 2-(3-fluorophenylamino)-4-trifluoromethylpyrimidine-5-carboxylic acid (0.54 g).

NMR (400 MHz, DMSO-d6) δ 6.90 (1H, t of d), 7.39 (1H, q), 7.55 (1H, d), 7.80 (1H, d of t), 9.10 (1H, s), 10.85 (1H, s), 13.7 (1H, br s).

(c). In a manner similar to Example 266(c) 2-(3-fluororophenylamino)-4-trifluoromethyl-pyrimidine-5-carboxylic acid (35 mg) and benzylamine (15 μl) afforded the title compound (35 mg).

NMR (400 MHz, DMSO-d6) δ 4.46 (2H, d), 6.87 (1H, t of d), 7.28 (1H, m), 7.35 (5H, m), 7.52 (1H, d), 7.78 (1H, d of t), 8.89 (1H, s), 9.15 (1H, t), 10.65 (1H, s). LC/MS, t=3.47 min, [MH⁺] 391.

### EXAMPLE 278

### 2-(3-Bromophenylamino)-4-trifluoromethylpyrimidine-5-carboxylic acid benzyl-amide

(a). In a manner similar to Example 266(a) methyl 2-chloro-4-trifluoromethylpyrimidine-5-carboxylate (0.5 g) and 3-bromoaniline (1.79 g) afforded methyl 2-(3-bromophenylamino)-4-trifluoromethyl-pyrimidine-5-carboxylate (0.68 g).

NMR (400 MHz, DMSO-d6) δ 3.88 (3H, s), 7.30 (2H, m), 7.72 (1H, d), 8.12 (1H, s), 9.11 (1H, s), 10.90 (1H, s). LC/MS, t=3.70 min, [M] 376 and 378.

(b). In a manner similar to Example 266(b) methyl 2-(3-bromophenylamino)-4-trifluoro-methylpyrimidine-5-carboxylate (0.68 g) afforded 2-(3-bromophenylamino)-4-trifluoromethylpyrimidine-5-carboxylic acid (0.57 g).

NMR (400 MHz, DMSO-d6) δ 7.30 (2H, m), 7.73 (1H, d), 8.15 (1H, s), 9.09 (1H, s), 10.80 (1H, s), 13.65 (1H, br s).

(c). In a manner similar to Example 266(c) 2-(3-bromophenylamino)-4-trifluoromethyl-pyrimidine-5-carboxylic acid (35 mg) and benzylamine (13 μl) afforded the title compound (23 mg).

NMR (400 Hz, DMSO-d6) δ 4.47 (2H, d), 7.2-7.4 (7H, m), 7.71 (1H, d), 8.11 (1H, s), 8.89 (1H, s), 9.15 (1H, t), 10.65 (1H, s).

LC/MS, t=3.64 min, [MH⁺] 451 and 453.

### EXAMPLE 279

### 2-(3-Bromophenylamino)-4-trifluoromethylpyrimidine-5-carboxylic acid N-benzyl-N-methylamide

In a manner similar to Example 266(c) 2-(3-bromophenylamino)-4-trifluoromethyl-pyrimidine-5-carboxylic acid (35 mg) and N-methylbenzylamine (15 μl) afforded the title compound (45 mg).

NMR (400 MHz, DMSO-d6) Rotamers in 65:35 ratio δ 2.89 (1.95H, s), 2.98 (1.05H, s), 4.58 (0.7H, br s), 4.76 (1.3H, br s), 7.28 (1H, d), 7.25-7.5 (6H, m), 7.76 (1H, t), 8.13 (0.35H, t), 8.19 (0.65H, t), 8.88 (0.35H, s), 8.95 (0.65H, s), 10.6 (0.35H, s), 10.65 (0.65H, s).

LC/MS, t=3.72 min, [MH⁺] 465 and 467.

### EXAMPLE 280

### 2-(2-Methoxyphenylamino)-4-trifluoromethylpyrimidine-5-carboxylic acid benzyl-amide

(a). In a manner similar to Example 266(a) benzyl 2-chloro-4-trifluoromethylpyrimidine-5-carboxylate (0.5 g) and 2-methoxyaniline (0.97 g) afforded benzyl 2-(2-methoxyphenyl-amino)-4-trifluoromethyl-pyrimidine-5-carboxylate (0.57 g).

NMR (400 MHz, CDCl₃) δ 3.93 (3H, s), 5.38 (2H, s), 6.93 (1H, d), 7.04 (1H, t), 7.09 (1H, t), 7.35-7.45 (4H, m), 8.26 (1H, br s), 8.49 (1H, br d), 9.06 (1H, s). LC/MS, t=3.42 min, [MH⁺] 404.

(b). In a manner similar to Example 266(b) benzyl 2-(2-methoxyphenylamino)-4-trifluoro-methylpyrimidine-5-carboxylate (0.55 g) afforded 2-(2-methoxyphenylamino)-4-trifluoro-methylpyrimidine-5-carboxylic acid (0.38 g).

NMR (400 MHz, DMSO-d6) δ 3.80 (3H, s), 6.98 (1H, t), 7.10 (1H, d), 7.22 (1H, t), 7.62 (1H, d), 8.94 (1H, s), 9.62 (1H, s), 13.5 (1H, s). LC/MS, t=3.03 min, [MH⁺] 314.

(c). In a manner similar to Example 266(c) 2-(2-methoxyphenylamino)-4-trifluoromethyl-pyrimidine-5-carboxylic acid (35 mg) and benzylamine (18 μl) afforded, after silica gel chromatography using 1:1 ethyl acetate:isohexane, the title compound (38 mg).

NMR (400 MHz, CDCl₃) δ 3.93 (3H, s), 4.65 (2H, d), 6.09 (1H, br s), 6.90 (1H, d), 7.05 (2H, m), 7.35 (5H, m), 8.25 (1H, s), 8.47 (1H, d), 8.75 (1H, s). LC/MS, t=3.14 min, [MH⁺] 1403.

### EXAMPLE 281

### 2-(2,3-Dichlorophenylamino)-4-trifluoromethylpyrimidine-5-carboxylic acid (pyridin-4-ylmethyl)amide

In a manner similar to Example 266(c) 2-(2,3-dichlorophenylamino)-4-trifluoromethyl-pyrimidine-5-carboxylic acid (24 mg) and 4-(aminomethyl)pyridine (10 μl) afforded the title compound (19 mg).

NMR (400 MHz, DMSO-d6) δ 4.48 (2H, d), 7.34 (2H, d), 7.41 (1H, t), 7.55 (2H, m), 8.52 (2H, d), 8.81 (1H, s), 9.23 (1H, t), 10.20 (1H, s). LC/MS, t=2.95 min, [MH⁺] 442 and 444.

### EXAMPLE 282

### 2-(2,4-Dichlorophenylamino)-4-trifluoromethylpyrimidine-5-carboxylic acid (pyridin-4-ylmethyl)amide

In a manner similar to Example 266(c) 2-(2,4-dichlorophenylamino)-4-trifluoromethyl-pyrimidine-155-carboxylic acid (30 mg) and 4-(aminomethyl)pyridine (10.5 μl) afforded the title compound (24 mg).

NMR (400 MHz, DMSO-d6) δ 4.48 (2H, d), 7.33 (2H, d), 7.48 (1H, d of d), 7.60 (1H, d), 7.75 (1H, s), 8.52 (2H, d), 8.80 (1H, s), 9.22 (1H, t), 10.10 (1H, s). LC/MS, t=3.00 min, [MH⁺] 442 and 444.

### EXAMPLE 283

### 2-(3,4-Dichlorophenylamino)-4-trifluoromethylpyrimidine-5-carboxylic acid (pyridin-4-ylmethyl)amide

In a manner similar to Example 266(c) 2-(3,4-dichlorophenylamino)-4-trifluoromethyl-pyrimidine-5-carboxylic acid (30 mg) and 4-(aminomethyl)pyridine (10.5 μl) afforded the title compound (32 mg).

NMR (400 MH, DMSO-d6) δ 4.50 (2H, d), 7.36 (2H, d), 7.62 (1H, d), 7.70 (1H, d of d), 8.18 (1H, s), 8.55 (2H, d), 8.99 (1H, s), 9.27 (1H, t), 10.75 (1H, s). LC/MS, t=3.17 min, [MH⁺] 442 and 444.

### EXAMPLE 284

### 2-(2,5-Dichlorophenylamino)-4-trifluoromethylpyrimidine-5-carboxylic acid (pyridin-4-ylmethyl)amide

(a). In a manner similar to Example 273(a) methyl 2-chloro-4-trifluoromethylpyrimidine-5-carboxylate (0.5 g) and 2,5-dichloroaniline (1.7 g) afforded methyl 2-(2,5-dichlorophenyl-amino)-4-trifluoromethylpyrimidine-5-carboxylate (681 mg).

LC/MS, t=3.73 min, [MH⁺] 366 and 368.

(b). In a manner similar to Example 266(b) methyl 2-(2,5-dichlorophenylamino)-4-trifluoro-methylpyrimidine-5-carboxylate (0.68 g) afforded 2-(2,5-dichlorophenylamino)-4-trifluoro-methylpyrimidine-5-carboxylic acid (0.48 g).

NMR (400 MHz, DMSO-d6) δ 7.36 (1H, d of d), 7.60 (1H, d), 7.76 (1H, d), 8.99 (1H, s), 10.3 (1H, s), 13.6 (1H, br s).

(c) In a manner similar to Example 266(c) 2-(2,5-dichlorophenylamino)-4-trifluoromethyl-pyrimidine-5-carboxylic acid (35 mg) and 4-(aminomethyl)pyridine (17 μl) afforded the title compound (35 mg).

NMR (400 MHz, DMSO-d6) δ 4.47 (2H, d), 7.33 (3H, m), 7.60 (1H, d), 7.73 (1H, d), 8.54 (2H, d), 8.84 (1H, s), 9.21 (1H, t), 10.10 (1H, s). LC/MS, t=2.96 min, [MH⁺] 442 and 444.

### EXAMPLE 285

### 2-(3-Fluorophenylamino)-4-trifluoromethylpyrimidine-5-carboxylic acid (pyridin-4-ylmethyl)amide

In a manner similar to Example 266(c) 2-(3-fluorophenylamino)-4-trifluoromethylpyrimidine-5-carboxylic acid (35 mg) and 4-(aminomethyl)pyridine (12 μl) afforded the title compound (28 mg).

NMR (400 MHz, DMSO-d6) δ 4.51 (2H, d), 6.88 (1H, t of d), 7.4 (3H, m), 7.55 (1H, d), 7.80 (1H, d of t), 8.56 (2H, d), 8.96 (1H, s), 9.26 (1H, t), 10.70 (1H, s). LC/MS, t=2.65 min, [MH⁺] 392.

### EXAMPLE 286

### 2-(3-Bromophenylamino)-4-trifluoromethylpyrimidine-5-carboxylic acid (pyridin-4-ylmethyl)amide

In a manner similar to Example 266(c) 2-(3-bromophenylamino)-4-trifluoromethylpyrimidine-5-carboxylic acid (35 mg) and 4-(aminomethyl)pyridine (12 μl) afforded the title compound (29 mg).

NMR (400 MHz, DMSO-d6) δ 4.48 (2H, d), 7.24 (1H, d), 7.34 (1H, t), 7.38 (2H, d), 7.73 (1H, d), 8.13 (1H, s), 8.56 (2H, d), 8.98 (1H, s), 9.26 (1H, t), 10.65 (1H, s).

LC/MS, t=2.91 min, [MH⁺] 452 and 454.

### EXAMPLE 287

### 2-(3,5-Dichlorophenylamino)-4-trifluoromethylprimidine-5-carboxylic acid (pyridin-4-ylmethyl)amide

In a manner similar to Example 266(c) 2-(3,5-dichlorophenylamino)-4-trifluoromethyl-pyrimidine-5-carboxylic acid (35 mg) and 4-(aminomethyl)pyridine (17 μl) afforded the title compound (39 mg).

NMR (400 MHz, DMSO-d6) δ 4.48 (2H, d), 7.26 (1H, s), 7.36 (2H, d), 7.89 (2H, s), 8.55 (2H, d), 9.03 (1H, s), 9.29 (1H, t), 10.85 (1H, s). LC/MS, t=2.96 min, [MH⁺] 442 and 444.

### EXAMPLE 288

### 2-(3-Fluorophenylamino)-4-trifluoromethylpyrimidine-5-carboxylic acid 4-fluorobenzyl-amide

In a manner similar to Example 266(c) 2-(3-fluorophenylamino)-4-trifluoromethyl-pyrimidine-5-carboxylic acid (35 mg) and 4-fluorobenzylamine hydrochloride (22.5 mg) afforded the title compound (31 mg).

NMR (400 MHz, DMSO-d6) δ 4.46 (2H, d), 6.88 (1H, t), 7.19 (2H, t), 7.35-7.45 (3H, m), 7.53 (1H, d), 7.78 (1H, d of t), 8.89 (1H, s), 9.15 (1H, t), 10.65 (1H, s). LC/MS, t=3.49 min, [MH⁺] 409.

### EXAMPLE 289

### 2-(3-Bromophenylamino)-4-trifluoromethylpyrimidine-5-carboxylic acid 4-fluorobenzyl-amide

In a manner similar to Example 266(c) 2-(3-bromophenylamino)-4-trifluoromethyl-pyrimidine-5-carboxylic acid (35 mg) and 4-fluorobenzylamine hydrochloride (19 mg) afforded the title compound (31 mg).

NMR (400 MHz, DMSO-d6) δ 4.44 (2H, d), 7.15-7.25 (3H, m), 7.31 (1H, t), 7.4 (2H, m), 7.71 (1H, d), 8.10 (1H, s), 8.88 (1H, s), 9.14 (1H, t), 10.60 (1H, s). LC/MS, t=3.65 min, [MH⁺] 469 and 471.

### EXAMPLE 290

### 2-(3,5-Dichlorophenylamino)-4-trifluoromethylpyrimidine-5-carboxylic acid 4-fluorobenzyl-amide

In a manner similar to Example 266(c) 2-(3,5-dichlorophenylamino)-4-trifluoromethyl-pyrimidine-5-carboxylic acid (33 mg) and 4-fluorobenzylamine hydrochloride (17 mg) afforded the title compound (33 mg).

NMR (400 MHz, DMSO-d6) δ 4.44 (2H, d), 7.19 (2H, t), 7.26 (1H, s), 7.40 (2H, t), 7.88 (2H, s), 8.94 (1H, s), 9.16 (1H, t), 10.80 (1H, s). LC/MS, t=3.87 min, [MH⁺] 459 and 457.

### EXAMPLE 291

### 2-(3,5-Dichlorophenylamino)-4-trifluoromethylpyrimidine-5-carboxylic acid 4-cyanobenzylamide

In a manner similar to Example 266(c) 2-(3,5-dichlorophenylamino)-4-trifluoromethyl-pyrimidine-5-carboxylic acid (35 mg) and 4-cyanobenzylamine (20 mg) afforded the title compound (33 mg).

NMR (400 MHz, DMSO-d6) δ 4.55 (2H, d), 7.26 (1H, s), 7.56 (2H, d), 7.84 (2H, d), 7.88 (2H, s), 8.99 (1H, s), 9.27 (1H, t), 10.80 (1H, s). LC/MS, t=3.74 min, [MH⁺] 466 and 468.

### EXAMPLE 292

### 2-(4-Cyano-phenylamino)-4-trifluoromethyl-pyrimidine-5-carboxylic acid benzylamide

(a). In a manner similar to Example 266(a) benzyl 2-chloro-4-trifluoromethylpyrimidine-5-carboxylate (0.5 g) and 4-aminobenzonitrile (0.93 g) afforded, after silica gel chromatography using 3:2 isohexane:ethyl acetate, benzyl 2-(4-cyanophenyl-amino)-4-trifluoromethylpyrimidine-5-carboxylate (323 mg).

NMR (400 MHz, CDCl₃) δ 5.39 (2H, s), 7.35-7.5 (5H, m), 7.68 (2H, d), 7.76 (1H, s), 7.84 (2H, d), 9.10 (1H, s). LC/MS CF100603-1, t=3.39 min, [MH⁺] 399.

(b). To a solution of benzyl 2-(4-cyanophenylamino)-4-trifluoromethyl-pyrimidine-5-carboxylate (323 mg) in dimethylformamide (6 ml) was added 10% palladium on charcoal (wet) and the mixture stirred under atmospheric hydrogenation conditions for 4 h. Catalyst was filtered through a 1 μM PTFE filter and filtrate evaporated under reduced pressure. The residual solid was triturated with ether, filtered and dried in vacuo at 50° C. to afford 2-(4-cyanophenylamino)-4-trifluoromethylpyrimidine-5-carboxylic acid (284 mg).

NMR (400 MHz, DMSO-d6) δ 7.84 (2H, d), 7.99 (2H, d), 9.13 (1H, s), 11.1 (1H, s), 13.8 (1H, br s).

LC/MS CF100887-1, t=2.78 min, [MH⁺] 309.

(c) In a manner similar to Example 266(c), 2-(4-cyanophenylamino)-4-trifluoromethylpyrimidine-5-carboxylic acid and benzylamine afforded the title compound LC/MS, t=3.02 min, [MH⁺] 398.

### EXAMPLE 293

### 2-(3-Chloro-phenylamino)-4-trifluoromethyl-pyrimidine-5-carboxylic acid (2-methyl-pyridin-4-ylmethyl)-amide hydrochloride

To a solution of 2-(3-chloro-phenylamino)-4-trifluoromethyl-pyrimidine-5-carboxylic acid (2-methyl-pyridin-4-ylmethyl)-amide (15 mg) in ethanol (2 ml) was added a few drops of concentrated hydrochloric acid. The solution was stirred at room temperature for 0.5 h and then evaporated under reduced pressure. Trituration with ether precipitated a white solid which was filtered off, washed with fresh ether and dried to afford the title compound (14 mg).

NMR (400 MHz, DMSO-d6) δ 2.72 (3H, s), 4.67 (2H, d), 7.12 (1H, d), 7.38 (1H, t), 7.67 (1H, d), 7.75 (1H, d), 7.79 (1H, s), 8.00 (1H, s), 8.71 (1H, d), 9.06 (1H, s), 9.49 (1H, t), 10.70 (1H, s)

LC/MS, t 2.62 min, [MH⁺] 422 and 424.

### EXAMPLE 294

### 2-(3-Chloro-phenylamino)-4-trifluoromethyl-pyrimidine-5-carboxylic acid (2-fluoro-pyridin-4-ylmethyl)-amide

### (a). 4-Bromomethyl-2-fluoro-pyridine

To a solution of 2-fluoro-4-methylpyridine (1.0 g, ex Lancaster) in carbon tetrachloride (10 ml) was added N-bromosuccinimide (1.6 g, ex Lancaster) and 1,1′-azobis (cyclohexanecarbonitrile) (100 mg, ex Aldrich). The mixture was then refluxed for 24 h. Carbon tetrachloride was removed under reduced pressure and the crude oily solid was used in the next stage without purification.

LC/MS, t=2.38 min, [MH⁺] 190 and 192.

### (b). (2-Fluoro-pyridin-4-ylmethyl)-carbamic acid tert-butyl ester

To crude 4-bromomethyl-2-fluoro-pyridine in an ice bath was added 25% ammonia solution (10 ml, ex BDH) and the mixture stirred at 0° for 5 h. Ammonia solution was removed under reduced pressure and the yellow oily solid residue dissolved in dichloromethane (10 ml) and dimethylformamide (1 ml). The solution was cooled in an ice bath and triethylamine (1.5 ml, ex BDH) was added followed by di-tert-butyl dicarbonate (1.0 g, ex Avocado). The solution was stirred at 0 for 1 h and then the dichloromethane removed under reduced pressure. The residue was dissolved in ethyl acetate and washed twice with water, dried (MgSO₄) and evaporated to give a yellow oil. This was purified by Biotage chromatography (100 g, silica column) eluting with 30% ethyl acetate in hexane to afford the title compound as a white solid (358 mg).

NMR (400 MHz, DMSO-d6) δ 1.40 (9H, s), 4.20 (2H, d), 6.97 (1H, s), 7.20 (1H, d), 7.60 (1H, t), 8.17 (1H, d) LC/MS, t=2.60 min, [M-Me2C═CH2+H]⁺ 171

### (c). C-(2-Fluoro-pyridin-4-yl)-methylamine dihydrochloride

(2-Fluoro-pyridin-4-ylmethyl)-carbamic acid tert-butyl ester (350 mg) was treated at room temperature with 4N hydrochloric acid in 1,4-dioxan (5 ml) and stirred for 2 h. The white precipitate was filtered, washed with fresh ether and dried to afford the title compound (200 mg).

NMR (400 MHz, DMSO-d6) δ 4.14 (2H, d), 7.38 (1H, s), 7.51 (1H, d), 8.28 (1H, d), 8.82 (3H, s).

### (d). 2-(3-Chloro-phenylamino)-4-trifluoromethyl-pyrimidine-5-carboxylic acid (2-fluoro-pyridin-4-ylmethyl)-amide

In a manner similar to Example 266(c) 2-(3-chloro-phenylamino)-4-trifluoromethyl-pyrimidine-5-carboxylic acid (75 mg) and C-(2-fluoro-pyridin-4-yl)-methylamine dihydrochloride (56 mg) afforded the title compound (85 mg).

NMR (400 MHz, DMSO-d6) δ 4.55 (2H, d), 7.10 (2H, m), 7.38 (2H, m), 7.66 (1H, m), 7.98 (1H, m), 8.21 (1H, d), 8.99 (1H, s), 9.29 (1H, t), 10.65 (1H, s) LC/MS, t=3.33 min, [MH^{+x]} 426 and 428.

Examples 295-343 were prepared in a manner similar to that in Example 266.

TABLE 7

| | | LCMS data |
|---|---|---|
| | | 1) Retention time |
| Ex. | | 2) MH+ |
| No. | Compound Name | 3) Formula consistent with MH+ |
| 295 | 2-(3-Methoxy-phenylamino)-4-trifluoromethyl- | 3.08 min |
| | pyrimidine-5-carboxylic acid benzylamide | 403 |
| | | C₂₀H₁₇F₃N₄O₂ |
| 296 | 2-(2-Methoxy-phenylamino)-4-trifluoromethyl- | 3.25 min |
| | pyrimidine-5-carboxylic acid N-benzyl-N-methyl- | 417 |
| | amide | C₂₁H₁₉F₃N₄O₂ |
| 297 | 2-(3-Methoxy-phenylamino)-4-trifluoromethyl- | 3.19 min |
| | pyrimidine-5-carboxylic acid N-benzyl-N-methyl- | 417 |
| | amide | C₂₁H₁₉F₃N₄O₂ |
| 298 | 2-(3-Chloro-phenylamino)-4-trifluoromethyl- | 3.75 min |
| | pyrimidine-5-carboxylic acid 4-chloro-benzylamide | 441 |
| | | C₁₉H₁₃³⁵Cl₂F₃N₄O |
| 299 | 2-(3-Chloro-phenylamino)-4-trifluoromethyl- | 3.68 min |
| | pyrimidine-5-carboxylic acid N-benzyl-N-ethyl- | 435 |
| | amide | C₂₁H₁₈³⁵ClF₃N₄O |
| 300 | 2-(2,3-Dichloro-phenylamino)-4-trifluoromethyl- | 3.63 min |
| | pyrimidine-5-carboxylic acid benzylamide | 441 |
| | | C₁₉H₁₃³⁵Cl₂F₃N₄O |
| 301 | 2-(3-Fluoro-phenylamino)-4-trifluoromethyl- | 3.55 min |
| | pyrimidine-5-carboxylic acid N-benzyl-N-methyl- | 405 |
| | amide | C₂₀H₁₆F₄N₄O |
| 302 | 2-(3-Chloro-phenylamino)-4-trifluoromethyl- | 4.07 min |
| | pyrimidine-5-carboxylic acid 4-isobutyl-benzylamide | 463 |
| | | C₂₃H₂₂³⁵ClF₃N₄O |
| 303 | 2-(3-Chloro-phenylamino)-4-trifluoromethyl- | 2.65 min |
| | pyrimidine-5-carboxylic acid (2-methyl-pyridin-4- | 422 |
| | ylmethyl)-amide | C₁₉H₁₅³⁵ClF₃N₅O |
| 304 | 2-(3-Bromo-phenylamino)-4-trifluoromethyl- | 2.68 min |
| | pyrimidine-5-carboxylic acid (2-methyl-pyridin-4- | 466 |
| | ylmethyl)-amide | C₁₉H₁₅⁷⁹BrF₃N₅O |
| 305 | 2-(2-Chloro-phenylamino)-4-trifluoromethyl- | 3.24 min |
| | pyrimidine-5-carboxylic acid N-benzyl-N-methyl- | 421 |
| | amide | C₂₀H₁₆³⁵ClF₃N₄O |
| 306 | 2-(3-Fluoro-phenylamino)-4-trifluoromethyl- | 3.37 min |
| | pyrimidine-5-carboxylic acid N-(4-cyano- | 416 |
| | benzyl)amide | C₂₀H₁₃F₄N₅O |
| 307 | 2-(2,4-Dichloro-phenylamino)-4-trifluoromethyl- | 3.00 min |
| | pyrimidine-5-carboxylic acid N-(pyrimidin-4- | 442 |
| | ylmethyl)-amide | C₁₈H₁₂³⁵Cl₂F₃N₅O |
| 308 | 2-(2-Chloro-phenylamino)-4-trifluoromethyl- | 3.11 min |
| | pyrimidine-5-carboxylic acid N-benzylamide | 407 |
| | | C₁₉H₁₄³⁵ClF₃N₄O |
| 309 | 2-(4-Chloro-phenylamino)-4-trifluoromethyl- | 3.25 min |
| | pyrimidine-5-carboxylic acid N-benzylamide | 407 |
| | | C₁₉H₁₄³⁵ClF₃N₄O |
| 310 | 2-(4-Chloro-phenylamino)-4-trifluoromethyl- | 3.35 min |
| | pyrimidine-5-carboxylic acid N-benzyl-N-methyl- | 421 |
| | amide | C₂₀H₁₆³⁵ClF₃N₄O |
| 311 | 2-(4-Methoxy-phenylamino)-4-trifluoromethyl- | 3.02 min |
| | pyrimidine-5-carboxylic acid N-benzylamide | 402 |
| | | C₂₀H₁₇F₃N₄O₂ |
| 312 | 2-(4-Methoxy-phenylamino)-4-trifluoromethyl- | 3.13 min |
| | pyrimidine-5-carboxylic acid N-benzyl-N-methyl- | 416 |
| | amide | C₂₁H₁₉F₃N₄O₂ |
| 313 | 2-(3-Cyano-phenylamino)-4-trifluoromethyl- | 3.13 min |
| | pyrimidine-5-carboxylic acid N-benzyl-N-methyl- | 412 |
| | amide | C₂₁H₁₆F₃N₅O |
| 314 | 2-(4-Cyano-phenylamino)-4-trifluoromethyl- | 3.12 min |
| | pyrimidine-5-carboxylic acid N-benzyl-N-methyl- | 412 |
| | amide | C₂₁H₁₆F₃N₅O |
| 315 | 2-(3-Chloro-phenylamino)-4-trifluoromethyl- | 3.58 min |
| | pyrimidine-5-carboxylic acid N-(3- | 437 |
| | methoxybenzyl)amide | C₂₀H₁₆³⁵ClF₃N₄O₂ |
| 316 | 2-(3-Chloro-phenylamino)-4-trifluoromethyl- | 3.72 min |
| | pyrimidine-5-carboxylic acid N-(2- | 441 |
| | chlorobenzyl)amide | C₁₉H₁₃³⁵Cl₂F₃N₄O |
| 317 | 2-(3-Chloro-phenylamino)-4-trifluoromethyl- | 3.75 min |
| | pyrimidine-5-carboxylic acid N-(3- | 441 |
| | chlorobenzyl)amide | C₁₉H₁₃³⁵Cl₂F₃N₄O |
| 318 | 2-(2-Chloro-phenylamino)-4-trifluoromethyl- | 3.68 min |
| | pyrimidine-5-carboxylic acid N-benzyl-N-ethyl- | 435 |
| | amide | C₂₁H₁₈³⁵ClF₃N₄O |
| 319 | 2-(2,4-Dichloro-phenylamino)-4-trifluoromethyl- | 3.79 min |
| | pyrimidine-5-carboxylic acid N-benzyl-N-methyl- | 455 |
| | amide | C₂₀H₁₅³⁵Cl₂F₃N₄O |
| 320 | 2-(3,4-Dichloro-phenylamino)-4-trifluoromethyl- | 3.87 min |
| | pyrimidine-5-carboxylic acid N-benzyl-N-methyl- | 455 |
| | amide | C₂₀H₁₅³⁵Cl₂F₃N₄O |
| 321 | 2-(2,5-Dichloro-phenylamino)-4-trifluoromethyl- | 3.68 min |
| | pyrimidine-5-carboxylic acid N-benzylamide | 441 |
| | | C₁₉H₁₃³⁵Cl₂F₃N₄O |
| 322 | 2-(3,5-Dichloro-phenylamino)-4-trifluoromethyl- | 3.93 min |
| | pyrimidine-5-carboxylic acid N-benzyl-N-methyl- | 455 |
| | amide | C₂₀H₁₅³⁵Cl₂F₃N₄O |
| 323 | 2-(2,5-Dichloro-phenylamino)-4-trifluoromethyl- | 3.78 min |
| | pyrimidine-5-carboxylic acid N-benzyl-N-methyl- | 455 |
| | amide | C₂₀H₁₅³⁵Cl₂F₃N₄O |
| 324 | 2-(3-Chloro-phenylamino)-4-trifluoromethyl- | 3.28 min |
| | pyrimidine-5-carboxylic acid N-(pyridin-2- | 408 |
| | ylmethyl)-amide | C₁₈H₁₃³⁵ClF₃N₅O |
| 325 | 2-(3-Chloro-phenylamino)-4-trifluoromethyl- | 3.07 min |
| | pyrimidine-5-carboxylic acid N-(pyridin-3- | 408 |
| | ylmethyl)-amide | C₁₈H₁₃³⁵ClF₃N₅O |
| 326 | 2-(3-Chloro-phenylamino)-4-trifluoromethyl- | 3.73 min |
| | pyrimidine-5-carboxylic acid N-(3,5-difluoro- | 443 |
| | benzyl)amide | C₁₉H₁₂³⁵ClF₅N₄O |
| 327 | 2-(3-Chloro-phenylamino)-4-trifluoromethyl- | 3.87 min |
| | pyrimidine-5-carboxylic acid N-(4-trifluoromethoxy- | 491 |
| | benzyl)amide | C₂₀H₁₃³⁵ClF₆N₄O₂ |
| 328 | 2-(3-Chloro-phenylamino)-4-trifluoromethyl- | 3.84 min |
| | pyrimidine-5-carboxylic acid N-(4-bromo- | 485 |
| | benzyl)amide | C₁₉H₁₃⁷⁹Br³⁵ClF₃N₄O |
| 329 | 2-(3-Bromo-phenylamino)-4-trifluoromethyl- | 3.54 min |
| | pyrimidine-5-carboxylic acid N-(4-cyano- | 476 |
| | benzyl)amide | C₂₀H₁₃⁷⁹BrF₃N₅O |
| 330 | 2-(2,3-Dichloro-phenylamino)-4-trifluoromethyl- | 3.54 min |
| | pyrimidine-5-carboxylic acid N-(4-cyano- | 466 |
| | benzyl)amide | C₂₀H₁₂³⁵Cl₂F₃N₅O |
| 331 | 2-(2,4-Dichloro-phenylamino)-4-trifluoromethyl- | 3.58 min |
| | pyrimidine-5-carboxylic acid N-(4-cyano- | 466 |
| | benzyl)amide | C₂₀H₁₂³⁵Cl₂F₃N₅O |
| 332 | 2-(2,5-Dichloro-phenylamino)-4-trifluoromethyl- | 3.57 min |
| | pyrimidine-5-carboxylic acid N-(4-cyano- | 466 |
| | benzyl)amide | C₂₀H₁₂³⁵Cl₂F₃N₅O |
| 333 | 2-(3,4-Dichloro-phenylamino)-4-trifluoromethyl- | 3.68 min |
| | pyrimidine-5-carboxylic acid N-(4-cyano- | 466 |
| | benzyl)amide | C₂₀H₁₂³⁵Cl₂F₃N₅O |
| 334 | 2-(2,6-Dichloro-phenylamino)-4-trifluoromethyl- | 2.54 min |
| | pyrimidine-5-carboxylic acid N-(pyridin-4- | 442 |
| | ylmethyl)-amide | C₁₈H₁₂³⁵Cl₂F₃N₅O |
| 335 | 2-(2,6-Dichloro-phenylamino)-4-trifluoromethyl- | 3.44 min |
| | pyrimidine-5-carboxylic acid N-(4-fluoro- | 459 |
| | benzyl)amide | C₁₉H₁₂³⁵Cl₂F₄N₄O |
| 336 | 2-(2,6-Dichloro-phenylamino)-4-trifluoromethyl- | 3.32 min |
| | pyrimidine-5-carboxylic acid N-(4-cyano- | 466 |
| | benzyl)amide | C₂₀H₁₂³⁵Cl₂F₃N₅O |
| 337 | 2-(3-Chloro-phenylamino)-4-trifluoromethyl- | 3.75 min |
| | pyrimidine-5-carboxylic acid N-(2-chloro-4-fluoro- | 459 |
| | benzyl)amide | C₁₉H₁₂³⁵Cl₂F₄N₄O |
| 338 | 2-(3-Chloro-phenylamino)-4-trifluoromethyl- | 3.76 min |
| | pyrimidine-5-carboxylic acid (3-chloro-4-fluoro- | 459 |
| | benzyl)amide | C₁₉H₁₂³⁵Cl₂F₄N₄O |
| 339 | 2-(3-Chloro-phenylamino)-4-trifluoromethyl- | 3.80 min |
| | pyrimidine-5-carboxylic acid N-(4-fluoro-2- | 493 |
| | trifluoromethyl-benzyl)amide | C₂₀H₁₂³⁵ClF₇N₄O |
| 340 | 2-(3-Chloro-phenylamino)-4-trifluoromethyl- | 3.79 min |
| | pyrimidine-5-carboxylic acid N-(4-fluoro-3- | 493 |
| | trifluoromethyl-benzyl)amide | C₂₀H₁₂³⁵ClF₇N₄O |
| 341 | 2-(3-Fluoro-phenylamino)-4-trifluoromethyl- | 2.89 min |
| | pyrimidine-5-carboxylic acid N-(pyrimidin-4- | 393 |
| | ylmethyl)-amide | C₁₇H₁₂F₄N₆O |
| 342 | 2-(3-Fluoro-phenylamino)-4-trifluoromethyl- | 2.48 min |
| | pyrimidine-5-carboxylic acid N-(2-methyl-pyridin-4- | 406 |
| | ylmethyl)-amide | C₁₉H₁₅F₄N₅O |
| 343 | 2-(3-Chloro-phenylamino)-4-trifluoromethyl- | 3.75 min |
| | pyrimidine-5-carboxylic acid N-(4-methyl- | 421 |
| | benzyl)amide | C₂₀H₁₆³⁵ClF₃N₄O |

Examples 344 to 379 were prepared in a manner analogous to Example 266.

TABLE 8

| | | LC/MS |
|---|---|---|
| | | 1)Retention time (min) |
| Ex. | | 2)MH⁺ |
| No. | Compound Name | 3)Formula |
| 344 | 2-(3-Fluoro-phenylamino)-4-trifluoromethyl- | 3.49 |
| | pyrimidine-5-carboxylic acid 4-fluoro-benzylamide | 409 |
| | | C₁₉H₁₃F₅N₄O |
| 345 | 2-(3-Bromo-phenylamino)-4-trifluoromethyl- | 3.65 |
| | pyrimidine-5-carboxylic acid 4-fluoro-benzylamide | 471 |
| | | C₁₉H₁₃⁸¹BrF₄N₄O |
| 346 | 2-(3-Chloro-phenylamino)-4-trifluoromethyl- | 3.66 |
| | pyrimidine-5-carboxylic acid 3,4-difluoro-benzylamide | 443 |
| | | C₁₉H₁₂³⁵ClF₅N₄O |
| 347 | 2-(3-Chloro-4-fluoro-phenylamino)-4- | 3.61 |
| | trifluoromethyl-pyrimidine-5-carboxylic acid 4- | 443 |
| | fluoro-benzylamide | C₁₉H₁₂³⁵ClF₅N₄O |
| 348 | 2-(3-Chloro-2-fluoro-phenylamino)-4- | 3.51 |
| | trifluoromethyl-pyrimidine-5-carboxylic acid 4- | 443 |
| | fluoro-benzylamide | C₁₉H₁₂³⁵ClF₅N₄O |
| 349 | 2-(5-Chloro-2-fluoro-phenylamino)-4- | 3.54 |
| | trifluoromethyl-pyrimidine-5-carboxylic acid 4- | 443 |
| | fluoro-benzylamide | C₁₉H₁₂³⁵ClF₅N₄O |
| 350 | 2-(3,5-Difluoro-phenylamino)-4-trifluoromethyl- | 3.55 |
| | pyrimidine-5-carboxylic acid 4-fluoro-benzylamide | 427 |
| | | C₁₉H₁₂F₆N₄O |
| 351 | 2-(3-Chloro-4-cyano-phenylamino)-4- | 3.52 |
| | trifluoromethyl-pyrimidine-5-carboxylic acid 4- | 450 |
| | fluoro-benzylamide | C₂₀H₁₂³⁵ClF₄N₅O |
| 352 | 2-(3-Methoxy-phenylamino)-4-trifluoromethyl- | 2.53 |
| | pyrimidine-5-carboxylic acid (pyridin-4- | 404 |
| | ylmethyl)-amide | C₁₉H₁₆F₃N₅O₂ |
| 353 | 2-(3-Bromo-phenylamino)-4-trifluoromethyl- | 3.37 |
| | pyrimidine-5-carboxylic acid (2-fluoro-pyridin- | 472 |
| | 4-ylmethyl)-amide | C₁₈H₁₂⁸¹BrF₄N₅O |
| 354 | 2-(3-Fluoro-phenylamino)-4-trifluoromethyl- | 3.18 |
| | pyrimidine-5-carboxylic acid (2-fluoro-pyridin- | 410 |
| | 4-ylmethyl)-amide | C₁₈H₁₂F₅N₅O |
| 355 | 2-(2,5-Dichloro-phenylamino)-4- | 3.41 |
| | trifluoromethyl-pyrimidine-5-carboxylic acid (2- | 460 |
| | fluoro-pyridin-4-ylmethyl)-amide | C₁₈H₁₁³⁵Cl₂F₄N₅O |
| 356 | 2-(3,5-Dichloro-phenylamino)-4- | 3.61 |
| | trifluoromethyl-pyrimidine-5-carboxylic acid (2- | 460 |
| | fluoro-pyridin-4-ylmethyl)-amide | C₁₈H₁₁³⁵Cl₂F₄N₅O |
| 357 | 2-(3,4-Dichloro-phenylamino)-4- | 3.54 |
| | trifluoromethyl-pyrimidine-5-carboxylic acid (2- | 460 |
| | fluoro-pyridin-4-ylmethyl)-amide | C₁₈H₁₁³⁵Cl₂F₄N₅O |
| 358 | 2-(2,6-Dichloro-phenylamino)-4- | 3.13 |
| | trifluoromethyl-pyrimidine-5-carboxylic acid (2- | 460 |
| | fluoro-pyridin-4-ylmethyl)-amide | C₁₈H₁₁³⁵Cl₂F₄N₅O |
| 359 | 2-(2,3-Dichloro-phenylamino)-4- | 3.38 |
| | trifluoromethyl-pyrimidine-5-carboxylic acid (2- | 460 |
| | fluoro-pyridin-4-ylmethyl)-amide | C₁₈H₁₁³⁵Cl₂F₄N₅O |
| 360 | 2-(2,4-Dichloro-phenylamino)-4- | 3.48 |
| | trifluoromethyl-pyrimidine-5-carboxylic acid (2- | 460 |
| | fluoro-pyridin-4-ylmethyl)-amide | C₁₈H₁₁³⁵Cl₂F₄N₅O |
| 361 | 2-(2-Chloro-phenylamino)-4-trifluoromethyl- | 3.24 |
| | pyrimidine-5-carboxylic acid benzyl-methyl- | 421 |
| | amide | C₂₀H₁₆³⁵ClF₃N₄O |
| 362 | 2-(3-Cyano-phenylamino)-4-trifluoromethyl- | 3.02 398 |
| | pyrimidine-5-carboxylic acid benzylamide | C₂₀H₁₄F₃N₅O |
| 363 | 2-(2,6-Dichloro-phenylamino)-4- | 3.42 |
| | trifluoromethyl-pyrimidine-5-carboxylic acid benzylamide | 441 |
| | | C₁₉H₁₃³⁵Cl₂F₃N₄O |
| 364 | 2-(2,3-Dichloro-phenylamino)-4- | 3.66 |
| | trifluoromethyl-pyrimidine-5-carboxylic acid 4- | 459 |
| | fluoro-benzylamide | C₁₉H₁₂³⁵Cl₂F₄N₄O |
| 365 | 2-(2,4-Dichloro-phenylamino)-4- | 3.71 |
| | trifluoromethyl-pyrimidine-5-carboxylic acid 4- | 459 |
| | fluoro-benzylamide | C₁₉H₁₂³⁵Cl₂F₄N₄O |
| 366 | 2-(2,5-Dichloro-phenylamino)-4- | 3.70 |
| | trifluoromethyl-pyrimidine-5-carboxylic acid 4- | 459 |
| | fluoro-benzylamide | C₁₉H₁₂³⁵Cl₂F₄N₄O |
| 367 | 2-(3,4-Dichloro-phenylamino)-4- | 3.80 |
| | trifluoromethyl-pyrimidine-5-carboxylic acid 4- | 459 |
| | fluoro-benzylamide | C₁₉H₁₂³⁵Cl₂F₄N₄O |
| 368 | 2-(3-Chloro-phenylamino)-4-trifluoromethyl- | 3.63 |
| | pyrimidine-5-carboxylic acid 2,4-difluoro-benzylamide | 443 |
| | | C₁₉H₁₂³⁵ClF₅N₄O |
| 369 | 2-(3-Fluoro-4-trifluoromethyl-phenylamino)-4- | 3.72 |
| | trifluoromethyl-pyrimidine-5-carboxylic acid 4- | 477 |
| | fluoro-benzylamide | C₂₀H₁₂F₈N₄O |
| 370 | 2-(3-Chloro-phenylamino)-4-trifluoromethyl- | 3.16 |
| | pyrimidine-5-carboxylic acid 4-carbamoyl-benzylamide | 450 |
| | | C₂₀H₁₅³⁵ClF₃N₅O₂ |
| 371 | 2-(3-Chloro-phenylamino)-4-trifluoromethyl- | 4.00 |
| | pyrimidine-5-carboxylic acid 4-tert-butyl-benzylamide | 463 |
| | | C₂₃H₂₂³⁵ClF₃N₄O |
| 372 | 2-(3-Chloro-phenylamino)-4-trifluoromethyl- | 3.77 |
| | pyrimidine-5-carboxylic acid N-Boc-4-amino-benzylamide | 522 |
| | | C₂₄H₂₃³⁵ClF₃N₅O₃ |
| 373 | 2-(3-Chloro-phenylamino)-4-trifluoromethyl- | 3.64 |
| | pyrimidine-5-carboxylic acid ((R)-1-phenyl- | 421 |
| | ethyl)-amide | C₂₀H₁₆³⁵ClF₃N₄O |
| 374 | 2-(3-Chloro-phenylamino)-4-trifluoromethyl- | 3.76 |
| | pyrimidine-5-carboxylic acid 3-chloro,4-fluoro-benzylamide | 459 |
| | | C₁₉H₁₂³⁵Cl₂F₄N₄O |
| 375 | 2-(3-Chloro-phenylamino)-4-trifluoromethyl- | 3.67 |
| | pyrimidine-5-carboxylic acid (4-fluoro-benzyl)- | 439 |
| | methyl-amide | C₂₀H₁₅³⁵ClF₄N₄O |
| 376 | 2-(3-Chloro-4-trifluoromethoxy-phenylamino)- | 3.82 |
| | 4-trifluoromethyl-pyrimidine-5-carboxylic acid | 509 |
| | 4-fluoro-benzylamide | C₂₀H₁₂³⁵ClF₇N₄O₂ |
| 377 | {3-[(4-Fluoro-benzylcarbamoyl)- | 3.18 |
| | trifluoromethyl-pyrimidin-2-ylamino]-phenyl}- | 449 |
| | acetic acid | C₂₁H₁₆F₄N₄O₃ |
| 378 | 3-Chloro-5-[(4-fluoro-benzylcarbamoyl)- | 3.62 |
| | trifluoromethyl-pyrimidin-2-ylamino]-benzoic | 469 |
| | acid | C₂₀H₁₃³⁵ClF₄N₄O₃ |
| 379 | 2-[3,5-Bis(trifluoromethyl)-phenylamino]-4- | 3.92 |
| | (trifluoromethyl)-pyrimidine-5-carboxylic acid | 527 |
| | 4-fluorobenzylamide | C₂₁H₁₂F₁₀N₄O |

### EXAMPLE 380

### 2-(3-Chlorophenylamino)-4-trifluoromethylpyridin-5-carboxylic acid (pyridin-4-ylmethyl) amide

To a solution of 6-(3-chlorophenylamino)-4-(trifluoromethyl)-nicotinic acid hydrochloride (Description 2) (0.2 g) in dimethylformamide (5 mL) were added N-methylmorpholine (283 μL), C-pyridin-4-yl-methylamine (62 μL), 1-hydroxybenzotriazole hydrate (104 mg), 1-(3-dimethylamino-propyl)-3-ethylcarbodiimide hydrochloride (118 mg). After stirring at room temperature for 6 h, dimethylformamide was evaporated under reduced pressure and dichloromethane added. The solution was washed with a 5% aqueous solution of potassium carbonate (5 mL), then with brine (2×3 mL) and was evaporated under reduced pressure. Chromatographic purification (silica gel; hexane, ethyl acetate 8:2) afforded the title compound (62 mg).

¹H NMR (300 MHz, DMSO-d6) δ 9.95 (1H, br s), 9.1 (1H, t), 8.55 (3H, m), 8.05 (1H, s), 7.5 (1H, d), 7.35 (3H, t), 7.22 (1H, s), 7.05 (1H, d), 4.5 (2H, d).

MS m/z (EI⁺): 406 and 408 (M⁺.), 299, 236. IR (KBr): 3467 cm-1, 3248, 1646.

### EXAMPLE 381

### 2-(3-Chlorophenylamino)-4-trifluoromethylpyridine-5-carboxylic acid benzylamide

In a manner similar to the method described above, 6-(3-chlorophenylamino)-4-(trifluoromethyl)-nicotinic acid hydrochloride (Description 2) (0.2 g) was reacted benzylamine (67 μL) to afforded 2-(3-chlorophenylamino)-4-trifluoromethylpyridine-5-carboxylic acid benzylamide (48 mg).

¹H NMR (300 MHz, DMSO-d6) δ 9.9 (1H, s) 9.0 (1H, t), 8.5 (1H, s), 8.02 (1H, s), 7.5 (1H, d), 7.15-7.4 (7H, m), 7.02 (1H, d), 4.45 (2H, d).

MS m/z (EI⁺): 405 and 407 (M⁺.), 336, 299, 236. IR (KBr): 3401 cm-1, 3308, 1648.

### EXAMPLE 382

### 6-(3-Chlorophenylamino)-N-(4-fluorobenzyl)-4-isopropyl-nicotinamide

To a solution of 6-(3-chlorophenylamino)-4-isopropyl-nicotinic acid (Description 4) (48 mg) in dimethylformamide (2.5 ml) was added successively N-ethylmorpholine (69 μl), 4-fluorobenzylamine (23 μl), 1-hydroxybenzotriazole hydrate (40 mg) and 1-(3-dimethylamino-propyl)-3-ethylcarbodiimide hydrochloride (40 mg). The solution was stirred for 3 h and allowed to stand overnight. Dimethylformamide was removed under reduced pressure and ethyl acetate (8 ml) added. The solution was washed sequentially with aqueous 5% sodium bicarbonate solution (5 ml), water (5 ml) and brine (2×5 ml). The dried (MgSO₄) solution was evaporated to afford the title compound (56 mg).

NMR (DMSO-d6) δ 1.15 (6H, d), 3.43 (1H, m), 4.41 (2H, d), 6.79 (1H, s), 6.93 (1H, d), 7.17 (2H, t), 7.28 (1H, t), 7.38 (2H, m), 7.46 (1H, d), 8.06 (1H, t), 8.21 (1H, s), 8.91 (1H, t), 9.44 (1H, s).

LC/MS t=3.5 min, [MH⁺] 398.

Compounds of Example 383 to 292 were prepared in a manner similar to that described in Example 382.

TABLE 9

| | | RT (min), (MH+) |
|---|---|---|
| Ex. | | Consistent with |
| No. | Compound Name | molecular formula |
| 383 | N-Benzyl-6-(3-chloro-phenylamino)-4- | 3.6 |
| | isopropyl-nicotinamide | 380 |
| | | C₂₂H₂₂³⁵ClN₃O |
| 384 | 6-(3-Chloro-phenylamino)-N-(4-cyano- | 3.3 |
| | benzyl)-4-isopropyl-nicotinamide | 405 |
| | | C₂₃H₂₁³⁵ClN₄O |
| 385 | 6-(3-Chloro-phenylamino)-4-isopropyl-N- | 3.5 |
| | (4-methoxy-benzyl)-nicotinamide | 410 |
| | | C₂₃H₂₄³⁵ClN₃O₂ |
| 386 | 6-(3-Chloro-phenylamino)-N-(3,4- | 3.6 |
| | difluoro-benzyl)-4-isopropyl-nicotinamide | 416 |
| | | C₂₂H₂₀³⁵ClF₂N₃O |
| 387 | N-(4-Carbamoyl-benzyl)-6-(3-chloro- | 3.0 |
| | phenylamino)-4-isopropyl-nicotinamide | 423 |
| | | C₂₃H₂₃³⁵ClN₄O₂ |
| 388 | 6-(3-Chloro-phenylamino)-N-(2,4- | 3.6 |
| | difluoro-benzyl)-4-isopropyl-nicotinamide | 416 |
| | | C₂₂H₂₀³⁵ClF₂N₃O |
| 389 | 6-(3-Chloro-phenylamino)-4-isopropyl-N- | 3.2 |
| | (4-methanesulfonyl-benzyl)-nicotinamide | 458 |
| | | C₂₃H₂₄³⁵ClN₃O₃S |
| 390 | N-(4-Acetylamino-benzyl)-6-(3-chloro- | 3.1 |
| | phenylamino)-4-isopropyl-nicotinamide | 437 |
| | | C₂₄H₂₅³⁵ClN₄O₂ |
| 391 | 6-(3-Chloro-phenylamino)-4-isopropyl-N- | 3.2 |
| | (4-methane-sulfonylamino-benzyl)- | 473 |
| | nicotinamide | C₂₃H₂₅³⁵ClN₄O₃S |
| 392 | 6-(3-Chloro-phenylamino)-4-isopropyl-N- | 3.1 |
| | (4-methylcarbamoyl-benzyl)-nicotinamide | 437 |
| | | C₂₄H₂₅³⁵ClN₄O₂ |

### EXAMPLE 393

### N-(4-Fluoro-benzyl)-4-isopropyl-6-(3-trifluoromethyl-phenylamino)-nicotinamide

A mixture of 6-chloro-N-(4-fluoro-benzyl)-4-isopropyl-nicotinamide (Description 6) (80 mg), 3-trifluoromethyl-aniline (63 mg), methanesulphonic acid (50 mg), and 1,4-dioxan (0.8 ml) were heated at 180° in the microwave apparatus for 30 min. The mixture was diluted with methanol (3 ml) and purified on the Biotage Horizon HPFC System to give N-(4-Fluoro-benzyl)-4-isopropyl-6-(3-trifluoromethyl-phenylamino)-nicotinamide (43 mg).

NMR (d⁶-DMSO) δ 1.20 (6H, d), 3.47 (1H, m), 4.42 (211, d), 6.85 (1H, s), 7.1-7.3 (3H, m), 7.4 (2H, br s), 7.5 (1H, m), 7.85 (1H, d), 8.25 (1H, s), 8.35 (1H, s), 8.95 (1H, br s), 9.65 (1H, s).

LC/MS t=3.69 min, [MH⁺] 432 consistent with molecular formula C₂₀H₅F₄N₃O

### EXAMPLE 394

### 6-(3-Chloro-4-fluoro-phenylamino)-N-(4-fluoro-benzyl)-4-isopropyl-nicotinamide

A mixture of 6-chloro-N-(4-fluoro-benzyl)-4-isopropyl-nicotinamide (Description 6) (100 mg), 3-chloro-4-fluoroaniline (47 mg), methanesulphonic acid (31 mg), and 1,4-dioxan (1 ml) was irradiated at 180° in the microwave apparatus for 30 min. The solution was evaporated, and the residue partitioned between ethyl acetate and brine. The organic layer was washed with brine and evaporated. The residue was purified on the Biotage Horizon HPFC System to give 6-(3-chloro-4-fluoro-phenylamino)-N-(4-fluoro-benzyl)-4-isopropyl-nicotinamide (41 mg) as a white solid.

NMR (d⁶-DMSO) δ 1.12 (6H, d), 3.42 (1H, multiplet), 4.40 (2H, d), 6.77 (1H, s), 7.19 (2H, t), 7.3-7.4 (3H, m), 7.45-7.5 (1H, m), 8.17 (1H, dd), 8.21 (1H, s), 8.9 (1H, t), 9.45 (1H, s).

LC/MS t=3.50 min [MH⁺] 416 consistent with the molecular formula C₂₂H₂₀³⁵ClF₂N₃O

### EXAMPLE 395

### 6-(2-Cyano-3-methyl-phenylamino)-N-(4-fluoro-benzyl)-4-isopropyl-nicotinamide

A mixture of 6-chloro-N-(4-fluoro-benzyl)-4-isopropyl-nicotinamide (Description 6) (100 mg), 2-amino-6-methyl-benzonitrile (43 mg), cesium carbonate (168 mg), tris(dibenzylideneacetone)dipalladium (0) (ex-Aldrich, 3.36 mg) and 4,5-bis(diphenylphosphino)-9,9-dimethylxanthene (ex-Aldrich, 2.3 mg) and 1,4-dioxan (1 ml) was heated to relux under nitrogen for 24 h. When cool, the mixture was diluted with ethyl acetate and filtered through a PTFE disc (1.0 M) disc and the filtrate evaporated. The residue was purified using the Biotage Horizon HPFC System and the resultant product was triturated with ether, washed with ether, and dried in vacuo at 40° to give 6-(2-cyano-3-methyl-phenylamino)-N-(4-fluoro-benzyl)-4-isopropyl-nicotinamide (15 mg).

NMR (d6-DMSO) δ 1.16 (6H, d), 2.46 (3H, s), 3.35-3.45 (1H, m), 4.36-4.47 (2H, m), 6.95 (1H, s), 7.08 (1H, d), 7.13 (1H, t), 7.35 (2H, m), 7.39 (2H, t), 7.68 (1H, d), 8.07 (1H, s), 8.9 (1H, m), 9.14 (1H, s). LC/MS t=3.27 min, [MH+] 403 consistent with molecular formula C₂₄H₂₃FN₄O

Compounds of Examples 396 to 403 were prepared in a manner similar to Example 393 (Method A) or Example 394 (Method B)

TABLE 10

| | | | Ret. Time |
|---|---|---|---|
| Example | | | [MH+] |
| No. | Compound Name | Method | Molecular Formula |
| 396 | N-(4-Fluoro-benzyl)-6-(3-fluoro- | A | 3.40 min |
| | phenylamino)-4-isopropyl-nicotinamide | | MH⁺ 382 |
| | | | C₂₂H₂₁F₂N₃O |
| 397 | 6-(4-Cyano-phenylamino)-N-(4- | A | 3.30 min |
| | fluoro-benzyl)-4-isopropyl-nicotinamide | | MH⁺ 389 |
| | | | C₂₃H₂₁FN₄O |
| 398 | 6-(3-Cyano-phenylamino)-N-(4- | A | 3.30 min |
| | fluoro-benzyl)-4-isopropyl-nicotinamide | | MH⁺ 389 |
| | | | C₂₃H₂₁FN₄O |
| 399 | 6-(4-Chloro-2-fluoro- | A | 3.60 min |
| | phenylamino)-N-(4-fluoro- | | MH⁺ 416 |
| | benzyl)-4-isopropyl-nicotinamide | | C₂₂H₂₀³⁵ClF₂N₃O |
| 400 | 6-(4-Bromo-2-chloro- | A | 3.79 min |
| | phenylamino)-N-(4-fluoro- | | MH⁺ 478 |
| | benzyl)-4-isopropyl-nicotinamide | | C₂₂H₂₀⁸¹BrClFN₃O |
| 401 | 6-(2,4-Dichloro-phenylamino)-N- | A | 3.73 min |
| | (4-fluoro-benzyl)-4-isopropyl-nicotinamide | | MH⁺ 433 |
| | | | C₂₂H₂₀Cl₂FN₃O |
| 402 | 6-(3-Chloro-4-cyano- | B | 3.30 min |
| | phenylamino)-N-(4-fluoro- | | MH⁺ 423 |
| | benzyl)-4-isopropyl-nicotinamide | | C₂₃H₂₀³⁵ClFN₄O |
| 403 | 6-(4-Bromo-3-fluoro- | B | 3.80 min |
| | phenylamino)-N-(4-fluoro- | | MH⁺ 462 |
| | benzyl)-4-isopropyl-nicotinamide | | C₂₂H₂₀⁸¹BrF₂N₃O |

Compounds of Examples 404 to 411 were prepared in a manner similar to Example 393 (Method A) or Example 394 (Method B)

TABLE 11

| | | | Ret. Time |
|---|---|---|---|
| Example | | | [MH+] |
| No. | Compound Name | Method | Molecular Formula |
| 404 | N-(4-Fluoro-benzyl)-4-isopropyl-6- | A | 3.7 min |
| | (3-trifluoromethoxy-phenylamino)-nicotinamide | | MH⁺ 448 |
| | | | C23H₂₁F₄N₃O₂ |
| 405 | 6-(3-Chloro-2-fluoro-phenylamino)- | B | 3.40 min |
| | N-(4-fluoro-benzyl)-4-isopropyl-nicotinamide | | MH⁺ 416 |
| | | | C₂₂H₂₀³⁵ClF2N₃O |
| 406 | 6-(3-Bromo-2-methyl-phenylamino)- | B | 3.50 min |
| | N-(4-fluoro-benzyl)-4-isopropyl-nicotinamide | | MH⁺ 458 |
| | | | C₂₃H₂₃⁸¹BrFN₃O |
| 407 | 6-(3-Chloro-2-methyl-phenylamino)- | B | 3.50 min |
| | N-(4-fluoro-benzyl)-4-isopropyl-nicotinamide | | MH⁺ 412 |
| | | | C₂₃H₂₃³⁵ClFN₃O |
| 408 | N-(4-Fluoro-benzyl)-4-isopropyl-6- | A | 3.90 min |
| | m-tolylamino-nicotinamide | | MH⁺ 378 |
| | | | C₂₃H₂₄FN₃O |
| 409 | N-(4-Fluoro-benzyl)-4-isopropyl-6- | A | 3.20 min |
| | (3-methoxy-phenylamino)-nicotinamide | | MH⁺ 394 |
| | | | C₂₃H₂₄FN₃O₂ |
| 410 | 6-(4-Bromo-2-fluoro-phenylamino)- | A | 3.70 min |
| | N-(4-fluoro-benzyl)-4-isopropyl-nicotinamide | | MH⁺ 462 |
| | | | C₂₂H₂₀⁸¹BrF₂N₃O |
| 411 | 6-(3,4-Dichloro-phenylamino)-N-(4- | B | 3.90 min |
| | fluoro-benzyl)-4-isopropyl-nicotinamide | | MH⁺ 433 |
| | | | C₂₂H₂₀³⁵Cl₂FN₃O |

Compounds of Examples 412 to 421 were prepared in a manner similar to Example 393 (Method A), Example 394 (Method B) or Example 395 (Method C).

TABLE 12

| | | | Ret. Time |
|---|---|---|---|
| Example | | | [MH⁺] |
| No. | Compound Name | Method | Molecular Formula |
| 412 | N-(4-Fluoro-benzyl)-4-isopropyl-6- | B | 3.60 min |
| | (2-methyl-3-trifluoromethyl- | | MH⁺ 446 |
| | phenylamino)-nicotinamide | | C₂₄H₂₃F₄N₃O |
| 413 | N-(4-Fluoro-benzyl)-6-(2-fluoro-3- | B | 3.70 min |
| | trifluoromethyl-phenylamino)-4- | | MH⁺ 450 |
| | isopropyl-nicotinamide | | C₂₃H₂₀F₅N₃O |
| 414 | 6-(2,3-Dichloro-phenylamino)-N-(4- | B | 3.70 min |
| | fluoro-benzyl)-4-isopropyl-nicotinamide | | MH⁺ 433 |
| | | | C₂₂H₂₀³⁵Cl₂FN₃O |
| 415 | N-(4-Fluoro-benzyl)-6-(3-fluoro-2- | B | 3.31 min |
| | methyl-phenylamino)-4-isopropyl-nicotinamide | | M⁺ 396 |
| | | | C₂₃H₂₃F₂N₃O |
| 416 | 6-(2-Bromo-3-methyl-phenylamino)- | B | 3.61 min |
| | N-(4-fluoro-benzyl)-4-isopropyl-nicotinamide | | MH⁺ 458 |
| | | | C₂₃H₂₃⁸¹BrFN₃O |
| 417 | 6-(3-Bromo-phenylamino)-N-(4- | A | 3.64 min |
| | fluoro-benzyl)-4-isopropyl-nicotinamide | | MH⁺ 444 |
| | | | C₂₂H₂₁⁸¹BrFN₃O |
| 418 | 6-(3-Chloro-2-cyano-phenylamino)- | C | 3.39 min |
| | N-(4-fluoro-benzyl)-4-isopropyl- | | MH⁺ 423 |
| | nicotinamide | | C₂₃H₂₀³⁵ClFN₄O |
| 419 | N-(4-Fluoro-benzyl)-6-(4-fluoro-3- | B | 3.71 min |
| | trifluoromethyl-phenylamino)-4- | | MH⁺ 450 |
| | isopropyl-nicotinamide | | C₂₃H₂₀F₅N₃O |
| 420 | 6-(3,4-Dibromo-phenylamino)-N-(4- | B | 3.90 min |
| | fluoro-benzyl)-4-isopropyl-nicotinamide | | MH⁺ 524 |
| | | | C₂₂H₂₀⁸¹Br₂FN₃O |
| 421 | 6-(3-Chloro-4-methyl-phenylamino)- | B | 3.74 min |
| | N-(4-fluoro-benzyl)-4-isopropyl-nicotinamide | | MH⁺ 412 |
| | | | C₂₃H₂₃³⁵ClFN₃O |

### EXAMPLE 422

### 6-(3-Chloro-phenylamino)-N-(1H-imidazol-2-ylmethyl)-4-trifluoromethyl-nicotinamide

PS-carbodiimide (0.31 g, 0.4 mmol, loading 1.31 mmol/g, ex Argonaut Technologies) and 1-hydroxy-7-azabenzotriazole (0.046 g, 0.34 mmol) were added to a solution of 6-(3-chlorophenylamino)-4-(trifluoromethyl)-nicotinic acid (Description 7) (0.07 g, 0.22 mmol) in dry dichloromethane (3 mL) and the mixture was stirred at room temperature overnight. The resin was filtered and washed repeatedly with dichloromethane, the solvent was then removed in vacuo. The solid residue was dissolved in anhydrous N-methylpyrrolidone (1 mL) and 2-aminomethyl imidazole (19 mg, 0.22 mmol) was added. The solution was heated in a sealed tube under microwave irradiation for 30 min at 140° C. (power-20-30 W). The reaction mixture was diluted with dichloromethane, washed with an aqueous solution of 10% K₂CO₃, dried over magnesium sulphate and evaporated under reduced pressure. Chromatographic purification through preparative HPLC on a Symmetry C₁₈ column, by gradient elution with a solvent system water/TFA 99.9:0.1 respectively (A) and CH₃CN/TFA 99.9:0.1 respectively (B) with the following gradient: 5% B (3 min); 5% B→95% B (1 min); 95% B (1 min); 95% B→5% B (2 min) afforded the title compound as its trifluoroacetate salt, which was suspended in dichloromethane and treated with 0.5 N NaOH. The organic layer was dried over Na₂SO₄ and evaporated under reduced pressure to give the title compound (50 mg, yield=57%).

¹H NMR (300 MHz, DMSO-d₆) δ: 9.90 (s, 1H); 9.01 (t br, 1H); 8.58 (s, 1H); 8.04 (t, 1H); 7.49 (ddd, 1H); 7.34 (dd, 1H); 7.17 (s, 1H); 7.06 (m, 2H); 7.04 (ddd, 1H); 4.48 (d, 2H).

MS m/z (ESI+): AQA; Spray 3.5 kV; Skimmer 30V; Probe 250° C.: 396 (MH+).

Compounds of Examples 423 to 437 were prepared in a manner similar to Example 562 (Method A), Example 563 (Method B), or Example 422 (Method D).

TABLE 13

| Ex | | | |
|---|---|---|---|
| No | Compound name | Method | ¹H NMR (Solvent) ppm and/or MS |
| 423 | N-(4-Fluoro-benzyl)-6-(3- | B | ¹H NMR (300 MHz, DMSO-d₆) δ: |
| | chloro-phenylamino)-4- | | 9.89 (s, 1H); 9.02 (t br, 1H); 8.47 (s, |
| | trifluoromethyl- | | 1H); 8.02 (dd, 1H); 7.50 (dd, 1H); |
| | nicotinamide | | 7.36 (m, 3H); 7.16 (m, 3H); 7.03 (dd, |
| | | | 1H); 4.42 (d, 2H). |
| | | | ESI Pos: AQA; Spray 3 kV; Source |
| | | | 20 V; Probe 250° C.: 424 (MH+). |
| 424 | N-(2-Fluoro-pyridin-4- | B | ESI Pos: AQA; Spray 3.5 kV; |
| | ylmethyl)-6-(3-chloro- | | Skimmer 30 V; Probe 250° C.: |
| | phenylamino)-4- | | 426 (MH+). |
| | trifluoromethyl- | | |
| | nicotinamide | | |
| 425 | N-(4-Fluoro-benzyl)-6-(3- | B | ESI Pos: AQA; Spray 3.5 kV; |
| | bromo-phenylamino)-4- | | Skimmer 30 V; Probe 250° C.: |
| | trifluoromethyl- | | 468 (MH+). |
| | nicotinamide | | |
| 426 | N-(2-Fluoro-pyridin-4- | B | ESI Pos: AQA; Spray 3.5 kV; |
| | ylmethyl)-6-(3-bromo- | | Skimmer 30 V; Probe 250° C.: |
| | phenylamino)-4- | | 469 (MH+). |
| | trifluoromethyl- | | |
| | nicotinamide | | |
| 427 | N-(2-Fluoro-pyridin-4- | B | ESI Pos: AQA; Spray 3.5 kV; |
| | ylmethyl)-6-(3-fluoro- | | Skimmer 30 V; Probe 250° C.: |
| | phenylamino)-4- | | 408 (MH+). |
| | trifluoromethyl- | | |
| | nicotinamide | | |
| 428 | N-(4-Fluoro-benzyl)-6-(3- | B | ESI Pos: AQA; Spray 3.5 kV; |
| | fluoro-phenylamino)-4- | | Skimmer 30 V; Probe 250° C.: |
| | trifluoromethyl- | | 408 (MH+). |
| | nicotinamide | | |
| 429 | N-(2-Fluoro-pyridin-4- | A | ¹H NMR (300 MHz, DMSO-d₆) δ: |
| | ylmethyl)-6-(2,3-dichloro- | | 9.36 (s br, 1H); 9.14 (t br, 1H); |
| | phenylamino)-4- | | 8.45 (s, 1H); 8.20 (d, 1H); 7.91 (dd, 1H); |
| | trifluoromethyl- | | 7.43-7.28 (m, 4H); 7.07 (s br, 1H); |
| | nicotinamide | | 4.51 (d, 2H). |
| | | | EI+; TSQ 700; source 180° C.; 70 V; |
| | | | 200 uA: 458 (M⁺.), 423, 332, 269, |
| | | | 236. |
| 430 | N-(2-Fluoro-pyridin-4- | B | ESI Pos: AQA; Spray 3.5 kV; |
| | ylmethyl)-6-(2,4-dichloro- | | Skimmer 30 V; Probe 250° C.: |
| | phenylamino)-4- | | 460 (MH+). |
| | trifluoromethyl- | | |
| | nicotinamide | | |
| 431 | N-(4-Fluoro-benzyl)-6- | B | ESI Pos: AQA; Spray 3.5 kV; |
| | (2,4-dichloro- | | Skimmer 30 V; Probe 250° C.: |
| | phenylamino)-4- | | 459 (MH+). |
| | trifluoromethyl- | | |
| | nicotinamide | | |
| 432 | N-(4-Fluoro-benzyl)-6- | B | ESI Pos: AQA; Spray 3.5 kV; |
| | (3,5-dichloro- | | Skimmer 30 V; Probe 250° C.: |
| | phenylamino)-4- | | 459 (MH+). |
| | trifluoromethyl- | | |
| | nicotinamide | | |
| 433 | N-(2-Fluoro-pyridin-4- | B | ESI Pos: AQA; Spray 3.5 kV; |
| | ylmethyl)-6-(3,5-dichloro- | | Skimmer 30 V; Probe 250° C.: |
| | phenylamino)-4- | | 459 (MH+). |
| | trifluoromethyl- | | |
| | nicotinamide | | |
| 434 | N-(4-Fluoro-benzyl)-6- | B | ¹H NMR (300 MHz, DMSO-d₆) δ: |
| | (3,4-dichloro- | | 10.01 (s br, 1H); 9.03 (t br, 1H); |
| | phenylamino)-4- | | 8.49 (s, 1H); 8.22 (dd, 1H); 7.55 (ABq, |
| | trifluoromethyl- | | 2H); 7.37 (m, 2H); 7.21-7.12 (m, |
| | nicotinamide | | 3H); 4.43 (d, 2H). |
| | | | ESI Pos: AQA; Spray 3 kV; Source |
| | | | 20 V; Probe 250° C.: 458 (MH+). |
| 435 | N-(2-Fluoro-pyridin-4- | B | ESI Pos: AQA; Spray 3.5 kV; |
| | ylmethyl)-6-(3,4-dichloro- | | Skimmer 30 V; Probe 250° C.: |
| | phenylamino)-4- | | 460 (MH+). |
| | trifluoromethyl- | | |
| | nicotinamide | | |
| 436 | N-(Pyridin-4-ylmethyl)-6- | A | ¹H NMR (300 MHz, DMSO-d₆) δ: |
| | (2-methyl-5-chloro- | | 9.07 (t br, 1H); 8.96 (s, 1H); 8.51 (m, |
| | phenylamino)-4- | | 2H); 8.45 (m, 1H); 7.87 (d, 1H); |
| | trifluoromethyl- | | 7.33 (m, 2H); 7.30 (s, 1H); 7.26 (d, 1H); |
| | nicotinamide | | 7.08 (dd, 1H); 4.46 (d, 2H); 2.24 (s, |
| | | | 3H). |
| | | | EI+; TSQ 700; source 180° C.; 70 V; |
| | | | 200 uA: 420 (M⁺.), 328. |
| 437 | N-(Pyridin-4-ylmethyl)-6- | A | ¹H NMR (300 MHz, DMSO-d₆) δ: |
| | (2-methyl-4-chloro- | | 9.04 (t br, 1H); 8.98 (s, 1H); 8.51 (m, |
| | phenylamino)-4- | | 2H); 8.37 (s, 1H); 7.63 (d, 1H); |
| | trifluoromethyl- | | 7.34 (d, 1H); 7.31 (m, 2H); 7.25 (dd, 1H); |
| | nicotinamide | | 7.16 (s, 1H); 4.45 (d, 2H); 2.23 (s, |
| | | | 3H). |
| | | | EI+; TSQ 700; source 180° C.; 70 V; |
| | | | 200 uA: 420 (M⁺.); 405; 313. |

TABLE 14

| Example | | | |
|---|---|---|---|
| No. | Compound name | Method | ¹H NMR (Solvent) ppm and/or MS |
| 438 | N-Phenethyl-6-(3- | B | ESI Pos: AQA; Spray 3.5 kV; |
| | chloro-phenylamino)-4- | | Skimmer 30 V; Probe 250° C.: 420 (MH+). |
| | trifluoromethyl- | | |
| | nicotinamide | | |
| 439 | N-(5-Methyl- | D | ESI Pos: AQA; Spray 3.5 kV; |
| | [1,3,4]oxadiazol-2- | | Skimmer 30 V; Probe 250° C.: 412 (MH+). |
| | ylmethyl)-6-(3-chloro- | | |
| | phenylamino)-4- | | |
| | trifluoromethyl- | | |
| | nicotinamide | | |
| 440 | N-Phenethyl-6-(3- | B | ESI Pos: AQA; Spray 3.5 kV; |
| | bromo-phenylamino)-4- | | Skimmer 30 V; Probe 250° C.: 464 (MH+). |
| | trifluoromethyl- | | |
| | nicotinamide | | |
| 441 | N-Phenethyl-6-(3- | B | ESI Pos: AQA; Spray 3.5 kV; |
| | fluoro-phenylamino)-4- | | Skimmer 30 V; Probe 250° C.: 404 (MH+). |
| | trifluoromethyl- | | |
| | nicotinamide | | |
| 442 | N-(5-Methyl- | D | ESI Pos: AQA; Spray 3.5 kV; |
| | [1,3,4]oxadiazol-2- | | Skimmer 30 V; Probe 250° C.: 446 (MH+). |
| | ylmethyl)-6-(2,4- | | |
| | dichloro-phenylamino)- | | |
| | 4-trifluoromethyl- | | |
| | nicotinamide | | |
| 443 | N-(5-Methyl-4H- | D | ESI Pos: AQA; Spray 3.5 kV; |
| | [1,2,4]triazol-3- | | Skimmer 30 V; Probe 250° C.: 445 (MH+). |
| | ylmethyl)-6-(2,4- | | |
| | dichloro-phenylamino)- | | |
| | 4-trifluoromethyl- | | |
| | nicotinamide | | |
| 444 | N-Phenethyl-6-(3,4- | B | ESI Pos: AQA; Spray 3.5 kV; |
| | dichloro-phenylamino)- | | Skimmer 30 V; Probe 250° C.: 454 (MH+). |
| | 4-trifluoromethyl- | | |
| | nicotinamide | | |
| 445 | N-Phenethyl-6-(3,5- | B | ESI Pos: AQA; Spray 3.5 kV; |
| | dichloro-phenylamino)- | | Skimmer 30 V; Probe 250° C.: 454 (MH+). |
| | 4-trifluoromethyl- | | |
| | nicotinamide | | |

### EXAMPLE 446

### 6-(3-Chloro-phenylamino)-4-isopropyl-N-pyrimidin-4-ylmethyl-nicotinamide

To a solution of 6-(3-chloro-phenylamino)-4-isopropyl-nicotinic acid (Description 4) (30 mg) in dimethylformamide (1.5 ml) was added successively N-ethylmorpholine (42 μl), pyrimidin-4-yl-methylamine (Ref.: Maury et al., Bull. Soc. Chim. Belg., 91(2), 153, (1982)) (14 mg), 1-hydroxybenzotriazole hydrate (25 mg) and 1-(3-dimethylamino-propyl)-3-ethylcarbodiimide hydrochloride (25 mg). The solution was stirred for 3 h and allowed to stand overnight. Dimethylformamide was removed under reduced pressure and ethyl acetate (5 ml) added. The solution was washed sequentially with 5% sodium bicarbonate solution (3 ml), water (3 ml), brine (2×3 ml), dried (MgSO₄), and evaporated to afford the title compound (25 mg).

NMR (DMSO-d6) δ 1.18 (6H, d), 3.45 (1H, m), 4.51 (2H, d), 6.82 (1H, s), 6.94 (1H, d), 7.29 (1H, t), 7.47 (1H, d), 7-52 (1H, d), 8.09 (1H, t), 8.36 (1H, s), 8.77 (1H, d), 9.04 (1H, t), 9.13 (1H, s), 9.48 (1H, s). LC/MS t 2.9 min, [MH⁺] 382 consistent with molecular formula C₂₀H₂₀³⁵ClN₅O

### EXAMPLE 447

### 6-(3-Chloro-phenylamino)-4-isopropyl-N-pyrazin-2-ylmethyl-nicotinamide

In a manner similar to Example 446, 6-(3-chloro-phenylamino)-4-isopropyl-nicotinic acid (Description 4) (30 mg) and pyrazin-2-yl-methylamine (Ref.: Hirschberg and Mattner, J. Med. Chem., 11(4), 911, (1968)) (14 mg) afforded the title compound (28 mg).

LC/MS t=3.0 min, [M⁺] 382 consistent with molecular formula C₂₀H₂₀³⁵ClN₅O

### EXAMPLE 448

### 6-(3-Chloro-phenylamino)-4-isopropyl-N-(6-methyl-pyridin-3-ylmethyl)-nicotinamide

In a manner similar to Example 446, 6-(3-chloro-phenylamino)-4-isopropyl-nicotinic acid (Description 4) (30 mg) and (6-methyl-pyridin-3-yl)-methylamine dihydrochloride (Description 13) (24.5 mg) afforded the title compound (17 mg).

LC/MS t=2.6 min, [MH⁺] 395 consistent with molecular formula C₂₂H₂₃³⁵ClN₄O.

Compounds of Examples 449 to 456 in the following tables were prepared in the same manner as for Example 393—Method A, or as for Example 394—Method B.

TABLE 15

| | | | Ret. Time |
|---|---|---|---|
| | | | [MH+] |
| Example | | | Molecular |
| No. | Compound Name | Method | Formula |
| 449 | 6-(2-Bromo-4-chloro-phenylamino)- | A | 3.8 |
| | N-(4-fluoro-benzyl)-4-isopropyl-nicotinamide | | 476 |
| | | | C₂₂H₂₀⁷⁹Br³⁵ClFN₃O |
| 450 | 6-(2-Chloro-4-fluoro-phenylamino)- | A | 3.4 |
| | N-(4-fluoro-benzyl)-4-isopropyl-nicotinamide | | 416 |
| | | | C₂₂H₂₀³⁵ClF₂N₃O |
| 451 | 6-(5-Chloro-2-fluoro-phenylamino)- | A | 3.6 |
| | N-(4-fluoro-benzyl)-4-isopropyl-nicotinamide | | 416 |
| | | | C₂₂H₂₀³⁵ClF₂N₃O |
| 452 | 6-(4-Cyano-2-methyl-phenylamino)- | A | 3.3 |
| | N-(4-fluoro-benzyl)-4-isopropyl-nicotinamide | | 403 |
| | | | C₂₄H₂₃FN₄O |
| 453 | 6-(2,5-Dichloro-phenylamino)-N-(4- | A | 3.2 |
| | fluoro-benzyl)-4-isopropyl-nicotinamide | | 432 |
| | | | C₂₂H₂₀³⁵Cl₂FN₃O |
| 454 | 6-(4-Bromo-3-chloro-phenylamino)- | A | 3.9 |
| | N-(4-fluoro-benzyl)-4-isopropyl-nicotinamide | | 476 |
| | | | C₂₂H₂₀⁷⁹Br³⁵ClFN₃O |
| 455 | N-(4-fluoro-benzyl)-6-(3-Fluoro-4- | B | 3.8 |
| | trifluormethyl-phenylamino)-4- | | 450 |
| | isopropyl-nicotinamide | | C₂₃H₂₀F₅N₃O |
| 456 | N-(4-fluoro-benzyl)-4-isopropyl-6- | A, but | 3.7 |
| | (2-methyl-5-trifluoromethyl- | crude | 446 |
| | phenylamino)-nicotinamide | product | C₂₄H₂₃F₄N₃O |
| | | purified | |
| | | using | |
| | | MDAP | |

### EXAMPLE 457

### 6-(3-Chloro-phenylamino)-N-(pyridin-4-ylmethyl)-2-trifluoromethyl-nicotinamide

N-methyl morpholine (0.25 mL, 2.27 mmol, 4.0 eq), 1-hydroxy-benzotriazole (120 mg, 0.88 mmol, 1.5 eq), N-(3-dimethylaminopropyl)-N-ethylcarbodiimide hydrochloride (130 mg, 0.68 mmol, 1.2 eq) and 4-(aminomethyl)-pyridine (0.076 mL, 0.73 mmol, 1.3 eq) were subsequently added to a solution of 6-(3-chloro-phenylamino)-2-trifluoromethyl-nicotinic acid hydrochloride (Description 18) (200 mg, 0.56 mmol, 1.0 eq) in anhydrous DMW (10 mL) and stirred at ambient temperature for 16 h. After evaporation of the solvent in vacuo, the mixture was diluted with ethyl acetate (10 mL) and washed subsequently with a saturated aqueous solution of NaHCO₃ (20 mL×2 times) and brine (20 mL). The organic phase was dried over sodium sulphate and concentrated in vacuo to afford a black residue that was purified by flash chromatography (silica gel, eluent gradient: from hexane/ethyl acetate 7:3 to hexane/ethyl acetate 6:4). The title compound was obtained as a grey solid (130 mg, yield=60%).

EI; TSQ 700; source 180 C; 70 V; 200 uA: 406(M⁺.), 337, 299.

¹H NMR (300 MHz, DMSO-d₆) δ: 9.86(s, 1H); 9.06 (t br, 1H); 8.53 (m, 2H); 8.02 (dd, 1H); 7.85 (d, 1H); 7.52 (ddd, 1H); 7.24 (dd, 1H); 7.33 (m, 2H); 7.13 (d, 1H); 7.02 (ddd, 1H); 4.46 (d, 2H).

Example 458 in was prepared as described for the Example 457, from the appropriate starting materials via similar intermediates, prepared in a similar manner to the intermediates described in Descriptions 14 to 18.

TABLE 16

| Example | | |
|---|---|---|
| No | Compound Name | ¹H NMR (Solvent) ppm and/or MS |
| 458 | 6-(3-Chloro- | EI; TSQ 700; source 180C; 70 V; 200 uA: |
| | phenylamino)-N-benzyl- | 405 (M⁺.), 336, 299. |
| | 2-trifluoromethyl- | ¹H NMR (300 MHz, DMSO-d₆) δ: 9.83 (s, |
| | nicotinamide | 1H); 8.94 (t br, 1H); 8.02 (dd, 1H); 7.79 (d, |
| | | 1H); 7.52 (dd, 1H); 7.39-7.22 (m, 6H); 7.10 (d, |
| | | 1H); 7.00 (dd, 1H); 4.43 (d, 2H) |

### EXAMPLE 459

### 2-(3-Chlorophenylamino)-4-trifluoromethylpyridine-5-carboxylic acid cyclohexylmethyl amide

To a solution of 6-(3-chlorophenylamino)-4-(trifluoromethyl)-nicotinic acid hydrochloride (Description 2) (0.2 g) in dimethylformamide (5 mL) were added N-methylmorpholine (283 mL), 4-aminomethylcyclohexane (80 μL), 1-hydroxybenzotriazole hydrate (104 mg), 1-(3-dimethylamino-propyl)-3-ethylcarbodiimide hydrochloride (118 mg). After stirring at room temperature for 6 h, dimethylformamide was evaporated under reduced pressure and dichloromethane added. The solution was washed with a 5% aqueous solution of potassium carbonate (5 mL), then with brine (2×3 mL) and was evaporated under reduced pressure. Chromatographic purification (silica gel; hexane, ethyl acetate 8:2) afforded the title compound (35 mg).

¹H NMR (300 MHz, DMSO-d6) δ 9.85 (1H, s) 8.45 (2H, m), 8.05 (1H, s), 7.5 (1H, d), 7.35 (1H, t), 7.15 (1H, s), 7.02 (1H, d), 3.1 (2H, t), 0.85-1.8 (1H, m).

MS m/z (EI⁺): 411 and 413 (MH⁺.), 328, 315, 299. IR (KBr): 3412 cm-1, 3309, 2925, 2852, 1648.

### EXAMPLE 460

### 6-(3-Chlorophenylamino)-N-cyclohexylmethyl-4 isopropylnicotinamide

A mixture of 6-chloro-N-cyclohexylmethyl-4-isopropyl-nicotinamide (Description 20) (50 mg) and 3-chloroaniline (90 μl) was heated under microwave conditions at 190° for 20 minutes. Ethyl acetate (5 ml) was added and the solution washed with dilute potassium carbonate solution (3 ml) and water (3 ml), dried (MgSO₄) and evaporated. The residue was triturated with isohexane to afford the title compound (60 mg).

NMR (DMSO-d6) δ 0.85-1.0 (2H, m), 1.1-1.25 (3H, m), 1.16 (6H, d), 1.51 (1H, m), 1.6-1.8 (5H, m), 3.06 (2H, t), 3.41 (1H, m), 6.78 (1H, s), 6.92 (1H, d), 7.27 (1H, t), 7.46 (1H, d), 8.06 (1H, t), 8.12 (1H, s), 8.33 (1H, t), 9.41 (1H, s).

LC/MS, t=3.7 min, Molecular ion observed [MH⁺]=386 consistent with the molecular formula C₂₂H₂₈³⁵ClN₃O.

### EXAMPLE 461

### N-Cyclohexylmethyl-6-(3,4-dichloro-phenylamino)-4-isopropyl-nicotinamide

A mixture of 6-chloro-N-cyclohexylmethyl-4-isopropyl-nicotinamide (Description 20) (50 mg), 3,4-dichloroaniline (Aldrich) (33 mg), sodium t-butoxide (46 mg), tris(dibenzylideneacetone)palladium (0) (3.2 mg), 2-(dicyclohexylphosphino)biphenyl (2.6 mg) and dimethoxyethane (1 ml) was irradiated under microwave conditions at 150° for 30 minutes. Solvent was evaporated under reduced pressure and ethyl acetate (5 ml) added. The mixture was washed with water (3 ml), dried (MgSO₄) and evaporated. The residue was purified by mass-directed autopurification techniques to afford the title compound (12.0 mg).

NMR (DMSO-d6) δ 0.85-1.0 (2H, m), 1.1-1.25 (3H, m), 1.16 (6H, d), 1.51 (1H, m), 1.6-1.8 (5H, m), 3.06 (2H, t), 3.41 (1H, m), 6.80 (1H, s), 7.50 (2H, m), 8.13 (1H, s), 8.25 (1H, s), 8.35 (1H, t), 9.62 (1H, s).

LC/MS t=3.9 min, [MH⁺] 420, consistent with molecular formula C₂₂H₂₇³⁵Cl₂N₃O

### EXAMPLE 462

### 6-(3-Bromo-phenylamino)-N-cyclohexylmethyl-4-isopropyl-nicotinamide

A mixture of 6-chloro-N-cyclohexylmethyl-4-isopropyl-nicotinamide (Description 20) (60 mg) and 3-bromoaniline (Aldrich) (0.5 ml) was irradiated under microwave conditions at 180° for 30 minutes. The mixture was dissolved in dichloromethane and passed down a 10 g SepPak column to remove excess 3-bromoaniline. Elution with 9:1 dichloromethane:ether removed the crude product which was further purified by MDAP to afford the title compound (13.6 mg).

NMR (DMSO-d6) δ 0.85-1.0 (2H, m), 1.1-1.25 (3H, m), 1.17 (6H, d), 1.52 (1H, m), 1.6-1.8 (5H, m), 3.06 (2H, t), 3.42 (1H, m), 6.78 (1H, s), 7.06 (1H, d), 7.22 (1H, t), 7.52 (1H, d), 8.13 (1H, s), 8.19 (1H, s), 8.33 (1H, t), 9.40 (1H, s).

LC/MS t=3.95 min, [MH⁺] 430, consistent with molecular formula C₂₂H₂₈⁷⁹BrN₃O

### EXAMPLE 463

### N-Cyclohexylmethyl-6-(2,4-dichlorophenylamino)-4-isopropyl-nicotinamide

A mixture of 6-chloro-N-cyclohexylmethyl-4-isopropyl-nicotinamide (Description 20) (50 mg), 2,4-dichloroaniline (33 mg), sodium t-butoxide (23 mg), tris(dibenzylideneacetone)palladium(0) (1.6 mg), 2-(dicyclohexylphosphino)biphenyl (1.3 mg) and dimethoxyethane (1 ml) was stirred under reflux for 18 hours. The solvent was evaporated under reduced pressure and ethyl acetate (5 ml) added. The mixture was washed with water (3 ml), dried (MgSO₄) and evaporated. The residue was purified by MDAP to afford the title compound (12 mg).

NMR (DMSO-d6) δ 0.8-1.0 (2H, m), 1.1-1.3 (3H, m), 1.17 (61, d), 1.50 (1H, m), 1.6-1.8 (5H, m), 3.05 (2H, t), 3.38 (1H, m), 7.08 (1H, s), 7.40 (1H, d), 7.65 (1H, s), 8.01 (1H, s), 8.07 (1H, d), 8.37 (1H, t), 8.93 (1H, br s).

LC/MS t=3.8 min, [MH⁺] 420, consistent with molecular formula C₂₂H₂₇³⁵Cl₂N₃O

### EXAMPLE 464

### 6-(3-Chloro-phenylamino)-N-cyclobutylmethyl-4-isopropyl-nicotinamide

A mixture of 6-chloro-N-cyclobutylmethyl-4-isopropyl-nicotinamide (Description 22) (80 mg) and 3-chloroaniline (0.5 ml) was irradiated under microwave conditions at 180° C. for 30 mins. The mixture was diluted with dichloromethane (2 ml) and chromatographed on silica gel. The excess aniline was removed by elution with dichloromethane and then elution with dichloromethane/ether (5:1) gave the title compound (38 mg).

NMR (DMSO-d6) δ 1.16 (6H, m), 1.74 (2H, m), 1.82 (2H, m), 2.00 (2H, m), 2.52 (1H, m excess), 3.23 (2H, t), 3.40 (1H, m), 6.78 (1H, s), 6.92 (1H, d), 7.27 (1H, t), 7.46 (1H, d), 8.04 (1H, s), 8.10 (1H, s), 8.33 (1H, t), 9.41 (1H, s)

LC/MS t=3.65 min, [MH⁺] 358 consistent with the molecular formula C₂₀H₂₄³⁵ClN₃O

The compounds of Examples 465 to 496 were prepared as set out in Table 17 below.

Preparative Method A: As for Example 460, with temperature and time of reaction, and any other variations included in the table.

Preparative Method B: As for Example 461, with temperature and time of reaction, and any other variations noted in the table.

Preparative Method C: As for Example 464, with temperature and time of reaction, and any other variations noted in the table.

Purification Method E: Purify by mass-directed autopurification techniques.

Purification Method F: The crude product was diluted with dichloromethane (2 ml) and the solution applied to a Sep-Pack column of silica gel. This was eluted firstly with dichloromethane, followed by dichloromethane/ether 5:1 to give pure product.

TABLE 17

| | | 1. Preparation | | 1. Retention |
|---|---|---|---|---|
| | | method A, B, or C | | Time (min). |
| | | 2. Reaction | Purification | 2. [MH⁺] |
| Ex. | | temperature (° C.), | method | 3. Molecular |
| No. | Compound Name | 3. Time. | E or F | formula |
| 465 | 6-(3-Chloro-phenyl- | A | E | 3.1 |
| | amino)-4-isopropyl-N- | 200° | | 388 |
| | (tetrahydro-pyran-4- | 1 hr | | C₂₁H₂₆³⁵ClN₃O₂ |
| | ylmethyl)-nicotinamide | | | |
| 466 | 6-(3-Bromo-phenyl- | A | E | 3.1 |
| | amino)-4-isopropyl-N- | 200° | | 432 |
| | (tetrahydro-pyran-4- | 30 min | | C₂₁H₂₆⁷⁹BrN₃O₂ |
| | ylmethyl)-nicotinamide | | | |
| 467 | N-Cyclohexylmethyl-4- | B | E | 3.4 |
| | isopropyl-6-(3-methoxy- | 150° | | 382 |
| | phenylamino)-nicotinamide | 30 min | | C₂₃H₃₁N₃O₂ |
| 468 | N-Cyclohexylmethyl-6-(3- | B | E | 3.6 |
| | fluoro-phenylamino)-4- | 150° | | 370 |
| | isopropyl-nicotinamide | 30 min | | C₂₂H₂₈FN₃O |
| 469 | 1-[6-(3-Chloro- | A | E | 3.1 |
| | phenylamino)-4-isopropyl- | 180° | | 360 |
| | pyridin-3-yl]-1-morpholin- | 30 min | | C₁₉H₂₂³⁵ClN₃O₂ |
| | 4-yl-methanone | | | |
| 470 | 6-(3-Bromo-phenylamino)- | A | E | 3.95 |
| | N-cyclohexylmethyl-4- | 180° | | 430 |
| | isopropyl-nicotinamide | 30 min | | C₂₂H₂₈⁷⁹BrN₃O |
| 471 | N-Cyclohexylmethyl-4- | A | E | 3.68 |
| | isopropyl-6-m-tolylamino- | 180° | | 366 |
| | nicotinamide | (1 hr) | | C₂₃H₃₁N₃O |
| 472 | N-Cyclohexylmethyl-4- | A | E | 3.7 |
| | isopropyl-6-(3- | 180° | | 420 |
| | trifluoromethyl- | 1 hr | | C₂₃H₂₈F₃N₃O |
| | phenylamino)-nicotinamide | | | |
| 473 | N-Cyclohexylmethyl-4- | A | E | 3.8 |
| | isopropyl-6-(3- | 180° | | 436 |
| | trifluoromethoxy- | 30 min | | C₂₃H₂₈F₃N₃O₂ |
| | phenylamino)-nicotinamide | | | |
| 474 | 6-(2,3-Dichloro- | B | E | 3.34 |
| | phenylamino)-4-isopropyl- | 150° | | 422 |
| | N-(tetrahydro-pyran-4- | 30 min | | C₂₁H₂₅³⁵Cl₂N₃O₂ |
| | ylmethyl)-nicotinamide | | | |
| 475 | 6-(2,4-Dichloro- | B | E | 3.39 |
| | phenylamino)-4-isopropyl- | 150° | | 422 |
| | N-(tetrahydro-pyran-4- | 30 min | | C₂₁H₂₅³⁵Cl₂N₃O₂ |
| | ylmethyl)-nicotinamide | | | |
| 476 | 6-(3,4-Dichloro- | B | E | 3.51 |
| | phenylamino)-4-isopropyl- | 150° | | 422 |
| | N-(tetrahydro-pyran-4- | 30 min | | C₂₁H₂₅³⁵Cl₂N₃O₂ |
| | ylmethyl)-nicotinamide | | | |
| 477 | 4-Isopropyl-N-(tetrahydro- | A | E | 3.2 |
| | pyran-4-ylmethyl)-6-(3- | 180° | | 422 |
| | trifluoromethyl- | 1 hr | | C₂₂H₂₆F₃N₃O₂ |
| | phenylamino)-nicotinamide | | | |
| 478 | 4-Isopropyl-N-(tetrahydro- | A | E | 3.3 |
| | pyran-4-ylmethyl)-6-(3- | 180° | | 438 |
| | trifluoromethoxy- | 30 min | | C₂₂H₂₆F₃N₃O₃ |
| | phenylamino)-nicotinamide | | | |
| 479 | 6-[(3-Chloro-phenyl)amino]- | A | E | 3.76 |
| | N-(cyclopentylmethyl)-4- | 180° | | 372 |
| | isopropyl-nicotinamide | 30 min | | C₂₁H₂₆N₃ClO |
| 480 | N-Cyclopentylmethyl-6-(3- | A | E | 3.69 |
| | fluorophenylamino)-4- | 180° | | 356 |
| | isopropyl-nicotinamide | 30 min | | C₂₁H₂₆N₃FO |
| 481 | N-Cyclopentylmethyl-4- | A | E | 3.82 |
| | isopropyl-6-(3- | 180° | | 406 |
| | trifluoromethyl- | 30 min | | C₂₁H₂₆N₃F₃O |
| | phenylamino)-nicotinamide | | | |
| 482 | N-Cyclopentylmethyl-4- | A | E | 3.52 |
| | isopropyl-6-m-tolylamino- | 180° | | 352 |
| | nicotinamide | 30 min | | C₂₂H₂₉ON₃ |
| 483 | N-Cyclopentylmethyl-4- | A | E | 3.86 |
| | isopropyl-6-(3- | 180° | | 422 |
| | trifluoromethoxy- | 30 min | | C₂₂H₂₆N₃O₂F₃ |
| | phenylamino)-nicotinamide | | | |
| 484 | 6-(3-Bromophenylamino)-N- | A | E | 3.86 |
| | cyclopentylmethyl-4- | 180° | | 422 |
| | isopropyl-nicotinamide | 30 min | | C₂₁H₂₆N₃OBr |
| 485 | N-Cyclopentylmethyl-4- | A | E | 3.81 |
| | isopropyl-6-(3- | 180° | | 418 |
| | methoxyphenylamino)nicotinamide | 30 min | | C₂₂H₂₉N₃O₂ |
| 486 | 6-(3-Cyano-phenylamino)-N- | A | E | 3.55 |
| | cyclopentylmethyl-4- | 180° | | 363 |
| | isopropyl-nicotinamide | 30 min | | C₂₂H₂₆N₄O |
| 487 | 6-(2-Chloro-4-fluoro- | A | E | 3.6 |
| | phenylamino)-N- | 180° | | 391 |
| | cyclopentylmethyl-4- | 30 min | | C₂₁H₂₅N₃ClFO |
| | isopropyl-nicotinamide | | | |
| 488 | 6-(2-Chloro-4-cyano- | A | E | 3.76 |
| | phenylamino)-N- | 180° | | 398 |
| | cyclopentylmethyl-4- | 30 min | | C₂₂H₂₅N₄ClO |
| | isopropyl-nicotinamide | | | |
| 489 | N-Cyclopentylmethyl-6-(2,4- | A | E | 3.70 |
| | dichloro-phenylamino)-4- | 180° | | 407 |
| | isopropyl-nicotinamide | 30 min | | C₂₁H₂₅N₃Cl₂O |
| 490 | N-Cyclopentylmethyl-6-(3,4- | A | E | 3.80 |
| | dichlorophenyl)amino)-4- | 180° | | 407 |
| | isopropyl-nicotinamide | 30 min | | C₂₁H₂₅N₃Cl₂O |
| 491 | 6-(3-Bromo-phenylamino)- | C | F | 3.70 |
| | N-cyclobutylmethyl-4- | 180° | | 402 |
| | isopropyl-nicotinamide | 30 min | | C₂₀H₂₄⁷⁹BrN₃O |
| 492 | N-Cyclobutylmethyl-6-(3- | C | F | 3.49 |
| | fluoro-phenylamino)-4- | 180° C. | | 342 |
| | isopropyl-nicotinamide | 30 min | | C₂₀H₂₄FN₃O |
| 493 | N-Cyclobutylmethyl-6-(3- | C | F | 3.53 |
| | trifluoromethyl- | 180° | | 392 |
| | phenylamino)-4-isopropyl- | 30 min | | C₂₁H₂₄F₃N₃O |
| | nicotinamide | | | |
| 494 | 6-(3-Cyano-phenylamino)- | C | F | 3.41 |
| | N-cyclobutylmethyl-4- | 180° | | 349 |
| | isopropyl-nicotinamide | 30 min | | C₂₁H₂₄N₄O |
| 495 | N-Cyclobutylmethyl-4- | C | F | 3.39 |
| | isopropyl-6-m-tolylamino- | 180° | | 338 |
| | nicotinamide | 1 hr | | C₂₁H₂₇N₃O |
| 496 | N-Cyclobutylmethyl-4- | C | F | 3.30 |
| | isopropyl-6-(3-methoxy- | 180° | | 354 |
| | phenylamino)-nicotinamide | 1 hr | | C₂₁H₂₇N₃O₂ |

The Examples 497 to 503 in Table 18 were prepared in a manner similar to as Example 460 with the reaction temperature and time given in the table. An asterisk in the third column signifies that the preparative method used was the same as that used in Example 504 and the product was purified by the method given in the fourth column.

Purification Method E: Purify by mass-directed autopurification techniques.

Purification Method F: The crude product was diluted with dichloromethane (2 ml) and the solution applied to a Sep-Pack column of silica gel. This was eluted firstly with dichloromethane, followed by dichloromethane/ether 5:1 to give pure product.

TABLE 18

| | | | | 1. Retention |
|---|---|---|---|---|
| | | | | Time (min). |
| | | 1. Reaction | Purification | 2. [MH⁺] |
| Ex. | | Temperature | method | 3. Molecular |
| No | Compound Name | 2. Reaction Time | E, or F | formula |
| 497 | 6-(3-Fluoro-phenylamino)- | 200° | E | 2.9 |
| | 4-isopropyl-N-(tetrahydro- | 1 hr | | 372 |
| | pyran-4-ylmethyl)- | | | C₂₁H₂₆FN₃O₂ |
| | nicotinamide | | | |
| 498 | 1-[6-(3-Fluoro- | 180° | E | 2.9 |
| | phenylamino)-4-isopropyl- | 30 min | | 344 |
| | pyridin-3-yl]-1-morpholin- | | | C₁₉H₂₂FN₃O₂ |
| | 4-yl-methanone | | | |
| 499 | 4-Isopropyl-6-(3-methoxy- | 180° | E | 2.7 |
| | phenylamino)-N- | 2 hr | | 384 |
| | (tetrahydro-pyran-4- | | | C₂₂H₂₉N₃O₃ |
| | ylmethyl)-nicotinamide | | | |
| 500 | 4-Isopropyl-N-(tetrahydro- | 180° | E | 2.93 |
| | pyran-4-ylmethyl)-6-m- | 1 hr | | 368 |
| | tolylamino-nicotinamide | | | C₂₂H₂₉N₃O₂ |
| 501 | 6-(3-Cyano-phenylamino)- | 180° | E | 2.8 |
| | 4-isopropyl-N-(tetrahydro- | 30 min | | 379 |
| | pyran-4-ylmethyl)- | | | C₂₂H₂₆N₄O₃ |
| | nicotinamide | | | |
| 502 | 6-[(3,4-Dichloro-phenyl)- | 180° C. | E | 3.51 |
| | methyl-amino]-4-isopropyl- | 2 hrs * | | 436 |
| | N-(tetrahydro-pyran-4- | | | C₂₂H₂₇³⁵Cl₂N₃O₂ |
| | ylmethyl)-nicotinamide | | | |
| 503 | 6-[(3-Bromo-phenyl)- | 180° C. | F | 3.31 |
| | methyl-amino]-4-isopropyl- | 2 hrs * | | 446 |
| | N-(tetrahydro-pyran-4- | | | C₂₂H₂₈⁷⁹BrN₃O₂ |
| | ylmethyl)-nicotinamide | | | |

### EXAMPLE 504

### 6-[(3-Fluoro-phenyl)-methyl-amino]-4-isopropyl-N-(tetrahydro-pyran-4-ylmethyl)-nicotinamide

A mixture of 6-chloro-4-isopropyl-N-(tetrahydro-pyran-4-ylmethyl)-nicotinamide (Description 24) (89 mg), 3-fluoro-N-methylaniline (75 mg) and methanesulphonic acid (72 mg) in dioxan (1 ml) was heated on microwave at 180° C. for 2 hours. The mixture was diluted with ethyl acetate (20 ml) and washed with sodium bicarbonate solution (20 ml) and water (2×20 ml) and evaporated to an oil. Purification by chromatography on silica gel (dichloromethane then dichloromethane/methanol 10:1) gave a solid which was triturated with ether/isohexane 1:1 to give the title compound (63 mg).

NMR (DMSO-d6) δ 1.05 (6H, d), 1.15 (2H, m), 1.60 (2H, d), 1.74 (1H, m), 3.10 (2H, t), 3.26 (2H, m), 3.34 (1H, m excess), 3.42 (3H, s), 3.84 (2H, m), 6.64 (1H, s), 7.02 (1H, m), 7.14 (2H, m), 7.43 (1H, q), 8.11 (1H, s), 8.35 (1H, t).

LC/MS t=2.97 min, Molecular ion observed [MH⁺]=386 consistent with the molecular formula C₂₂H₂₈FN₃O₂

All examples prepared in Table 19 were prepared by the same method as given for Example 504, with variations in reaction time, and purification method given in the table.

Purification Method E: Purify by mass-directed autopurification techniques.

Purification Method F: The crude product was diluted with dichloromethane (2 ml) and the solution applied to a Sep-Pack column of silica gel. This was eluted firstly with dichloromethane, followed by dichloromethane/methanol 10:1 to give pure product.

TABLE 19

| | | | | 1. Retention Time |
|---|---|---|---|---|
| | | | | (min). |
| | | | | 2. [MH⁺] |
| Ex. | | Reaction | | 3. Molecular |
| No | Compound Name | time | Purification, E or F | formula |
| 505 | 4-Isopropyl-6-(methyl- | 1 hr | E, then | 2.67 |
| | phenyl-amino)-N- | | silica gel | 368 |
| | (tetrahydro-pyran-4- | | chromatography, | C₂₂H₂₉N₃O₂ |
| | ylmethyl)-nicotinamide | | CH₂Cl₂:MeOH, | |
| | | | 50:1, then 25:1 | |
| 506 | 6-[(3-Chloro-phenyl)- | 2 hrs | E | 3.22 |
| | methyl-amino]-4-isopropyl- | | | 402 |
| | N-(tetrahydro-pyran-4- | | | C₂₂H₂₈³⁵ClN₃O₂ |
| | ylmethyl)-nicotinamide | | | |
| 507 | 6-[(4-Chloro-phenyl)- | 2 hrs | E | 3.20 |
| | methyl-amino]-4-isopropyl- | | | 402 |
| | N-(tetrahydro-pyran-4- | | | C₂₂H₂₈³⁵ClN₃O₂ |
| | ylmethyl)-nicotinamide | | | |

### EXAMPLE 508

### 6-(3-Chloro-phenylamino)-N-cyclobutyl-4-isopropyl-nicotinamide

To a solution of 6-(3-chloro-phenylamino)-4-isopropyl-nicotinic acid (Description 4) (48 mg) in dimethylformamide (2.5 ml) was added successively N-ethylmorpholine (69 μl), cyclobutylamine (17 μl), 1-hydroxybenzotriazole hydrate (40 mg) and 1-(3-dimethylamino-propyl)-3-ethylcarbodiimide hydrochloride (40 mg). The solution was stirred for 3 hours and allowed to stand overnight. Dimethylformamide was removed under reduced pressure and ethyl acetate (8 ml) added. The solution was washed sequentially with 5% sodium bicarbonate solution (5 ml), water (5 ml) and brine (2×5 ml), dried (MgSO₄) and evaporated to afford the title compound (40 mg).

NMR (DMSO-d6) δ 1.16 (6H, d), 1.65 (2H, m), 1.99 (2H, m), 2.2 (2H, m), 3.40 (1H, m), 4.35 (1H, m), 6.77 (1H, s), 6.92 (1H, d), 7.28 (1H, t), 7.46 (1H, d), 8.06 (1H, t), 8.13 (1H, s), 8.56 (1H, d), 9.42 (1H, s).

LC/MS t=3.51 min, [MH⁺] 344, consistent with molecular formula C₁₉H₂₂³⁵ClN₃O

The compounds in Tables 20, 21, and 22 were synthesized by the method used to prepare Example 508.

TABLE 20

| | | 1. Retention Time |
|---|---|---|
| | | (min). |
| | | 2. [MH⁺] |
| Ex. | | 3. Molecular |
| No. | Compound Name | formula |
| 509 | 6-(3-Chloro-phenylamino)-N- | 3.47 |
| | cyclopropylmethyl-4-isopropyl-nicotinamide | 344 |
| | | C₁₉H₂₂³⁵ClN₃O |
| 510 | 6-(3-Chloro-phenylamino)-N-(2-ethyl-butyl)-4- | 3.8 |
| | isopropyl-nicotinamide | 374 |
| | | C₂₁H₂₈³⁵ClN₃O |
| 511 | 6-(3-Chloro-phenylamino)-N-cyclohexyl-4- | 3.7 |
| | isopropyl-nicotinamide | 372 |
| | | C₂₁H₂₆³⁵ClN₃O |
| 512 | 6-(3-Chloro-phenylamino)-N-(1-hydroxy- | 3.46 |
| | cyclohexylmethyl)-4-isopropyl-nicotinamide | 402 |
| | | C₂₂H₂₈³⁵ClN₃O₂ |
| 513 | 1-[6-(3-Chloro-phenylamino)-4-isopropyl- | 3.57 |
| | pyridin-3-yl]-1-piperidin-1-yl-methanone | 358 |
| | | C₂₀H₂₄³⁵ClN₃O |

TABLE 21

| | | 1. Retention Time (min). |
|---|---|---|
| | | 2. [MH⁺] |
| Ex. No | Compound Name | 3. Molecular formula |
| 514 | 6-(3-Chloro-phenylamino)-N-(2,2-dimethyl- | 3.6 |
| | propyl)-4-isopropyl-nicotinamide | 360 |
| | | C₂₀H₂₆³⁵ClN₃O |
| 515 | 6-(3-Chloro-phenylamino)-4-isopropyl-N-(2- | 3.0 |
| | methoxy-ethyl)-nicotinamide | 348 |
| | | C₁₈H₂₂³⁵ClN₃O₂ |
| 516 | 6-(3-Chloro-phenylamino)-4-isopropyl-N- | 3.0 |
| | (tetrahydro-pyran-4-yl)-nicotinamide | 374 |
| | | C₂₀H₂₄³⁵ClN₃O₂ |
| 517 | 6-(3-Chloro-phenylamino)-4-isopropyl-N-[(R)-1- | 3.30 |
| | (tetrahydro-furan-2-yl)methyl]-nicotinamide | 374 |
| | | C₂₀H₂₄³⁵ClN₃O₂ |
| 518 | N-((R)-1-{1-[6-(3-Chloro-phenylamino)-4- | 2.77 |
| | isopropyl-pyridin-3-yl]-methanoyl}-pyrrolidin-3- | 401 |
| | yl)-acetamide | C₂₁H₂₅³⁵ClN₄O₂ |
| 519 | 1-[6-(3-Chloro-phenylamino)-4-isopropyl- | 3.1 |
| | pyridin-3-yl]-1-(4-methane-sulfonyl-piperazin-1- | 437 |
| | yl)-methanone | C₂₀H₂₅³⁵ClN₄O₃S |
| 520 | 6-(3-Chloro-phenylamino)-N-(1,1-dioxo- | 3.0 |
| | tetrahydro-1l⁶-thiophen-3-yl)-4-isopropyl- | 408 |
| | nicotinamide | C₁₉H₂₂³⁵ClN₃O₃S |

TABLE 22

| | | 1. Retention Time (min). |
|---|---|---|
| | | 2. [MH⁺] |
| Ex. No | Compound Name | 3. Molecular formula |
| 521 | 6-(3-Chloro-phenylamino)-4-isopropyl-N-[(S)-1- | 3.30 |
| | (tetrahydro-furan-2-yl)methyl]-nicotinamide | 374 |
| | | C₂₀H₂₄³⁵ClN₃O₂ |
| 522 | 6-(3-Chloro-phenylamino)-N-(1,1-dioxo- | 2.9 |
| | hexahydro-1l⁶-thiopyran-4-yl)-4-isopropyl- | 422 |
| | nicotinamide | C₂₀H₂₄³⁵ClN₃O₃S |
| 523 | 1-[6-(3-Chloro-phenylamino)-4-isopropyl- | 2.18 |
| | pyridin-3-yl]-1-(4-methyl-piperazin-1-yl)- | 373 |
| | methanone | C₂₀H₂₅³⁵ClN₄O |
| 524 | 6-(3-Chloro-phenylamino)-N-(2-dimethylamino- | 2.20 |
| | ethyl)-4-isopropyl-nicotinamide | 361 |
| | | C₁₉H₂₅³⁵ClN₄O |
| 525 | N-((S)-1-{1-[6-(3-Chloro-phenylamino)-4- | 2.77 |
| | isopropyl-pyridin-3-yl]-methanoyl}-pyrrolidin-3- | 401 |
| | yl)-acetamide | C₂₁H₂₅³⁵ClN₄O₂ |
| 526 | N-(1-{1-[6-(3-Chloro-phenylamino)-4-isopropyl- | 2.9 |
| | pyridin-3-yl]-methanoyl}-piperidin-4-yl)- | 451 |
| | methanesulfonamide | C₂₁H₂₇³⁵ClN₄O₃S |

### EXAMPLE 527

### 4-tert-Butyl-6-(3-chloro-phenylamino)-N-cyclohexylmethyl-nicotinamide

A solution of 4-tert-butyl-6-chloro-N-cyclohexylmethyl-nicotinamide (Description 28) (41 mg), 3-chloroaniline (21 μl) and methanesulphonic acid (17 μl) in dioxan (0.5 ml) was irradiated under microwave conditions at 180° for 30 minutes. Solvent was evaporated under reduced pressure and the residue purified by MDAP to afford the title compound (35 mg).

NMR (DMSO-d6) δ 0.85-1.0 (2H, m), 1.1-1.25 (3H, m), 1.35 (9H, s), 1.55 (1H, m), 1.6-1.8 (5H, m), 3.03 (2H, t), 6.87 (1H, s), 6.92 (1H, d), 7.27 (1H, t), 7.46 (1H, d), 7.95 (1H, s), 8.03 (1H, t), 8.36 (1H, t), 9.39 (1H, s).

LC/MS t=4.20 min, [MH⁺] consistent with molecular formula C₂₃H₃₀³⁵ClN₃O

The compounds prepared in Table 23 were prepared in a manner similar to Example 527 from the intermediates in Description 28 or Description 29, with the reaction time given in Table 23.

TABLE 23

| | | | 1. Retention Time (min). |
|---|---|---|---|
| Ex. | | Reaction time | 2. [MH⁺] |
| No. | Compound Name | (minutes) | 3. Molecular formula |
| 528 | 4-tert-Butyl-6-(2,4-dichloro- | 75 | 4.35 |
| | phenylamino)-N-cyclohexylmethyl- | | 434 |
| | nicotinamide | | C₂₃H₂₉³⁵Cl₂N₃O |
| 529 | 4-tert-Butyl-6-(3-chloro- | 30 | 3.40 |
| | phenylamino)-N-(tetrahydro-pyran- | | 402 |
| | 4-ylmethyl)-nicotinamide | | C₂₂H₂₈³⁵ClN₃O₂ |
| 530 | 4-tert-Butyl-6-(3-fluoro- | 30 | 3.21 |
| | phenylamino)-N-(tetrahydro-pyran- | | 386 |
| | 4-ylmethyl)-nicotinamide | | C₂₂H₂₈FN₃O₂ |
| 531 | 4-tert-Butyl-6-(2-chloro-3- | 30 | 3.40 |
| | fluorophenylamino)-N-(tetrahydro- | | 420 |
| | pyran-4-ylmethyl)-nicotinamide | | C₂₂H₂₇³⁵ClFN₃O₂ |
| 532 | 4-tert-Butyl-6-(2,4-di-chloro- | 60 | 3.40 |
| | phenylamino)-N-(tetrahydro-pyran- | | 436 |
| | 4-ylmethyl)-nicotinamide | | C₂₂H₂₇³⁵Cl₂N₃O₂ |

### EXAMPLE 533

### 6-(3,5-Dichloro-phenylamino)-4-isopropyl-N-(tetrahydro-pyran-4-ylmethyl)-nicotinamide

A mixture of 6-chloro-4-isopropyl-N-(tetrahydro-pyran-4-ylmethyl)-nicotinamide (Description 24) (100 mg), 3,5-dichloroaniline (ex-Aldrich, 109 mg), methanesulfonic acid (44 μl) in 1,4-dioxane (1 ml) was irradiated under microwave conditions at 180° C. for 30 minutes, The crude mixture was purified using MDAP to afford 6-(3,5-dichloro-phenylamino)-4-isopropyl-N-(tetrahydro-pyran-4-ylmethyl)-nicotinamide (50 mg)

NMR (CDCl₃) δ1.21-1.29 (6H, m), 1.35-1.48 (2H, m), 1.35-1.49 (2H, m), 1.71 (2H, d), 1.86-1.99 (1H, m), 3.34-3.49 (4H, m), 3.50-3.61 (1H, m), 4.03 (2H, d), 6.10 (1H, bs), 6.75 (1H, bs), 7.08 (1H, bs), 7.10-7.16 (1H, m), 7.41-7.45 (2H, m), 8.26 (1H, s)

TABLE 24

| | | | | RT (min), (MH⁺), |
|---|---|---|---|---|
| Ex. | | | Purification | Consistent with the |
| No | Compound Name | Method | method | molecular formula |
| 534 | 6-(5-Chloro-2-fluoro- | G | E | 3.13 |
| | phenylamino)-4-isopropyl- | | | 406 |
| | N-(tetrahydro-pyran-4- | | | C₂₁H₂₅³⁵ClFN₃O₂ |
| | ylmethyl)-nicotinamide | | | |
| 535 | 6-(3-Chloro-4-fluoro- | G | E | 3.13 |
| | phenylamino)-4-isopropyl- | | | 406 |
| | N-(tetrahydro-pyran-4- | | | C₂₁H₂₅³⁵ClFN₃O₂ |
| | ylmethyl)-nicotinamide | | | |
| 536 | 6-(3-Chloro-4- | G | E | 3.62 |
| | trifluoromethoxy- | | | 472 |
| | phenylamino)-4-isopropyl- | | | C₂₂H₂₅³⁵ClF₃N₃O₃ |
| | N-(tetrahydro-pyran-4- | | | |
| | ylmethyl)-nicotinamide | | | |
| 537 | 6-(3-Chloro-4-cyano- | G | E | 3.10 |
| | phenylamino)-4-isopropyl- | | | 413 |
| | N-(tetrahydro-pyran-4- | | | C₂₂H₂₅³⁵ClN₄O₂ |
| | ylmethyl)-nicotinamide | | | |
| 538 | 6-(3-Fluoro-5- | G | E | 3.20 |
| | trifluoromethyl- | | | 440 |
| | phenylamino)-4-isopropyl- | | | C₂₂H₂₅F₄N₃O₂ |
| | N-(tetrahydro-pyran-4- | | | |
| | ylmethyl)-nicotinamide | | | |
| 539 | 6-(2-Fluoro-3- | G | E | 3.40 |
| | trifluoromethyl- | | | 440 |
| | phenylamino)-4-isopropyl- | | | C₂₂H₂₅F₄N₃O₂ |
| | N-(tetrahydro-pyran-4- | | | |
| | ylmethyl)-nicotinamide | | | |
| 540 | 6-(4-Bromo-2-chloro- | G | E | 3.41 |
| | phenylamino)-4-isopropyl- | | | 468 |
| | N-(tetrahydro-pyran-4- | | | C₂₁H₂₅⁷⁹Br³⁵ClN₃O₂ |
| | ylmethyl)-nicotinamide | | | |
| 541 | 6-(2-Bromo-4-chloro- | G | E | 3.39 |
| | phenylamino)-4-isopropyl- | | | 468 |
| | N-(tetrahydro-pyran-4- | | | C₂₁H₂₅⁷⁹Br³⁵ClN₃O₂ |
| | ylmethyl)-nicotinamide | | | |
| 542 | 4-Isopropyl-6-(2-methyl-3- | G | E | 3.09 |
| | trifluoromethyl- | | | 436 |
| | phenylamino)-N- | | | C₂₃H₂₈F₃N₃O₂ |
| | (tetrahydro-pyran-4- | | | |
| | ylmethyl)-nicotinamide | | | |
| 543 | 6-(3-chloro-4-methyl- | G | H | 3.24 |
| | phenylamino)-4-isopropyl- | | | 402 |
| | N-(tetrahydro-pyran-4- | | | C₂₂H₂₈³⁵ClN₃O₂ |
| | ylmethyl)-nicotinamide | | | |
| 544 | 6-(4-Bromo-3-methyl- | G | A | 2.48 |
| | phenylamino)-4-isopropyl- | | | 446 |
| | N-(tetrahydro-pyran-4- | | | C₂₂H₂₈⁷⁹BrN₃O₂ |
| | ylmethyl)-nicotinamide | | | |
| 545 | 6-(2,5-Dichloro- | G | E | 3.28 |
| | phenylamino)-4-isopropyl- | NB | | 422 |
| | N-(tetrahydro-pyran-4- | Irradiation | | C₂₁H₂₅³⁵Cl₂N₃O₂ |
| | ylmethyl)-nicotinamide | time | | |
| | | was 60 min. | | |
| 546 | 4-Isopropyl-6-(2-methyl-5- | G | E | 3.23 |
| | trifluoromethyl- | | | 436 |
| | phenylamino)-N- | | | C₂₃H₂₈F₃N₃O₂ |
| | (tetrahydro-pyran-4- | | | |
| | ylmethyl)-nicotinamide | | | |
| 547 | 6-(2-Bromo-4-chloro- | G | E | 3.97 |
| | phenylamino)-N- | | | 452 |
| | cyclopentylmethyl-4- | | | C₂₁H₂₅⁷⁹Br³⁵ClN₃O |
| | isopropyl-nicotinamide | | | |
| 548 | 6-(4-Bromo-3-chloro- | G | H | 3.48 |
| | phenylamino)-4-isopropyl- | | | 466 |
| | N-(tetrahydro-pyran-4- | | | C₂₁H₂₅⁷⁹Br³⁵ClN₃O₂ |
| | ylmethyl)-nicotinamide | | | |
| 549 | 6-(4-Chloro-2-fluoro- | G | E | 3.7 |
| | phenylamino)-N- | | | 390 |
| | cyclopentylmethyl-4- | | | C₂₁H₂₅³⁵ClFN₃O |
| | isopropyl-nicotinamide | | | |
| 550 | N-Cyclopentylmethyl-6-(3- | G | H | 3.8 |
| | fluoro-4-trifluoromethyl- | | | 424 |
| | phenylamino)-4-isopropyl- | | | C₂₂H₂₅F₄N₃O |
| | nicotinamide | | | |
| 551 | 6-(4-Cyano-2-methyl- | B | H | 3.43 |
| | phenylamino)-N- | | | 377 |
| | cyclopentylmethyl-4- | | | C₂₃H₂₈N₄O |
| | isopropyl-nicotinamide | | | |

Preparative Method B As for the preparation of Example 461
Preparative Method G As for the preparation of Example 533
Purification Method A: Purify by trituration as for Example 460.
Purification Method E: Purify by mass-directed autopreparative technique.
Purification Method H: Purify using the Biotage Horizon system detailed at the beginning of the experimental section.

TABLE 25

| | | | RT (min), (MH+), |
|---|---|---|---|
| Ex. | | Purification | Consistent with the |
| No. | Compound Name | method | molecular formula |
| 552 | 6-(3-Chloro-2-fluoro-phenylamino)-4- | E | 3.05 |
| | isopropyl-N-(tetrahydro-pyran-4- | | 406 |
| | ylmethyl)-nicotinamide | | C₂₁H₂₅³⁵ClFN₃O₂ |
| 553 | 6-(3-Fluoro-4-trifluoromethyl- | E | 3.40 |
| | phenylamino)-4-isopropyl-N-(tetrahydro- | | 440 |
| | pyran-4-ylmethyl)-nicotinamide | | C₂₂H₂₅F₄N₃O₂ |
| 554 | 6-(4-Cyano-3-trifluoromethyl- | E | 3.29 |
| | phenylamino)-4-isopropyl-N-(tetrahydro- | | 447 |
| | pyran-4-ylmethyl)-nicotinamide | | C₂₃H₂₅F₃N₄O₂ |
| 555 | 6-(4-Cyano-2-fluoro-phenylamino)-4- | E | 2.92 |
| | isopropyl-N-(tetrahydro-pyran-4- | | 397 |
| | ylmethyl)-nicotinamide | | C₂₂H₂₅FN₄O₂ |
| 556 | 6-(4-fluoro-3-methyl-phenylamino)-4- | H | 2.83 |
| | isopropyl-N-(tetrahydro-pyran-4- | | 386 |
| | ylmethyl)-nicotinamide | | C₂₂H₂₈FN₃O₂ |
| 557 | 6-(5-Chloro-2-methyl-phenylamino)-4- | E | 3.02 |
| | isopropyl-N-(tetrahydro-pyran-4- | | 402 |
| | ylmethyl)-nicotinamide | | C₂₂H₂₈³⁵ClN₃O₂ |
| 558 | 6-(3-Fluoro-4-methyl-phenylamino)-4- | H | 3.03 |
| | isopropyl-N-(tetrahydro-pyran-4- | | 386 |
| | ylmethyl)-nicotinamide | | C₂₂H₂₈FN₃O₂ |
| 559 | 6-(3,4-Dimethyl-phenylamino)-4- | H | 2.85 |
| | isopropyl-N-(tetrahydro-pyran-4- | | 382 |
| | ylmethyl)-nicotinamide | | C₂₃H₃₁N₃O₂ |
| 560 | 6-(3-Bromo-4-methyl-phenylamino)-4- | H | 3.32 |
| | isopropyl-N-(tetrahydro-pyran-4- | | 446 |
| | ylmethyl)-nicotinamide | | C₂₂H₂₈⁷⁹BrN₃O₂ |

All compounds in table 25 were prepared as for Example 533 and purified by the technique given in the table.
Purification Method E: Purify by mass-directed autopreparative technique.
Purification Method H: Purify using the Biotage Horizon system detailed at the beginning of the experimental section.

### EXAMPLE 561

### 6-(3-Chloro-phenylamino)-N-(4-hydroxy-tetrahydro-pyran-4-ylmethyl)-4-isopropyl-nicotinamide

This was prepared by the same method used to prepare Example 508 from Description 30.

LC/MS t=2.89 min, [MH⁺] 404 C₂₁H₂₆³⁵ClN₃O₃

### EXAMPLE 562

### 6-(2,3-Dichloro-phenylamino)-N-(cyclobutyl)-4-trifluoromethyl-nicotinamide

N-methylmorpholine (48 uL, 0.43 mmol), cyclobutylamine (13 mg, 0.18 mmol), 1-hydroxybenzotriazole (30 mg, 0.22 mmol), 1-(3-dimethylamino-propyl)-3-ethylcarbodiimide hydrochloride (32 mg, 0.17 mmol) were added to a solution of 6-(2,3-dichloro-phenylamino)-4-trifluoromethyl nicotinic acid (Description 10) (50 mg, 0.14 mmol) in dimethylformamide (3 mL). After stirring at room temperature for 6 h, dimethylformamide was evaporated under reduced pressure and dichloromethane was added. The solution was washed with an aqueous solution of NaHCO₃ 5% (5 mL), with water (10 mL), then with brine (2×3 mL) and was evaporated under reduced pressure. The crude residue was triturated with diethyl ether, filtered and dried under vacuum to afford the title compound (46 mg, yield=81%).

¹H NMR (300 MHz, DMSO-d₆) δ: 9.27 (s br, 1H); 8.66 (d br, 1H); 8.27 (s, 1H); 7.90 (dd, 1H); 7.42-7.31 (m, 3H); 4.30 (m, 1H); 2.21 (m, 2H); 1.97 (m, 2H); 1.66 (m, 2H).

MS m/z (EI+); TSQ 700; source 180° C.; 70 V; 200 uA: 403 (M^{+.}), 375, 332.

### EXAMPLE 563

### 6-(2,4-Dichloro-phenylamino)-N-(tetrahydropyran-4-ylmethyl)-4-trifluoromethyl-nicotinamide

1-Hydroxy-7-azabenzotriazole (33 mg, 0.24 mmol), tetrahydropyran-4-ylmethylamine (17 mg, 0.14 mmol) and PS-carbodiimide (218 mg, 0.28 mmol, loading 1.31 mmol/g, ex Argonaut Technologies) were added to a solution of 6-(2,4-dichloro-phenylamino)-4-trifluoromethyl nicotinic acid (Description 12) (75 mg, 0.21 mmol) in 3 mL of dichloromethane. After orbital shaking at room temperature overnight, the resin was filtered and washed repeatedly with dichloromethane; the filtrate was treated with an aqueous solution of NaHC O₃ 5%. The organic layer was separated through Phase Separator cartridge, dried over sodium sulphate and evaporated in vacuo. The solid residue was triturated with acetonitrile, filtered and dried under vacuum to afford the title compound (44 mg, yield=46%).

¹H NMR (300 MHz, DMSO-d6) δ: 9.18 (s, 1H); 8.48 (t br, 1H); 8.27 (s, 1H); 7.98 (d, 1H); 7.66 (d, 1H); 7.42 (dd, 1H); 7.37 (s, 1H); 3.84 (dd, 2H); 3.26 (dd, 2H); 3.10 (dd, 110; 1.74 (m, 1H); 1.60 (d br, 2H); 1.18 (m, 2H).

MS m/z (EI+); TSQ 700; source 180° C.; 70 V; 200 uA: 447 (M^{+.}), 412, 333, 314.

### EXAMPLE 564

### 6-(3-Chloro-phenylamino)-N-(1,1-dioxo-tetrahydrothiophene-3-ylmethyl)-4-trifluoromethyl-nicotinamide

PS-carbodiimide (1.6 g, 2 mmol, loading 1.31 mmol/g, ex Argonaut Technologies) and 1-hydroxy-benzotriazole (0.2 g, 1.5 mmol) were added to a solution of 6-(3-chlorophenylamino)-4-trifluoromethyl nicotinic acid (Description 7) (0.35 g, 1 mmol) in dry dichloromethane (15 mL) and the mixture was stirred at room temperature overnight. The resin was filtered and washed repeatedly with dichloromethane, the solvent was then removed under reduced pressure. The solid residue was dissolved in anhydrous tetrahydrofuran (3.5 mL) and PS-diisopropylethylamine (300 mg, 1.16 mmol, loading 3.88 mmol/g, ex Argonaut Technologies), (1,1-dioxo-tetrahydrothiophen-3-yl)methylamine (0.185 g, 1 mmol) and 1-butyl-3-methylimidazolium hexafluorophosphate (72 uL, 0.35 mmol) were added. The mixture was heated in a sealed tube under microwaves irradiation for 40 min at 140° C. (power=25-30W), then the resin was filtered and washed with THF (15 mL) and dichloromethane (15 mL) and the filtrate was evaporated under reduced pressure. The residue was dissolved in dichloromethane, washed with an aqueous solution of K₂CO₃ 10%, dried over magnesium sulphate and evaporated under reduced pressure. Purification by flash chromatography on silica gel (initial eluent: DCM, final eluent: DCM/MeOH 98:2) yielded the title compound (210 mg, yield=47%).

¹H NMR (300 MHz, CDCl₃) δ: 8.41 (s, 1H); 8.38 (s, 1H); 7.73 (dd, 1H); 7_{—}37 (d br, 1H); 7.36 (t br, 1H); 7.21 (dd, 1H); 7.04 (s, 1H); 6.98 (d br, 1H); 3.60-3.39 (m, 2H); 3.24-3.12 (m, 2H); 3.02 (ddd, 1H); 2.90-2.70 (m, 2H); 2.38-2.26 (m, 1H); 2.09-1.87 (m, 1H).

MS m/z (EI+); TSQ 700; source 180° C.; 70 V; 200 uA: 447 (M^{+.}); 299; 236.

TABLE 26

| Ex.. No | Compound name | Method | ¹H NMR (solvent) ppm and/or MS |
|---|---|---|---|
| 565 | N-Cyclohexylmethyl-6- | B | ESI Pos: AQA; Spray 3.5 kV; |
| | phenylamino-4- | | Skimmer 30 V; Probe 250° C.: |
| | trifluoromethyl- | | 378 (MH+). |
| | nicotinamide | | |
| 566 | N-Cyclopentylmethyl-6- | B | ESI Pos: AQA; Spray 3.5 kV; |
| | phenylamino-4- | | Skimmer 30 V; Probe 250° C.: |
| | trifluoromethyl- | | 364 (MH+). |
| | nicotinamide | | |
| 567 | N-Cyclobutylmethyl-6- | B | ESI Pos: AQA; Spray 3.5 kV; |
| | phenylamino-4- | | Skimmer 30 V; Probe 250° C.: |
| | trifluoromethyl- | | 350 (MH+). |
| | nicotinamide | | |
| 568 | N-Cyclobutyl-6-(3-chloro- | B | ¹H NMR (300 MHz, DMSO-d₆) δ: |
| | phenylamino)-4- | | 9.87 (s, 1H); 8.66 (d br, 1H); |
| | trifluoromethyl- | | 8.40 (s, 1H); 8.01 (dd, 1H); 7.49 (dd, |
| | nicotinamide | | 1H); 7.34 (dd, 1H); 7.16 (s, 1H); |
| | | | 7.02 (dd, 1H); 4.31 (m, 1H); |
| | | | 2.22 (m, 2H); 1.99 (m, 2H); 1.67 (m, |
| | | | 2H). |
| | | | ESI Pos: AQA; Spray 3 kV; Source |
| | | | 20 V; Probe 250° C.: 370 (MH+). |
| 569 | N-(Tetrahydropyran-4- | B | ESI Pos: AQA; Spray 3.5 kV; |
| | ylmethyl)-6-(3-chloro- | | Skimmer 30 V; Probe 250° C.: |
| | phenylamino)-4- | | 414 (MH+). |
| | trifluoromethyl- | | |
| | nicotinamide | | |
| 570 | N-Cyclobutylmethyl-6-(3- | B | ESI Pos: AQA; Spray 3.5 kV; |
| | chloro-phenylamino)-4- | | Skimmer 30 V; Probe 250° C.: |
| | trifluoromethyl- | | 383 (MH+). |
| | nicotinamide | | |
| 571 | N-Isobutyl-6-(3-chloro- | B | ESI Pos: AQA; Spray 3.5 kV; |
| | phenylamino)-4- | | Skimmer 30 V; Probe 250° C.: |
| | trifluoromethyl- | | 372 (MH+). |
| | nicotinamide | | |
| 572 | N-Cyclopentylmethyl-6- | B | ESI Pos: AQA; Spray 3.5 kV; |
| | (3-chloro-phenylamino)-4- | | Skimmer 30 V; Probe 250° C.: |
| | trifluoromethyl- | | 398 (MH+). |
| | nicotinamide | | |
| 573 | N-Cyclopropylmethyl-6- | B | ESI Pos: AQA; Spray 3.5 kV; |
| | (3-chloro-phenylamino)-4- | | Skimmer 30 V; Probe 250° C.: |
| | trifluoromethyl- | | 370 (MH+). |
| | nicotinamide | | |
| 574 | N-Cyclohexylmethyl-6-(3- | B | ESI Pos: AQA; Spray 3.5 kV; |
| | bromo-phenylamino)-4- | | Skimmer 30 V; Probe 250° C.: |
| | trifluoromethyl- | | 456 (MH+). |
| | nicotinamide | | |
| 575 | N-Cycloheptylmethyl-6- | B | ESI Pos: AQA; Spray 3.5 kV; |
| | (3-bromo-phenylamino)-4- | | Skimmer 30 V; Probe 250° C.: |
| | trifluoromethyl- | | 470 (MH+). |
| | nicotinamide | | |
| 576 | N-(Tetrahydropyran-4- | B | ESI Pos: AQA; Spray 3.5 kV; |
| | ylmethyl)-6-(3-bromo- | | Skimmer 30 V; Probe 250° C.: |
| | phenylamino)-4- | | 458 (MH+). |
| | trifluoromethyl- | | |
| | nicotinamide | | |
| 577 | N-Cyclobutyl-6-(3-bromo- | B | ESI Pos: AQA; Spray 3.5 kV; |
| | phenylamino)-4- | | Skimmer 30 V; Probe 250° C.: |
| | trifluoromethyl- | | 414 (MH+). |
| | nicotinamide | | |
| 578 | N-Cyclobutylmethyl-6-(3- | B | ESI Pos: AQA; Spray 3.5 kV; |
| | bromo-phenylamino)-4- | | Skimmer 30 V; Probe 250° C.: |
| | trifluoromethyl- | | 427 (MH+). |
| | nicotinamide | | |
| 579 | N-Isobutyl-6-(3-bromo- | B | ESI Pos: AQA; Spray 3.5 kV; |
| | phenylamino)-4- | | Skimmer 30 V; Probe 250° C.: |
| | trifluoromethyl- | | 415 (MH+). |
| | nicotinamide | | |
| 580 | N-Cyclopentylmethyl-6- | B | ESI Pos: AQA; Spray 3.5 kV; |
| | (3-bromo-phenylamino)-4- | | Skimmer 30 V; Probe 250° C.: |
| | trifluoromethyl- | | 442 (MH+). |
| | nicotinamide | | |
| 581 | N-Cyclopropylmethyl-6- | B | ESI Pos: AQA; Spray 3.5 kV; |
| | (3-bromo-phenylamino)-4- | | Skimmer 30 V; Probe 250° C.: |
| | trifluoromethyl- | | 414 (MH+). |
| | nicotinamide | | |
| 582 | N-Cyclobutylmethyl-6-(2- | B | ESI Pos: AQA; Spray 3.5 kV; |
| | fluoro-phenylamino)-4- | | Skimmer 30 V; Probe 250° C.: |
| | trifluoromethyl- | | 368 (MH+). |
| | nicotinamide | | |
| 583 | N-Cycloheptylmethyl-6- | B | ESI Pos: AQA; Spray 3.5 kV; |
| | (3-fluoro-phenylamino)-4- | | Skimmer 30 V; Probe 250° C.: |
| | trifluoromethyl- | | 410 (MH+). |
| | nicotinamide | | |
| 584 | N-(Tetrahydropyran-4- | B | ESI Pos: AQA; Spray 3.5 kV; |
| | ylmethyl)-6-(3-fluoro- | | Skimmer 30 V; Probe 250° C.: |
| | phenylamino)-4- | | 398 (MH+). |
| | trifluoromethyl- | | |
| | nicotinamide | | |
| 585 | N-Cyclobutyl-6-(3-fluoro- | B | ESI Pos: AQA; Spray 3.5 kV; |
| | phenylamino)-4- | | Skimmer 30 V; Probe 250° C.: |
| | trifluoromethyl- | | 354 (MH+). |
| | nicotinamide | | |
| 586 | N-Cyclohexylmethyl-6-(3- | B | ESI Pos: AQA; Spray 3.5 kV; |
| | fluoro-phenylamino)-4- | | Skimmer 30 V; Probe 250° C.: |
| | trifluoromethyl- | | 396 (MH+). |
| | nicotinamide | | |
| 587 | N-Cyclobutylmethyl-6-(3- | B | ESI Pos: AQA; Spray 3.5 kV; |
| | fluoro-phenylamino)-4- | | Skimmer 30 V; Probe 250° C.: |
| | trifluoromethyl- | | 367 (MH+). |
| | nicotinamide | | |
| 588 | N-Cyclopentylmethyl-6- | B | ESI Pos: AQA; Spray 3.5 kV; |
| | (3-fluoro-phenylamino)-4- | | Skimmer 30 V; Probe 250° C.: |
| | trifluoromethyl- | | 382 (MH+). |
| | nicotinamide | | |
| 589 | N-Isobutyl-6-(3-fluoro- | B | ESI Pos: AQA; Spray 3.5 kV; |
| | phenylamino)-4- | | Skimmer 30 V; Probe 250° C.: |
| | trifluoromethyl- | | 356 (MH+). |
| | nicotinamide | | |
| 590 | N-Cyclopropylmethyl-6- | B | ESI Pos: AQA; Spray 3.5 kV; |
| | (3-fluoro-phenylamino)-4- | | Skimmer 30 V; Probe 250° C.: |
| | trifluoromethyl- | | 354 (MH+). |
| | nicotinamide | | |
| 591 | N-(1,1-Dioxo-tetrahydro- | C | ESI Pos: AQA; Spray 3.5 kV; |
| | thiophen-3-ylmethyl)-6- | | Skimmer 30 V; Probe 250° C.: |
| | (3-fluoro-phenylamino)-4- | | 432 (MH+). |
| | trifluoromethyl- | | |
| | nicotinamide | | |
| 592 | N-Cyclobutylmethyl-6-(4- | B | ESI Pos: AQA; Spray 3.5 kV; |
| | fluoro-phenylamino)-4- | | Skimmer 30 V; Probe 250° C.: |
| | trifluoromethyl- | | 368 (MH+). |
| | nicotinamide | | |
| 593 | N-(Tetrahydropyran-4- | B | ESI Pos: AQA; Spray 3.5 kV; |
| | ylmethyl)-6-(2,3-dichloro- | | Skimmer 30 V; Probe 250° C.: |
| | phenylamino)-4- | | 448 (MH+). |
| | trifluoromethyl- | | |
| | nicotinamide | | |
| 594 | N-Cyclohexylmethyl-6- | B | ESI Pos: AQA; Spray 3.5 kV; |
| | (2,3-dichloro- | | Skimmer 30 V; Probe 250° C.: |
| | phenylamino)-4- | | 446 (MH+). |
| | trifluoromethyl- | | |
| | nicotinamide | | |
| 595 | N-Cycloheptylmethyl-6- | B | ESI Pos: AQA; Spray 3.5 kV; |
| | (2,3-dichloro- | | Skimmer 30 V; Probe 250° C.: |
| | phenylamino)-4- | | 460 (MH+). |
| | trifluoromethyl- | | |
| | nicotinamide | | |
| 596 | N-Cyclohexylmethyl-6- | B | ESI Pos: AQA; Spray 3.5 kV; |
| | (2,4-dichloro- | | Skimmer 30 V; Probe 250° C.: |
| | phenylamino)-4- | | 447 (MH+). |
| | trifluoromethyl- | | |
| | nicotinamide | | |
| 597 | N-Cycloheptylmethyl-6- | B | ESI Pos: AQA; Spray 3.5 kV; |
| | (2,4-dichloro- | | Skimmer 30 V; Probe 250° C.: |
| | phenylamino)-4- | | 460 (MH+). |
| | trifluoromethyl- | | |
| | nicotinamide | | |
| 598 | N-Cyclobutyl-6-(2,4- | B | ESI Pos: AQA; Spray 3.5 kV; |
| | dichloro-phenylamino)-4- | | Skimmer 30 V; Probe 250° C.: |
| | trifluoromethyl- | | 404 (MH+). |
| | nicotinamide | | |
| 599 | N-Cyclopentylmethyl-6- | B | ESI Pos: AQA; Spray 3.5 kV; |
| | (2,4-dichloro- | | Skimmer 30 V; Probe 250° C.: |
| | phenylamino)-4- | | 432 (MH+). |
| | trifluoromethyl- | | |
| | nicotinamide | | |
| 600 | N-Cyclobutylmethyl-6- | B | ESI Pos: AQA; Spray 3.5 kV; |
| | (2,4-dichloro- | | Skimmer 30 V; Probe 250° C.: |
| | phenylamino)-4- | | 418 (MH+). |
| | trifluoromethyl- | | |
| | nicotinamide | | |
| 601 | N-Isobutyl-6-(2,4- | B | ESI Pos: AQA; Spray 3.5 kV; |
| | dichloro-phenylamino)-4- | | Skimmer 30 V; Probe 250° C.: |
| | trifluoromethyl- | | 406 (MH+). |
| | nicotinamide | | |
| 602 | N-Cyclopropylmethyl-6- | B | ESI Pos: AQA; Spray 3.5 kV; |
| | (2,4-dichloro- | | Skimmer 30 V; Probe 250° C.: |
| | phenylamino)-4- | | 404 (MH+). |
| | trifluoromethyl- | | |
| | nicotinamide | | |
| 603 | N-(1,1-Dioxo-tetrahydro- | C | ¹H NMR (300 MHz, CDCl₃) δ: |
| | thiophen-3-ylmethyl)-6- | | 8.38 (s, 1H); 8.08 (d, 1H); 7.47 (s, 1H); |
| | (2,4-dichloro- | | 7.41 (t br, 1H); 7.40 (d, 1H); |
| | phenylamino)-4- | | 7.23 (dd, 1H); 7.04 (s, 1H); |
| | trifluoromethyl- | | 3.60-3.39 (m, 2H); 3.24-3.12 (m, 2H); |
| | nicotinamide | | 3.01 (ddd, 1H); 2.90-2.72 (m, 2H); |
| | | | 2.38-2.26 (m, 1H); 2.09-1.87 (m, 1H). |
| | | | EI+; TSQ 700; source 180° C.; 70 V; |
| | | | 200 uA: 481 (M^{+.}); 446; 333; 270. |
| 604 | N-Cyclohexylmethyl-6- | B | ESI Pos: AQA; Spray 3.5 kV; |
| | (3,5-dichloro- | | Skimmer 30 V; Probe 250° C.: |
| | phenylamino)-4- | | 446 (MH+). |
| | trifluoromethyl- | | |
| | nicotinamide | | |
| 605 | N-(Tetrahydropyran-4- | B | ESI Pos: AQA; Spray 3.5 kV; |
| | ylmethyl)-6-(3,5-dichloro- | | Skimmer 30 V; Probe 250° C.: |
| | phenylamino)-4- | | 448 (MH+). |
| | trifluoromethyl- | | |
| | nicotinamide | | |
| 606 | N-Cyclobutyl-6-(3,5- | B | ESI Pos: AQA; Spray 3.5 kV; |
| | dichloro-phenylamino)-4- | | Skimmer 30 V; Probe 250° C.: |
| | trifluoromethyl- | | 404 (MH+). |
| | nicotinamide | | |
| 607 | N-Cyclopentylmethyl-6- | B | ESI Pos: AQA; Spray 3.5 kV; |
| | (3,5-dichloro- | | Skimmer 30 V; Probe 250° C.: |
| | phenylamino)-4- | | 432 (MH+). |
| | trifluoromethyl- | | |
| | nicotinamide | | |
| 608 | N-Cyclobutylmethyl-6- | B | ESI Pos: AQA; Spray 3.5 kV; |
| | (3,5-dichloro- | | Skimmer 30 V; Probe 250° C.: |
| | phenylamino)-4- | | 418 (MH+). |
| | trifluoromethyl- | | |
| | nicotinamide | | |
| 609 | N-Isobutyl-6-(3,5- | B | ESI Pos: AQA; Spray 3.5 kV; |
| | dichloro-phenylamino)-4- | | Skimmer 30 V; Probe 250° C.: |
| | trifluoromethyl- | | 406 (MH+). |
| | nicotinamide | | |
| 610 | N-Cyclopropylmethyl-6- | B | ESI Pos: AQA; Spray 3.5 kV; |
| | (3,5-dichloro- | | Skimmer 30 V; Probe 250° C.: |
| | phenylamino)-4- | | 404 (MH+). |
| | trifluoromethyl- | | |
| | nicotinamide | | |
| 611 | N-Isobutyl-6-(3,4- | B | ¹H NMR (300 MHz, DMSO-d₆) δ: |
| | dichloro-phenylamino)-4- | | 9.98 (s br, 1H); 8.47 (t br, 1H); |
| | trifluoromethyl- | | 8.41 (s, 1H); 8.20 (s, 1H); 7.55 (s, 2H); |
| | nicotinamide | | 7.17 (s, 1H); 3.05 (dd, 2H); |
| | | | 1.80 (m, 1H); 0.90 (d, 6H). |
| | | | ESI Pos: AQA; Spray 3 kV; Source |
| | | | 20 V; Probe 250° C.: 406 (MH+). |
| 612 | N-Cyclobutyl-6-(3,4- | B | ESI Pos: AQA; Spray 3.5 kV; |
| | dichloro-phenylamino)-4- | | Skimmer 30 V; Probe 250° C.: |
| | trifluoromethyl- | | 404 (MH+). |
| | nicotinamide | | |
| 613 | N-(Tetrahydropyran-4- | B | ESI Pos: AQA; Spray 3.5 kV; |
| | ylmethyl)-6-(3,4-dichloro- | | Skimmer 30 V; Probe 250° C.: |
| | phenylamino)-4- | | 448 (MH+). |
| | trifluoromethyl- | | |
| | nicotinamide | | |
| 614 | N-Cyclopentylmethyl-6- | B | ESI Pos: AQA; Spray 3.5 kV; |
| | (3,4-dichloro- | | Skimmer 30 V; Probe 250° C.: |
| | phenylamino)-4- | | 432 (MH+). |
| | trifluoromethyl- | | |
| | nicotinamide | | |
| 615 | N-Cyclobutylmethyl-6- | B | ESI Pos: AQA; Spray 3.5 kV; |
| | (3,4-dichloro- | | Skimmer 30 V; Probe 250° C.: |
| | phenylamino)-4- | | 418 (MH+). |
| | trifluoromethyl- | | |
| | nicotinamide | | |
| 616 | N-Cyclopropylmethyl-6- | B | ESI Pos: AQA; Spray 3.5 kV; |
| | (3,4-dichloro- | | Skimmer 30 V; Probe 250° C.: |
| | phenylamino)-4- | | 404 (MH+). |
| | trifluoromethyl- | | |
| | nicotinamide | | |
| 617 | N-Cyclohexylmethyl-6- | B | ESI Pos: AQA; Spray 3.5 kV; |
| | (3,4-dichloro- | | Skimmer 30 V; Probe 250° C.: |
| | phenylamino)-4- | | 446 (MH+). |
| | trifluoromethyl- | | |
| | nicotinamide | | |
| 618 | N-Cyclobutylmethyl-6-(2- | A | ¹H NMR (300 MHz, DMSO-d₆) δ: |
| | fluoro-4-chloro- | | 9.42 (s, 1H); 8.42 (t br, 1H); 8.28 (s, |
| | phenylamino)-4- | | 1H); 8.17 (dd, 1H); 7.48 (dd, 1H); |
| | trifluoromethyl- | | 7.35 (s, 1H); 7.27 (d br, 1H); |
| | nicotinamide | | 3.23 (dd, 2H); 2.48 (m, 1H); |
| | | | 2.04-1.91 (m, 2H); 1.89-1.64 (m, 4H). |
| | | | EI+; TSQ 700; source 180° C.; 70 V; |
| | | | 200 uA: 401 (M^{+.}) 366, 333, 317. |
| 619 | N-Cyclopentylmethyl-6- | A | ¹H NMR (300 MHz, DMSO-d₆) δ: |
| | (2-fluoro-4-chloro- | | 9.42 (s, 1H); 8.47 (t br, 1H); 8.29 (s, |
| | phenylamino)-4- | | 1H); 8.17 (dd, 1H); 7.48 (dd, 1H); |
| | trifluoromethyl- | | 7.35 (s, 1H); 7.27 (d br, 1H); |
| | nicotinamide | | 3.14 (dd, 2H); 2.08 (m, 1H); |
| | | | 1.75-1.42 (m, 6H); 1.29-1.15 (m, 2H). |
| | | | EI+; TSQ 700; source 180° C.; 70 V; |
| | | | 200 uA: 415 (M^{+.}) 346, 333, 317. |
| 620 | N-(Tetrahydropyran-4- | A | ¹H NMR (300 MHz, DMSO-d₆) δ: |
| | ylmethyl)-6-(2-fluoro-4- | | 9.44 (s, 1H); 8.50 (t br, 1H); 8.32 (s, |
| | chloro-phenylamino)-4- | | 1H); 8.17 (dd, 1H); 7.48 (dd, 1H); |
| | trifluoromethyl- | | 7.36 (s, 1H); 7.29 (d br, 1H); |
| | nicotinamide | | 3.84 (dd, 2H); 3.26 (dd, 2H); 3.11 (dd, |
| | | | 2H); 1.74 (m, 1H); 1.60 (m, 2H); |
| | | | 1.19 (m, 2H). |
| | | | EI+; TSQ 700; source 180° C.; 70 V; |
| | | | 200 uA: 431 (M^{+.}); 346; 333; 317. |
| 621 | N-Cyclobutylmethyl-6-(2- | A | ¹H NMR (300 MHz, DMSO-d₆) δ: |
| | chloro-4-fluoro- | | 9.13 (s, 1H); 8.39 (t br, 1H); 8.19 (s, |
| | phenylamino)-4- | | 1H); 7.80 (dd, 1H); 7.51 (dd, 1H); |
| | trifluoromethyl- | | 7.24 (dt, 1H); 7.20 (s, 1H); |
| | nicotinamide | | 3.22 (dd, 2H); 2.55-2.42 (m, 1H); |
| | | | 2.04-1.63 (m, 6H). |
| | | | EI+; TSQ 700; source 180° C.; 70 V; |
| | | | 200 uA: 401 (M^{+.}); 366; 317; 298; |
| | | | 254. |
| 622 | N-Cyclopentylmethyl-6- | A | ¹H NMR (300 MHz, DMSO-d₆) δ: |
| | (2-chloro-4-fluoro- | | 9.13 (s, 1H); 8.42 (t br, 1H); 8.20 (s, |
| | phenylamino)-4- | | 1H); 7.81 (dd, 1H); 7.52 (dd, 1H); |
| | trifluoromethyl- | | 7.24 (dt, 1H); 7.20 (s, 1H); |
| | nicotinamide | | 3.13 (dd, 2H); 2.07 (m, 1H); |
| | | | 1.75-1.42 (m, 6H); 1.30-1.15 (m, 2H). |
| | | | EI+; TSQ 700; source 180° C.; 70 V; |
| | | | 200 uA: 415 (M^{+.}); 380; 346; 317; |
| | | | 298; 254. |
| 623 | N-(Tetrahydropyran-4- | A | ¹H NMR (300 MHz, DMSO-d₆) δ: |
| | ylmethyl)-6-(2-chloro-4- | | 9.14 (s, 1H); 8.45 (t br, 1H); 8.23 (s, |
| | fluoro-phenylamino)-4- | | 1H); 7.81 (dd, 1H); 7.51 (dd, 1H); |
| | trifluoromethyl- | | 7.24 (dt, 1H); 7.20 (s, 1H); |
| | nicotinamide | | 3.84 (dd, 2H); 3.25 (dd, 2H); 3.10 (dd, |
| | | | 2H); 1.73 (m, 1H); 1.59 (m, 2H); |
| | | | 1.18 (m, 2H). |
| | | | EI+; TSQ 700; source 180° C.; 70 V; |
| | | | 200 uA: 431.1 (M^{+.}), 346, 333, 317. |
| 624 | N-Cyclobutylmethyl-6- | A | ¹H NMR (300 MHz, DMSO-d₆) δ: |
| | (2,4-difluoro- | | 9.28 (s, 1H); 8.39 (t br, 1H); 8.23 (s, |
| | phenylamino)-4- | | 1H); 7.95 (m, 1H); 7.31 (ddd, 1H); |
| | trifluoromethyl- | | 7.21 (s, 1H); 7.08 (t br, 1H); |
| | nicotinamide | | 3.24 (dd, 2H); 2.55-2.42 (m, 1H); |
| | | | 2.04-1.63 (m, 6H). |
| | | | EI+; TSQ 700; source 180° C.; 70 V; |
| | | | 200 uA: 385 (M^{+.}); 366; 317; 301. |
| 625 | N-Cyclopentylmethyl-6- | A | ¹H NMR (300 MHz, DMSO-d₆) δ: |
| | (2,4-difluoro- | | 9.29 (s, 1H); 8.45 (t br, 1H); 8.24 (s, |
| | phenylamino)-4- | | 1H); 7.96 (dt, 1H); 7.32 (ddd, 1H); |
| | trifluoromethyl- | | 7.22 (s, 1H); 7.09 (t br, 1H); |
| | nicotinamide | | 3.13 (dd, 2H); 2.08 (m, 1H); |
| | | | 1.75-1.42 (m, 6H); 1.30-1.16 (m, 2H). |
| | | | EI+; TSQ 700; source 180° C.; 70 V; |
| | | | 200 uA: 399 (M^{+.}); 380; 330; 317; |
| | | | 301; 298. |
| 626 | N-(Tetrahydropyran-4- | B | ESI Pos: AQA; Spray 3.5 kV; |
| | ylmethyl)-6-(2-methoxy- | | Skimmer 30 V; Probe 250° C.: |
| | 5-chloro-phenylamino)-4- | | 445 (MH+). |
| | trifluoromethyl- | | |
| | nicotinamide | | |
| 627 | N-Cyclobutylmethyl-6-(2- | B | ESI Pos: AQA; Spray 3.5 kV; |
| | methoxy-5-chloro- | | Skimmer 30 V; Probe 250° C.: |
| | phenylamino)-4- | | 415 (MH+). |
| | trifluoromethyl- | | |
| | nicotinamide | | |
| 628 | N-(Tetrahydropyran-4- | A | ESI Pos: AQA; Spray 3.5 kV; |
| | ylmethyl)-6-(2-hydroxy-5- | | Skimmer 30 V; Probe 250° C.: |
| | chloro-phenylamino)-4- | | 430 (MH+). |
| | trifluoromethyl- | | |
| | nicotinamide | | |
| 629 | N-Cyclohexylmethyl-6-(2- | A | ¹H NMR (300 MHz, DMSO-d₆) δ: |
| | methyl-4-chloro- | | 8.89 (s br, 1H); 8.36 (t br, 1H); |
| | phenylamino)-4- | | 8.21 (s, 1H); 7.62 (d, 1H); 7.33 (d, 1H); |
| | trifluoromethyl- | | 7.24 (dd, 1H); 7.12 (s, 1H); |
| | nicotinamide | | 3.04 (dd, 2H); 2.23 (s, 3H); |
| | | | 1.76-1.39 (m, 6H); 1.29-1.05 (m, 3H); |
| | | | 0.99-0.83 (m, 2H). |
| | | | EI+; TSQ 700; source 180° C.; 70 V; |
| | | | 200 uA: 425 (M^{+.}); 410; 342; 329; |
| | | | 313. |
| 630 | N-(Tetrahydropyran-4- | A | ¹H NMR (300 MHz, DMSO-d₆) δ: |
| | ylmethyl)-6-(2-methyl-4- | | 8.91 (s br, 1H); 8.42 (t br, 1H); |
| | chloro-phenylamino)-4- | | 8.23 (s, 1H); 7.63 (d, 1H); 7.32 (d, 1H); |
| | trifluoromethyl- | | 7.24 (dd, 1H); 7.12 (s, 1H); |
| | nicotinamide | | 3.84 (m, 2H); 3.26 (m, 2H); 3.09 (dd, |
| | | | 2H); 2.23 (s, 3H); 1.82-1.65 (m, |
| | | | 1H); 1.58 (d br, 2H); 1.18 (dq, 2H). |
| | | | EI+; TSQ 700; source 180° C.; 70 V; |
| | | | 200 uA: 427 (M^{+.}); 412; 313. |

Compounds of Examples 565 to 630 described in Table 26 were prepared as described in Example 562 (Method A), Example 563 (Method B) and Example 564 (Method C). The method used is indicated in the third column.

TABLE 27
Compounds of Examples 631 to 635 described in Table 27 were
prepared as described in Example 562 (Method A), Example 563
(Method B) and Example 564 (Method C). The method used is
indicated in the third column.

| Ex. No. | Compound name | Method | ¹H NMR (solvent) ppm and/or MS |
|---|---|---|---|
| 631 | N-(Tetrahydropyran-4- | B | ESI Pos: AQA; Spray 3.5 kV; |
| | ylmethyl)-6- | | Skimmer 30 V; Probe 250° C.: 380 (MH+). |
| | phenylamino-4- | | |
| | trifluoromethyl- | | |
| | nicotinamide | | |
| 632 | N-Cyclopropylmethyl-6- | B | ESI Pos: AQA; Spray 3.5 kV; |
| | phenylamino-4- | | Skimmer 30 V; Probe 250° C.: 336 (MH+). |
| | trifluoromethyl- | | |
| | nicotinamide | | |
| 633 | N-Cycloheptylmethyl-6- | B | ESI Pos: AQA; Spray 3.5 kV; |
| | (3,5-dichloro- | | Skimmer 30 V; Probe 250° C.: 460 (MH+). |
| | phenylamino)-4- | | |
| | trifluoromethyl- | | |
| | nicotinamide | | |
| 634 | N-(Tetrahydropyran-4- | A | ¹H NMR (300 MHz, DMSO-d₆) δ: |
| | ylmethyl)-6-(2-methyl- | | 8.89 (s br, 1H); 8.45 (t br, 1H); |
| | 5-chloro-phenylamino)- | | 8.30 (s, 1H); 7.86 (d, 1H); 7.26 (s, 1H); |
| | 4-trifluoromethyl- | | 7.25 (d, 1H); 7.07 (dd, 1H); 3.84 (m, |
| | nicotinamide | | 2H); 3.27 (m, 2H); 3.10 (dd, 2H); |
| | | | 2.23 (s, 3H); 1.83-1.68 (m, 1H); |
| | | | 1.60 (m, 2H); 1.27-1.10 (m, 2H). |
| | | | EI+; TSQ 700; source 180° C.; 70 V; |
| | | | 200 uA: 427 (M^{+.}); 412; 313. |
| 635 | N-Cyclobutylmethyl-6- | A | ESI Pos: AQA; Spray 3.5 kV; |
| | (2-hydroxy-5-chloro- | | Skimmer 30 V; Probe 250° C.: 400 (MH+). |
| | phenylamino)-4- | | |
| | trifluoromethyl- | | |
| | nicotinamide | | |

### EXAMPLE 654

### N-(5-Oxo-pyrrolidin-3-ylmethyl)-6-(2,4-dichloro-phenylamino)-4-trifluoromethyl-nicotinamide

PS-carbodiimide (0.305 g, 0.4 mmol, loading 1.31 mmol/g, ex Argonaut Technologies) and 1-hydroxy-7-azabenzotriazole (0.046 g, 0.34 mmol) were added to a solution of 6-(2,4-dichlorophenylamino)-4-(trifluoromethyl)-nicotinic acid (Description 12) (0.08 g, 0.22 mmol) in dry dichloromethane (5 mL) and the mixture was stirred at room temperature overnight. The resin was filtered and washed repeatedly with dichloromethane, the solvent was then removed in vacuo.

The solid residue was dissolved in anhydrous N-methylpyrrolidone (1 mL) and 4-aminomethyl-pyrrolidin-2-one (23 mg, 0.20 mmol) was added. The solution was heated in a sealed tube under microwaves irradiation for 30 min at 140° C. (power=50 W). The reaction mixture was diluted with dichloromethane, washed with an aqueous solution of K₂CO₃ 10%, dried over magnesium sulphate and evaporated under reduced pressure.

Chromatographic purification through preparative HPLC on a Symmetry C₁₈ column, by gradient elution with a solvent system water/TFA 99.9:0.1 respectively (A) and CH₃CN/TFA 99.9:0.1 respectively (B) with the following gradient: 5% B (3 min); 5% B→95% B (11 min); 95% B (1 min); 95% B→5% B (2 min) afforded the title compound as its trifluoroacetate salt that was suspended in dichloromethane and treated with NaOH 0.5 N. The organic layer was dried over Na₂SO₄ and evaporated under reduced pressure to give the title compound (42 mg, yield=47%).

Compounds 636 to 659 of Table 28 were prepared as described in Example 562 (Method A), or Example 563 (Method B) or Example 654 (Method D). The method used is given in the third column of Table 28.

TABLE 28

| | | | 1h NMR(Solvent) ppm |
|---|---|---|---|
| Ex. No. | Compound name | Method | and/or MS |
| 636 | N-Cycloheptylmethyl-6- | B | ESI Pos: AQA; Spray |
| | phenylamino-4-trifluoromethyl- | | 3.5 kV; Skimmer 30 V; |
| | nicotinamide | | Probe 250° C.: 392 (MH+). |
| 637 | N-Cyclobutyl-6-phenylamino-4- | B | ESI Pos: AQA; Spray |
| | trifluoromethyl-nicotinamide | | 3.5 kV; Skimmer 30 V; |
| | | | Probe 250° C.: 336 (MH+). |
| 638 | N-Isobutyl-6-phenylamino-4- | B | ESI Pos: AQA; Spray |
| | trifluoromethyl-nicotinamide | | 3.5 kV; Skimmer 30 V; |
| | | | Probe 250° C.: 338 (MH+). |
| 639 | N-(3-Dimethylamino-2,2-dimethyl- | B | ESI Pos: AQA; Spray |
| | propyl)-6-(3-chloro-phenylamino)-4- | | 3.5 kV; Skimmer 30 V; |
| | trifluoromethyl-nicotinamide | | Probe 250° C.: 429 (MH+). |
| 640 | N-(3-Hydroxy-2,2-dimethyl-propyl)- | B | ESI Pos: AQA; Spray |
| | 6-(3-chloro-phenylamino)-4- | | 3.5 kV; Skimmer 30 V; |
| | trifluoromethyl-nicotinamide | | Probe 250° C.: 402 (MH+). |
| 641 | N-(2-Methoxy-2-methyl-propyl)-6- | D | ESI Pos: AQA; Spray |
| | (3-chloro-phenylamino)-4- | | 3.5 kV; Skimmer 30 V; |
| | trifluoromethyl-nicotinamide | | Probe 250° C.: 402 (MH+). |
| 642 | N-([1,4]dioxan-2-ylmethyl)-6-(3- | B | ESI Pos: AQA; Spray |
| | chloro-phenylamino)-4- | | 3.5 kV; Skimmer 30 V; |
| | trifluoromethyl-nicotinamide | | Probe 250° C.: 416 (MH+). |
| 643 | N-(Piperidin-2-ylmethyl)-6-(3- | B | ESI Pos: AQA; Spray |
| | chloro-phenylamino)-4- | | 3.5 kV; Skimmer 30 V; |
| | trifluoromethyl-nicotinamide | | Probe 250° C.: 427 (MH+). |
| 644 | N-(1-Benzyl-5-oxo-pyrrolidin-3- | D | ESI Pos: AQA; Spray |
| | ylmethyl)-6-(3-chloro- | | 3.5 kV; Skimmer 30 V; |
| | phenylamino)-4-trifluoromethyl- | | Probe 250° C.: 503 (MH+). |
| | nicotinamide | | |
| 645 | N-(5-Oxo-pyrrolidin-3-ylmethyl)-6- | D | ESI Pos: AQA; Spray |
| | (3-chloro-phenylamino)-4- | | 3.5 kV; Skimmer 30 V; |
| | trifluoromethyl-nicotinamide | | Probe 250° C.: 413 (MH+). |
| 646 | N-Methylcarbamoylmethyl-6-(3- | D | ESI Pos: AQA; Spray |
| | chloro-phenylamino)-4- | | 3.5 kV; Skimmer 30 V; |
| | trifluoromethyl-nicotinamide | | Probe 250° C.: 387 (MH+). |
| 647 | N-(1-Ethyl-pyrrolidin-2-ylmethyl)- | D | ESI Pos: AQA; Spray |
| | 6-(3-chloro-phenylamino)-4- | | 3.5 kV; Skimmer 30 V; |
| | trifluoromethyl-nicotinamide | | Probe 250° C.: 427 (MH+). |
| 648 | N-(2,2,6,6-Tetramethyl-piperidin-4- | D | ESI Pos: AQA; Spray |
| | ylmethyl)-6-(3-chloro- | | 3.5 kV; Skimmer 30 V; |
| | phenylamino)-4-trifluoromethyl- | | Probe 250° C.: 455 (MH+). |
| | nicotinamide | | |
| 649 | N-(2,2-Dimethyl-[1,3]dioxolan-4- | B | ESI Pos: AQA; Spray |
| | ylmethyl)-6-(3-chloro- | | 3.5 kV; Skimmer 30 V; |
| | phenylamino)-4-trifluoromethyl- | | Probe 250° C.: 430 (MH+). |
| | nicotinamide | | |
| 650 | N-(Tetrahydropyran-4-ylmethyl)-6- | B | ESI Pos: AQA; Spray |
| | (2-fluoro-phenylamino)-4- | | 3.5 kV; Skimmer 30 V; |
| | trifluoromethyl-nicotinamide | | Probe 250° C.: 398 (MH+). |
| 651 | N-(Tetrahydropyran-4-ylmethyl)-6- | B | ESI Pos: AQA; Spray |
| | (4-fluoro-phenylamino)-4- | | 3.5 kV; Skimmer 30 V; |
| | trifluoromethyl-nicotinamide | | Probe 250° C.: 398 (MH+). |
| 652 | N-(3-Dimethylamino-2,2-dimethyl- | B | ESI Pos: AQA; Spray |
| | propyl)-6-(2,4-dichloro- | | 3.5 kV; Skimmer 30 V; |
| | phenylamino)-4-trifluoromethyl- | | Probe 250° C.: 463 (MH+). |
| | nicotinamide | | |
| 653 | N-([1,4]dioxan-2-ylmethyl)-6-(2,4- | B | ESI Pos: AQA; Spray |
| | dichloro-phenylamino)-4- | | 3.5 kV; Skimmer 30 V; |
| | trifluoromethyl-nicotinamide | | Probe 250° C.: 450 (MH+). |
| 654 - | N-(5-Oxo-pyrrolidin-3-ylmethyl)-6- | D | ESI Pos: AQA; Spray |
| see above | (2,4-dichloro-phenylamino)-4- | | 3.5 kV; Skimmer 30 V; |
| for full | trifluoromethyl-nicotinamide | | Probe 250° C.: 447 (MH+). |
| write up | | | |
| 655 | N-Methylcarbamoylmethyl-6-(2,4- | D | ESI Pos: AQA; Spray |
| | dichloro-phenylamino)-4- | | 3.5 kV; Skimmer 30 V; |
| | trifluoromethyl-nicotinamide | | Probe 250° C.: 421 (MH+). |
| 656 | N-(2,2-Dimethyl-[1,3]dioxolan-4- | B | ESI Pos: AQA; Spray |
| | ylmethyl)-6-(2,4-dichloro- | | 3.5 kV; Skimmer 30 V; |
| | phenylamino)-4-trifluoromethyl- | | Probe 250° C.: 464 (MH+). |
| | nicotinamide | | |
| 657 | N-Cycloheptylmethyl-6-(3,4- | B | ESI Pos: AQA; Spray |
| | dichloro-phenylamino)-4- | | 3.5 kV; Skimmer 30 V; |
| | trifluoromethyl-nicotinamide | | Probe 250° C.: 460 (MH+). |
| 658 | N-(Tetrahydropyran-4-ylmethyl)-6- | A | EI+; TSQ 700; source |
| | (2,4-difluoro-phenylamino)-4- | | 180° C.; 70 V; 200 uA: |
| | trifluoromethyl-nicotinamide | | 415 (M^{+.}). |
| 659 | N-Cyclohexylmethyl-6-(2-methyl-5- | A | ESI Pos: AQA; Spray |
| | chloro-phenylamino)-4- | | 3.5 kV; Skimmer 30 V; |
| | trifluoromethyl-nicotinamide | | Probe 250° C.: 431 (MH+). |

### EXAMPLE 660

### N-(2,3-Dihydroxy-propyl)-6-(3-chloro-phenylamino)-4-trifluoromethyl-nicotinamide

N-(2,2-dimethyl-[1,3]dioxolan-4-ylmethyl)-6-(3-chloro-phenylamino)-4-trifluoromethyl-nicotinamide (Example 649) (30 mg, 0.07 mmol), was dissolved in tetrahydrofuran (4 mL) and stirred overnight at ambient temperature in the presence of Et₂O/HCl (3 mL). Evaporation of the solvent in vacuo afforded the title compound as a white solid (27 mg, yield=99%).

¹H NMR (300 MHz, DMSO-d₆) δ: 9.90 (s, 1H); 8.45 (s, 1H); 8.41 (t br, 1H); 8.02 (dd, 1H); 7.50 (ddd, 1H); 7.34 (dd, 1H); 7.17 (s, 1H); 7.03 (ddd, 1H); 3.65-3.30 (m, 7H); 3.14 (ddd, 1H).

MS m/z (ESI+): AQA; Spray 3.5 kV; Skimmer 30V; Probe 250° C.: 390 (MH⁺).

### EXAMPLE 661

### N-(2,3-Dihydroxy-propyl)-6-(2,4-dichloro-phenylamino)-4-trifluoromethyl-nicotinamide

The title compound was prepared in a similar manner to that described in the Example 660, starting from N-(2,2-dimethyl-[1,3]dioxolan-4-ylmethyl)-6-(2,4-dichloro-phenylamino)-4-trifluoromethyl-nicotinamide (Example 657) (40 mg, 0.09 mmol) and the title compound was obtained as a white solid (35 mg, yield=96%).

¹H NMR (300 MHz, CDCl₃) δ: 8.36 (s, 1H); 8.02 (d, 1H); 7.66 (s br, 1H); 7.35 (d, 1H); 7.18 (dd, 1H); 7.11 (t br, 1H); 7.05 (s, 1H); 3.89 (s br, 1H); 3.77 (s br, 1H); 3.59-3.47 (m, 3H); 3.42 (ddd, 1H). MS m/z (ESI+): AQA; Spray 3.5 kV; Skimmer 30V; Probe 250° C.: 424 (MH⁺).

### EXAMPLE 662

### 6-(3-Chloro-phenylamino)-N-(tetrahydro-pyran-4-ylmethyl)-2-trifluoromethyl-nicotinamide

N-methyl morpholine (0.14 mL, 1.27 mmol, 2.5 eq), 1-hydroxy-benzotriazole (110 mg, 0.76 mmol, 1.5 eq), N-(3-dimethylaminopropyl)-N-ethylcarbodiimide hydrochloride (120 mg, 0.61 mmol, 1.2 eq) and tetrahydropyran-4-ylmethyl amine (77 mg, 0.66 mmol, 1.3 eq) were subsequently added to a solution of 6-(3-chloro-phenylamino)-2-trifluoromethyl-nicotinic acid hydrochloride (180 mg, 0.51 mmol, 1.0 eq) in anhydrous DCM (12 mL) and stirred at ambient temperature for 12 h. After evaporation of the solvent in vacuo, the mixture was diluted with ethyl acetate (50 mL) and washed subsequently with a saturated aqueous solution of NaHCO₃ (20 mL×2 times) and brine (25 mL). The organic phase was dried over sodium sulphate and concentrated in vacuo to afford a black residue that was purified by flash chromatography (silica gel, eluent gradient: from hexane/ethyl acetate 1:9 to pure ethyl acetate). The title compound was obtained as a brown solid (130 mg, yield=61%).

EI; TSQ 700; source 180 C; 70 V; 200 uA: 413 (M+.); 315; 299. ¹H NMR (300 MHz, DMSO-d₆) δ: 9.80(s, 1H); 8.48 (t br, 1H); 8.02 (dd, 1H); 7.72 (d, 1H); 7.51 (dd, 1H); 7.31 (dd, 1H); 7.09 (d, 1H); 7.00 (dd, 1H); 3.89 (m, 2H); 3.27 (m, 2H); 3.09 (dd, 2H); 1.75 (m, 1H); 1.60 (m, 2H); 1.20 (m, 2H).

TABLE 29
Compounds of Example 663 to 667 were prepared as described Example 662,
from the appropriate starting materials via similar intermediates, prepared in
a similar manner to the intermediates described in Descriptions 17 to 20 and 33.

| Ex. No | Compound Name | ¹H NMR (Solvent) ppm and/or MS |
|---|---|---|
| 663 | 6-(3-Chloro-phenylamino)-N- | EI; TSQ 700; source 180 C.; 70 V; 200 uA: 411 |
| | cyclohexylmethyl-2- | (M+.), 315, 299. |
| | trifluoromethyl-nicotinamide | ¹H NMR (300 MHz, DMSO-d₆) δ: |
| | | 9.80 (s, 1H); 8.38 (t br, 1H); 8.01 (dd, 1H); |
| | | 7.72 (d, 1H); 7.51 (dd, 1H); 7.32 (dd, 1H); |
| | | 7.08 (d, 1H); 7.00 (dd, 1H); 3.05 (dd, 2H); |
| | | 1.77-1.57 (m, 5H); 1.57-1.41 (m, 1H); |
| | | 1.30-1.10 (m, 3H); 1.02-0.83 (m, 2H). |
| 664 | 6-(3-Chloro-phenylamino)-N- | EI; TSQ 700; source 180 C.; 70 V; 200 uA: |
| | cyclobutylmethyl-2- | 383 (M+.); 315; 299. |
| | trifluoromethyl-nicotinamide | ¹H NMR (300 MHz, DMSO-d₆) δ: |
| | | 9.80 (s, 1H); 8.40 (t br, 1H); 8.00 (dd, 1H); |
| | | 7.71 (d, 1H); 7.50 (dd, 1H); 7.30 (dd, 1H); |
| | | 7.08 (d, 1H); 7.00 (dd, 1H); 3.21 (dd, 2H); |
| | | 2.50 (m, 1H); 2.00 (m, 2H); 1.95-1.68 (m, |
| | | 4H). |
| 665 | 6-(3-Chloro-phenylamino)-N- | EI; TSQ 700; source 180 C.; 70 V; 200 uA: |
| | cyclopentylmethyl-2- | 397 (M+.); 315; 299. |
| | trifluoromethyl-nicotinamide | ¹H NMR (300 MHz, DMSO-d₆) δ: |
| | | 9.80 (s, 1H); 8.42 (t br, 1H); 8.02 (dd, 1H); |
| | | 7.71 (d, 1H); 7.52 (dd, 1H); 7.33 (dd, 1H); |
| | | 7.09 (d, 1H); 7.00 (dd, 1H); 3.14 (dd, 2H); |
| | | 2.08 (m, 1H); 1.76-1.43 (m, 6H); 1.32-1.16 (m, |
| | | 2H). |

### EXAMPLE 666

### 6-(3-Chloro-phenylamino)-2-isopropyl-N-(tetrahydro-pyran-4-ylmethyl)-nicotinamide

N-methyl morpholine (0.14 mL, 1.27 mmol, 2.5 eq), 1-hydroxy-benzotriazole (100 mg, 0.74 mmol, 1.5 eq), N-(3-dimethylaminopropyl)-N-ethylcarbodiimide hydrochloride (115 mg, 0.6 mmol, 1.2 eq) were subsequently added to a solution of 6-(3-chloro-phenylamino)-2-isopropyl-nicotinic acid hydrochloride (Description 39) (0.16 g, 0.49 mmol, 1.0 eq) in anhydrous DCM (5 mL). After stirring 1 h at room temperature, tetrahydropyran-4-ylmethyl amine (77 mg, 0.66 mmol, 1.3 eq) was added and the resulting solution was stirred at room temperature overnight. Solvent was evaporated in vacuo, the residue was dissolved in ethyl acetate (50 mL) and washed with a saturated aqueous solution of NaHCO₃ and with brine: the organic phase was dried over Na₂SO₄ and concentrated in vacuo to yield a solid that was triturated with hexane/diethyl ether 9:1 and filtered. The title compound was obtained as a white solid (170 mg, yield=89%).

EI; TSQ 700; source 180 C; 70 V; 200 uA: 387(M+.), 289, 273, 243.

¹H NMR (300 MHz, DMSO-d₆) δ: 9.39(s, 1H); 8.29 (dd, 1H); 8.21 (t br, 1H); 7.50 (d, 1H); 7.46 (dd, 1H); 7.27 (dd, 1H); 6.91 (dd, 1H); 6.65 (d, 1H); 3.86 (m, 2H); 3.45 (m, 1H); 3.27 (m, 2H); 3.10 (dd, 2H); 1.76 (m, 1H); 1.60 (m, 2H); 1.22 (d, 6H); 1.29-1.12 (m, 2H).

### EXAMPLE 667

### 6-(3-Chloro-phenylamino)-N-(1,1-dioxo-tetrahydrothriophen-3-ylmethyl)-2-isopropyl-nicotinamide

A mixture of 6-(3-chloro-phenylamino)-2-isopropyl-nicotinic acid hydrochloride (Description 39) (166 mg, 0.5 mmol, 1.0 eq), 1-hydroxy-benzotriazole (100 mg, 0.74 mmol, 1.5 eq), PS-dicyclohexylcarbodiimide (760 mg, 1.0 mmol, 2.0 eq, loading=1.31 mmol/g) and PS-diisopropylethylamine (154 mg, 0.6 mmol, 1.2 eq, loading=3.88 mmol/g) was stirred at room temperature overnight. The resins were filtered, washed with DCM and tetrahydrofuran (30 mL) and the filtrate was concentrated in vacuo. The residue was dissolved in 2.5 mL of anhydrous THF and C-(1,1-dioxo-tetrahydro-1l⁶-thiophen-3-ylmethyl amine (108 mg, 0.72 mmol, 1.44 eq) and 1-butyl-3-methylimidazolium hexafluorophosphate (53 uL) were then added. The mixture was heated under microwaves irradiation at 140° C. for 20 min, the solvent was removed in vacuo, the residue diluted with ethyl acetate (30 mL) and 5% Na₂CO₃ (aq) (20 mL). The organic phase was then washed with brine (20 mL) and evaporated in vacuo to afford a solid that was purified by flash chromatography (silica gel, eluent: DCM/MeOH/NH₄OH 97:3:0.3). The title compound was obtained as a solid (140 mg, yield=66%).

EI; TSQ 700; source 180 C; 70 V; 200 uA: 421 (M+.); 273.

¹H NMR (300 MHz, DMSO-d₆) δ: 9.41(s, 1H); 8.36 (t br, 1H); 8.28 (dd, 1H); 7.55 (d, 1H); 7.45 (dd, 1H); 7.27 (dd, 1H); 6.91 (dd, 1H); 6.67 (d, 1H); 3.49-3.15 (m, 5H); 3.07 (m, 1H); 2.85 (dd, 1H); 2.63(m, 1H); 2.23 (m, 1H); 1.86 (m, 1H); 1.09 (d, 6H).

TABLE 30
All the Examples described in Table 30 were prepared as described for the
Example 666 and 667, from the appropriate starting materials via similar
intermediates, prepared in a similar manner to the intermediates described
in Descriptions 34 to 39. In particular, the compounds of the Examples
668 to 672 and 674 to 676 were prepared according to the same experimental procedure as
described for the Example 666, whereas the compounds of the Examples 673 and 677 were
prepared according to the same experimental procedure as described for the Example 668.

| Ex. No | Compound Name | ¹H NMR (Solvent) ppm and/or MS |
|---|---|---|
| 668 | 6-(3-Chloro-phenylamino)- | EI; TSQ 700; source 180 C.; 70 V; 200 uA: |
| | N-cyclopentylmethyl-2- | 371 (M+.), 289, 273. |
| | isopropyl-nicotinamide | ¹H NMR (300 MHz, DMSO-d₆) δ: 9.38 (s, 1H); |
| | | 8.29 (dd, 1H); 8.19 (t br, 1H); 7.48 (d, 1H); 7.45 (dd, |
| | | 1H); 7.27 (dd, 1H); 6.91 (dd, 1H); 6.66 (d, 1H); |
| | | 3.44 (m, 1H); 3.13 (dd, 2H); 2.16-2.04 (m, 1H); |
| | | 1.76-1.42 (m, 6H); 1.32-1.19 (m, 2H); 1.22 (d, 6H). |
| 669 | 6-(3-Chloro-phenylamino)- | EI; TSQ 700; source 180 C.; 70 V; 200 uA: |
| | N-cyclohexylmethyl-2- | 385 (M+.), 289, 273. |
| | isopropyl-nicotinamide | ¹H NMR (300 MHz, DMSO-d₆) δ: 9.37 (s, 1H); |
| | | 8.28 (dd, 1H); 8.14 (t br, 1H); 7.49 (d, 1H); |
| | | 7.46 (dd, 1H); 7.27 (dd, 1H); 6.90 (dd, 1H); 6.65 (d, |
| | | 1H); 3.45 (m, 1H); 3.05 (dd, 2H); 1.76-1.56 (m, |
| | | 4H); 1.57-1.43 (m, 1H); 1.22 (d, 6H); 1.22-1.10 (m, |
| | | 4H); 0.94 (m, 2H). |
| 670 | 6-(2,4-Dichloro- | EI; TSQ 700; source 180 C.; 70 V; 200 uA: |
| | phenylamino)-N- | 391 (M+.); 356, 322; 307. |
| | cyclobutylmethyl-2- | ¹H NMR (300 MHz, DMSO-d₆) δ: 8.52 (s, 1H); |
| | isopropyl-nicotinamide | 8.23 (d, 1H); 8.15 (t br, 1H); 7.58 (d, 1H); 7.47 (d, |
| | | 1H); 7.37 (dd, 1H); 6.86 (d, 1H); 3.39 (m, 1H); |
| | | 3.23 (dd, 2H); 2.50 (m, 1H); 2.06-1.63 (m, 6H); |
| | | 1.13 (d, 6H). |
| 671 | 6-(2,4-Dichloro- | EI; TSQ 700; source 180 C.; 70 V; 200 uA: |
| | phenylamino)-N- | 405 (M+.); 370; 307; 288. |
| | cyclopentylmethyl-2- | ¹H NMR (300 MHz, DMSO-d₆) δ: 8.53 (s, 1H); |
| | isopropyl-nicotinamide | 8.23 (d, 1H); 8.19 (t br, 1H); 7.58 (d, 1H); 7.48 (d, |
| | | 1H); 7.37 (dd, 1H); 6.87 (d, 1H); 3.39 (m, 1H); |
| | | 3.13 (dd, 2H); 2.11 (m, 1H); 1.75-1.41 (m, 6H); |
| | | 1.23 (m, 2H); 1.14 (d, 6H). |
| 672 | 6-(2,4-Dichloro- | EI; TSQ 700; source 180 C.; 70 V; 200 uA: |
| | phenylamino)-N- | 421 (M+.); 386; 307; 288; 271. |
| | (tetrahydro-pyran-4- | ¹H NMR (300 MHz, DMSO-d₆) δ: 8.53 (s, 1H); |
| | ylmethyl)-2-isopropyl- | 8.23 (d, 1H); 8.20 (t br, 1H); 7.58 (d, 1H); 7.51 (d, |
| | nicotinamide | 1H); 7.37 (dd, 1H); 6.87 (d, 1H); 3.85 (m, 2H); |
| | | 3.39 (m, 1H); 3.26 (m, 2H); 3.10 (dd, 2H); 1.75 (m, |
| | | 1H); 1.60 (m, 2H); 1.28-1.07 (m, 2H); 1.13 (d, 6H). |
| 673 | 6-(2,4-Dichloro- | EI; TSQ 700; source 180 C.; 70 V; 200 uA: |
| | phenylamino)-N-(1,1- | 455 (M+.), 420, 307. |
| | dioxo-tetrahydrothiophen- | ¹H NMR (300 MHz, DMSO-d₆) δ: 8.14 (d, 1H); |
| | 3-ylmethyl)-2-isopropyl- | 7.51 (d, 1H); 7.49 (d, 1H); 7.32 (d, 1H); 6.78 (d, |
| | nicotinamide | 1H); 3.40-3.10 (m, 5H); 3.04 (m, 1H); 2.80 (dd, |
| | | 1H); 2.63 (m, 1H); 2.23 (m, 1H); 1.82 (m, 1H); |
| | | 1.09 (d, 6H). |
| 674 | 6-(3-Fluoro-phenylamino)- | EI; TSQ 700; source 180 C.; 70 V; 200 uA: |
| | N-cyclobutylmethyl-2- | 341 (M+.); 257. |
| | isopropyl-nicotinamide | ¹H NMR (300 MHz, DMSO-d₆) δ: 9.38 (s, 1H); |
| | | 8.15 (t br, 1H); 8.00 (d, 1H); 7.46 (d, 1H); |
| | | 7.34-7.21 (m, 2H); 6.67 (m, 1H); 6.65 (d, 1H); 3.44 (m, |
| | | 1H); 3.23 (dd, 2H); 2.50 (m, 1H); 2.07-1.64 (m, |
| | | 6H); 1.21 (d, 6H). |
| 675 | 6-(3-Fluoro-phenylamino)- | EI; TSQ 700; source 180 C.; 70 V; 200 uA: |
| | N-cyclopentylmethyl-2- | 355 (M+.); 273; 257; 227. |
| | isopropyl-nicotinamide | ¹H NMR (300 MHz, DMSO-d₆) δ: 9.38 (s, 1H); |
| | | 8.19 (t br, 1H); 8.01 (ddd, 1H); 7.47 (d, 1H); |
| | | 7.34-7.22 (m, 2H); 6.67 (m, 1H); 6.66 (d, 1H); 3.44 (m, |
| | | 1H); 3.14 (dd, 2H); 2.11 (m, 1H); 1.76-1.43 (m, |
| | | 6H); 1.25 (m, 2H); 1.22 (d, 6H). |
| 676 | 6-(3-Fluoro-phenylamino)- | EI; TSQ 700; source 180 C.; 70 V; 200 uA: |
| | N-(tetrahydro-pyran-4- | 371 (M+.); 273; 257; 227. |
| | ylmethyl)-2-isopropyl- | ¹H NMR (300 MHz, DMSO-d₆) δ: 9.39 (s, 1H); |
| | nicotinamide | 8.20 (t br, 1H); 8.00 (d, 1H); 7.50 (d, 1H); |
| | | 7.34-7.20 (m, 2H); 6.67 (m, 1H); 6.66 (d, 1H); 3.84 (m, |
| | | 2H); 3.45 (m, 1H); 3.36-3.00 (m, 2H); 3.11 (dd, |
| | | 2H); 1.76 (m, 1H); 1.61 (m, 2H); 1.33-1.04 (m, 2H); |
| | | 1.21 (d, 6H). |
| 677 | 6-(3-Fluoro-phenylamino)- | ESI POS, spray 3.5 KV/source: 30 V/PROBE: |
| | N-(1,1-dioxo- | 250 C.: 406 (MH+). |
| | tetrahydrothiophen-3- | ¹H NMR (300 MHz, DMSO-d₆) δ: 9.44 (s, 1H); |
| | ylmethyl)-2-isopropyl- | 8.36 (t br, 1H); 8.00 (ddd, 1H); 7.55 (d, 1H); |
| | nicotinamide | 7.35-7.22 (m, 2H); 6.68 (m, 1H); 6.67 (d, 1H); |
| | | 3.35-3.14 (m, 5H); 3.07 (m, 1H); 2.85 (dd, 1H); 2.64 (m, |
| | | 1H); 2.23 (m, 1H); 1.86 (m, 1H); 1.22 (d, 6H). |

### EXAMPLE 678

### 6-(4-Cyano-2-methyl-phenylamino)-4-isopropyl-N-(tetrahydro-pyran-4-ylmethyl)-nicotinamide

A mixture of 6-chloro-4-isopropyl-N-(tetrahydro-pyran-4-ylmethyl)-nicotinamide (Description 24) (100 mg), 4-amino-3-methyl benzonitrile (2 eq), cesium carbonate (168 mg), tris(dibenzylideneacetone)palladium(0) (Pd₂(dba)₃) (3.4 mg), 4,5-bis(diphenylphosphino)-9,9-dimethylxanthene (Xantphos) (2.3 mg) in 1,4-dioxane (1 ml) was irradiated under microwave conditions at 150° C. for 30 minutes. Further quantities of cesium carbonate (168 mg), Pd₂(dba)₃ (3.4 mg) and Xantphos (2.3 mg) were added and the mixture was again subjected to microwave conditions at 150° C. for 30 minutes. Ethyl acetate was added and the mixture was washed with water. The ethyl acetate layer was dried (sodium sulphate) and the solvent was removed under reduced pressure. The residue was purified using MDAP to give the title compound (20 mg)

NMR (MeOD) δ 1.25(6H, d), 1.29-1.43 (2H, m), 1.70 (2H, d), 1.81-1.93 (1H, m), 2.3393H, s), 3.21-3.50 (5H, m), 3.98 (2H, dd), 7.01 (1H, s), 7.49 (1H, dd), 7.55 (1H, bs), 8.02 (1H, d), 8.09 (1H, s)

LC/MS, t=2.89 min, Molecular ion observed [MH⁺] 393 consistent with the molecular formula C₂₃H₂₈N₄O₂

### EXAMPLE 679

### 6-(5-Chloro-2-cyano-phenylamino)-4-isopropyl-N-(tetrahydro-pyran-4-ylmethyl)-nicotinamide

A mixture of 6-chloro-4-isopropyl-N-(tetrahydro-pyran-4-ylmethyl)-nicotinamide (Description 24) (100 mg), 2-amino-4-chlorobenzonitrile (61 mg), cesium carbonate (154 mg), tris(dibenzylideneacetone)palladium(0) (3.2 mg), 4,5-bis(diphenylphosphino)-9,9-dimethyl xanthene (Xantphos) (2.2 mg) and dioxan (1 ml) was stirred under reflux under nitrogen for 24 hours. The mixture was allowed to cool and insoluble material filtered and washed with ethyl acetate. The filtrate was evaporated under reduced pressure and the residue purified by trituration with ether followed by recrystallisation from methanol to give the title compound as a yellow solid (53 mg).

NMR (DMSO-d6) δ 1.2-1.3 (2H, m), 1.21 (6H, d), 1.62 (2H, d), 1.77 (1H, m), 3.15 (2H, t), 3.29 (2H, t), 3.33 (1H, m), 3.86 (2H, d), 7.05 (1H, s), 7.36 (1H, d), 7.46 (1H, s), 8.36 (1H, d), 8.79 (1H, t), 9.00 (1H, s), 9.74 (1H, s).

LC/MS t=2.3 min, [MH⁺] 413 consistent with the molecular formula C₂₂H₂₅³⁵ClN₄O₂.

### EXAMPLE 680

### 6-(2-cyano-5-methyl-phenylamino)-4-isopropyl-N-(tetrahydro-pyran-4-ylmethyl)-nicotinamide

In a manner similar to Example 679, 6-chloro-4-isopropyl-N-(tetrahydro-pyran-4-ylmethyl)-nicotinamide (Description 24) (100 mg) and 2-amino-4-methylbenzonitrile (44.5 mg) afforded the title compound (38 mg).

LC/MS t=1.9 min, [MH⁺] 393 consistent with the molecular formula C₂₃H₂₈N₄O₂.

### EXAMPLE 681

### 6-(3-Chloro-phenylamino)-N-(1,1-dioxo-tetrahydro-1l⁶-thiophen-3-ylmethyl)-4-isopropyl-nicotinamide

In a manner similar to that described in Example 508, 6-(3-chloro-phenylamino)-4-isopropyl-nicotinic acid (Description 4) (30 mg) and C-(1,1-dioxo-tetrahydro-1l⁶-thiophen-3-yl)-methylamine hydrochloride (Argyle et al, J Chem Soc (C), 1967, 2156) (23 mg) afforded the title compound (32 mg).

LC/MS t=3.0 min, [MH⁺] 422 consistent with C₂₀H₂₄³⁵ClN₃O₃S

### EXAMPLE 682

### N-Cyclobutylmethyl-4 isopropyl-6-(3-trifluoromethoxy-phenylamino)-nicotinamide

In a manner similar to Example 464, 6-chloro-N-cyclobutylmethyl-4-isopropyl-nicotinamide (Description 22) (80 mg) and 3-trifluoromethoxyaniline (0.5 ml) gave the title compound (41 mg)

LC/MS, t=3.73 min, Molecular ion observed [MH⁺]=408 consistent with the molecular formula C₂₁H₂₄F₃N₃O₂

TABLE 31
Examples 683 to 691 were prepared by the method given in column 3 and purified
by the procedure given in column 4
Preparation method G: As for the preparation of Example 533
Preparation method J: As for the preparation of Example 504
Purification method E: Mass-directed autopreparative technique
Purification method H: Biotage Horizon

| | | | | 1. Retention time (min). |
|---|---|---|---|---|
| | | Preparation | Purification | 2. [MH+] |
| Ex. no | Compound Name | method | Method | 3. Molecular Formula |
| 683 | 6-(2,3-Difluoro-phenylamino)-4- | G | E | 2.82 min |
| | isopropyl-N-(tetrahydro-pyran-4- | | | 390 |
| | ylmethyl)-nicotinamide | | | C₂₁H₂₅F₂N₃O₂ |
| 684 | 6-(3,5-Bis-trifluoromethyl- | G | E | 3.60 min |
| | phenylamino)-4-isopropyl-N- | | | 490 |
| | (tetrahydro-pyran-4-ylmethyl)- | | | C₂₃H₂₅F₆N₃O₂ |
| | nicotinamide | | | |
| 685 | 6-(2,4-Difluoro-phenylamino)-4- | G | E | 2.70 min |
| | isopropyl-N-(tetrahydro-pyran-4- | | | 390 |
| | ylmethyl)-nicotinamide | | | C₂₁H₂₅F₂N₃O₂ |
| 686 | 6-(3-Ethynyl-phenylamino)-4- | G | E | 2.88 min |
| | isopropyl-N-(tetrahydro-pyran-4- | | | 378 |
| | ylmethyl)-nicotinamide | | | C₂₃H₂₇N₃O₂ |
| 687 | 6-(2-Fluoro-4-trifluoromethyl- | G | E | 3.82 |
| | phenylamino)-N- | | | 424 |
| | cyclopentylmethyl-4-isopropyl- | | | C₂₂H₂₅F₄N₃O |
| | nicotinamide | | | |
| 688 | 6-(3-cyano-4-methyl- | J | H | 2.90 |
| | phenylamino)-4-isopropyl-N- | | | 393 |
| | (tetrahydropyran-4-ylmethyl)- | | | C₂₃H₂₈N₄O₂ |
| | nicotinamide | | | |
| 689 | 6-(3-cyano-4-fluoro- | J | Ethyl | 2.80 |
| | phenylamino)-4-isopropyl-N- | | acetate | 397 |
| | (tetrahydropyran-4-ylmethyl)- | | trituration | C₂₂H₂₅FN₄O₂ |
| | nicotinamide | | of crude | |
| | | | product | |
| 690 | 6-(3-bromo-4-trifluoromethoxy- | J | Ether | 3.60 |
| | phenylamino)-4-isopropyl-N- | | trituration | 516 |
| | (tetrahydropyran-4-ylmethyl)- | | of the | C₂₂H₂₅⁷⁹BrF₃N₃O₃ |
| | nicotinamide | | crude | |
| | | | product | |
| 691 | 6-(4-Chloro-2-fluoro- | J | H | 3.58 |
| | phenylamino)-N- | | | 376 |
| | cyclobutylmethyl-4-isopropyl- | | | C₂₀H₂₃³⁵ClFN₃O |
| | nicotinamide | | | |

TABLE 32
Examples 692 to 737 in this table were prepared by the method and reaction time
given in column 3 and purified by the procedure given in column 4.
Method G: Examples were prepared as for Example 533
Method K: Examples were prepared as for Example 679.
Purification method E: mass-directed auto-preparative technique
Purification method H: Biotage Horizon
Purification method L: the reaction was evaporated, taken up in 1:1 DCM/MeOH, filtered,
evaporated, and the residue triturated with MeOH

| | | Method/ | | RT (min), (MH+) |
|---|---|---|---|---|
| | | Reaction | Purification | Consistent with |
| Ex. No. | Compound Name | Time | Method | molecular formula |
| 692 | 6-(5-Bromo-2-methyl- | G | E | 3.0 |
| | phenylamino)-4-isopropyl-N- | 30 min | | 446 |
| | (tetrahydro-pyran-4-ylmethyl)- | | | C₂₂H₂₈⁷⁹BrN₃O₂ |
| | nicotinamide | | | |
| 693 | 6-(2-Bromo-5-fluoro- | G | E | 3.0 |
| | phenylamino)-4-isopropyl-N- | 1 hour | 450 | |
| | (tetrahydro-pyran-4-ylmethyl)- | | | C₂₁H₂₅⁷⁹BrFN₃O₂ |
| | nicotinamide | | | |
| 694 | 6-(2-Fluoro-5-trifluoromethyl- | G | E | 3.2 |
| | phenylamino)-4-isopropyl-N- | 30 min | | 440 |
| | (tetrahydro-pyran-4-ylmethyl)- | | | C₂₂H₂₅F₄N₃O₂ |
| | nicotinamide | | | |
| 695 | 6-(2-Chloro-5-trifluoromethyl- | G | E | 3.4 |
| | phenylamino)-4-isopropyl-N- | 1 hour | | 456 |
| | (tetrahydro-pyran-4-ylmethyl)- | | | C₂₂H₂₅³⁵ClF₃N₃O₂ |
| | nicotinamide | | | |
| 696 | 6-(2-Bromo-5-trifluoromethyl- | G | E | 3.4 |
| | phenylamino)-4-isopropyl-N- | 1 hour | | 500 |
| | (tetrahydro-pyran-4-ylmethyl)- | | | C₂₂H₂₅⁷⁹BrF₃N₃O₂ |
| | nicotinamide | | | |
| 697 | 6-(3-Bromo-4-cyano- | G | E | 3.10 |
| | phenylamino)-4-isopropyl-N- | 30 min | | 459 |
| | (tetrahydro-pyran-4-ylmethyl)- | | | C₂₂H₂₅⁸¹BrN₄O₂ |
| | nicotinamide | | | |
| 698 | 6-(2-Bromo-4-trifluoromethoxy- | G | E | 3.40 |
| | phenylamino)-4-isopropyl-N- | 30 min | | 518 |
| | (tetrahydro-pyran-4-ylmethyl)- | | | C₂₂H₂₅⁸¹BrF₃N₃O₃ |
| | nicotinamide | | | |
| 699 | 6-(3-Chloro-2-methyl- | G | E | 2.29 |
| | phenylamino)-4-isopropyl-N- | 30 min | | 402 |
| | (tetrahydro-pyran-4-ylmethyl)- | | | C₂₂H₂₈³⁵ClN₃O₂ |
| | nicotinamide | | | |
| 700 | 6-(3,5-Difluoro-phenylamino)-4- | G | E | 3.06 |
| | isopropyl-N-(tetrahydro-pyran-4- | 30 min | | 390 |
| | ylmethyl)-nicotinamide | | | C₂₁H₂₅F₂N₃O₂ |
| 701 | 6-(2-Chloro-4-fluoro- | G | E | 2.86 |
| | phenylamino)-4-isopropyl-N- | 30 min | | 406 |
| | (tetrahydro-pyran-4-ylmethyl)- | | | C₂₁H₂₅³⁵ClFN₃O₂ |
| | nicotinamide | | | |
| 702 | 6-(4-Chloro-2-methyl- | G | E | 2.90 |
| | phenylamino)-4-isopropyl-N- | 30 min | | 402 |
| | (tetrahydro-pyran-4-ylmethyl)- | | | C₂₂H₂₈³⁵ClN₃O₂ |
| | nicotinamide | | | |
| 703 | 6-(2-Fluoro-3-trifluoromethyl- | G | H | 3.72 |
| | phenylamino)-N- | 30 Min | | 424 |
| | cyclopentylmethyl-4-isopropyl- | | | C₂₂H₂₅F₄N₃O |
| | nicotinamide | | | |
| 704 | 6-(2-Methyl-4-chloro- | G | H | 3.50 |
| | phenylamino)-N- | 30 Min | | 386 |
| | cyclopentylmethyl-4-isopropyl- | | | C₂₂H₂₈³⁵ClN₃O |
| | nicotinamide | | | |
| 705 | 6-(3-Chloro-4-cyano- | G | H | 3.68 |
| | phenylamino)-N- | 30 Min | | 397 |
| | cyclopentylmethyl-4-isopropyl- | | | C₂₂H₂₅³⁵ClN₄O |
| | nicotinamide | | | |
| 706 | 6-(4-bromo-2-chloro | G | E | 3.91 |
| | phenylamino)-N- | 30 Min | | 450 |
| | cyclopentylmethyl-4-isopropyl- | | | C₂₁H₂₅⁷⁹Br³⁵ClN₃O |
| | nicotinamide | | | |
| 707 | N-Cyclobutylmethyl-6-(2,4- | G | F | 3.24 |
| | difluoro-phenylamino)-4- | 1 hour | | 360 |
| | isopropyl-nicotinamide | | | C₂₀H₂₃F₂N₃O |
| 708 | N-Cyclobutylmethyl-6-(2,4- | G | F | 3.75 |
| | dichloro-phenylamino)-4- | 1 hour | | 392 |
| | isopropyl-nicotinamide | | | C₂₀H₂₃³⁵Cl₂N₃O |
| 709 | N-Cyclobutylmethyl-6-(3,4- | G | Crude | 3.89 |
| | dichloro-phenylamino)-4- | 1 hour | product | 392 |
| | isopropyl-nicotinamide | | purified by | C₂₀H₂₃³⁵Cl₂N₃O |
| | | | trituration | |
| | | | with 1:1 | |
| | | | DCM/Ether | |
| 710 | N-Cyclobutylmethyl-6-(2,3- | G | H | 3.68 |
| | dichloro-phenylamino)-4- | 1 hour | | 392 |
| | isopropyl-nicotinamide | | | C₂₀H₂₃³⁵Cl₂N₃O |
| 711 | 6-(2-Chloro-4-fluoro- | G | F | 3.37 |
| | phenylamino)-N- | 1 hour | | 376 |
| | cyclobutylmethyl-4-isopropyl- | | | C₂₀H₂₃³⁵ClFN₃O |
| | nicotinamide | | | |
| 2712 | 6-(3-Chloro-4-fluoro- | G | H | 3.63 |
| | phenylamino)-N- | 1 hour | | 376 |
| | cyclobutylmethyl-4-isopropyl- | | | C₂₀H₂₃³⁵ClFN₃O |
| | nicotinamide | | | |
| 713 | 6-(4-Bromo-2-chloro- | G | H | 3.81 |
| | phenylamino)-N- | 1 hour | | 436 |
| | cyclobutylmethyl-4-isopropyl- | | | C₂₀H₂₃⁷⁹Br³⁵ClN₃O |
| | nicotinamide | | | |
| 714 | 6-(2-Bromo-4-chloro- | G | H | 3.75 |
| | phenylamino)-N- | 1 hour | | 436 |
| | cyclobutylmethyl-4-isopropyl- | | | C₂₀H₂₃⁷⁹Br³⁵ClN₃O |
| | nicotinamide | | | |
| 715 | N-cyclobutylmethyl-6-(2-fluoro-3- | G | H | 3.64 |
| | trifluoromethyl-phenylamino)-4- | 1 hour | | 410 |
| | isopropyl-nicotinamide | | | C₂₁H₂₃F₄N₃O |
| 716 | 6-(4-Chloro-2-methyl- | G | H | 3.35 |
| | phenylamino)-N- | 1 hour | | 372 |
| | cyclobutylmethyl-4-isopropyl- | | | C₂₁H₂₆³⁵ClN₃O |
| | nicotinamide | | | |
| 717 | 6-(2-Chloro-4-cyano- | K | L | 3.41 |
| | phenylamino)-N- | 3 hours | | 383 |
| | cyclobutylmethyl-4-isopropyl- | | | C₂₁H₂₃³⁵ClN₄O |
| | nicotinamide | | | |
| 718 | 6-(4-Cyano-2-fluoro- | K | L | 3.32 |
| | phenylamino)-N- | 4 hours | | 367 |
| | cyclobutylmethyl-4-isopropyl- | | | C₂₁H₂₃FN₄O |
| | nicotinamide | | | |
| 719 | 6-(4-Cyano-2-methyl- | K | L | 3.24 |
| | phenylamino)-N- | 4 hours | | 363 |
| | cyclobutylmethyl-4-isopropyl- | | | C₂₂H₂₆N₄O |
| | nicotinamide | | | |
| 720 | 6-(2-Chloro-4-trifluoromethyl- | K | E | 3.86 |
| | phenylamino)-N- | 4 hours | | 426 |
| | cyclobutylmethyl-4-isopropyl- | | | C₂₁H₂₃³⁵ClF₃N₃O |
| | nicotinamide | | | |
| 721 | N-Cyclobutylmethyl-6-(3,5- | G | H | 4.01 |
| | dichloro-phenylamino)-4- | 1 hour | | 392 |
| | isopropyl-nicotinamide | | | C₂₀H₂₃³⁵Cl₂N₃O |
| 722 | N-Cyclobutylmethyl-6-(2,5- | G | H | 3.78 |
| | dichloro-phenylamino)-4- | 1 hour | | 392 |
| | isopropyl-nicotinamide | | | C₂₀H₂₃³⁵Cl₂N₃O |
| 723 | N-Cyclobutylmethyl-6-(3,5- | G | H | 3.57 |
| | difluoro-phenylamino)-4- | 1 hour | | 360 |
| | isopropyl-nicotinamide | | | C₂₀H₂₃F₂N₃O |
| 724 | 6-(5-Chloro-2-fluoro- | G | H | 3.62 |
| | phenylamino)-N- | 1 hour | | 376 |
| | cyclobutylmethyl-4-isopropyl- | | | C₂₀H₂₃³⁵ClFN₃O |
| | nicotinamide | | | |
| 725 | 6-(2-Chloro-5-fluoro- | G | H | 3.56 |
| | phenylamino)-N- | 1 hour | | 376 |
| | cyclobutylmethyl-4-isopropyl- | | | C₂₀H₂₃³⁵ClFN₃O |
| | nicotinamide | | | |
| 726 | 6-(3-Chloro-phenylamino)-N- | G | H | 3.28 |
| | isobutyl-4-isopropyl-nicotinamide | 30 min | | 346 |
| | | | | C₁₉H₂₄³⁵ClN₃O |
| 727 | N-Isobutyl-4-isopropyl-6-(3- | G | H | 3.53 380 |
| | trifluoromethyl-phenylamino)- | 30 min | | C₂₀H₂₄F₃N₃O |
| | nicotinamide | | | |
| 728 | 6-(3,4-Dichloro-phenylamino)-N- | G | H | 3.72 |
| | isobutyl-4-isopropyl-nicotinamide | 30 min | | 380 |
| | | | | C₁₉H₂₃³⁵Cl₂N₃O |
| 729 | 6-(2-Fluoro-3-trifluoromethyl- | G | H | 3.37 |
| | phenylamino)-N-isobutyl-4- | 30 min | | 398 |
| | isopropyl-nicotinamide | | | C₂₀H₂₃F₄N₃O |
| 730 | 6-(3-Bromo-2-methyl- | G | H | 3.44 |
| | phenylamino)-N-isobutyl-4- | 30 min | | 406 |
| | isopropyl-nicotinamide | | | C₂₀H₂₆⁸¹BrN₃O |
| 731 | 6-(2,4-Dichloro-phenylamino)-N- | G | H | 3.70 |
| | isobutyl-4-isopropyl-nicotinamide | 30 min | | 380 |
| | | | | C₁₉H₂₃³⁵Cl₂N₃O |
| 732 | 6-(2-Chloro-5-fluoro- | G | H | 3.60 |
| | phenylamino)-N-isobutyl-4- | 30 min | | 364 |
| | isopropyl-nicotinamide | | | C₁₉H₂₃³⁵ClFN₃O |
| 733 | 6-(3,5-Difluoro-phenylamino)-N- | G | H | 3.56 |
| | isobutyl-4-isopropyl-nicotinamide | 30 min | | 348 |
| | | | | C₁₉H₂₃F₂N₃O |
| 734 | 6-(5-Chloro-2-fluoro- | G | H | 3.60 |
| | phenylamino)-N-isobutyl-4- | 30 min | | 364 |
| | isopropyl-nicotinamide | | | C₁₉H₂₃³⁵ClFN₃O |
| 735 | 6-(3-Bromo-phenylamino)-N- | G | H | 3.63 |
| | isobutyl-4-isopropyl-nicotinamide | 30 min | | 392 |
| | | | | C₁₉H₂₄⁸¹BrN₃O |
| 736 | 6-(2,4-Dichloro-phenylamino)-N- | G | E | 3.2 |
| | (1,1-dioxo-tetrahydro-1l⁶- | 30 min | | 456 |
| | thiophen-3-ylmethyl)-4-isopropyl- | | | C₂₀H₂₃³⁵Cl₂N₃O₃S |
| | nicotinamide | | | |
| 737 | 6-(4-Bromo-3-fluoro- | G | E | 3.2 |
| | phenylamino)-N-(1,1-dioxo- | 30 min | | 484 |
| | tetrahydro-1l⁶-thiophen-3- | | | C₂₀H₂₃⁷⁹BrFN₃O₃S |
| | ylmethyl)-4-isopropyl- | | | |
| | nicotinamide | | | |

Examples in table 33 were prepared by the method and reaction time given in column 3 and purified by the procedure given in column 4.

Method G: Examples were prepared as for Example 533

Method K: Examples were prepared as for Example 679.

Purification method E: mass-directed auto-preparative technique

Purification method H: Biotage Horizon

TABLE 33

| | | Method/ | | RT (min), (MH+) |
|---|---|---|---|---|
| Example | | Reaction | Purification | Consistent with |
| No. | Compound Name | Time | Method | molecular formula |
| 738 | 6-(2-Chloro-5-fluoro- | G | E | 3.1 |
| | phenylamino)-4-isopropyl-N- | 1 hour | | 406 |
| | (tetrahydro-pyran-4-ylmethyl)- | | | C₂₁H₂₅³⁵ClFN₃O₂ |
| | nicotinamide | | | |
| 739 | 6-(2-Chloro-5-methyl- | G | E | 3.0 |
| | phenylamino)-4-isopropyl-N- | 30 min | | 402 |
| | (tetrahydro-pyran-4-ylmethyl)- | | | C₂₂H₂₈³⁵ClN₃O₂ |
| | nicotinamide | | | |
| 740 | 6-(2-Fluoro-5-methyl- | G | E | 2.8 |
| | phenylamino)-4-isopropyl-N- | 30 min | | 386 |
| | (tetrahydro-pyran-4-ylmethyl)- | | | C₂₂H₂₈FN₃O₂ |
| | nicotinamide | | | |
| 741 | 6-(5-Fluoro-2-methyl- | G | E | 2.7 |
| | phenylamino)-4-isopropyl-N- | 1 hour | | 386 |
| | (tetrahydro-pyran-4-ylmethyl)- | | | C₂₂H₂₈FN₃O₂ |
| | nicotinamide | | | |
| 742 | 6-(3-Bromo-2-methyl- | G | E | 2.98 |
| | phenylamino)-4-isopropyl-N- | 30 min | | 448 |
| | (tetrahydro-pyran-4-ylmethyl)- | | | C₂₂H₂₈⁸¹BrN₃O₂ |
| | nicotinamide | | | |
| 743 | 4-Isopropyl-6-(2-methyl-4- | G | E | 3.14 |
| | trifluoromethoxy-phenylamino)-N- | 30 min | | 452 |
| | (tetrahydro-pyran-4-ylmethyl)- | | | C₂₃H₂₈F₃N₃O₃ |
| | nicotinamide | | | |
| 744 | 6-(3-Fluoro-2-methyl- | G | E | 2.70 |
| | phenylamino)-4-isopropyl-N- | 30 min | | 386 |
| | (tetrahydro-pyran-4-ylmethyl)- | | | C₂₂H₂₈FN₃O₂ |
| | nicotinamide | | | |
| 745 | 6-(3-Bromo-5-trifluoromethyl- | G | E | 3.59 |
| | phenylamino)-4-isopropyl-N- | 30 min | | 501 |
| | (tetrahydro-pyran-4-ylmethyl)- | | | C₂₂H₂₅⁸¹BrF₃N₃O₂ |
| | nicotinamide | | | |
| 746 | 6-(4-Cyano-phenylamino)-4- | G | E | 3.60 |
| | isopropyl-N-(tetrahydro-pyran-4- | 30 min | | 490 |
| | ylmethyl)-nicotinamide | | | C₂₂H₂₆N₄O₂ |
| 747 | 6-(4-Chloro-2-fluoro- | G | E | 2.72 |
| | phenylamino)-4-isopropyl-N- | 30 min | | 379 |
| | (tetrahydro-pyran-4-ylmethyl)- | | | C₂₁H₂₅³⁵ClFN₃O₂ |
| | nicotinamide | | | |
| 748 | 6-(4-bromo-2-fluoro- | G | E | 3.75 |
| | phenylamino)-N- | 30 min | | 434 |
| | cyclopentylmethyl-4-isopropyl- | | | C₂₁H₂₅⁷⁹BrFN₃O |
| | nicotinamide | | | |
| 749 | 6-(2-Bromo-4-trifluoromethoxy- | G | H | 3.84 |
| | phenylamino)-N- | 1 hour | | 486 |
| | cyclobutylmethyl-4-isopropyl- | | | C₂₁H₂₃⁷⁹BrF₃N₃O₂ |
| | nicotinamide | | | |
| 750 | N-Cyclobutylmethyl-6-(2-fluoro- | K | E | 3.71 |
| | 4-trifluoromethyl-phenylamino)-4- | 8 hours | | 410 |
| | isopropyl-nicotinamide | | | C₂₁H₂₃F₄N₃O |

### EXAMPLE 751

### 6-(4-Cyano-2-fluoro-phenylamino)-N-cyclopentylmethyl-4-isopropyl-nicotinamide

Prepared in a manner similar to Example 679 from 6-chloro-N-cyclopentylmethyl-4-isopropyl-nicotinamide (Description 26) and 4-cyano-2-fluoro-aniline, to give the title compound (16 mg).

NMR (DMSO-d6) δ 1.16 (6H, d), 1.23 (2H, m), 1.51-1.68 (6H, m), 2.11 (1H, m), 3.17 (2H, s), 4.11 (1H, s), 7.25 (1H, s), 7.61 (1H, d), 7.80 (1H, d), 8.12 (1H, s), 8.43 (1H, s), 8.72 (1H, t), 9.37 (1H, s).

LC/MS t=3.4 min, [MH⁺] 381, consistent with molecular formula C₂₂H₂₅FN₄O

### EXAMPLE 752

### 6-(4-Bromo-3-trifluoromethyl-phenylamino)-4-isopropyl-N-(tetrahydropyran-4-ylmethyl)-nicotinamide

A mixture of 6-chloro-4-isopropyl-N-(tetrahydropyran-4-ylmethyl)-nicotinamide (Description 24) (100 mg), 4-bromo-3-trifluoromethyl-(ex Lancaster, 162 mg), methanesulfonic acid (44 μl) in 1,4-dioxane (1 ml) was irradiated under microwave conditions at 180° for 30 minutes. After removal of the 1,4-dioxane under reduced pressure, the mixture was partitioned between ethyl acetate (5 ml) and brine (2 ml) and the aqueous layer separated. The organic layer was evaporated under reduced pressure and the residue purified using the Biotage Horizon system. Purification afforded the title compound as a white solid (47 mg).

NMR (DMSO-d6) δ 1.16-1.23 (8H, d, m), 1.60-1.63 (2H, d), 1.75 (1H, m), 3.10 (2H, t), 3.28 (2H, t), 3.41 (1H, m), 3.85 (2H, d), 6.80 (1H, s), 7.73 (1H, d), 7.83 (1H, d), 8.16 (1H, s), 8.38-8.42 (2H, m), 9.70 (1H, s).

LC/MS t=3.5 min, [MH⁺] 500, consistent with molecular formula C₂₂H₂₅⁷⁹Br F₃N₃O₂

### EXAMPLE 753

### 6-(4-Fluoro-3-trifluoromethyl-phenylamino)-4-isopropyl-N-(tetrahydropyran-4-ylmethyl)-nicotinamide

Prepared in a manner similar to Example 752 from 6-chloro-4-isopropyl-N-(tetrahydropyran-4-ylmethyl)-nicotinamide (Description 24) and 4-fluoro-3-trifluoromethyl-aniline (ex Lancaster, 120 mg). Purified by trituration with ether to afford the title compound as a white solid (121 mg).

NMR (DMSO-d6) δ 1.09-1.24 (8H, d, m), 1.60-1.63 (2H, d), 1.76 (1H, m), 3.10 (2H, t), 3.28 (2H, t), 3.41 (1H, m), 3.85 (2H, d), 6.78 (1H, s), 7.42 (1H, t), 7.86 (1H, d), 8.13 (1H, s), 8.30 (1H, d), 8.40 (1H, t), 9.60 (1H, s).

LC/MS t=3.3 min, [MH⁺] 440, consistent with molecular formula C₂₂H₂₅F₄N₃O₂

### EXAMPLE 754

### 6-(3,4-Dibromo-phenylamino)-4-isopropyl-N-(tetrahydropyran-4-ylmethyl)-nicotinamide

Prepared in a manner similar to Example 752 from 6-chloro-4-isopropyl-N-(tetrahydropyran-4-ylmethyl)-nicotinamide (Description 24) and 3,4-dibromoaniline (169 mg). Purified using the Biotage Horizon system to afford the title compound as a white solid (76 mg).

NMR (DMSO-d6) δ 1.09-1.23 (8H, d, m), 1.60-1.63 (2H, d), 1.76 (1H, m), 3.10 (2H, t), 3.28 (2H, t), 3.41 (1H, m), 3.85 (2H, d), 6.78 (1H, s), 7.48 (1H, d), 7.59 (1H, d), 8.15 (1H, s), 8.38 (2H, t), 9.52 (1H, s).

LC/MS t=3.5 min, [MH⁺] 510, consistent with molecular formula C₂₁H₂₅⁷⁹Br₂N₃O₂

### EXAMPLE 755

### 6-(4-Bromo-3-fluoro-phenylamino)-4-isopropyl-N-(tetrahydropyran-4-ylmethyl)-nicotinamide

Prepared in a manner similar to Example 752 from 6-chloro-4-isopropyl-N-(tetrahydropyran-4-ylmethyl)-nicotinamide (Description 24) and 4-bromo-3-fluoro-aniline (128 mg). Purified by trituration with ether to afford the title compound as a white solid (88 mg).

NMR (DMSO-d6) δ 1.15-1.25 (8H, d, m), 1.60-1.63 (2H, d), 1.76 (1H, m), 3.10 (2H, t), 3.28 (2H, t), 3.41 (1H, m), 3.85 (2H, d), 6.81 (1H, s), 7.30 (1H, d), 7.54 (1H, t), 8.04 (1H, d), 8.15 (1H, s), 8.40 (1H, t), 9.64 (1H, s).

LC/MS t=3.3 min, [MH⁺] 450, consistent with molecular formula C₂₁H₂₅F⁷⁹BrN₃O₂

### EXAMPLE 756

### 6-(2-Chloro-4-trifluoromethyl-phenylamino)-N-cyclopentylmethyl-4-isopropyl-nicotinamide

Prepared in a manner similar to Example 752 from 6-chloro-N-cyclopentylmethyl-4-isopropyl-nicotinamide and 2-chloro-4-trifluoromethylaniline, to give the title compound (30 mg).

NMR (DMSO-d6) δ 1.18 (8H, m), 1.50-1.68 (6H, m), 2.11 (1H, m), 3.16 (2H, s), 3.37 (1H, s), 7.29 (1H, s), 7.64 (1H, d), 7.83 (1H, s), 8.09 (1H, s), 8.43 (1H, s), 8.52 (1H, d), 8.80 (1H, s).

LC/MS t=4.0 min, [MH⁺] 440, consistent with molecular formula C₂₂H₂₅³⁵ClF₃N₃O

### EXAMPLE 757

### 6-(3,4-Difluoro-phenylamino)-4-isopropyl-N-(tetrahydropyran-4-ylmethyl)-nicotinamide

A mixture of 6-chloro-4-isopropyl-N-(tetrahydropyran-4-ylmethyl)-nicotinamide (Description 24) (100 mg), 3,4-difluoroaniline (ex Lancaster, 87 mg), methanesulfonic acid (44 μl) in 1,4-dioxane (1 ml) was irradiated under microwave conditions at 180° for 30 minutes. The solid was dissolved in methanol then evaporated under reduced pressure. The mixture was partitioned between ethyl acetate (5 ml) and brine (2 ml) whereby a solid remained at the interface. The solid was filtered off and washed with water and ethyl acetate to afford the title compound (43 mg).

NMR (DMSO-d6) δ 1.16-1.25 (8H, d, m), 1.60-1.62 (2H, d), 1.75 (1H, m), 3.10 (2H, t), 3.28 (2H, 5 t), 3.41 (1H, m), 3.85 (2H, d), 6.85 (1H, s), 7.29 (1H, d), 7.37 (1H, q), 7.97 (1H, s), 8.08 (1H, s), 8.45 (1H, t), 9.80 (1H, s).

LC/MS t=3.0 min, [MH⁺] 390, consistent with molecular formula C₂₁H₂₅F₂N₃O₂

### EXAMPLE 758

### 6-(4-Chloro-3-trifluoromethyl-phenylamino)-4-isopropyl-N-(tetrahydropyran-4-ylmethyl)-nicotinamide

Prepared in a manner similar to Example 749 from 6-chloro-4-isopropyl-N-(tetrahydropyran-4-ylmethyl)-nicotinamide (Description 24) (100 mg) and 4-chloro-3-trifluoromethyl-aniline (ex Lancaster, 131 mg). Purified by trituration with ether to afford the title compound as a white solid (79 mg).

NMR (DMSO-d6) δ1.16-1.24 (8H, d, m), 1.60-1.63 (2H, d), 1.76 (1H, m), 3.11 (2H, t), 3.28 (2H, t), 3.41 (1H, m), 3.85 (2H, d), 6.80 (1H, s), 7.58 (1H, d), 7.91 (1H, d), 8.16 (1H, s), 8.39 (1H, s), 8.41 (1H, t), 9.70 (1H, s).

LC/MS t=3.5 min, [MH⁺] 456, consistent with molecular formula C₂₂H₂₅³⁵ClF₃N₃O₂

### EXAMPLE 759

### 6-(4-Methyl-3-trifluoromethyl-phenylamino)-4-isopropyl-N-(tetrahydropyran-4-ylmethyl)-nicotinamide

Prepared in a manner similar to Example 749 from 6-chloro-4-isopropyl-N-(tetrahydropyran-4-ylmethyl)-nicotinamide (Description 24) (100 mg) and 4-methyl-3-trifluoromethylaniline (ex Lancaster, 118 mg). Purified by trituration with ether to afford the title compound as a white solid (105 mg).

NMR (DMSO-d6) δ1.15-1.24 (8H, d, m), 1.60-1.63 (2H, d), 1.76 (1H, m), 2.36 (3H, s), 3.11 (2H, t), 3.28 (2H, t), 3.41 (1H, m), 3.85 (2H, d), 6.76 (1H, s), 7.31 (1H, d), 7.76 (1H, d), 8.13 (1H, s), 8.18 (1H, s), 8.37 (1H, t), 9.45 (1H, s).

LC/MS t=3.2 min, [MH⁺] 436, consistent with molecular formula C₂₃H₂₈F₃N₃O₂

### EXAMPLE 760

### 6-(2-Chloro-4-trifluoromethoxy-phenylamino)-4-isopropyl-N-(tetrahydropyran-4-ylmethyl)-nicotinamide

Prepared in a manner similar to Example 749 from 6-chloro-4-isopropyl-N-(tetrahydropyran-4-ylmethyl)-nicotinamide (Description 24) (100 mg) and 2-chloro-4-trifluoromethoxyaniline (ex Acros, 142 mg). Purified using the Biotage Horizon system detailed at the beginning of the experimental section and by trituration with ether to afford the title compound as a white solid (20 mg).

NMR (DMSO-d6) δ 1.16-1.23 (8H, d, m), 1.59-1.62 (2H, d), 1.75 (1H, m), 3.11 (2H, t), 3.28 (2H, t), 3.37 (1H, m), 3.84 (2H, d), 7.09 (1H, s), 7.34 (1H, d), 7.58 (1H, s), 8.04 (1H, s), 8.20 (1H, d), 8.38 (1H, t), 8.66 (1H, s).

LC/MS t=3.4 min, [MH⁺] 472, consistent with molecular formula C₂₂H₂₅³⁵ClF₃N₃O₃

### EXAMPLE 761

### 6-(2-Cyano-3-methyl-phenylamino)-4-isopropyl-N-(tetrahydropyran-4-ylmethyl)-nicotinamide

Prepared in a manner similar to Example 679 from 6-chloro-4-isopropyl-N-(tetrahydropyran-4-ylmethyl)-nicotinamide (Description 24) (100 mg) and 2-cyano-3-methylaniline (ex Fluka, 44 mg) to give the title compound (60 mg).

NMR (DMSQ-d6) δ1.16-1.23 (8H, d, m), 1.59-1.62 (2H, d), 1.75 (1H, m), 2.46 (3H, s), 3.11 (2H, t), 3.28 (2H, t), 3.37 (1H, m), 3.84 (2H, d), 6.96 (1H, s), 7.07 (1H, d), 7.48 (1H, t), 7.67 (1H, d), 8.03 (1H, s), 8.39 (1H, t), 9.13 (1H, s).

LC/MS t=2.7 min, [MH⁺] 393, consistent with molecular formula C₂₃H₂₈N₄O₂

### EXAMPLE 762

### 6-(3-Chloro-2-cyano-phenylamino)-4-isopropyl-N-(tetrahydropyran-4-ylmethyl)-nicotinamide

Prepared in a manner similar to Example 679 from 6-chloro-4-isopropyl-N-(tetrahydropyran-4-ylmethyl)-nicotinamide (Description 24) (100 mg) and 3-chloro-2-cyanoaniline (ex Lancaster, 51 mg) to give the title compound (64 mg).

NMR (DMSO-d6) δ 1.17-1.23 (8H, d, m), 1.59-1.62 (2H, d), 1.75 (1H, m), 3.11 (2H, t), 3.28 (2H, t), 3.37 (1H, m), 3.85 (2H, d), 7.03 (1H, s), 7.32 (1H, d), 7.60 (1H, t), 7.87 (1H, d), 8.07 (1H, s), 8.42 (1H, t), 9.41 (1H, s).

LC/MS t=2.8 min, [MH⁺] 413, consistent with molecular formula C₂₂H₂₅³⁵ClN₄O₂

### EXAMPLE 763

### 6-(3-Chloro-phenylamino)-4(1-hydroxy-methyl-ethyl)-N-(tetrahydropyran-4-ylmethyl)-nicotinamide

### a) 6-Chloro-1,1,dimethyl-1H-furo[3,4-c]pyridin-3-one

To a solution of 2,2,6,6,-tetramethylpiperidine (ex Aldrich, 13.44 g) in tetrahydrofuran (90 ml) at −55° C. under nitrogen was added dropwise 1.6M butyl lithium in hexane (ex Aldrich, 80 ml). After 30 minutes a solution of 6-chloronicotinic acid (ex Aldrich, 5 g) in tetrahydrofuran (40 ml) was added dropwise and the solution stirred at −71° C. for 2 hours.

The solution was treated with acetone (23 ml) and then allowed to warmn to room temperature. The solvent was removed under reduced pressure and the residue dissolved in water (100 ml) and acidified to pH 3 with concentrated hydrochloric acid. The precipitated white solid was filtered off washed with water and dried to afford the title compound (4.42 g).

NMR (DMSO-d6) δ1.65 (6H, s), 8.11 (1H, s), 8.91 (1H, s)

LC/MS t=2.0 min, [MH⁺] 198, consistent with molecular formula C₉H₈³⁵ClNO₂

### b) 6-(3-Chloro-phenylamino)-1,1,dimethyl-1H-furo[3,4-c]pyridin-3-one

A mixture of 6-Chloro-1,1,dimethyl-1H-furo[3,4-c]pyridin-3-one (100 mg), 3-chloroaniline (ex Lancaster, 318 mg), methanesulfonic acid (65 μl) in 1,4-dioxane (1 ml) was irradiated under microwave conditions at 180° for 30 minutes. The solid was dissolved in methanol then evaporated under reduced pressure and the residue partitioned between ethyl acetate (5 ml) and water (2 ml) and the aqueous layer separated. The organic layer was dried over anhydrous magnesium sulphate, filtered and evaporated under reduced pressure. Purified by trituration with ether to afford the title compound as a white solid (30 mg).

NMR (DMSO-d6) δ 1.61 (6H, s), 6.91 (1H, s), 7.04 (1H, d), 7.34 (1H, t), 7.55 (1H, d), 7.93 (1H, t), 8.69 (1H, s), 9.96 (1H, s).

LC/MS t=3.3 min, [MH⁺] 289, consistent with molecular formula C₁₅H₁₃³⁵ClN₂O₂

### c) 6-(3-Chloro-phenylamino)-4(1-hydroxy-methyl-ethyl)-N-(tetrahydropyran-4-ylmethyl)-nicotinamide

To a solution of 4-aminomethyltetrahydropyran (ex Combi-Blocks, Inc, 60 mg) in dry dichloromethane (2 ml) under nitrogen, was added dropwise 2.0M trimethylaluminium in hexane (ex Aldrich, 280 μl) and the solution stirred for 15 minutes. Then a solution of 6-(3-Chloro-phenylamino)-1,1,dimethyl-1H-furo[3,4-c]pyridin-3-one (70 mg) in dry dichloromethane (2 ml) was added and the mixture stirred at 40° C. overnight. A further portion of 4-aminomethyltetrahydropyran (80 mg) and 2.0M trimethylaluminium in hexane (380 μl) in dry dichloromethane (3 ml) was added and the mixture stirred for 48 h.

The solvent was evaporated under reduced pressure and the residue partitioned between ethyl acetate (10 ml) and water (5 ml) and the aqueous layer separated. The organic layer was dried over anhydrous magnesium sulphate, filtered and evaporated under reduced pressure. Purified using the Biotage Horizon system detailed at the beginning of the experimental section to afford the title compound as a white solid (40 mg).

NMR (DMSO-d6) δ1.18-1.23 (2H, m), 1.47 (6H, s), 1.62-1.65 (2H, d), 1.80 (1H, m), 3.11 (2H, t), 3.28 (2H, t), 3.85 (2H, d), 6.06 (1H, s), 6.93 (1H, d), 7.05 (1H, s), 7.28 (1H, t), 7.48 (1H, d), 8.07 (1H, s), 8.17 (1H, s), 8.67 (1H, t), 9.53 (1H, s).

LC/MS t=3.0 min, [MH⁺] 404, consistent with molecular formula C₂₁H₂₆³⁵ClN₃O₃

### EXAMPLE 764

The compound below was prepared as for Example 533 from the intermediate of Description 29.

| | | | RT (min), (MH+) |
|---|---|---|---|
| | | | Consistent |
| | | Purification | with molecular |
| Name | Method | Method | formula |
| 4-tert-Butyl-6-(3,4-dichloro- | G | E | 3.6 |
| phenylamino)-N- | | | 436 |
| (tetrahydro-pyran-4- | | | C₂₂H₂₇³⁵Cl₂N₃O₂ |
| ylmethyl)-nicotinamide | | | |

### EXAMPLE 765

### Preparation of Nanomilled Compound

2.5 g of compound of example 176 was weighed into a 10 ml centrifuge tube. 25 ml of 0.3 mm yttrium zirconium (YTZ) ceramic milling beads (Manufacturer: Tosob, Japan; Supplier: Glen Creston Ltd., batch no. 5280130030)”) was weighed into a 50 ml milling pot. 22.5 ml of aqueous 1.5% HPMC was measured with a measuring cylinder into a 100 ml beaker. This solution was homogenised for 3 seconds with an Ultra Turrax T25 homogeniser. Approximately 200 mg of the 2.5 g of the compound was added to the HPMC solution and homogenised at the lowest speed setting until the powder was wetted. This was repeated until all the compound had been added. The speed of the homogeniser was then increased to maximum and the suspension was homogenised for a further 3 minutes. This suspension was allowed to stand for 30 minutes in order to allow some of the foam to disperse. The suspension was then poured into the 50 ml pot containing the YTZ milling beads, stirring to release any trapped air. The lid to the pot was then fitted and the pot sealed with some Nesco film. This procedure was repeated for a second 50 ml nanomilling pot and both pots were placed on a Retsch mill and milled for a total of 8 hours.

The milling pots were removed from the Retsch mill and left to cool and for the foam to disperse overnight. In the morning the suspension and bead mixture was passed through a 200μ, 40 mm diameter screen. The contents from each 50 ml pot was washed with aqueous 1.5% HPMC: 10% of the original suspension volume (i.e. 2.5 ml). The suspension from the 2 pots was combined to make 1 batch. The suspension obtained from the method above was named the concentrate.

A sample of the concentrate was diluted 1 in 4 with aqueous 1.5% BPMC to give a nominal concentration of 25 mg/ml. This first dilution was assayed by HPLC. The concentration of the concentrate was calculated to be 91.21 mg/ml.

### HPLC Conditions

Column: Symmetry C₁₈ 5μ 3.9×150 mm column; flow rate 1.0 ml/min; column temp 40° C.; UV detection at 280 nm.

Mobile phase gradient: A: water+0.1% trifluoro acetic acid (TFA)
B: acetonitrile+0.1% TFA

TABLE A
HPLC gradient

| Time (min.) | A (%) | B (%) |
|---|---|---|
| 0 | 90 | 10 |
| 15 | 10 | 90 |
| 20 | 10 | 90 |
| 20.1 | 90 | 10 |
| 30 | 90 | 10 |

A particle size analysis was carried out on the Lecotrac laser particle size analyser. The results are shown in Table B along with the results from the starting material for comparison:

TABLE B
Particle Size Analysis

| | Pre-nanomilling | | | |
|---|---|---|---|---|
| | 50% | | Post-nanomilling | |
| | percentile | 95% percentile | 50% | 95% |
| Compound | (μ) | (μ) | percentile (μ) | percentile (μ) |
| Example 176 | 13.15 | 68.7 | 0.33 | 1.78 |

A dilution of nominally 15.0 mg/ml was prepared using 21.36 ml of the concentrate and (100-20.34) ml=83.64 ml of diluent (aqueous 1.5% HPMC).

Compounds of Examples 19, 34, 194, 217, 228, 247 were nanomilled on a 1 g scale using the process described above and the particle size analysed pre and post nanomilling. The results are given in Table C.

TABLE C

| | Pre-nanomilling | | Post-nanomilling | |
|---|---|---|---|---|
| | 50% | | 50% | 95% |
| | percentile | 95% percentile | percentile | percentile |
| Compound | (μ) | (μ) | (μ) | (μ) |
| Ex 247 | 13.2 | 68.7 | 0.64 | 2.53 |
| Ex 217 | 5.70 | 34.9 | 0.34 | 1.30 |
| Ex 19 | 5.22 | 25.5 | 0.40 | 1.40 |
| Ex 228 | 4.65 | 47.1 | 0.44 | 1.69 |
| Ex 194 | 6.78 | 33.7 | 0.56 | 1.97 |
| Ex 34 | 10.46 | 32.7 | 0.18 | 0.56 |

Formulations for pharmaceutical use incorporating compounds of the present invention either pre or post nanomilling can be prepared in various forms and with numerous excipients.

Examples of such formulations are given below.

### EXAMPLE 766

### Inhalant Formulation

The CB2 modulator and PDE4 inhibitor used in the combination of the invention (1 mg to 100 mg) is aerosolized from a metered dose inhaler to deliver the desired amount of drug per use.

### EXAMPLE 767

### Tablet Formulation

| Tablets/Ingredients | Per Tablet |
|---|---|
| 1. Active ingredient (CB2 modulator + PDE4 inhibitor) | 40 mg |
| 2. Corn Starch | 20 mg |
| 3. Alginic acid | 20 mg |
| 4. Sodium Alginate | 20 mg |
| 5. Mg stearate | 1.3 mg |

### Procedure for Tablet Formulation:

Ingredients 1, 2, 3 and 4 are blended in a suitable mixer/blender. Sufficient water is added portion-wise to the blend with careful mixing after each addition until the mass is of a consistency to permit its conversion to wet granules. The wet mass is converted to granules by passing it through an oscillating granulator using a No. 8 mesh (2.38 mm) screen. The wet granules are then dried in an oven at 140° F. (60° C.) until dry. The dry granules are lubricated with ingredient No. 5, and the lubricated granules are compressed on a suitable tablet press.

### EXAMPLE 768

### Parenteral Formulation

A pharmaceutical composition for parenteral administration is prepared by dissolving an appropriate amount of a CB2 modulator and a PDE4 inhibitor in polyethylene glycol with heating. This solution is then diluted with water for injections Ph Eur. (to 100 ml). The solution is then rendered sterile by filtration through a 0.22 micron membrane filter and sealed in sterile containers.

What is claimed is:

1. A method of treating a human or animal subject suffering from a condition which is mediated by the activity of CB2 receptors or a condition which is mediated by PDE4 which comprises administering to said subject a therapeutically effective combination of one or more CB2 modulators and one or more PDE4 inhibitors.

2.-3. (canceled)

4. The method according to claim 1, in which the CB2 modulator is selected from a compound of formula (I): 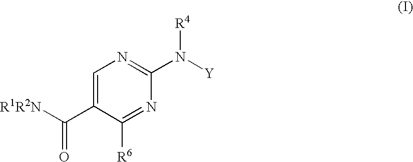
wherein
Y is phenyl, optionally substituted with one, two or three substituents;
R¹ is selected from hydrogen, C₁₋₆ alkyl, Con cycloalkyl and halosubstitutedC₁₋₆ alkyl;
R² is (CH₂)ₘR³ where m is 0 or 1;
or R¹ and R² together with N to which they are attached form an optionally substituted 4- to 8-membered non-aromatic heterocyclyl ring;
R³ is an optionally substituted 4- to 8-membered non-aromatic heterocyclyl group, an optionally substituted C₃₋₈ cycloalkyl group, an optionally substituted straight or branched C₁₋₁₀ alkyl, a C₅₋₇ cycloalkenyl or R⁵;
R⁴ is selected from hydrogen, C₁₋₆ alkyl, C₃₋₆ cycloalkyl, or halosubstitutedC₁₋₆ alkyl, COCH₃, and SO₂Me;
R⁵ is 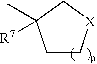
wherein p is 0, 1 or 2 and X is CH₂, O, S, SO or SO₂;
R⁶ is methyl, chloro or CHxFn wherein n is 1, 2, or 3, x is 0, 1 or 2 and n and x add up to 3,
R⁷ is OH, C₁₋₆alkoxy, NR^{8a}R^{8b}, NHCOR⁹, NHSO₂R⁹, SOqR⁹;
R^{8a} is H or C₁₋₆alkyl;
R^{8b} is H or C₁₋₆alkyl;
R⁹ is C₁₋₆alkyl; and
q is 0, 1 or 2;
or a compound of formula (II): 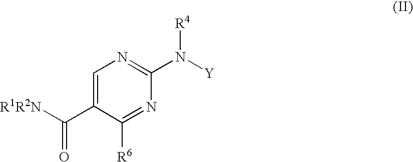
wherein
Y is phenyl, substituted with one, two or three substituents;
R¹ is selected from hydrogen, C₁₋₆ alkyl, C₃₋₈ cycloalkyl, and halosubstitutedC₁₋₆ alkyl;
R² is C(R⁷)₂R³;
R³ is an optionally substituted 5- to 6-membered aromatic heterocyclyl group, or group A: 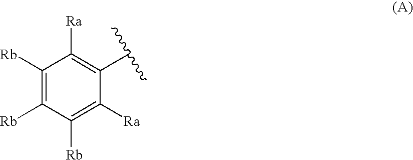
R⁴ is selected from hydrogen, C₁₋₆ alkyl, C₃₋₇ cycloalkyl, and halosubstitutedC₁₋₆ alkyl, COCH₃, or SO₂Me;
R⁶ is methyl, chloro or CHxFn wherein n is 1, 2, or 3, x is 0, 1 or 2 and n and x add up to 3;
R⁶ can be independently selected from hydrogen, fluoro, chloro or trifluoromethyl;
Rb can be independently be selected from hydrogen, C₁₋₆ alkyl, C₁₋₆ alkoxy, haloC₁₋₆ alkoxy, hydroxy, cyano, halo, sulfonyl, CONH₂, COOH or NHCOOC₁₋₈alkyl; and
R⁷ can be independently hydrogen or C₁₋₈ alkyl,
with the proviso that the compound is not
2-(4-tert-butyl-phenylamino)-4-trifluoromethyl-pyrimidine-5-carboxylic acid benzylamide;
2-(4-tert-butyl-phenylamino)-4-trifluoromethyl-pyrimidine-5-carboxylic acid benzyl-methyl-amide;
2-(3-Chloro-phenylamino)-4-trifluoromethyl-pyrimidine-5-carboxylic acid 2-methoxy-benzylamide; or
2-(3-Chloro-phenylamino)-4-trifluoromethyl-pyrimidine-5-carboxylic acid 2-bromo-benzylamide;
or a compound of formula (III): 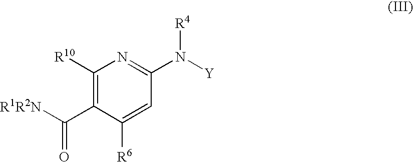
wherein
Y is phenyl, substituted with one, two or three substituents;
R¹ is selected from hydrogen, C₁₋₆ alkyl, C₃₋₇ cycloalkyl, or halosubstitutedC₁₋₆ alkyl;
R² is (CH₂)mR³;
R³ is an unsubstituted or substituted 5- to 6-membered aromatic heterocyclyl group, or group A: 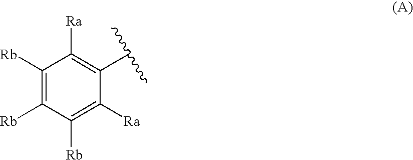
R⁴ is selected from hydrogen, C₁₋₆ alkyl, C₃₋₇ cycloalkyl, or halosubstitutedC₁₋₆ alkyl, COCH₃, and SO₂Me;
R⁶ is unsubstituted or substituted (C₁₋₆)alkyl or chloro and R¹⁰ is hydrogen or R¹⁰ is unsubstituted or substituted (C₁₋₆)alkyl or chloro and R⁶ is hydrogen;
Ra can be independently selected from hydrogen, fluoro, chloro or trifluoromethyl;
Rb can independently be selected from hydrogen, C₁₋₆ alkyl, C₁₋₆ alkoxy, halo substituted C₁₋₆ alkoxy, hydroxy, cyano, halo, sulfonyl, CONH₂ COOH, SO₂CH₃, NHCOCH₁, NHSO₂CH₃ and CONHCH₃; and
m is 1 or 2;
or a compound of formula (IV); 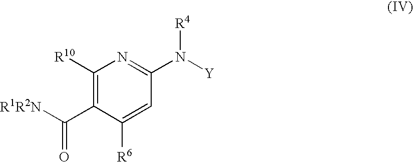
wherein
Y is phenyl, unsubstituted or substituted with one, two or three substituents;
R¹ is selected from hydrogen, C₁₋₆ alkyl, C₃₋₆ cycloalkyl, or halosubstitutedC₁₋₆ alkyl;
R² is (CH₂)ₘR³ where m is 0 or 1;
or R¹ and R² together with N to which they are attached form an optionally substituted 4- to 8-membered non-aromatic heterocyclyl ring;
R³ is a 4- to 8-membered non-aromatic heterocyclyl group, a C₃₋₈ cycloalkyl group, a straight or branched C₁₋₁₀ alkyl, a C₂₋₁₀alkenyl, a C₃₋₈ cycloalkenyl, a C₂₋₁₀alkynyl, or a C₃₋₈cycloalkynyl any of which can be unsubstituted or substituted or R⁵;
R⁴ is selected from hydrogen, C₁₋₆ alkyd, C₃₋₆ cycloalkyl, or halosubstitutedC₁₋₆ alkyl, COCH₃, or SO₂Me;
R⁵ is 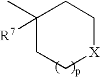
wherein p is 0, 1 or 2, and X is CH₂, O, or S;
R⁶ is a substituted or unsubstituted (C₁₋₆)alkyl or chloro and R¹⁰ is hydrogen or R¹⁰ is a substituted or unsubstituted (C₁₋₆)alkyl or chloro and R⁶ is hydrogen;
R⁷ is OH, C₁₋₆alkoxy, NR^{8a}R^{8b}, NHCOR⁹, NHSO₂R⁹ or SOqR⁹;
R^{8a} is H or C₁₋₆alkyl;
R^{8b} is H or C₁₋₆alkyl;
R⁹ is C₁₋₆alkyl; and
q is 0, 1 or 2;
or a pharmaceutically acceptable derivative thereof.

5. A method according to claim 1 in which the PDE4 inhibitor is selected from cilomilast, AWD-12-281, NCS-613, D-4418, CI-1018, V-11294A, roflumilast or T-4401, and pharmaceutically acceptable derivatives thereof.

6. The method of claim 1 wherein the condition is an immune disorder, an inflammatory disorder, pain, rheumatoid arthritis, multiple sclerosis, osteoarthritis, osteoporosis, lung disorders, for example asthma, bronchitis, emphysema, allergic rhinitis, respiratory distress syndrome, pigeon fancier's disease, farmer's lung, chronic obstructive pulmonary disease, (COPD) and cough, or a disorder which can be treated with a bronchodilator.

7. A pharmaceutical composition comprising one or more CB2 modulators and one or more PDE4 inhibitors adapted for use in human or veterinary medicine.
